SEQUENCE LISTING (1 sequence)

SEQ ID NO: 1 - protein, 7 aa, Artificial Sequence
  Synthetic construct
SFFYLRS

(19) United States
(12) Patent Application Publication
(10) Pub. No.: US 2019/0302026 A1
(43) Pub. Date: Oct. 3, 2019

(54) Uniform, Functionalized, Cross-Linked Nanostructures for Monitoring pH

(71) Applicants: Karen L. Wooley, College Station, TX (US); Richard B. Dorshow, St. Louis, MO (US); John N. Freskos, Clayton, MO (US); William L. Neumann, Kirkwood, MO (US); Jeng J. Shieh, Chesterfield, MO (US); Nam S. Lee, College Station, TX (US); Yun Lin, College Station, TX (US); Guorong Sun, Bryan, TX (US)

(72) Inventors: Karen L. Wooley, College Station, TX (US); Richard B. Dorshow, St. Louis, MO (US); John N. Freskos, Clayton, MO (US); William L. Neumann, Kirkwood, MO (US); Jeng J. Shieh, Chesterfield, MO (US); Nam S. Lee, College Station, TX (US); Yun Lin, College Station, TX (US); Guorong Sun, Bryan, TX (US)

(21) Appl. No.: 16/381,988

(22) Filed: Apr. 11, 2019

Related U.S. Application Data
(63) Continuation of application No. 15/902,759, filed on Feb. 22, 2018, now abandoned.
Application No. 15/902,759 is a continuation of application No. 14/721,907, filed on May 26, 2015, now abandoned.
Application No. 14/721,907 is a continuation of application No. 13/697,986, filed on Apr. 30, 2013, now abandoned.
(60) Provisional application No. 61/334,723, filed on May 14, 2010.

(51) Int. Cl. G01N 21/64; G01N 21/75; G01N 33/84; G01N 33/58; C08F 8/00; A61B 5/145; A61K 49/00; A61B 5/1455

(52) U.S. Cl. CPC G01N 21/6486; G01N 21/75; G01N 33/84; G01N 33/582; A61K 49/0093; A61B 5/14539; A61K 49/0082; A61K 49/0021; A61B 5/1455; C08F 8/00

(57) ABSTRACT

Fluorescence methods and systems that use an optical agent for measuring the pH of a fluid. The optical agent is a photonic nanostructure having a supramolecular structure, such as a shell cross-linked micelle that incorporates at least one linking group that includes one or more photoactive moieties.

### CROSS-REFERENCE TO RELATED APPLICATIONS

This application claims the benefit of U.S. Provisional Application No. 61/334,723, entitled “UNIFORM, FUNCTIONALIZED, CROSS-LINKED NANOSTRUCTURES FOR MONITORING PH”, filed May 14, 2010, which is incorporated by reference to the extent not inconsistent herewith.

### INCORPORATION BY REFERENCE OF RELATED APPLICATIONS

The following related applications are hereby incorporated by reference to the extent not inconsistent with the disclosure herewith: International Application No. PCT/US2008/012575, filed Nov. 7, 2008; U.S. Provisional Application No. 60/986,171 filed Nov. 7, 2007; and U.S. Provisional Application No. 61/106,842 filed Oct. 20, 2008.

### STATEMENT REGARDING FEDERAL FUNDING

This invention was made, at least in part, with government support under U.S. grant Nos. HL080729 and HHSN268201000046C awarded by the National Institutes of Health. The government has certain rights in the invention.

### BACKGROUND

The development of polymeric nanostructures from block copolymer aqueous supramolecular assemblies has gained significant attention due to their diverse promising applications. It has been recognized that their chemical composition and also their size and morphology each require precise tuning. Benefiting from the advances of living/controlled polymerization methodologies to afford varied block copolymer structures, together with extensive investigation of their aqueous assembly, polymeric nanostructures with diverse morphologies have been established. In addition to conventional morphologies, such as spheres, cylinders and vesicles, nanostructures with novel morphologies, including bowls, discs, helices, and toroids, have been reported. Moreover, Janus, multicompartment, onion, and large compound micelles, from higher-order inter- and/or intra-micellar phase segregation, have been created.

Multicompartment micelles (MCMs) represent intra-micellar phase-segregated block copolymer supramolecular assemblies, in which the core domains are heterogeneous and compartmentalized. Although a variety of star terpolymer and linear block polymers have already been explored as precursors to prepare MCMs, most lacked functionalities for facile and practical chemical transformations. Self-assembled nanostructures are a class of nanomaterials having chemical and physical properties potentially beneficial for biomedical applications. Amphiphilic polymer micelle supramolecular structures, for example, have been proposed as a versatile nanomaterials platform for encapsulating, solubilizing, and facilitating delivery of poorly water soluble drugs, including chemotherapeutic agents. Incorporation of targeting ligands into amphiphilic polymer micelle supramolecular structures has promise to provide an effective route for targeted delivery of pharmaceuticals to specific cell types, tissues and organs. Although use of micelle supramolecular structures for drug formulation and delivery applications is currently the subject of considerable research, the development of self-assembled nanostructures for other biomedical applications is substantially less well developed.

Polymer micelle supramolecular structures are typically formed via entropically driven self-assembly of amphiphilic polymers in a solution environment. For example, when block copolymers, having spatially segregated hydrophilic and hydrophobic domains are provided in aqueous solution at a concentration above critical micelle concentration (CMC) the polymers aggregate and self-align such that hydrophobic domains form a central hydrophobic core and hydrophilic domains self-align into an exterior hydrophilic corona region exposed to the aqueous phase. The core-corona structure of amphiphilic polymer micelles provides useful physical properties, as the hydrophobic core provides a shielded phase capable of solubilizing hydrophobic molecules, and the exterior corona region is at least partially solvated, thus imparting colloidal stability to these nanostructures.

A number of amphiphilic polymer systems, including block copolymers and cross-linked block copolymer assemblies, have been specifically engineered and developed for biomedical applications, such as drug formulation and delivery applications. The following references provide examples of amphiphilic polymer drug delivery systems, including block copolymer drug delivery systems, which are hereby incorporate by reference in their entireties: (1) Li, Yali; Sun, Guorong; Xu, Jinqi; Wooley, Karen L., “Shell Cross-linked Nanoparticles: a Progress Report on their Design for Drug Delivery; Nanotechnology in Therapeutics” (2007), 381-407; (2) Qinggao Ma, Edward E. Remsen, Tomasz, Kowalewski, Jacob Schaefer, Karen Wooley, “Environmentally-responsive, Entirely, Hydrophilic, Shell Cross-linked (SCK) Nanoparticles” Nano Lett. 2001, 1, 651-655; (3) Jones, M.-C.; Leroux, J.-C., “Polymeric Micelles: A New Generation of Colloidal Drug Carriers” Eur. J. Pharm. Biopharm. 1999, 48, 101-111; and (4) Kwon, G. S.; Naito, M.; Kataoka, K.; Yokoyama, M.; Sakurai, Y.; Okano, T. “Block Copolymer Micelles as Vehicles for Hydrophobic Drugs” Colloids and Surfaces, B: Biointerfaces 1994, 2, 429-434.

### SUMMARY

Described herein are optical agents, including compositions, preparations and formulations, for monitoring the pH of a fluid. Optical agents described herein include photonic nanostructures and nano-assemblies including supramolecular structures, such as shell-cross-linked micelles, that incorporate at least one linking group comprising one or more photoactive moieties that provide functionality as optical agents for a range of pH monitoring applications. Optical agents described herein comprise supramolecular structures having linking groups imparting useful optical and structural functionality. In an embodiment, for example, the presence of linking groups function to covalently cross link polymer components to provide a cross-linked shell stabilized supramolecular structure, and also impart useful optical functionality, for example by functioning as a fluorophore.

In an embodiment, a method of measuring the pH of a fluid is provided, the method comprising: administering to the fluid an effective amount of an optical agent, the optical agent comprising: cross-linked block copolymers, wherein each of the block copolymers comprises one or more hydrophilic blocks and one or more hydrophobic blocks; and linking groups covalently cross linking at least a portion the hydrophilic blocks of the block copolymers, wherein at least a portion of the linking groups comprise one or more photoactive moieties; wherein the optical agent forms a supramolecular structure in aqueous solution, the supramolecular structure having one or more interior hydrophobic cores and one or more covalently cross-linked hydrophilic shells, wherein the one or more interior hydrophobic cores comprise the hydrophobic blocks of the block copolymers, and the one or more covalently cross-linked hydrophilic shells comprise the hydrophilic blocks of the block copolymers; exposing the optical agent to electromagnetic radiation; wherein the optical agent emits fluorescence in response to the exposure to the electromagnetic radiation; measuring a first fluorescence intensity at a first wavelength from the optical agent exposed to electromagnetic radiation; measuring a second fluorescence intensity at a second wavelength from the optical agent exposed to electromagnetic radiation; wherein the second wavelength differs from the first wavelength; calculating a fluorescence intensity ratio of the first fluorescence intensity at the first wavelength to the second fluorescence intensity at the second wavelength; and comparing the calculated fluorescence intensity ratio to a reference fluorescence intensity ratio.

The methods described herein can be practiced in many different environments. In an embodiment, for example, the pH of the fluid is measured in vivo. In another embodiment, the pH of the fluid is measured in vitro.

The methods described herein can comprise additional steps. In an embodiment, for example, the method of measuring the pH of a fluid further comprises generating the reference fluorescence intensity ratio by measuring fluorescence intensities at a plurality of wavelengths for one or more reference sample fluids having a known pH.

The methods described herein can be practiced using an array of optical agent supramolecular structures. In an embodiment, the optical agent supramolecular structure comprises a nanoparticle or shell cross-linked micelle. In another embodiment, the optical agent supramolecular structure comprises a shell cross-linked micelle or nanoparticle having a globular, spherical, cylindrical, rod, disc, toroidal, spheroidal, vesicle, or multicompartment morphology. In an aspect, the optical agent supramolecular structure comprises a shell cross-linked micelle or nanoparticle having a multicompartment morphology. In another aspect, the optical agent supramolecular structure comprises a shell cross-linked micelle or nanoparticle having a cylindrical or rod morphology. In an aspect, the optical agent supramolecular structure comprises a shell cross-linked micelle or nanoparticle having a rod morphology. In a related embodiment, one or more dimensions of the supramolecular structure is controlled by selection of the hydrophobic blocks of the block copolymer, the hydrophilic blocks of the block copolymer, aqueous solution composition, or any combination thereof.

The pH monitoring method described herein can be practiced employing several fluorescence detection techniques. In an embodiment, for example, the first fluorescence intensity and the second fluorescence intensity are measured by measuring a local maximum of the fluorescence intensity. In a related embodiment, the first fluorescence intensity and the second fluorescence intensity are measured by measuring integrated intensities of the fluorescence at a preselected range of wavelengths about the first wavelength and the second wavelength.

The pH monitoring methods described herein are compatible with a wide range of wavelengths of electromagnetic radiation. In an embodiment, for example, the optical agent is exposed to electromagnetic radiation of wavelength selected from the range of 350 nanometers to 1300 nanometers. In another embodiment, the first wavelength is selected from the range of 350 nanometers to 1300 nanometers. In a related embodiment, the second wavelength is selected from the range of 350 nanometers to 1300 nanometers.

The pH monitoring methods described herein enable fluorescence ratio calculation for fluorescence intensities detected over a broad range of wavelengths of electromagnetic radiation. In an embodiment, for example, the first wavelength differs from the second wavelength by an amount greater than or equal to 5 nanometers. In a related embodiment, the first wavelength differs from the second wavelength by an amount selected from the range of 5 nanometers to 600 nanometers.

The calculated fluorescence intensity ratio of pH monitoring methods described herein is useful for measuring the pH of a fluid over a broad range of fluorescence intensity ratios. In an embodiment, the calculated fluorescence intensity ratio ranges from 0.3 to 3. In an aspect, the calculated fluorescence intensity ratio ranges from 0.01 to 100, optionally from 1 to 50, and optionally from 0.1 to 10.

Many fluids are compatible with the pH monitoring methods described herein. In an embodiment, for example, the fluid comprises a bodily fluid of an animal, an organic solvent, a cell extract, a cell lysate, or a water source. In an aspect, the fluid comprises a bodily fluid of an animal comprising blood, plasma, cerebrospinal fluid, aqueous humour, pleural fluid, pericardial fluid, lymph chyme, chyle, bile, synovial fluid, peritoneal fluid, stool, water, prostatic fluid, amniotic fluid, milk, urine, vomit, cerumen, gastic acid, breast milk, mucus, saliva, sebum, semen, sweat, tears, or vaginal secretion fluid. In a related embodiment, the optical agent is administered to a bodily fluid of an animal subject. In an aspect, the animal is a mammal. In a related aspect, the animal is a human.

The pH monitoring methods described herein are effective in monitoring the pH of a fluid over a broad range of pH values. In an embodiment, for example, the pH of the fluid ranges from 1.0 to 13.0. In an aspect, the pH of the fluid ranges from 5.0 to 9.0. In another aspect, the pH of the fluid ranges from 1.0 to 7.5, optionally from 7.0 to 13.0.

A wide range of photoactive moieties are compatible with the pH monitoring methods described herein. In an embodiment, the one or more photoactive moieties comprise a group corresponding to a pyrazine, a thiazole, a phenylxanthene, a phenothiazine, a phenoselenazine, a cyanine, an indocyanine, a squaraine, a dipyrrolo pyrimidone, an anthraquinone, a tetracene, a quinoline, an acridine, an acridone, a phenanthridine, an azo dye, a rhodamine, a phenoxazine, an azulene, an aza-azulene, a triphenyl methane dye, an indole, a benzoindole, an indocarbocyanine, a Nile Red dye, or a benzoindocarbocyanine. In an aspect, the one or more photoactive moieties comprise a group corresponding to a pyrazine, an azulene, or an aza-azulene. In a related embodiment, the one or more photoactive moieties do not comprise a group corresponding to a pyrazine.

The composition of the optical agents of the pH monitoring methods described herein greatly affects the photo-physical properties of the optical agents. Selection of the block copolymers and linking groups of the optical agents, for example, can affect the fluorescence intensities and supramolecular structures of the optical agents described herein. In an embodiment, for example, the stoichiometric ratio of the linking groups to monomers of the hydrophilic blocks of the optical agent is selected over a range of 1:100 to 99:100, optionally 1:100 to 50:100, still optionally 50:100 to 99:100, also optionally 1:100 to 25:100, preferably 25:100 to 75:100. In another embodiment, the hydrophilic blocks of the cross-linked block copolymers comprise poly(ethylene oxide) or poly(acrylic acid). In an embodiment, for example, the cross-linking density is less than 20%. In another embodiment, the cross-linking density is selected from the range of 1% to 20%, optionally from 5% to 7%, optionally from 5% to 14%, optionally from 5% to 10%, optionally from 9% to 14%. In an aspect, the hydrophobic blocks of the cross-linked block copolymers comprise polystyrene. In a related embodiment, the cross-linked block copolymers of the optical agent are triblock copolymers. In an aspect, the cross-linked block copolymers of the optical agent are triblock copolymers further comprising central reactivity blocks for covalently linking to the linking groups. In another embodiment, the central reactivity blocks comprise an activated ester group. In an aspect, the central reactivity blocks comprise poly(N-acryloxysuccinimide).

In an embodiment, for example, the hydrophilic block comprises poly(acrylic acid) (PAA) and there are from 10 to 500 repeating units. In an embodiment, the hydrophobic block comprises poly(acetoxystyrene) having from 10 to 300 repeating units. In an embodiment, the hydrophobic block comprises poly(p-hydroxystyrene) having from 10 to 300 repeating units. In an embodiment, the hydrophilic block comprises poly(ethylene oxide) (PEO) having from 10 to 300 repeating units. In an embodiment, the hydrophilic block comprises PNAS in aqueous solution having from 10 to 300 repeating units. In an embodiment, the hydrophobic block comprises polystyrene (PS) having from 10 to 600 repeating units.

In an embodiment, for example, the hydrophobic block is a poly(p-hydroxystyrene) polymer block; a polystyrene polymer block; a poly(p-hydroxystyrene) polymer block; a polyacrylate polymer block; a poly(propylene glycol) polymer block; a poly(ester) polymer block; a polylactic acid polymer block; a poly(tert-butylacrylate) polymer block; a poly(N-acryloxysuccinimide) polymer block; or a copolymer thereof. In a related embodiment, the hydrophilic block is a poly(acrylic acid) polymer block; a poly(ethylene glycol) polymer block; a poly(acetoxystyrene) polymer block; or a copolymer thereof.

Specific classes of triblock copolymers and linking groups can be used to construct optical agents of the pH monitoring methods described herein. In an embodiment, for example, the block copolymers are of formula (FX23):

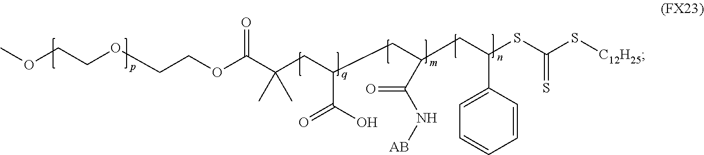

wherein:
each AB is independently LG or Bm;
each LG is the linking group;
each Bm is independently an amino acid, a peptide, a protein, a nucleoside, a nucleotide, an enzyme, a carbohydrate, a glycomimetic, an oligomer, a lipid, a polymer, an antibody, an antibody fragment, a mono- or polysaccharide comprising 1 to 50 carbohydrate units, a glycopeptide, a glycoprotein, a peptidomimetic, a drug, a steroid, a hormone, an aptamer, a receptor, a metal chelating agent, a polynucleotide comprising 2 to 50 nucleic acid units, a peptoid comprising 2 to 50 N-alkylaminoacetyl residues, a glycopeptide comprising 2 to 50 amino acid and carbohydrate units, or a polypeptide comprising 2 to 30 amino acid units;
each m is independently an integer selected from the range of 1 to 500;
each n is independently an integer selected from the range of 1 to 500;
each p is independently an integer selected from the range of 0 to 500; and
each q is independently an integer selected from the range of 0 to 500.

In a related embodiment, the linking groups are of formula (FX24) or (FX25):

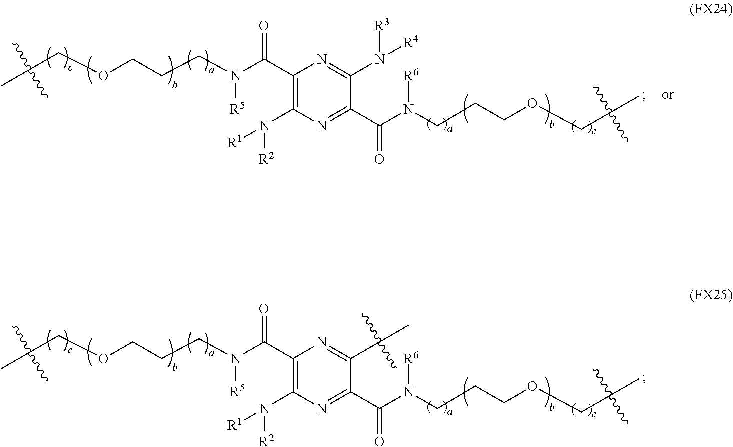

wherein: each a is independently an integer selected from the range of 0 to 10; each b is independently an integer selected from the range of 0 to 500; each c is independently an integer

wherein: each a is independently an integer selected from the range of 0 to 10; each b is independently an integer selected from the range of 0 to 500; each c is independently an integer selected from the range of 1 to 10; each of R¹-R⁴ is independently a hydrogen, C₁-C₂₀ alkyl, C₃-C₂₀ cycloalkyl, C₅-C₂₀ alkylaryl, C₁-C₁₀ polyhydroxyalkyl, C₁-C₁₀ polyalkoxyalkyl,

—CH₂(CH₂OCH₂)_{y}CH₂OH, —CH₂(CHOH)ₓR⁶⁰, or —(CH₂CH₂O)_{y}R⁶¹; each of x and y is independently an integer selected from the range of 1 to 100; and each of R⁵, R⁶, R⁶⁰ and R⁶¹ is independently hydrogen, C₁-C₁₀ alkyl, C₃-C₁₀ cycloalkyl, C₅-C₁₀ heteroaryl or C₅-C₁₀ aryl.

In an aspect, the linking groups are of formula (FX26) or (FX27):

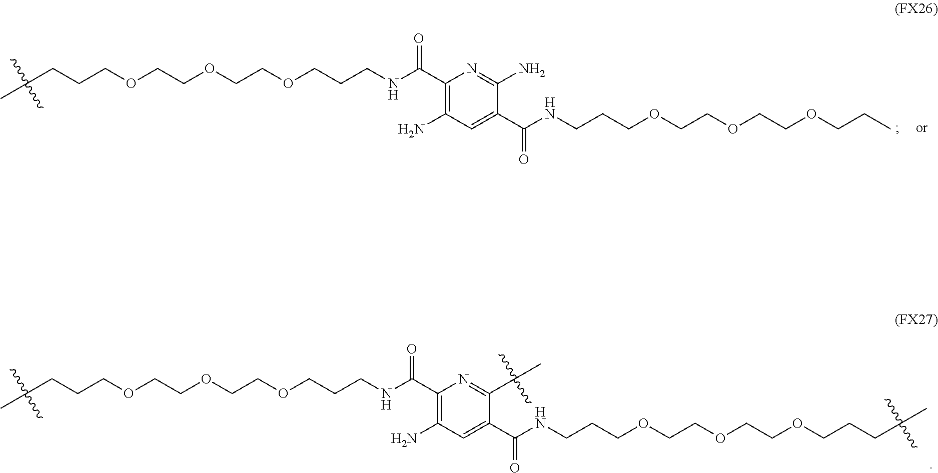 [SMILES: *CCCOCCOCCOCCCNC(=O)c1nc(N)c(C(=O)NCCCOCCOCCOCCC)cc1N ; *CCCOCCOCCOCCCNC(=O)c1cc(N)c(C(=O)NCCCOCCOCCOCCC*)nc1*]

In an embodiment, the linking groups are of formula (FX28) or (FX29):

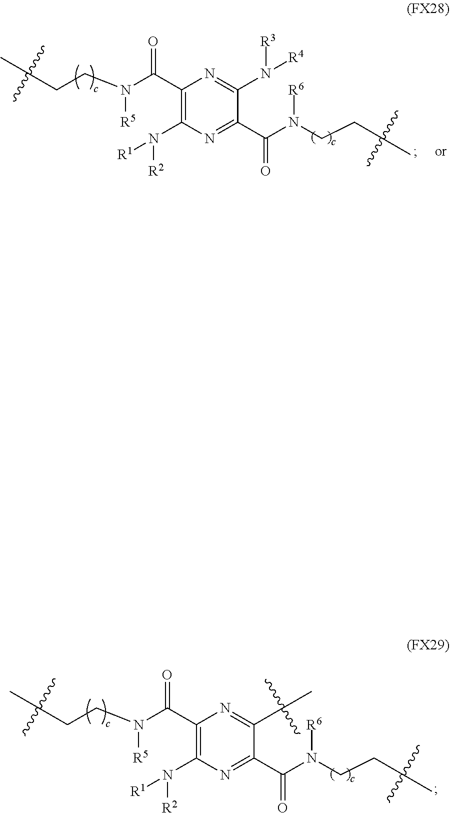

wherein: each c is independently an integer selected from the range of 1 to 10; each of R¹-R⁴ is independently a hydrogen, C₁-C₂₀ alkyl, C₃-C₂₀ cycloalkyl, C₅-C₂₀ alkylaryl, C₁-C₁₀ polyhydroxyalkyl, C₁-C₁₀ polyalkoxyalkyl, —CH₂(CH₂OH₂)_{y}CH₂OH, —CH₂(CHOH)ₓR⁶⁰, or
—(CH₂CHO)_{y}R⁶¹; each of x and y is independently an integer selected from the range of 1 to 100; and each of R⁵, R⁶, R⁶⁰ and R⁶¹ is independently hydrogen, C₁-C₁₀ alkyl, C₃-C₁₀ cycloalkyl, C₅-C₁₀ heteroaryl or C₅-C₁₀ aryl.

In an aspect, the linking groups are of formula (FX30) or (FX31):

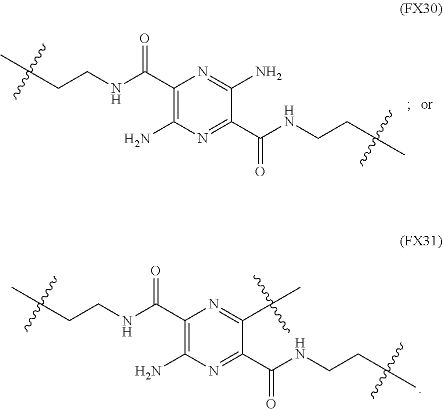 [SMILES: *CCNC(=O)c1nc(N)c(C(=O)NCC*)nc1N ; *CCNC(=O)c1nc(N)c(C(=O)NCC*)nc1*]

In an embodiment, a device for measuring the pH of a fluid is provided, the device comprising: an optical source for providing electromagnetic radiation; an electromagnetic radiation delivery system in optical communication with the optical source, for providing at least a portion of the electromagnetic radiation to an optical agent administered to the fluid, thereby exciting fluorescence from the optical agent in the fluid; wherein the optical agent comprises: cross-linked block copolymers, wherein each of the block copolymers comprises one or more hydrophilic blocks and one or more hydrophobic blocks; and linking groups covalently cross linking at least a portion the hydrophilic blocks of the block copolymers, wherein at least a portion of the linking groups comprise one or more photoactive moieties; wherein the optical agent forms a supramolecular structure in aqueous solution, the supramolecular structure having one or more interior hydrophobic cores and one or more covalently cross-linked hydrophilic shells, wherein the one or more interior hydrophobic cores comprise the hydrophobic blocks of the block copolymers, and the one or more covalently cross-linked hydrophilic shells comprise the hydrophilic blocks of the block copolymers; an electromagnetic radiation collection system in optical communication with the fluid for collecting at least a portion of the fluorescence from the optical agent and providing at least a portion of the fluorescence to a detector; the detector for receiving at least a portion of the fluorescence from the electromagnetic radiation collection system; wherein the detector measures a first fluorescence intensity at a first wavelength and a second fluorescence intensity at a second wavelength; wherein the second wavelength differs from the first wavelength; and a processor in optical or electronic communication with the detector; wherein the processor is programmed to: calculate a fluorescence intensity ratio of the first fluorescence intensity at the first wavelength to the second fluorescence intensity at the second wavelength; and compare the calculated fluorescence intensity ratio to a reference fluorescence intensity ratio.

A variety of additional device components are useful for the pH monitoring devices described herein. In an embodiment, for example, the pH monitoring device further comprises a fiber optic, catheter, endoscope, ear clip, hand band, head band, forehead sensor, surface coil, or finger probe for providing at least a portion of the electromagnetic radiation to the optical agent administered to the fluid and/or for collecting at least a portion of the fluorescence from the optical agent and providing at least a portion of the fluorescence to the detector.

The pH monitoring devices described herein are useful for monitoring of pH in many different environments. In an embodiment, for example, the pH of the fluid is measured in vivo. In a related embodiment, the pH of the fluid is measured in vitro.

The processors of the pH monitoring devices described herein can perform additional useful functions. In an embodiment, the device measures the reference fluorescence intensity ratio by measuring fluorescence intensities at a plurality of wavelengths for one or more reference sample fluids having a known pH.

The pH monitoring devices described herein are useful with an array of optical agent supramolecular structures. In an embodiment, the optical agent supramolecular structure comprises a nanoparticle or shell cross-linked micelle. In an aspect, the optical agent supramolecular structure comprises a shell cross-linked micelle or nanoparticle having a globular, spherical, cylindrical, disc, toroidal, spheroidal, vesicle, or multicompartment morphology. In another aspect, the optical agent supramolecular structure comprises a shell cross-linked micelle or nanoparticle having a multicompartment morphology. In an aspect, the optical agent supramolecular structure comprises a shell cross-linked micelle or nanoparticle having a cylindrical or rod morphology. In another aspect, the optical agent supramolecular structure comprises a shell cross-linked micelle or nanoparticle having a rod morphology. In a related embodiment, one or more dimensions of the supramolecular structure is controlled by selection of the hydrophobic blocks of the block copolymer, the hydrophilic blocks of the block copolymer, aqueous solution composition, or any combination thereof.

The pH monitoring devices described herein can be practiced employing several fluorescence detection techniques. In an embodiment, for example, the detector measures the first fluorescence intensity and the second fluorescence intensity by measuring a local maximum of the fluorescence intensity. In a related embodiment, the detector measures the first fluorescence intensity and the second fluorescence intensity by measuring integrated intensities of the fluorescence a preselected range of wavelengths about the first wavelength and the second wavelength.

The pH monitoring devices described herein are compatible with a wide range of wavelengths of electromagnetic radiation. In an embodiment, the electromagnetic radiation provided to the optical agent has wavelengths selected from the range of 350 nanometers to 1300 nanometers. In a related embodiment, the first wavelength is selected from the range of 350 nanometers to 1300 nanometers. In a related aspect, the second wavelength is selected from the range of 350 nanometers to 1300 nanometers.

The pH monitoring devices described herein enable fluorescence ratio calculation for fluorescence intensities detected over a broad range of wavelengths of electromagnetic radiation. In an embodiment, for example, the first wavelength differs from the second wavelength by an amount greater than or equal to 5 nanometers. In another embodiment, the first wavelength differs from the second wavelength by an amount selected from the range of 5 nanometers to 600 nanometers.

The calculated fluorescence intensity ratio of pH monitoring devices described herein is useful for measuring the pH of a fluid over a broad range of fluorescence intensity ratios. In an embodiment, for example, the calculated fluorescence intensity ratio ranges from 0.3 to 3. In an aspect, the calculated fluorescence intensity ratio ranges from 0.01 to 100, optionally from 1 to 50, and optionally from 0.1 to 10.

Many fluids are compatible with the pH monitoring devices described herein. In an embodiment, for example, the fluid comprises a bodily fluid of an animal, an organic solvent, a cell extract, a cell lysate, or a water source. In an aspect, the fluid comprises a bodily fluid of an animal comprising blood, plasma, cerebrospinal fluid, aqueous humour, pleural fluid, pericardial fluid, lymph chyme, chyle, bile, synovial fluid, peritoneal fluid, stool, water, prostatic fluid, amniotic fluid, milk, urine, vomit, cerumen, gastic acid, breast milk, mucus, saliva, sebum, semen, sweat, tears, or vaginal secretion fluid. In a related embodiment, the optical agent is administered to a bodily fluid of an animal subject. In an aspect, the animal is a mammal. In a related aspect, the animal is a human.

The pH monitoring devices described herein are effective for monitoring the pH of a fluid over a broad range of pH values. In an embodiment, the pH of the fluid ranges from 1.0 to 13.0. In a related embodiment, the pH of the fluid ranges from 5.0 to 9.0. In an aspect, the pH of the fluid ranges from 1.0 to 7.5, optionally from 7.0 to 13.0.

A wide range of photoactive moieties are compatible with the pH monitoring devices described herein. In an embodiment, the one or more photoactive moieties comprise a group corresponding to a pyrazine, a thiazole, a phenylxanthene, a phenothiazine, a phenoselenazine, a cyanine, an indocyanine, a squaraine, a dipyrrolo pyrimidone, an anthraquinone, a tetracene, a quinoline, an acridine, an acridone, a phenanthridine, an azo dye, a rhodamine, a phenoxazine, an azulene, an aza-azulene, a triphenyl methane dye, an indole, a benzoindole, an indocarbocyanine, a Nile Red dye, or a benzoindocarbocyanine. In an aspect, the one or more photoactive moieties comprise a group corresponding to a pyrazine, an azulene, or an aza-azulene. In a related embodiment, the one or more photoactive moieties do not comprise a group corresponding to a pyrazine.

The composition of the optical agents of the pH monitoring devices described herein greatly affects the photo-physical properties of the optical agents. Selection of the block copolymers and linking groups of the optical agents, for example, can affect the fluorescence intensities and supramolecular structures of the optical agents described herein. In an embodiment, for example, the stoichiometric ratio of the linking groups to monomers of the hydrophilic blocks of the optical agent is selected over a range of 1:100 to 99:100, optionally 1:100 to 50:100, still optionally 50:100 to 99:100, also optionally 1:100 to 25:100, preferably 25:100 to 75:100. In another embodiment, the hydrophilic blocks of the cross-linked block copolymers comprise poly(ethylene oxide) or poly(acrylic acid). In an embodiment, for example, the cross-linking density is less than 20%. In another embodiment, the cross-linking density is selected from the range of 1% to 20%, optionally from 5% to 7%, optionally from 5% to 14%, optionally from 5% to 10%, optionally from 9% to 14%. In an aspect, the hydrophobic blocks of the cross-linked block copolymers comprise polystyrene. In a related embodiment, the cross-linked block copolymers of the optical agent are triblock copolymers. In an aspect, the cross-linked block copolymers of the optical agent are triblock copolymers further comprising central reactivity blocks for covalently linking to the linking groups. In another embodiment, the central reactivity blocks comprise an activated ester group. In an aspect, the central reactivity blocks comprise poly(N-acryloxysuccinimide).

In an embodiment, for example, the hydrophilic block comprises poly(acrylic acid) (PAA) and there are from 10 to 500 repeating units. In an embodiment, the hydrophobic block comprises poly(acetoxystyrene) having from 10 to 300 repeating units. In an embodiment, the hydrophobic block comprises poly(p-hydroxystyrene) having from 10 to 300 repeating units. In an embodiment, the hydrophilic block comprises poly(ethylene oxide) (PEO) having from 10 to 300 repeating units. In an embodiment, the hydrophilic block comprises PNAS in aqueous solution having from 10 to 300 repeating units. In an embodiment, the hydrophobic block comprises polystyrene (PS) having from 10 to 600 repeating units.

In an embodiment, for example, the hydrophobic block is a poly(p-hydroxystyrene) polymer block; a polystyrene polymer block; a poly(p-hydroxystyrene) polymer block; a polyacrylate polymer block; a poly(propylene glycol) polymer block; a poly(ester) polymer block; a polylactic acid polymer block; a poly(tert-butylacrylate) polymer block; a poly(N-acryloxysuccinimide) polymer block; or a copolymer thereof. In a related embodiment, the hydrophilic block is a poly(acrylic acid) polymer block; a poly(ethylene glycol) polymer block; a poly(acetoxystyrene) polymer block; or a copolymer thereof.

Specific classes of triblock copolymers and linking groups can be used to construct optical agents used in conjunction with the pH monitoring devices described herein. In an embodiment, for example, the block copolymers are of formula (FX23):

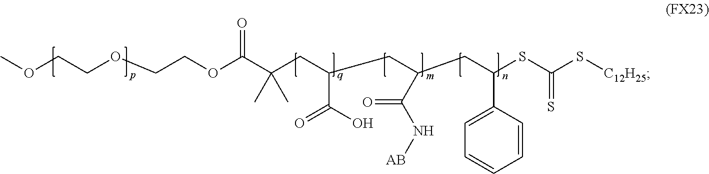

wherein:
each AB is independently LG or Bm;
each LG is the linking group;
each Bm is independently an amino acid, a peptide, a protein, a nucleoside, a nucleotide, an enzyme, a carbohydrate, a glycomimetic, an oligomer, a lipid, a polymer, an antibody, an antibody fragment, a mono- or polysaccharide comprising 1 to 50 carbohydrate units, a glycopeptide, a glycoprotein, a peptidomimetic, a drug, a steroid, a hormone, an aptamer, a receptor, a metal chelating agent, a polynucleotide comprising 2 to 50 nucleic acid units, a peptoid comprising 2 to 50 N-alkylaminoacetyl residues, a glycopeptide comprising 2 to 50 amino acid and carbohydrate units, or a polypeptide comprising 2 to 30 amino acid units;
each m is independently an integer selected from the range of 1 to 500;
each n is independently an integer selected from the range of 1 to 500;
each p is independently an integer selected from the range of 0 to 500; and
each q is independently an integer selected from the range of 0 to 500.

In a related embodiment, the linking groups are of formula (FX24) or (FX25):

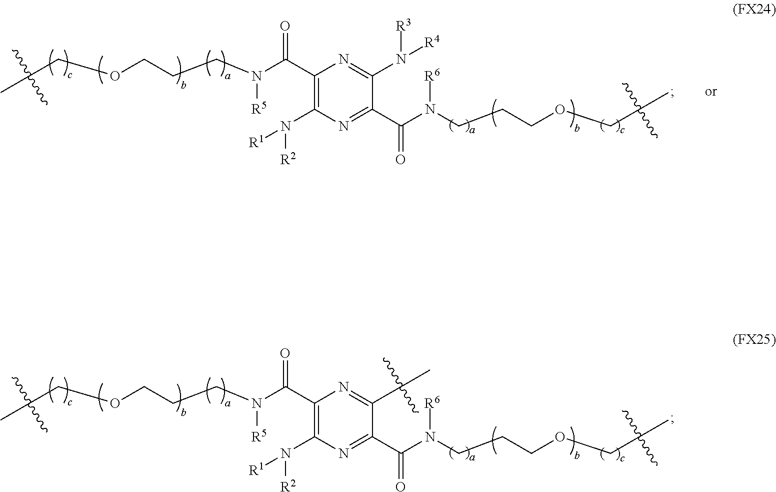

wherein: each a is independently an integer selected from the range of 0 to 10; each b is independently an integer selected from the range of 0 to 500; each c is independently an integer selected from the range of 1 to 10; each of R¹-R⁴ is independently a hydrogen, C₁-C₂₀ alkyl, C₃-C₂₀ cycloalkyl, C₅-C₂₀ alkylaryl, C₁-C₁₀ polyhydroxyalkyl, C₁-C₁₀ polyalkoxyalkyl,

—CH₂(CH₂OH₂)_{y}CH₂OH, —CH₂(CHOH)ₓR⁶⁰, or —(CH₂CH₂O)_{y}R⁶¹; each of x and y is independently an integer selected from the range of 1 to 100; and each of R⁵, R⁶, R⁶⁰ and R⁶¹ is independently hydrogen, C₁-C₁₀ alkyl, C₃-C₁₀ cycloalkyl, C₅-C₁₀ heteroaryl or C₅-C₁₀ aryl.

In an aspect, the linking groups are of formula (FX26) or (FX27):

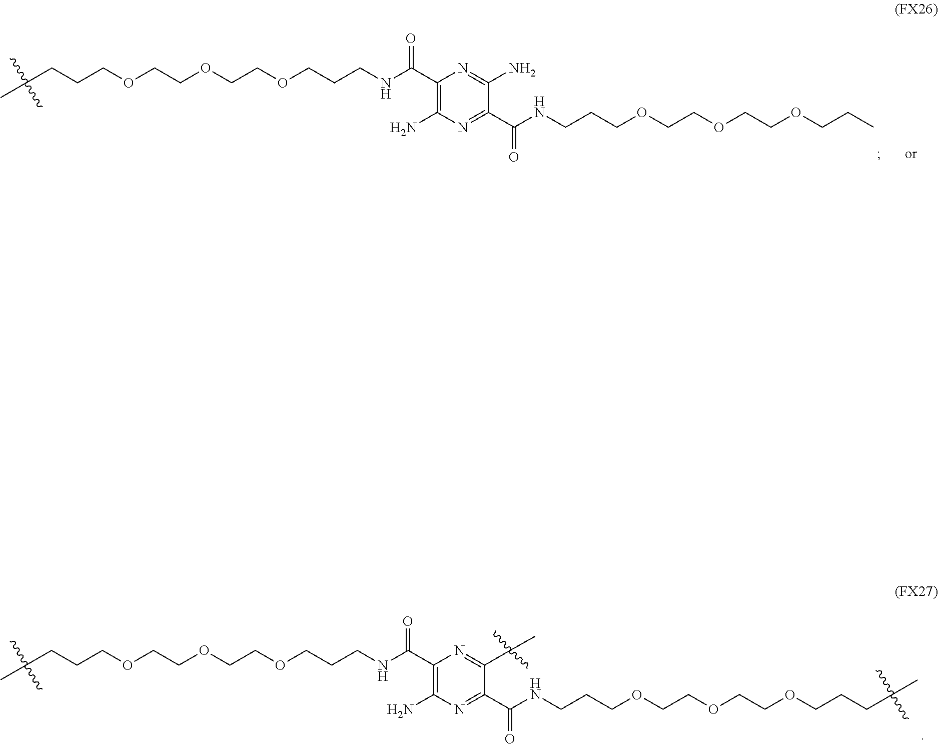 [SMILES: *CCCOCCOCCOCCCNC(=O)c1nc(N)c(C(=O)NCCCOCCOCCOCCC)nc1N ; *CCCOCCOCCOCCCNC(=O)c1nc(N)c(C(=O)NCCCOCCOCCOCCC*)nc1*]

In an embodiment, the linking groups are of formula (FX28) or (FX29):

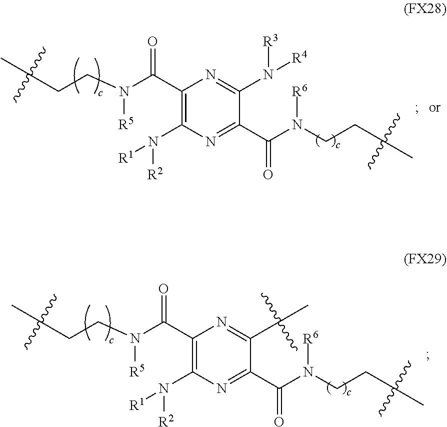

wherein: each c is independently an integer selected from the range of 1 to 10; each of R¹-R⁴ is independently a hydrogen, C₁-C₂₀ alkyl, C₀₃-C₂₀ cycloalkyl, C₅-C₂₀ alkylaryl, C₁-C₁₀ polyhydroxyalkyl, C₁-C₁₀ polyalkoxyalkyl, —CH₂(CH₂OCH₂)CH₂CHOH, —CH₂(CHOH)ₓR⁶⁰, or
—(CH₂CH₂O)_{y}R⁶¹; each of x and y is independently an integer selected from the range of 1 to 100; and each of R⁵, R⁶, R⁶⁰ and R⁶¹ is independently hydrogen, C₁-C₁₀ alkyl, C₃-C₁₀ cycloalkyl, C₅-C₁₀ heteroaryl or C₅-C₁₀ aryl.

In an aspect, the linking groups are of formula (FX30) or (FX31):

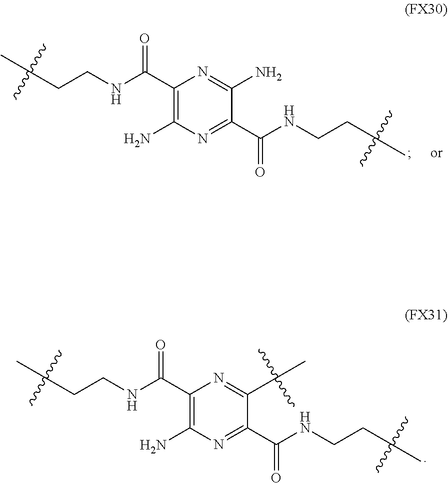 [SMILES: *CCNC(=O)c1nc(N)c(C(=O)NCC*)nc1N ; *CCNC(=O)c1nc(N)c(C(=O)NCC*)nc1*]

The present invention also provides optical agents, including compositions, preparations and formulations, for imaging, visualization, diagnostic monitoring and phototherapeutic applications. Optical agents of the present invention include photonic nanostructures and nanoassemblies including supramolecular structures, such as shell-cross-linked micelles, that incorporate at least one linking group comprising one or more photoactive moieties that provide functionality as exogenous agents for a range of biomedical applications. Optical agents of the present invention comprise supramolecular structures having linking groups imparting useful optical and structural functionality. In an embodiment, for example, the presence of linking groups function to covalently cross link polymer components to provide a cross-linked shell stabilized supramolecular structure, and also impart useful optical functionality, for example by functioning as a chromophore, fluorophore, photosensitizer, and/or a photoreactive species. Some optical agents of the present invention further comprise one or more targeting ligands covalently or non-covalently associated with a photonic nanostructure or nanoassembly, thereby providing specificity for administering, targeting and/or localizing the optical agent to a specific biological environment, such as a specific organ, tissue, cell type or tumor site. Optical agents of the present invention optionally include bioconjugates.

Optical agents of the present invention are useful for a variety of in vivo, in vitro and ex vivo biomedical diagnostic, visualization and imaging applications, such as tomographic imaging, monitoring and evaluating organ functioning, anatomical visualization, coronary angiography, fluorescence endoscopy, and the detection and imaging of tumors. In an embodiment, for example, photonic nanostructures and nanoassemblies of the present invention comprising shell-cross-linked micelles provide compositions for chemical and physiological sensing applications, for example, enabling the in situ monitoring of pH and/or the monitoring of organ function in a patient. Alternatively, photonic nanostructures and nanoassemblies of the present invention comprising shell-cross-linked micelles provide organic optical probes and contrast agents for optical imaging methods, including multiphoton imaging, and photoacoustic imaging. Optical agents of the present invention are useful for a variety of therapeutic applications including phototherapeutic treatment methods, image guided surgery, administration and target specific delivery of therapeutic agents, and endoscopic procedures and therapies. In an embodiment, for example, photonic nanostructures and nanoassemblies of the present invention comprising shell-cross-linked micelles provide optical agents for absorbing electromagnetic radiation provided to a target biological environment, organ or tissue, and transferring it internally to a phototherapeutic agent capable of providing a desired therapeutic effect.

In one aspect, the present invention provides an optical agent that includes a cross-linked supramolecular structure having bifunctional linking groups for covalently cross linking polymer components and for providing useful optical functionality. An optical agent of this aspect comprises cross-linked block copolymers, each of which comprises a hydrophilic block and a hydrophobic block. Further, the optical agent of this aspect comprises linking groups that covalently cross link at least a portion of the hydrophilic blocks of the block copolymers. With regard to some optical agents, at least a portion of the linking groups connecting hydrophilic blocks of the block copolymers include one or more photoactive moieties, such as fluorophores or photosensitizers capable of excitation in the visible region (e.g. 400 nm to 750 nm) and/or the near infrared region (e.g., 750-1300 nm). The compositions of block copolymer and linking group components are selected such that the optical agent forms a supramolecular structure in aqueous solution. This resulting supramolecular structure has an interior hydrophobic core that includes the hydrophobic blocks of the block copolymers. Also, the resulting supramolecular structure has a covalently cross-linked hydrophilic shell that includes the hydrophilic blocks of the block copolymers. In an embodiment, the optical agent forms a supramolecular structure in aqueous solution comprising an optically functional micelle, a vesicle, a bilayer, a folded sheet, a tubular micelle, a toroidal micelle or a discoidal micelle. Optical agents of the present invention include, for example, shell-cross-linked micelles, optionally having cross sectional dimensions selected from the range of 5 nanometers to 100 nanometers capable of functioning as a chromophore, fluorophore or phototherapeutic agent, and optionally capable of excitation in the visible region (e.g. 400 nm to 750 nm) and/or the near infrared region (e.g., 750-1300 nm). Selection of the physical dimensions of micelle-based optical agents of the present invention may be based on a number of factors such as, toxicity, immune response, biocompatibility and/or bioclearance considerations.

Selection of the composition of linking groups and extent of cross linking, at least in part, determines the optical, physical, physiological and chemical properties of supramolecular structures and assemblies of optical agents of the present invention, such as their excitation wavelengths, emission wavelengths, Stokes shifts, quantum yields, cross sectional dimensions, extent of cross linking, stability, biocompatibility, physiological clearance rate upon administration to a patient, etc. Useful photoactive moieties of the linking groups for optical agents of the present invention include dyes, fluorophores, chromophores, photosensitizers, photoreactive agents, phototherapeutic agents, and conjugates, complexes, fragments and derivatives thereof. In an embodiment, for example, the stoichiometric ratio of the linking groups to monomers of the hydrophilic blocks is selected over the range of 0.1:100 to 75:100, optionally 1:100 to 75:100, optionally 10:100 to 75:100 and optionally 30:100 to 75:100.

In an embodiment attractive for diagnostic, imaging and physiological sensing applications, at least a portion of the linking groups of the present optical agents comprise one or more chromophores and/or fluorophores. Useful linking groups of this aspect include visible dyes and/or near infrared dyes, including fluorescent dyes. In an embodiment, for example, the linking groups are chromophore and/or fluorophore functional groups capable of excitation upon absorption of electromagnetic radiation having wavelengths selected over the range of 400 nanometers to 1300 nanometers, and optionally capable of emission of electromagnetic radiation having wavelengths selected over the range of 400 nanometers to 1300 nanometers. Incorporation of linking groups that are excited upon absorption of electromagnetic radiation having wavelengths over the range of about 400 nanometers to about 1200 nanometers, optionally for some applications 400 nm to 900 nm, and optionally for some applications 700 nm to 900 nm, is particularly useful for certain diagnostic and therapeutic applications as electromagnetic radiation of these wavelengths is effectively transmitted by some biological samples and environments (e.g., biological tissue). In an embodiment, an optical agent of the invention includes one or more fluorophores having a Stokes shift selected over the range of, for example, 10 nanometers to 200 nanometers, optionally for some applications 20 nm to 200 nm, and optionally for some applications 50 nm to 200 nm. Useful photoactive moieties of the linking groups for optical agents of the present invention include, but are not limited to, a phenylxanthene, a phenothiazine, a phenoselenazine, a cyanine, an indocyanine, a squaraine, a dipyrrolo pyrimidone, an anthraquinone, a tetracene, a quinoline, a pyrazine, an acridine, an acridone, a phenanthridine, an azo dye, a rhodamine, a phenoxazine, an azulene, an azaazulene, a triphenyl methane dye, an indole, a benzoindole, an indocarbocyanine, a Nile Red dye, a benzoindocarbocyanine, and conjugates, complexes, fragments and derivatives thereof. In an embodiment, an optical agent of the present invention comprises a pyrazine-based linking group that cross links the hydrophilic blocks of the block copolymers, optionally a pyrazine-based amino linking group, such as a pyrazine-based diamino linking group or a pyrazine-based tetra amino linking group.

A range of linking chemistry is useful in the shell-cross-linked supramolecular structures of optical agents of the present invention. Cross linking can be achieved, for example, via chemical reaction between the hydrophilic blocks of copolymers and cross linking reagents(s) containing one or more amine, imine, sulfhydryl, azide, carbonyl, imidoester, succinimidyl ester, carboxylic acid, hydroxyl, thiol, thiocyanate, acrylate, or halo group. Cross linking can be achieved, for example, via chemical reaction between cross linking reagents(s) and the hydrophilic block of the copolymer containing one or more monomers having one or more ester sites for conjugation to the linking group via amidation. In an embodiment the hydrophilic block of the copolymer includes N-acryloxysuccinimde monomers for conjugation to the linking groups. In some embodiments, for example, the hydrophilic block of the copolymers are cross-linked via carboxamide or disulfide linkages between the at least a portion of the monomers of the hydrophilic blocks and the linking groups. Linking groups of the present invention optionally include spacer moieties, such as a C₁-C₃₀ poly(ethylene glycol) (PEG) spacer, or substituted or unsubstituted C₁-C₃₀ alkyl chain. Linking groups of the present invention optionally include one or more amino acid groups or derivatives thereof. In an embodiment, for example, an optical agent of the present invention incorporates linking groups having one or more basic amino acid groups or derivatives thereof including, but not limited to, arginine, lysine, histidine, ornithine, and homoarginine. Use of linking groups containing one or more basic amino acids is beneficial in the present invention for achieving high extents of cross linking between monomers of the hydrophilic groups of the block copolymers.

In an embodiment attractive for phototherapeutic applications, the photoactive moiety(ies) of the linking groups for the optical agents comprise(s) one or more photoreactive moieties such as phototherapeutic agents or precursors of phototherapeutic agents, optionally capable of excitation via absorption of electromagnetic radiation having wavelengths in the visible region (e.g. 400 nm to 750 nm) and/or the near infrared region (e.g., 750-1300 nm). In some embodiments, for example, the linking groups are capable of absorbing electromagnetic radiation and initiating a desired therapeutic effect such as the degradation of a tumor or other lesion. In an embodiment, for example, an optical agent of the present invention comprises linking groups containing one or more photosensitizer that absorbs visible or near infrared radiation and undergoes cleavage of photolabile bonds and/or energy transfer processes that generate reactive species (e.g., radicals, ions, nitrene, carbene etc.) capable of achieving a desired therapeutic effect. In an embodiment, an optical agent comprises a phototherapeutic agent comprising linking groups that generates reactive species (e.g., radicals, ions, nitrene, carbene etc.) upon absorption of electromagnetic radiation having wavelengths selected over the range of 400 nanometers to 1200 nanometers, optionally for some applications 400 nm to 900 nm, and optionally for some applications 700 nm to 900 nm.

Useful photoreactive moieties for linking groups of optical agents of this aspect of the present invention include, but are not limited to, Type-1 or Type-2 phototherapeutic agents such as: a cyanine, an indocyanine, a phthalocyanine, a rhodamine, a phenoxazine, a phenothiazine, a phenoselenazine, a fluorescein, a porphyrin, a benzoporphyrin, a squaraine, a corrin, a croconium, an azo dye, a methine dye, an indolenium dye, a halogen, an anthracyline, an azide, a C₁-C₂₀ peroxyalkyl, a C₁-C₂₀ peroxyaryl, a C₁-C₂₀ sulfenatoalkyl, a sulfenatoaryl, a diazo dye, a chlorine, a naphthalocyanine, a methylene blue, a chalcogenopyrylium analogue, and conjugates, complexes, fragments and derivatives thereof.

Selection of the composition of block copolymers in part determines the optical, physical, physiological and chemical properties of supramolecular structures and assemblies of optical agents of the present invention, such as the excitation wavelengths, emission wavelengths, Stokes shifts, quantum yields, cross sectional dimensions, extent of cross linking, stability, biocompatibility, physiological clearance rate upon administration to a patient etc. In an embodiment, the present invention provides an optical agent that is a supramolecular structure or assembly, such as a shell-cross-linked micelle composition, wherein at least a portion of the polymer components comprise diblock copolymers each having a hydrophilic block directly or indirectly linked to a hydrophobic block. In the context of this description, directly linked refers to block copolymers wherein the hydrophilic and hydrophobic block are linked to each other directly via a covalent bond, and indirectly linked refers to block copolymers wherein the hydrophilic and hydrophobic block are linked to each other indirectly via a spacer or linking group. Hydrophilic blocks and hydrophobic blocks of block copolymers of the present invention can have a wide range of lengths, for example, lengths selected over the range of 10 to 250 monomers. Hydrophilic blocks of supramolecular structures and assemblies of the present optical agents are capable of effective cross linking between the block copolymers, for example using EDC (1-Ethyl-3-[3-dimethylaminopropyl]carbodiimide hydrochloride) coupling reactions or photoinitiated cross linking reactions. Useful hydrophilic blocks of optical agents of the present invention include, but are not limited to, a poly(acrylic acid) polymer block, a poly(N-(acryloyloxy)succinimide) polymer block; a poly(N-acryloylmorpholine) polymer block; a poly(ethylene glycol) polymer block; or a copolymer thereof. Useful hydrophobic blocks of optical agents of the present invention include, but are not limited to, a poly(p-hydroxystyrene) polymer block; a polystyrene polymer block; a polyacrylate polymer block, a poly(propylene glycol) polymer block; a poly(amino acid) polymer block; a poly(ester) polymer block; a poly (ε-caprolactone) polymer block, and a phospholipid; poly(p-vinyl benzaldehyde) block and a poly(phenyl vinyl ketone) block; poly(p-vinyl benzaldehyde) block and a poly(methyl vinyl ketone) block; or a copolymer thereof.

In an embodiment, the hydrophobic block is selected from but not limited to poly(methyl acrylate), poly(ε-caprolactone), poly(lactic acid), poly(glycolic acid), polylactide, and polyglycolide. In an embodiment, the hydrophilic block is selected from but not limited to poly(acrylic acid), poly(aminoethyl acrylamide), poly(oligoethylene oxide acrylate), and poly(N-acryloxysuccinimide).

Hydrophilic blocks and hydrophobic blocks of the present invention optionally have a composition specifically engineered to provide additional chemical and/or physical properties useful for selected biomedical applications, such as in situ sensing and monitoring of organ function or physiological condition(s). In an embodiment, the hydrophilic blocks, hydrophobic blocks or both of the block copolymers comprise functional groups responsive to a specific chemical environment or physiological state, such that the supramolecular structure undergoes a change in structure, such as swelling or contracting, in response to a change in the chemical environment or physiological state. In a specific optical agent of the present invention, for example, the hydrophilic block, hydrophobic block or both comprises one or more acidic or basic functional groups responsive to pH, wherein the supramolecular structure undergoes a change in volume in response to a change in the pH of the aqueous solution. This feature of the certain optical agents of the present invention is used in some methods for sensing and/or monitoring a chemical environment or physiological state, for example for in situ pH monitoring.

Optical agents of the present invention optionally include bioconjugates capable of targeted administration and delivery, such as tissue-specific, organ-specific, cell-specific and tumor-specific administration and delivery. In an embodiment, for example, an optical agent of the present invention further comprises one or more targeting ligands coupled to the supramolecular structure or assembly, such as a shell-cross-linked micelle. Targeting ligands of the present invention may be covalently bonded to, or non-covalently associated with, the hydrophilic blocks of at least a portion of the block copolymers of the present optical agents. Useful targeting ligands include, but are not limited to, a peptide, a protein, an oligonucelotide, an antibody, carbohydrate, hormone, a lipid, a drug and conjugates, complexes, fragments and derivatives thereof.

Compositions of the invention include formulations and preparations comprising one or more of the present optical agents provided in an aqueous solution, such as a pharmaceutically acceptable formulation or preparation. Optionally, compositions of the present invention further comprise one or more pharmaceutically acceptable surfactants, buffers, electrolytes, salts, carriers and/or excipients. Optical agents of the present invention include supramolecular structures and assemblies, including shell-cross-linked micelles, wherein a therapeutic agent is physically associated with or covalently linked to one or more of the blocks of the copolymers. In an embodiment, the optical agent of the present invention further comprises one or more therapeutic agents at least partially encapsulated by the supramolecular structure, such as a hydrophobic drug or combination of hydrophobic drugs, hydrophobic biologic agent, or hydrophobic phototherapeutic agent. The present invention includes, for example, optical agents wherein a therapeutic agent is non-covalently associated with the hydrophobic core. Therapeutic agents of this aspect of the present invention optionally include phototherapeutic agents, such as Type-1 or Type-2 phototherapeutic agents, or chemotherapy agents.

In another aspect, the present invention provides an optical imaging method. In this method, an effective amount of an optical agent of the present invention is administered to a mammal (e.g., a patient undergoing treatment). In this aspect, at least one photoactive moiety of the optical agent includes at least one chromophore and/or fluorophore, optionally capable of excitation via absorption of electromagnetic radiation having wavelengths in the visible region (e.g. 400 nm to 750 nm) and/or the near infrared region (e.g., 750-1300 nm). The optical agent that has been administered is exposed to electromagnetic radiation. Electromagnetic radiation transmitted, scattered or emitted by the optical agent is then detected. In some embodiments, fluorescence may be excited from the optical agent (e.g., due to the electromagnetic radiation exposure), optionally via multiphoton excitation processes. Use of electromagnetic radiation having wavelengths selected over the range of 400 nanometers to 1300 nanometers may be useful for some in situ optical imaging methods of the present invention, including biomedical applications for imaging organs, tissue and/or tumors, anatomical visualization, optical guided surgery and endoscopic procedures.

In another aspect, the present invention provides a method of providing photodynamic therapy. In this method, an effective amount of an optical agent of the present invention is administered to a mammal (e.g., a patient undergoing treatment). In this aspect, at least one photoactive moiety of the optical agent includes one or more phototherapeutic agents, optionally capable of excitation via absorption of electromagnetic radiation having wavelengths in the visible region (e.g. 400 nm to 750 nm) and/or the near infrared region (e.g., 750-1300 nm). The optical agent that has been administered is exposed to electromagnetic radiation. In some embodiments, the optical agent may be targeted to a selected organ, tissue or tumor site in the mammal, for example by incorporation of an appropriate targeting ligand in the optical agent. Use of electromagnetic radiation having wavelengths selected over the range of 400 nanometers to 1300 nanometers may be useful for some phototherapeutic treatment methods of the present invention. Exposure of the optical agent to electromagnetic radiation activates the phototherapeutic agent(s) causing, for example, release of the phototherapeutic agent and/or cleavage of one or more photolabile bonds of the phototherapeutic agent, thereby generating one or more reactive species (e.g., free radicals, ions etc.).

In another aspect, the present invention provides a method of monitoring a physiological state or condition. In this method, an effective amount of an optical agent of the present invention is administered to a mammal (e.g., a patient undergoing treatment). Further, the optical agent that has been administered is exposed to electromagnetic radiation. In addition, electromagnetic radiation transmitted, scattered or emitted by the optical agent is detected. In some embodiments, a change in the wavelengths or intensities of electromagnetic radiation emitted by the optical agent that has been administered to the mammal may be detected, measured and/or monitored as a function of time. In some embodiments, the hydrophilic block, hydrophobic block or both comprise(s) one or more functional groups responsive to pH, and wherein the supramolecular structure undergoes a change in structure in response to a change in a physiological condition or chemical environment that causes a measurable change in the intensities or wavelengths of electromagnetic radiation emitted by the optical agent administered to the mammal. In one embodiment, for example, the change in structure in response to the change in physiological condition or chemical environment quenches or enhances fluorescence of the optical agent, or alternatively changes the emission wavelengths of fluorescence of the optical agent. Methods of this aspect of the present invention include in situ pH monitoring methods and methods of monitoring renal function in the mammal, wherein the optical agent is cleared renally by the mammal.

In another aspect, the present invention provides a method for making an optical agent. In this method, a plurality of block copolymers are dissolved in organic solvents, an aqueous solution, or a mixture thereof, wherein each of the block copolymers comprises a hydrophilic block and a hydrophobic block, and wherein the block copolymers self-assemble in the aqueous solution to form a supramolecular structure, such as a micelle structure. The block copolymers of the supramolecular structure are then contacted with a cross linking reagent comprising one or more photoactive moieties, optionally contacted with a pyrazine-based amino cross linker such as a pyrazine-based diamino or tetraamino cross linker. Optionally, at least a portion of the monomers of the hydrophilic group comprise N-acryloxysuccinimide (NAS) monomers. Further, at least a portion of the hydrophilic blocks of the block copolymers of the supramolecular structure are cross-linked via linking groups generated from the cross linking reagent, thereby making the optical agent. In some embodiments, the block copolymers self-assemble in the aqueous solution to form a micelle structure, which is subsequently cross-linked to form a shell-cross-linked micelle. Optionally, the cross linking may be carried out via EDC coupling reactions or via photoinitiated cross linking reactions. Optionally, the cross linking may achieve an extent of cross linking of the hydrophilic blocks of the copolymers selected over the range of 1 to 75%, and optionally 20 to 75%. In some embodiments, the dissolving may be carried out at a pH greater than 7. In such embodiments, the pH of the block copolymers dissolved in the aqueous solution may be subsequently slowly decreased to a pH of about 7.

In another aspect, the invention provides an optical agent for use in a medical optical imaging procedure. In an embodiment, a procedure of the present invention comprises: (i) administering to a mammal an effective amount of the optical agent as described herein, wherein the one or more photoactive moieties comprise one or more chromophores and/or fluorophores; (ii) exposing the optical agent administered to the mammal to electromagnetic radiation; and (iii) detecting electromagnetic radiation transmitted, scattered or emitted by the optical agent.

In another aspect, the invention provides an optical agent for use in a medical photodynamic therapy procedure. In an embodiment, a procedure of the present invention comprises: (i) administering to a mammal an effective amount of the optical agent as described herein, wherein the one or more photoactive moieties comprise one or more phototherapeutic agents; and (ii) exposing the optical agent administered to the mammal to electromagnetic radiation.

In another aspect, the invention provides an optical agent for use in a medical procedure for monitoring a physiological state or condition. In an embodiment, a procedure of the present invention comprises: (i) administering to a mammal an effective amount of the optical agent as described herein; (ii) exposing the optical agent administered to the mammal to electromagnetic radiation; and (iii) detecting electromagnetic radiation transmitted, scattered or emitted by the optical agent.

In another aspect, the invention provides a shell-cross-linked micelle comprising: (i) cross-linked block copolymers, wherein each of the block copolymers comprises a poly(acrylic acid) polymer block directly or indirectly bonded to a hydrophobic block; and (ii) pyrazine-containing linking groups covalently cross linking at least a portion the poly(acrylic acid) polymer blocks of the block copolymers; wherein the pyrazine-containing linking groups are bound to monomers of the poly(acrylic acid) polymer block by carboxamide bonds. In an embodiment of this aspect, the mole ratio of the pyrazine-containing linking groups to monomers of the poly(acrylic acid) polymer block is selected over a range of 1:100 to 75:100.

### BRIEF DESCRIPTION OF THE FIGURES

FIG. 1 illustrates an example of SCK formation. Amphiphilic block copolymers self-assemble into micelles having a hydrophobic core. The block copolymers are then functionalized to form cross linking between the individual polymers. The cross linking of the copolymers forms a shell surrounding the hydrophobic core.

FIG. 2 provides examples of bifunctional optical probe moieties useful for photonic shell cross linking in the present methods and compositions.

FIG. 3 provides a schematic diagram illustrating a synthetic pathway for the formation of photonic shell containing SCKs via cross linking chemistry with a photonic linking group of the present invention.

FIG. 4A illustrates an exemplary photonic shell cross-linked nanoparticle structure.

FIG. 4B illustrates effects of raising and/or lowering the pH on a photonic shell cross-linked nanoparticle.

FIG. 5 shows assembly of micelles from poly(acrylic acid)-b-poly(p-hydroxystyrene) in water, with an adjustment of the solution pH, followed by the construction of pH-responsive SCKs upon shell cross-linking with fluorophores.

FIG. 6A shows a representative AFM image of a photonic SCK micelle of the present invention, having an average height of 8 nm. FIG. 6B shows the hydrodynamic diameter of 2 (left), 3 (middle), and 4 (right) as a function of pH. FIG. 6C shows normalized fluorescence emission of SCKs 3 and 4, and PAA/cross-linker as a function of pH. For each data set, the fluorescence intensity of the cross-linker as a small molecule is normalized to the value that would be observed for the cross-linker in solution at the concentration of cross-linker within the SCK shells.

FIG. 7 illustrates the swelling/deswelling of photonic SCKs as a function of pH.

FIG. 8 shows normalized fluorescence emission of SCKs 3 and 4 as a function of pH and fluorophore loading (left: 6.25 mol % pyrazine relative to acrylic acid residues, right: 12.5 mol % pyrazine relative to acrylic acid residues).

FIG. 9 depicts data showing the fluorescence measurements of II-a and II-b as a function of pH.

FIG. 10 depicts a synthetic scheme showing synthesis of Photonic Cross-Linker of Examples 1 and 2.

FIG. 11 depicts a synthetic scheme showing synthesis of Photonic Cross-Linker Example 3.

FIG. 12 depicts a synthetic scheme showing synthesis of Photonic Cross-Linker Example 4.

FIG. 13 depicts a synthetic scheme showing synthesis of Photonic Shell Cross-Linked Nanoparticle Example 5.

FIG. 14 depicts a synthetic scheme showing synthesis of Photonic Shell Cross-Linked Nanoparticle Example 6.

FIG. 15 depicts a synthetic scheme showing synthesis of Photonic Shell Cross-Linked Nanoparticle Example 7.

FIG. 16 depicts a synthetic scheme showing synthesis of Photonic Shell Cross-Linked Nanoparticle Example 8.

FIG. 17 depicts data showing the optical absorbance and fluorescence of Photonic Cross-Linker Example 2 as a function of pH.

FIG. 18 depicts data showing the optical absorbance and fluorescence of Shell Cross-Linked Nanoparticle Example 5 as a function of pH.

FIG. 19 depicts data showing the optical absorbance and fluorescence of Photonic Cross-Linker Example 3 as a function of pH.

FIG. 20 depicts data showing the optical absorbance and fluorescence of Shell Cross-Linked Nanoparticle Example 6 as a function of pH.

FIG. 21 depicts data showing the optical absorbance and fluorescence of Shell Cross-Linked Nanoparticle Example 7 as a function of pH.

FIG. 22 depicts data showing the optical absorbance and fluorescence of Shell Cross-Linked Nanoparticle Example 8 as a function of pH.

FIG. 23 provides TEM images of micelles generated from compounds 4 of Example 5 and vesicles generated from compound 5 of Example 5.

FIG. 24 provides a schematic showing construction of photophysically-functionalized, cross-linked multicompartment nanostructures.

FIG. 25 provides data and images showing characterization of MCNs prepared from PEO₄₅-b-PNAS₁₀₅-b-PS₄₅ precursors and cross-linked with cross linker 1 of FIG. 24 in pH 7.2 5 mM PBS buffer (with 5 mM of NaCl). Panels A-C and D-F show hydrodynamic diameter histograms as measured by DLS, TEM micrograph, and cryo-TEM micrograph of MCNs 4a and 4b of FIG. 24, respectively.

FIG. 26 provides data and images showing characterization of MCNs prepared from PEO₄₅-b-PNAS₁₀₅-b-PS₄₅ precursors and cross-linked with 2 of FIG. 24 in pH 7.2 5 mM PBS buffer (with 5 mM of NaCl). Panels A-C and D-F show hydrodynamic diameter histograms measured by DLS, TEM micrograph, and cryo-TEM micrograph of 5a and 5b of FIG. 24, respectively.

FIG. 27 provides data showing pH-responsive photo-physical properties of cross-linked MCNs. Panels A-B provide UV-Vis (top) and fluorescence emission spectra of MCNs prepared from cross-linking with cross linker 1 of FIG. 24 at nominal 20% and 50% cross-linking extents, respectively. Panels C-D provide UV-Vis (top) and fluorescence emission spectra of MCNs prepared from cross-linking with cross linker 2 of FIG. 24 at nominal 20% and 50% cross-linking extents, respectively.

FIG. 28 provides data showing characterizations of MCMs in DMF/H₂O (v:v=1:1). Panel A provides Intensity-average weighted (left) and number-average weighted (right) hydrodynamic diameter distribution of MCMs by DLS. Panel B provides TEM image of MCMs after 24 h of storage at room temperature (stained with PTA).

FIG. 29 provides data showing characterization of MCNs cross-linked/functionalized by 1, 4a and 4b of FIG. 24. Panel A provides DLS histograms of intensity-averaged hydrodynamic diameters for 4a (left) and 4b (right) in buffer solutions (5 mM with 5 mM of NaCl) at different pH values. Panel B provides TEM micrographs (stained negatively with PTA) of 4a collected after drop deposition onto carbon-coated copper grids from pH 5.8 (left), pH 7.2 (middle), and pH 8.6 (right) buffer solutions (5 mM with 5 mM of NaCl), respectively. Panel C provides TEM micrographs (stained negatively with PTA) of 4b collected after drop deposition onto carbon-coated copper grids from pH 5.8 (left), pH 7.2 (middle), and pH 8.6 (right) buffer solutions (5 mM with 5 mM of NaCl), respectively.

FIG. 30 provides data showing characterizations of MCNs 5a and 5b of FIG. 24 cross-linked/functionalized by 2 of FIG. 24. Panel A provides Histograms of intensity-averaged hydrodynamic diameter for 5a (left) and 5b (right) in buffer solutions (5 mM with 5 mM of NaCl) at different pH values. Panel B provides TEM micrographs (stained with PTA) of 5a in pH 5.8 (left), pH 7.2 (middle), and pH 8.6 (right) buffer solutions (5 mM with 5 mM of NaCl), respectively. Panel C provides TEM micrographs (stained with PTA) of 5b in pH 5.8 (left), pH 7.2 (middle), and pH 8.6 (right) buffer solutions (5 mM with 5 mM of NaCl), respectively.

FIG. 31 provides a structural stability comparison between MCMs and MCNs after nine months of storage in organic/aqueous media (DMF/H₂O, initial v:v=1:1). Panel A provides a TEM image of pre-established MCMs without any covalent stabilization (stained with PTA). Panels B and C provide TEM images of cross-linked 4a and 4b of FIG. 24, respectively (stained with PTA).

FIG. 32 provides UV-Vis (left) and fluorescence emission (right) spectra of small molecule cross-linkers 1 (panel A) and 2 (panel B) of FIG. 24 in buffer solutions (5 mM with 5 mM of NaCl) at the surveyed pH values.

FIG. 33 provides data showing acylation of 2 of FIG. 24 with NAS. Panel A provides a schematic drawing for the reaction of 2 with NAS. Panel B provides HPLC analyses of 3 (top) and the reaction mixture after 48 h (bottom). Panels C and D provide UV-Vis and fluorescence emission spectra of acylated 3 in buffer solutions (5 mM with 5 mM of NaCl) at the surveyed pH values, respectively.

FIG. 34 provides data relating to small MCMs assembled from PEO₄₅-b-P(NAS₉₅-co-AA₁₀)-b-PS₄₅ precursors. Panel A provides an ¹H NMR spectrum of PEO₄₅-b-P(NAS₉₅-co-AA₁₀)-b-PS₄₅ block copolymer precursor. Panel B provides intensity-average weighted (top) and number-average weighted (bottom) hydrodynamic diameter distributions of MCMs in DMF/H₂O (v:v=1:1) by DLS. Panel C provides a TEM image of MCMs in DMF/H₂O (v:v=1:1) after 24 h of storage at room temperature (stained with PTA).

FIG. 35 provides a comparison of emission spectra and photophysical properties of SCKs vs. shell-cross-linked rods.

FIG. 36 provides chemical structures for cross-linking chromophores A, B and C of Example 7.

FIG. 37 provides UV-vis absorbance (top) and fluorescence emission (bottom) spectra of SC-rods cross-linked with cross-linking chromophore A of Example 7 with 2%, 6% and 9% cross-linking density in the presence of stoichiometric amount of EDCl.

FIG. 38 provides UV-vis absorbance (top) and fluorescence emission (bottom) spectra of SC-rods cross-linked with cross-linking chromophore A of Example 7 with 2%, 5% and 9% cross-linking density in the presence of 2 molar excess EDCl.

FIG. 39 provides UV-vis absorbance (top) and fluorescence emission (bottom) spectra of SC-rods cross-linked with cross-linking chromophore B of Example 7 with 2%, 7% and 12% cross-linking density in the presence of stoichiometric amount of EDCl.

FIG. 40 provides UV-vis absorbance (top) and fluorescence emission (bottom) spectra of SC-rods cross-linked with cross-linking chromophore B of Example 7 with 2%, 7% and 10% cross-linking density in the presence of 2 molar excess EDCl.

FIG. 41 provides UV-vis absorbance (top) and fluorescence emission (bottom) spectra of SC-rods cross-linked with cross-linking chromophore C of Example 7 with 2%, 7% and 14% cross-linking density in the presence of stoichiometric amount of EDCl.

FIG. 42 provides UV-vis absorbance (top) and fluorescence emission (bottom) spectra of SC-rods cross-linked with cross-linking chromophore C of Example 7 with 2%, 6% and 3% cross-linking density in the presence of 2 molar excess EDCl.

FIG. 43 provides transmission electron micrograph (TEM) images of SCK A series of Example 7 with 50 molar excess EDCl.

FIG. 44 provides UV-vis absorbance (top) and fluorescence emission (bottom) spectra of SCKs cross-linked with cross-linking chromophore A of Example 7 with 1%, 5% and 10% cross-linking density in the presence of 2 molar excess EDCl.

FIG. 45 provides UV-vis absorbance (top) and fluorescence emission (center: λₑₓ 424 nm, bottom: λₑₓ 386 nm) spectra of SCKs cross-linked with cross-linking chromophore A of Example 7 with 2%, 8% and 14% cross-linking density in the presence of 35 molar excess EDCl.

FIG. 46 provides UV-vis absorbance (top) and fluorescence emission (center: λₑₓ 424 nm, bottom: λₑₓ 386 nm) spectra of SCKs cross-linked with cross-linking chromophore A of Example 7 with 2%, 7% and 13% cross-linking density in the presence of 75 molar excess EDCl.

FIG. 47 provides UV-vis absorbance (top) and fluorescence emission (center: λₑₓ 424 nm, bottom: λₑₓ 386 nm) spectra of SCKs cross-linked with cross-linking chromophore A of Example 7 with 2%, 7% and 13% cross-linking density in the presence of stoichiometric amount of EDCl.

FIG. 48 provides UV-vis absorbance (top) and fluorescence emission (center: λₑₓ 424 nm, bottom: λₑₓ 386 nm) spectra SCKs with 2, 7 or 13% cross-linking density (cross-linking chromophore A of Example 7) after reacting with additional stoichiometric amount of EDCl.

FIG. 49 provides a conjugation reaction scheme for conjugation of an SH-PEO₃ₖ block copolymer with the LCB peptide Ser-Phe-Phe-Tyr-Leu-Arg-Ser (SEQ. ID. NO. 1) for providing targeting functionality to block copolymers.

FIG. 50 provides a co-assembly reaction scheme for co-assembly of LCB-PEO₃ₖ/mPEO₂ₖ block copolymers wherein the LCB peptide is Ser-Phe-Phe-Tyr-Leu-Arg-Ser (SEQ. ID. NO. 1) for providing targeting functionality to block copolymers.

FIG. 51 provides a conjugation reaction scheme for conjugation of a PEO₄₅-b-PNAS₁₀₅ block copolymer with the LCB peptide Ser-Phe-Phe-Tyr-Leu-Arg-Ser (SEQ. ID. NO. 1) for providing targeting functionality to block copolymers.

FIG. 52 provides a scheme for producing multicompartment nanostructures and nanoparticles from PEO-b-PNAS-b-PS block copolymers. FIG. 52 also provides an image of the multicompartment nanostructures and nanoparticles produced by this method.

FIG. 53 provides fluorescence data for multicompartment nanostructures produced by the scheme of FIG. 52, wherein m is 105 and n is 45. The fluorescence data is presented for pH 5.9, 6.5, 7.2, 7.7, and 8.4. FIG. 53 also provides an image showing the multicompartment nanostructures produced by the scheme of FIG. 52, wherein m is 105 and n is 45.

FIG. 54 provides fluorescence data for nanoparticles produced by the scheme of FIG. 52, wherein m is 50 and n is 30. The fluorescence data is presented for pH 5.9, 6.5, 7.2, 7.7, and 8.4. FIG. 54 also provides an image showing the nanoparticles produced by the scheme of FIG. 52, wherein m is 50 and n is 30.

FIG. 55 provides chemical structures of chromophoric cross-linkers A, B and C of Example 9, block copolymers and schematic illustrations of the respective shell-cross-linked nano-objects.

FIG. 56 provides plots showing normalized fluorescence emission spectra of SCR-A of Example 9 at 2%, 6% and 10% (left, middle, right) cross-linking density with the addition of 0, stoichiometric amount and 2 molar excess amount of EDCl (top, middle, bottom).

FIG. 57 provides a schematic representation of SCR-A of Example 9 having two distinct local environments: End-caps mimic the environment found in the spherical polymer assemblies while the linear portion of the rods display an opportunity to engage the anilino amine of the chromophoric cross-linker in acylation reactions to impart blue-shifted fluorescence emission.

FIG. 58 provides plots showing the relationship between solution pH and the fluorescence intensity ratio at two fixed wavelengths (496 nm and 560 nm) for SCR-A, SCR-B and SCR-C of Example 9 (left, middle, right, respectively) with stoichiometric (top) or 2 molar excess amounts of EDCl (bottom). The excitation wavelength was 433 nm.

FIG. 59 provides TEM images of SCR-A2% (left), SCR-A 6% (middle), and SCR-A 10% (right) of Example 9 at pH 4.6 (top) and pH 7.4 (bottom).

FIG. 60 provides plots showing the relationship between solution pH and the fluorescence intensity ratio at two fixed wavelengths (496 nm and 560 nm) for SCK-A of Example 9 with stoichiometric, 35 molar excess and 75 molar excess amount of EDCl (left, middle, right). The excitation wavelength was either 433 nm or 386 nm.

FIG. 61 provides plots showing the relationship between solution pH and the fluorescence intensity ratio at two fixed wavelengths (496 nm and 560 nm) for SCKs of Example 9 with one and two cycles of shell-cross-linking reactions by addition of stoichiometric amounts of EDCl during each cycle (left, right). The excitation wavelength was either 433 nm or 386 nm.

FIG. 62 provides bar graphs showing the relationship between solution pH and the fluorescence intensity ratio at two fixed wavelengths (496 nm and 560 nm) for sc-SCK-A, sc-SCK-B, Ic-SCK-A and Ic-SCK-B of Example 9 (left, excited at 433 nm, the maximum absorbance wavelength of A and B; right, excited at 386 nm, the maximum absorbance wavelength of the nanostructures).

FIG. 63 provides a schematic representation of the core-shell interface of SCR-A of Example 9 showing hydrogen bonding and phenyl esters between core and shell functionalities (area A) to form arylamino amide derivatives at high pH (area B) as well as the desired cross-linking adduct (area C).

FIG. 64 provides a scheme for construction of photophysically-functionalized MCNs of Example 10 by supramolecular assembly of triblock terpolymers in solution followed by cross-linking with chromophores.

FIG. 65 provides data showing characterization of MCMs of Example 10 in DMF/H₂O (v:v=1:1, polymer concentrations 0.5 mg/mL). Panel A) provides an intensity-average weighted (top) and number-average weighted (bottom) hydrodynamic diameter distribution of “as prepared” MCMs by DLS (the scale of x-axis is logarithmic). Panel B) provides a TEM image (collected after drop deposition onto carbon-coated copper grids) of “as prepared” MCMs after 24 h of storage at room temperature (stained negatively with PTA). Panel C) provides a TEM image of “as prepared” MCMs without any covalent stabilization after 3 months of storage at room temperature (stained negatively with PTA). Panel D) provides a TEM image of “as prepared” MCMs without any covalent stabilization after 9 months of storage at room temperature (stained negatively with PTA).

FIG. 66 provides data showing characterization of small MCMs of Example 10 assembled from PEO₄₅-b-P(NAS₉₅-co-AA₁₀)-b-PS₄₅ precursors in DMF/H₂O (v:v=1:1, polymer concentrations were ˜0.5 mg/mL). Panel A) provides an intensity-average weighted (top) and number-average weighted (bottom) hydrodynamic diameter distribution of “as prepared” MCMs by DLS (the scale of x-axis was presented by logarithmic). Panel B) provides a TEM image (collected after drop deposition onto carbon-coated copper grids) of “as prepared” MCMs after 24 h of storage at room temperature (stained negatively with PTA).

FIG. 67 provides data showing characterization of MCNs of Example 10 prepared from PEO₄₅-b-PNAS₁₀₅-b-PS₄₅ precursors and cross-linked with 2 in pH 7.2, 5 mM PBS buffer (with 5 mM of NaCl, polymer concentrations were 0.2-0.3 mg/mL). Panels A-C) and D-F) show hydrodynamic diameter histograms as measured by DLS (the scale of x-axis is logarithmic), TEM micrograph (stained negatively with PTA), and cryogenic TEM micrograph of compounds 4a and 4b, respectively.

FIG. 68 provides data showing characterization of MCNs 4a and 4b of Example 10 (polymer concentrations were 0.2-0.3 mg/mL) cross-linked/functionalized by 2. Panel A) provides DLS histograms of intensity-averaged hydrodynamic diameters for 4a (left) and 4b (right) in buffer solutions (5 mM with 5 mM of NaCl) at different pH values. Panel B) provides high-resolution TEM micrographs (stained negatively with PTA) of 4a collected after drop deposition onto carbon-coated copper grids from pH 5.8 (left), pH 7.2 (middle), and pH 8.6 (right) buffer solutions (5 mM with 5 mM of NaCl), respectively. Panel C) provides high-resolution TEM micrographs (stained negatively with PTA) of 4b collected after drop deposition onto carbon-coated copper grids from pH 5.8 (left), pH 7.2 (middle), and pH 8.6 (right) buffer solutions (5 mM with 5 mM of NaCl), respectively.

FIG. 69 provides data showing characterization of MCNs of Example 10 prepared from PEO₄₅-b-PNAS₁₀₅-b-PS₄₅ precursors and cross-linked with 3 in pH 7.2 5 mM PBS buffer (with 5 mM of NaCl, polymer concentrations were 0.2-0.3 mg/mL). Panels A-C) and D-F) provide hydrodynamic diameter histograms measured by DLS (the scale of x-axis was presented by logarithmic), TEM micrograph (stained negatively with PTA), and cryo-TEM micrograph of 5a and 5b, respectively.

FIG. 70 provides data showing characterization of MCNs 5a and 5b of Example 10 (polymer concentrations were 0.2-0.3 mg/mL) cross-linked/functionalized by 3. Panel A) provides DLS histograms of intensity-averaged hydrodynamic diameters for 5a (left) and 5b (right) in buffer solutions (5 mM with 5 mM of NaCl) at different pH values. Panel B) provides high-resolution TEM micrographs (stained negatively with PTA) of 5a collected after drop deposition onto carbon-coated copper grids from pH 5.8 (left), pH 7.2 (middle), and pH 8.6 (right) buffer solutions (5 mM with 5 mM of NaCl), respectively. Panel C) provides high-resolution TEM micrographs (stained negatively with PTA) of 5b collected after drop deposition onto carbon-coated copper grids from pH 5.8 (left), pH 7.2 (middle), and pH 8.6 (right) buffer solutions (5 mM with 5 mM of NaCl), respectively.

FIG. 71 provides tapping mode AFM images of MCNs of Example 10 in water (polymer concentrations were 0.2-0.3 mg/mL). Panel A) provides height (top) and phase (bottom) images of 4a on mica. Panel B) provides height (top) and phase (bottom) images of 4b on mica. Panel C) provides height (top) and phase (bottom) images of 5a on mica. Panel D) provides height (top) and phase (bottom) images of 5b on mica. The AFM samples were prepared by spin casting the corresponding MCN solution in water on freshly cleaved mica.

FIG. 72 provides a plot showing SAXS profiles of MCNs of Example 10 in pH 7.2 PBS buffer solutions (5 mM with 5 mM of NaCl). Unmarked arrows point to the positions of Bragg peaks corresponding to the internal order within the MCNs, while arrows with asterisks (*) mark the positions of possible form factor peaks associated with the overall size of the MCNs.

FIG. 73 provides plots showing pH-responsive photo-physical properties of cross-linked MCNs of Example 10. Panels A-B) provides UV-Vis (left) and fluorescence emission spectra (middle, excitation at λ_{abs,max} of MCNs, solid line; right, excitation at 433 nm, dashed line) of MCNs prepared from cross-linking with 2 at nominal 20% and 50% cross-linking extents, respectively. Panels C-D) provide UV-Vis (left) and fluorescence emission spectra (middle, excitation at λ_{abs,max} of MCNs, solid line; right, excitation at 433 nm, dashed line) of MCNs prepared from cross-linking with 3 at nominal 20% and 50% cross-linking extents, respectively.

FIG. 74 provides plots showing photo-physical properties of photonic SCK nanoparticles of Example 10. Panels A-B) provide UV-Vis (left) and fluorescence emission spectra (middle, excitation at λ_{abs,max} of SCKs, solid line; right, excitation at 433 nm, dashed line) of SCK 4a and SCK 5a, prepared from cross-linking with 2 and 3 at nominal 20% cross-linking extents, respectively. Panel C) provides fluorescence emissions of MCNs and SCKs as a function of environmental pH values (the y-axis is presented by the ratio between 495 nm emission intensity and 555 nm emission intensity).

FIG. 75 provides data showing characterization of MCNs 4a and 4b of Example 10, polymer concentrations were 0.2-0.3 mg/mL, cross-linked/functionalized by 2. Panel A) provides TEM micrographs (stained negatively with PTA) of 4a collected after drop deposition onto carbon-coated copper grids from pH 5.8 (left), pH 7.2 (middle), and pH 8.6 (right) buffer solutions (5 mM with 5 mM of NaCl), respectively. Panel B) provides TEM micrographs (stained negatively with PTA) of 4b collected after drop deposition onto carbon-coated copper grids from pH 5.8 (left), pH 7.2 (middle), and pH 8.6 (right) buffer solutions (5 mM with 5 mM of NaCl), respectively.

FIG. 76 provides data showing characterization of MCNs 5a and 5b of Example 10, polymer concentrations were 0.2-0.3 mg/mL, cross-linked/functionalized by 3. Panel A) provides TEM micrographs (stained negatively with PTA) of 5a collected after drop deposition onto carbon-coated copper grids from in pH 5.8 (left), pH 7.2 (middle), and pH 8.6 (right) buffer solutions (5 mM with 5 mM of NaCl), respectively. Panel B) provides TEM micrographs (stained negatively with PTA) of 5b collected after drop deposition onto carbon-coated copper grids from in pH 5.8 (left), pH 7.2 (middle), and pH 8.6 (right) buffer solutions (5 mM with 5 mM of NaCl), respectively.

FIG. 77 provides data showing structural stability of MCNs of Example 10 after nine months of storage in organic/aqueous media (DMF/H₂O, initial v:v=1:1). Panels A-B) provide TEM images of cross-linked MCNs 4a and 4b, respectively (stained negatively with PTA).

FIG. 78 provides plots showing UV-Vis (left) and fluorescence emission (right) spectra of small molecule cross-linker 2 (panel A) and 3 (panel B) of Example 10 in buffer solutions (5 mM with 5 mM of NaCl) at the surveyed pH values.

FIG. 79 provides data showing acylation of compound 3 of Example 10 with NAS. Panel A) provides a schematic for the reaction of 3 with NAS. Panel B) provides a plot showing HPLC analyses of 3 (top) and the reaction mixture after 48 h (bottom). Panels C-D) provide UV-Vis and fluorescence emission spectra of acylated 3 in buffer solutions (5 mM with 5 mM of NaCl) at the surveyed pH values, respectively.

FIG. 80 provides data showing characterization of SCKs 4a of Example 10 (polymer concentrations were 0.2-0.3 mg/mL) cross-linked/functionalized by 2 at nominal 20% of cross-linking extents. Panels A-C) provide DLS histograms (top, the scale of x-axis was presented by logarithmic) of intensity- and number-averaged hydrodynamic diameters and TEM micrographs (bottom, stained negatively with PTA) of 4a in pH 5.8 (panel A), pH 7.2 (panel B), and pH 8.6 (panel C) buffer solutions (5 mM with 5 mM of NaCl), respectively.

FIG. 81 provides data showing characterization of SCKs 5a of Example 10 (polymer concentrations were 0.2-0.3 mg/mL) cross-linked/functionalized by 3 at nominal 20% cross-linking extents. Panels A-C) provide DLS histograms (top, the scale of x-axis was presented by logarithmic) of intensity- and number-averaged hydrodynamic diameters and TEM micrographs (bottom, stained negatively with PTA) of 5a in pH 5.8 (panel A), pH 7.2 (panel B), and pH 8.6 (panel C) buffer solutions (5 mM with 5 mM of NaCl), respectively.

### DETAILED DESCRIPTION

In general the terms and phrases used herein have their art-recognized meaning, which can be found by reference to standard texts, journal references and contexts known to those skilled in the art. The following definitions are provided to clarify their specific use in the context of the invention.

“Optical agent” generally refers to compositions, preparations and/or formulations for coupling electromagnetic radiation into and/or out of an environment and/or sample. For some applications, for example, the present optical agents are administered to a biological environment or sample, such as a patient, mammal, an organ, tissue, tumor, tumor site, excised tissue or cell material, cell extract, fluid and/or biological fluid, colloid and/or suspension, for coupling electromagnetic radiation into and/or out of a biological sample. In some embodiments, optical agents of the present invention absorb, transmit and/or scatter electromagnetic radiation provided to a biologic sample and/or biological environment. In some embodiments, optical agents of the present invention are excited by electromagnetic radiation provided to a biologic sample and/or biological environment, and emit electromagnetic radiation via fluorescence, phosphorescence, chemiluminescence and/or photoacoustic processes. In some embodiments, optical agents of the present invention absorb electromagnetic radiation provided to a biologic sample and/or biological environment, and become activated, for example via photofragmentation or other a photoinitiated chemical reaction, including photocleavage of one or more photolabile bonds or photofragmentation to generate reactive species such as nitrenes, carbene, free radicals and/or ions. In some embodiments, optical agents of the present invention absorb electromagnetic radiation provided to a biologic sample and/or biological environment and radiatively or non-radiatively transfer at least a portion of the absorbed energy to a moiety, molecule, complex or assembly in proximity.

Optical agents of the present invention include, but are not limited to, contrast agents, imaging agents, dyes, photosensitizer agents, photoactivators, and photoreactive agents; and conjugates, complexes, bioconjugates, and derivatives thereof. Optical agents of the present invention include photonic nanostructures and nanoassemblies including supramolecular structures, such as micelles, shell-cross-linked micelles, vesicles, bilayers, folded sheets and tubular micelles, that incorporate at least one linking group comprising a photoactive moiety, such as a fluorophores, chromophores, photosensitizers, and photoreactive moieties.

“Supramolecular structure” refers to structures comprising an assembly of molecules that are covalently linked, physically associated or both covalently linked, and physically associated. Supramolecular structures include assemblies of molecules, such as amphiphilic polymers, including block copolymers having a hydrophilic block and hydrophobic group. In some supramolecular structures of the present invention, hydrophilic portions of the block copolymers are oriented outward toward a continuous aqueous phase and form a hydrophilic shell or corona phase, and hydrophobic portions of the block copolymers are oriented inward and form a hydrophobic inner core. Supramolecular structures of the present invention include, but are not limited to, rods, micelles, vesicles, bilayers, folded sheets, tubular micelles, toroidal micelles and discoidal micelles. Supramolecular structures of the present invention include self-assembled structures. Supramolecular structures include cross-linked structures, such as shell-cross-linked micelle structures.

“Polymer” refers to a molecule comprising a plurality of repeating chemical groups, typically referred to as monomers. Polymers may include any number of different monomer types provided in a well-defined sequence or random distribution. A “copolymer”, also commonly referred to as a heteropolymer, is a polymer formed when two or more different types of monomers are linked in the same polymer. “Block copolymers” are a type of copolymer comprising blocks or spatially segregated domains, wherein different domains comprise different polymerized monomers having different compositions, chemical properties and/or physical properties. In a block copolymer, adjacent blocks are constitutionally different, i.e. adjacent blocks comprise constitutional units derived from different species of monomer or from the same species of monomer but with a different composition or sequence distribution of constitutional units. Different blocks (or domains) of a block copolymer may reside on different ends or the interior of a polymer (e.g. [A][B]), or may be provide in a selected sequence ([A][B][A][B]). “Diblock copolymer” refers to block copolymer having two different polymer blocks. “Triblock copolymer” refers to block copolymer having three different polymer blocks. “Polyblock copolymer” refers to block copolymer having at least two different polymer blocks, such as two, three, four, five etc. different polymer blocks. Optical agents of the present invention include supramolecular structures comprising diblock copolymers, triblock copolymers and polyblock copolymers. Optionally, block copolymers of the present invention comprise a PEG block (i.e., (CH₂CH₂O)_{b}—).

“Photoactive moiety” generally refers to a component of a molecule having optical functionality. Photoactive moieties include, for example, functional groups and substituents that function as a fluorophore, a chromophore, a photosensitizer, and/or a photoreactive moiety in the present compositions and methods. Photoactive moieties are capable of undergoing a number of processes upon absorption of electromagnetic radiation including fluorescence, activation, cleavage of one or more photolabile bonds and energy transfer processes. Photoreactive in this context refers to compositions and components thereof that are activated by absorption of electromagnetic radiation and, subsequently undergo chemical reaction or energy transfer processes. The present invention includes optical agents comprising supramolecular structures, such as shell cross-linked micelles, having linking groups comprising photoactive moieties that are excited upon absorption of electromagnetic radiation having wavelengths in the near UV region (e.g., 200 nm to 400 nm), visible region (e.g. 350 nm to 750 nm), and/or the near infrared region (e.g., 750-1300 nm).

As used herein “hydrophilic” refers to molecules and/or components (e.g., functional groups, monomers of block polymers etc.) of molecules having at least one hydrophilic group, and hydrophobic refers to molecules and/or components (e.g., functional groups of polymers, and monomers of block copolymers etc.) of molecules having at least one hydrophobic group. Hydrophilic molecules or components thereof tend to have ionic and/or polar groups, and hydrophobic molecules or components thereof tend to have nonionic and/or nonpolar groups. Hydrophilic molecules or components thereof tend to participate in stabilizing interactions with an aqueous solution, including hydrogen boding and dipole-dipole interactions. Hydrophobic molecules or components tend not to participate in stabilizing interactions with an aqueous solution and, thus often cluster together in an aqueous solution to achieve a more stable thermodynamic state. In the context of block copolymer of the present invention, a hydrophilic block is more hydrophilic than a hydrophobic group of an amphiphilic block copolymer, and a hydrophobic group is more hydrophobic than a hydrophilic block of an amphiphilic polymer.

As used herein, the term “fluorescence intensity ratio” refers to a ratio of fluorescence intensities, wherein the fluorescence intensities are measured at two different wavelengths. The term “reference fluorescence intensity ratio” refers to a measured fluorescence intensity ratio for a specific compound at a known pH value. The term “local maximum” in the context of fluorescence intensity refers to a local maximum of a fluorescence intensity spectrum which can correspond to a local peak maximum intensity and/or a local shoulder maximum intensity. The term “integrated intensity” in the context of fluorescence intensity refers to the integrated intensity of a local maximum peak or shoulder in a fluorescence intensity spectrum. An integrated fluorescence intensity can be calculated for one or more local maxima by modeling and de-convoluting a measured fluorescence intensity spectrum.

As used herein, the term “animal” refers to a major group of mostly multicellular, eukaryotic organisms of the kingdom Animalia or Metazoa, as are known to those in the art. The term “animal” encompasses mammals and humans.

As used herein, the term “water source” refers to any source of water meant for animal and/or human consumption. Examples of water sources include, but are not limited to, municipal water supplies, natural springs, rivers, lakes, reservoirs, etc.

As used herein the term “electromagnetic radiation source” refers to any device or collection of devices which produces electromagnetic radiation. Electromagnetic radiation sources of the present invention can produce electromagnetic radiation which is polarized. Electromagnetic radiation sources of the present invention can produce electromagnetic radiation which is linearly, elliptically or circularly polarized. Electromagnetic radiation sources of the present invention can be lasers. Examples of lasers which can be used with the devices and methods of the present invention include, but are not limited to, a gas laser, a chemical laser, an eximer laser, a solid-state laser, a photonic crystal laser, a semiconductor laser, a dye laser, and a free electron laser. Electromagnetic radiation sources of the present invention can be configured to generate electromagnetic radiation emission, such as fluorescence, from an optical agent in a fluid.

As used herein the term “detector” refers to any element capable of detecting electromagnetic radiation, such as fluorescence from an optical agent. Detectors of the present invention can produce a signal corresponding to the electromagnetic radiation which contacts the detector. In some embodiments, this signal can be read by a processor (such as a personal computer) or other recording device. Electromagnetic radiation detectors of the present invention can be two-dimensional detectors capable of detecting electromagnetic radiation which has been dispersed onto the detector. Electromagnetic detectors of the present invention can comprise, but are not limited to, a CCD, a CMOS, a MOS, an active pixel sensor, a microchannel plate, a photoconductive film, an LED, a fiber optic, a photodiode, a photomultiplier tube, a phototransistor, a photoelectric sensor, a photoionization detector, a photomultiplier, or a photoresistor.

As used herein the term “electromagnetic radiation delivery system” refers to optical elements configured to deliver electromagnetic radiation from an electromagnetic radiation source to a sample or subject, such as a fluid containing an optical agent or a fluid of a subject to which an optical agent has been administered. Similarly, the term “electromagnetic radiation collection system” refers to optical elements configured to collect at least a portion of electromagnetic radiation emitted from a sample or subject, such as a fluid containing an optical agent or a fluid of a subject to which an optical agent has been administered.

As used herein the term “processor” refers to any device capable of receiving, storing, and manipulating a signal communicated to it. Processors of the present invention are also capable of being programmed to send control signals to elements of the invention to control those elements. A processor can be programmed, for example, to control an electromagnetic radiation source, a sample holder, and/or an electromagnetic radiation detector. Processors of the present invention can comprise, but are not limited to, personal computers configured to interact with components of the invention, as are well-known in the art.

“Targeting ligand” refers to a component that provides targeting and/or molecular recognition functionality. Targeting ligands useful in the present compositions and methods include one or more biomolecules or bioactive molecules, and fragments and/or derivatives thereof, such as hormones, amino acids, peptides, peptidomimetics, proteins, nucleosides, nucleotides, nucleic acids, enzymes, carbohydrates, glycomimetics, lipids, albumins, mono- and polyclonal antibodies, receptors, inclusion compounds such as cyclodextrins, and receptor binding molecules. Some examples of targeting peptides are described in WO/2008/108941, which is expressly incorporated by reference herein. Specific targeting ligands include peptides known in the art for targeting, such as the leukemia cell binding peptide Ser-Phe-Phe-Tyr-Leu-Arg-Ser (SFFYLRS, SEQ. ID. NO. 1). Other examples of targeting ligands are provided, for example, in: Rossin et al., Journal of Nuclear Medicine, Vol. 46, No. 7, July 2005, pg. 1210; Zhang et al., Journal of Polymer Science: Part A: Polymer Chemistry, Vol. 46, 2008, pg. 7578; Liu et al., Biomarcromolecules, 2001, 2, 362-368; Zhang et al., Bioconjugate Chemistry, Vol. 19, No. 9, 2008, pg. 1880; each of which is expressly incorporated by reference herein.

When used herein, the term “diagnosis”, “diagnostic” and other root word derivatives are as understood in the art and are further intended to include a general monitoring, characterizing and/or identifying a state of health or disease. The term is meant to encompass the concept of prognosis. For example, the diagnosis of cancer can include an initial determination and/or one or more subsequent assessments regardless of the outcome of a previous finding. The term does not necessarily imply a defined level of certainty regarding the prediction of a particular status or outcome.

As defined herein, “contacting” means that a compound used in the present invention is provided such that is capable of making physical contact with another element, such as a microorganism, a microbial culture or a substrate. In another embodiment, the term “contacting” means that the compound used in the present invention is introduced into a subject receiving treatment, and the compound is allowed to come in contact in vivo.

As used herein, the term “cross-linking density” refers to the amount of covalently incorporated cross-linkers in a nanostructure network disclosed herein.

### ABBREVIATIONS

AFM Atomic Force Microscopy

Arg Arginine

DMF Dimethyl formamide

DLS Dynamic Light Scattering

DP Degree of Polymerization

Dz Intensity averaged hydrodynamic diameter

Dn Number averaged hydrodynamic diameter

EDC-HCl 1-(3-Dimethylaminopropyl)-3-ethylcarbodiimide hydrochloride

ESI Electrospray Ionization

EtOAc Ethyl Acetate

GPC Gel Permeation Chromatography

HOBt-H2O 1-Hydroxybenzotriazole hydrate

HRMS High Resolution Mass Spectrometry

IR Infrared Spectroscopy

LCMS Liquid Chromatography-Mass Spectrometry

MeOH Methanol

Mn Number Average Molecular Weight

MS Mass Spectrometry

MWCO Molecular Weight Cut-Off

NMR Nuclear Magnetic Resonance Spectroscopy

PBS Phosphate Buffered Saline

PDI Polydispersity Index

PMA Phosphomolybdic acid stain

PTA Phosphotungstic acid stain

SCK Shell Cross-Linked Nanoparticle

TEA Triethylamine

TEM Transmission Electron Microscopy

TFA Trifluoroacetic acid

THF Tetrahydrofuran

TLC Thin Layer Chromatography

Alkyl groups include straight-chain, branched and cyclic alkyl groups. Alkyl groups include those having from 1 to 30 carbon atoms. Alkyl groups include small alkyl groups having 1 to 3 carbon atoms. Alkyl groups include medium length alkyl groups having from 4-10 carbon atoms. Alkyl groups include long alkyl groups having more than 10 carbon atoms, particularly those having 10-30 carbon atoms. Cyclic alkyl groups include those having one or more rings. Cyclic alkyl groups include those having a 3-, 4-, 5-, 6-, 7-, 8-, 9- or 10-member carbon ring and particularly those having a 3-, 4-, 5-, 6-, or 7-member ring. The carbon rings in cyclic alkyl groups can also carry alkyl groups. Cyclic alkyl groups can include bicyclic and tricyclic alkyl groups. Alkyl groups are optionally substituted. Substituted alkyl groups include among others those which are substituted with aryl groups, which in turn can be optionally substituted. Specific alkyl groups include methyl, ethyl, n-propyl, iso-propyl, cyclopropyl, n-butyl, s-butyl, t-butyl, cyclobutyl, n-pentyl, branched-pentyl, cyclopentyl, n-hexyl, branched hexyl, and cyclohexyl groups, all of which are optionally substituted. Substituted alkyl groups include fully halogenated or semihalogenated alkyl groups, such as alkyl groups having one or more hydrogens replaced with one or more fluorine atoms, chlorine atoms, bromine atoms and/or iodine atoms. Substituted alkyl groups include fully fluorinated or semifluorinated alkyl groups, such as alkyl groups having one or more hydrogens replaced with one or more fluorine atoms. An alkoxyl group is an alkyl group linked to oxygen and can be represented by the formula R—O.

Alkenyl groups include straight-chain, branched and cyclic alkenyl groups. Alkenyl groups include those having 1, 2 or more double bonds and those in which two or more of the double bonds are conjugated double bonds. Alkenyl groups include those having from 2 to 20 carbon atoms. Alkenyl groups include small alkenyl groups having 2 to 3 carbon atoms. Alkenyl groups include medium length alkenyl groups having from 4-10 carbon atoms. Alkenyl groups include long alkenyl groups having more than 10 carbon atoms, particularly those having 10-20 carbon atoms. Cyclic alkenyl groups include those having one or more rings. Cyclic alkenyl groups include those in which a double bond is in the ring or in an alkenyl group attached to a ring. Cyclic alkenyl groups include those having a 3-, 4-, 5-, 6-, 7-, 8-, 9- or 10-member carbon ring and particularly those having a 3-, 4-, 5-, 6- or 7-member ring. The carbon rings in cyclic alkenyl groups can also carry alkyl groups. Cyclic alkenyl groups can include bicyclic and tricyclic alkyl groups. Alkenyl groups are optionally substituted. Substituted alkenyl groups include among others those which are substituted with alkyl or aryl groups, which groups in turn can be optionally substituted. Specific alkenyl groups include ethenyl, prop-1-enyl, prop-2-enyl, cycloprop-1-enyl, but-1-enyl, but-2-enyl, cyclobut-1-enyl, cyclobut-2-enyl, pent-1-enyl, pent-2-enyl, branched pentenyl, cyclopent-1-enyl, hex-1-enyl, branched hexenyl, cyclohexenyl, all of which are optionally substituted. Substituted alkenyl groups include fully halogenated or semihalogenated alkenyl groups, such as alkenyl groups having one or more hydrogens replaced with one or more fluorine atoms, chlorine atoms, bromine atoms and/or iodine atoms. Substituted alkenyl groups include fully fluorinated or semifluorinated alkenyl groups, such as alkenyl groups having one or more hydrogens replaced with one or more fluorine atoms.

Aryl groups include groups having one or more 5- or 6-member aromatic or heteroaromatic rings. Aryl groups can contain one or more fused aromatic rings. Heteroaromatic rings can include one or more N, O, or S atoms in the ring. Heteroaromatic rings can include those with one, two or three N, those with one or two 0, and those with one or two S, or combinations of one or two or three N, O or S. Aryl groups are optionally substituted. Substituted aryl groups include among others those which are substituted with alkyl or alkenyl groups, which groups in turn can be optionally substituted. Specific aryl groups include phenyl groups, biphenyl groups, pyridinyl groups, and naphthyl groups, all of which are optionally substituted. Substituted aryl groups include fully halogenated or semihalogenated aryl groups, such as aryl groups having one or more hydrogens replaced with one or more fluorine atoms, chlorine atoms, bromine atoms and/or iodine atoms. Substituted aryl groups include fully fluorinated or semifluorinated aryl groups, such as aryl groups having one or more hydrogens replaced with one or more fluorine atoms.

Arylalkyl groups are alkyl groups substituted with one or more aryl groups wherein the alkyl groups optionally carry additional substituents and the aryl groups are optionally substituted. Specific alkylaryl groups are phenyl-substituted alkyl groups, e.g., phenylmethyl groups. Alkylaryl groups are alternatively described as aryl groups substituted with one or more alkyl groups wherein the alkyl groups optionally carry additional substituents and the aryl groups are optionally substituted. Specific alkylaryl groups are alkyl-substituted phenyl groups such as methylphenyl. Substituted arylalkyl groups include fully halogenated or semihalogenated arylalkyl groups, such as arylalkyl groups having one or more alkyl and/or aryl having one or more hydrogens replaced with one or more fluorine atoms, chlorine atoms, bromine atoms and/or iodine atoms.

Optional substitution of any alkyl, alkenyl and aryl groups includes substitution with one or more of the following substituents: halogens, —CN, —COOR, —OR, —COR, —OCOOR, —CON(R)₂, —OCON(R)₂, —N(R)₂, —NO₂, —SR, —SO₂R, —SO₂N(R)₂ or —SOR groups. Optional substitution of alkyl groups includes substitution with one or more alkenyl groups, aryl groups or both, wherein the alkenyl groups or aryl groups are optionally substituted. Optional substitution of alkenyl groups includes substitution with one or more alkyl groups, aryl groups, or both, wherein the alkyl groups or aryl groups are optionally substituted. Optional substitution of aryl groups includes substitution of the aryl ring with one or more alkyl groups, alkenyl groups, or both, wherein the alkyl groups or alkenyl groups are optionally substituted.

Optional substituents for alkyl, alkenyl and aryl groups include among others:

—COOR where R is a hydrogen or an alkyl group or an aryl group and more specifically where R is methyl, ethyl, propyl, butyl, or phenyl groups all of which are optionally substituted;

—COR where R is a hydrogen, or an alkyl group or an aryl groups and more specifically where R is methyl, ethyl, propyl, butyl, or phenyl groups all of which groups are optionally substituted;

—CON(R)₂ where each R, independently of each other R, is a hydrogen or an alkyl group or an aryl group and more specifically where R is methyl, ethyl, propyl, butyl, or phenyl groups all of which groups are optionally substituted; R and R can form a ring which may contain one or more double bonds;

—OCON(R)₂ where each R, independently of each other R, is a hydrogen or an alkyl group or an aryl group and more specifically where R is methyl, ethyl, propyl, butyl, or phenyl groups all of which groups are optionally substituted; R and R can form a ring which may contain one or more double bonds;

—N(R)₂ where each R, independently of each other R, is a hydrogen, or an alkyl group, acyl group or an aryl group and more specifically where R is methyl, ethyl, propyl, butyl, or phenyl or acetyl groups all of which are optionally substituted; or R and R can form a ring which may contain one or more double bonds.

—SR, —SO₂R, or —SOR where R is an alkyl group or an aryl groups and more specifically where R is methyl, ethyl, propyl, butyl, phenyl groups all of which are optionally substituted; for —SR, R can be hydrogen;

—OCOOR where R is an alkyl group or an aryl groups;

—SO₂N(R)₂ where R is a hydrogen, an alkyl group, or an aryl group and R and R can form a ring;

—OR where R═H, alkyl, aryl, or acyl; for example, R can be an acyl yielding —OCOR* where R* is a hydrogen or an alkyl group or an aryl group and more specifically where R* is methyl, ethyl, propyl, butyl, or phenyl groups all of which groups are optionally substituted;

Specific substituted alkyl groups include haloalkyl groups, particularly trihalomethyl groups and specifically trifluoromethyl groups. Specific substituted aryl groups include mono-, di-, tri, tetra- and pentahalo-substituted phenyl groups; mono-, di-, tri-, tetra-, penta-, hexa-, and hepta-halo-substituted naphthalene groups; 3- or 4-halo-substituted phenyl groups, 3- or 4-alkyl-substituted phenyl groups, 3- or 4-alkoxy-substituted phenyl groups, 3- or 4-RCO-substituted phenyl, 5- or 6-halo-substituted naphthalene groups. More specifically, substituted aryl groups include acetylphenyl groups, particularly 4-acetylphenyl groups; fluorophenyl groups, particularly 3-fluorophenyl and 4-fluorophenyl groups; chlorophenyl groups, particularly 3-chlorophenyl and 4-chlorophenyl groups; methylphenyl groups, particularly 4-methylphenyl groups, and methoxyphenyl groups, particularly 4-methoxyphenyl groups.

As used herein, the term “alkylene” refers to a divalent radical derived from an alkyl group as defined herein. Alkylene groups in some embodiments function as bridging and/or spacer groups in the present compositions.

As used herein, the term “cycloalkylene” refers to a divalent radical derived from a cycloalkyl group as defined herein. Cycloalkylene groups in some embodiments function as bridging and/or spacer groups in the present compositions.

As used herein, the term “alkenylene” refers to a divalent radical derived from an alkenyl group as defined herein. Alkenylene groups in some embodiments function as bridging and/or spacer groups in the present compositions.

As used herein, the term “cylcoalkenylene” refers to a divalent radical derived from a cylcoalkenyl group as defined herein. Cycloalkenylene groups in some embodiments function as bridging and/or spacer groups in the present compositions.

As used herein, the term “alkynylene” refers to a divalent radical derived from an alkynyl group as defined herein. Alkynylene groups in some embodiments function as bridging and/or spacer groups in the present compositions.

As to any of the above groups which contain one or more substituents, it is understood, that such groups do not contain any substitution or substitution patterns which are sterically impractical and/or synthetically non-feasible. In addition, the compounds of this invention include all stereochemical isomers arising from the substitution of these compounds.

Pharmaceutically acceptable salts comprise pharmaceutically-acceptable anions and/or cations. Pharmaceutically-acceptable cations include among others, alkali metal cations (e.g., Li⁺, Na⁺, K⁺), alkaline earth metal cations (e.g., Ca²⁺, Mg²⁺), non-toxic heavy metal cations and ammonium (NH₄⁺) and substituted ammonium (N(R′)₄⁺, where R′ is hydrogen, alkyl, or substituted alkyl, i.e., including, methyl, ethyl, or hydroxyethyl, specifically, trimethyl ammonium, triethyl ammonium, and triethanol ammonium cations). Pharmaceutically-acceptable anions include among other halides (e.g., Cl⁻, Br⁻), sulfate, acetates (e.g., acetate, trifluoroacetate), ascorbates, aspartates, benzoates, citrates, and lactate.

The compounds of this invention may contain one or more chiral centers. Accordingly, this invention is intended to include racemic mixtures, diasteromers, enantiomers and mixture enriched in one or more stereoisomer. The scope of the invention as described and claimed encompasses the racemic forms of the compounds as well as the individual enantiomers and non-racemic mixtures thereof.

Many of the compositions described herein are at least partially present as an ion when provided in solution and as will be understood by those having skill in the art the present compositions include these partially or fully ionic forms. A specific example relates to acidic and basic groups, for example on the polymer back bone of block copolymers and in linking groups, that will be in an equilibrium in solution with respect to ionic and non-ionic forms. The compositions and formula provided herein include all fully and partially ionic forms that would be present in solution conditions of pH ranging from 1-14, and optionally pH ranging from 3-12 and optionally pH ranging from 6-8. The compositions and formula provided herein include all fully and partially ionic forms that would be present under physiological conditions.

Before the present methods are described, it is understood that this invention is not limited to the particular methodology, protocols, cell lines, and reagents described, as these may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a”, “an”, and “the” include plural reference unless the context clearly dictates otherwise. Thus, for example, reference to “a cell” includes a plurality of such cells and equivalents thereof known to those skilled in the art, and so forth. As well, the terms “a” (or “an”), “one or more” and “at least one” can be used interchangeably herein. It is also to be noted that the terms “comprising”, “including”, and “having” can be used interchangeably.

As used herein, the term “treating” includes preventative as well as disorder remittent treatment. As used herein, the terms “reducing”, “suppressing” and “inhibiting” have their commonly understood meaning of lessening or decreasing.

In certain embodiments, the present invention encompasses administering optical agents useful in the present invention to a patient or subject. A “patient” or “subject”, used equivalently herein, refers to an animal. In particular, an animal refers to a mammal, preferably a human. The subject either: (1) has a condition remediable or treatable by administration of an optical agent of the invention; or (2) is susceptible to a condition that is preventable by administering an optical agent of this invention.

Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference for the purpose of describing and disclosing the chemicals, cell lines, vectors, animals, instruments, statistical analysis and methodologies which are reported in the publications which might be used in connection with the invention. Nothing herein is to be construed as an admission that the invention is not entitled to antedate such disclosure by virtue of prior invention.

This invention disclosure relates to the generally to the concept of integrating photoactive molecules (e.g., fluorophores, chromophores, photosensitizers and photoreactive functionalities) into polymer micelles through physical association and covalent cross linking chemistry. The resulting nanosystems are useful for in vivo imaging, visualization, monitoring and phototherapy applications.

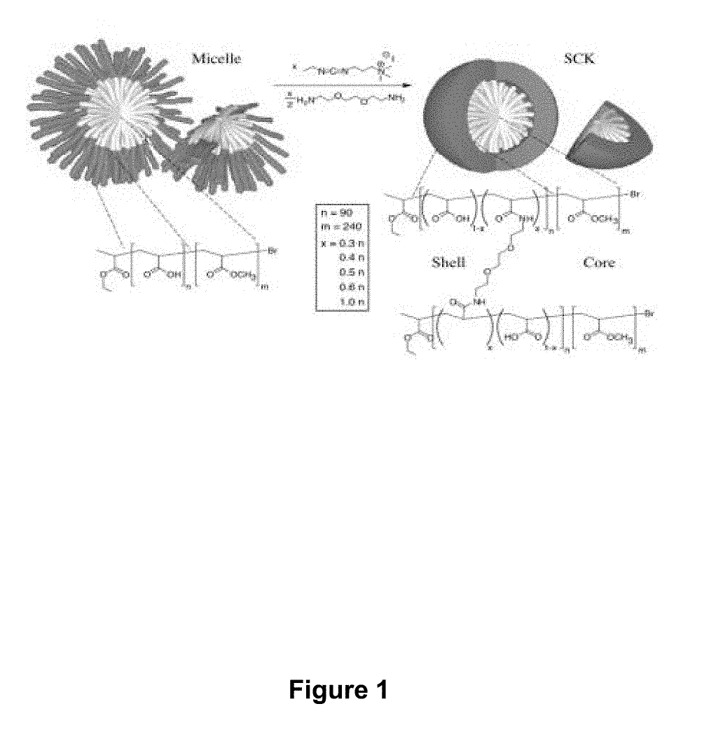

A body of research now exists regarding the supramolecular assembly of amphiphilic block copolymers into micelles which can be covalently shell-cross-linked (SCK) to form core-shell type nanoparticles. FIG. 1 provides a schematic representation showing an example of the formation of a shell-cross-linked micelle structure. Aspects of the present invention include the chemical nature of the block copolymers used to form the precursor micelles and the corresponding contributions to the overall morphology and environmental responsiveness of the resulting SCK. Further synthetic elaboration of these systems can be accomplished in a pre- or post-SCK fashion with incorporation of tissue targeting and/or imaging appendages on the exterior of the nanostructure. In addition, chemistry has been developed to attach functionality within the excavated core of SCK nanoparticles.

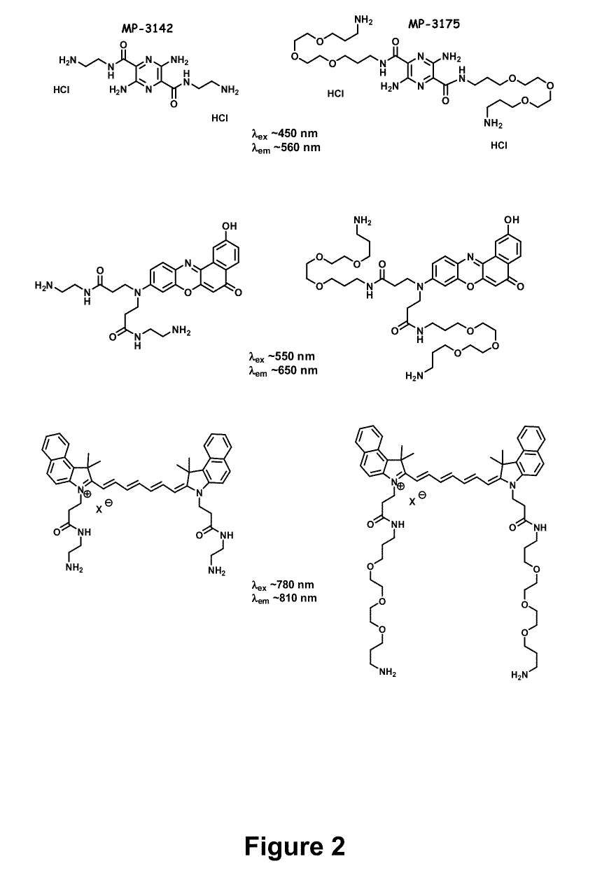

The methods and compositions of the present invention uses bi-functional optical probe molecules as photonic linkage systems for the micelle cross linking step in SCK formation. The resultant SCK nanostructures have a covalently stabilized shell that contains a specified number of copies of the optical probe. The optical probe molecule can be varied extensively, for example, from bifunctional pyrazines, to Nile Red derivatives, to indocyanine derivatives to cover yellow-green to red to NIR excitation and emission, respectively. FIG. 2 provides examples of bifunctional optical probes moieties for photonic shell cross linking in the present methods and compositions.

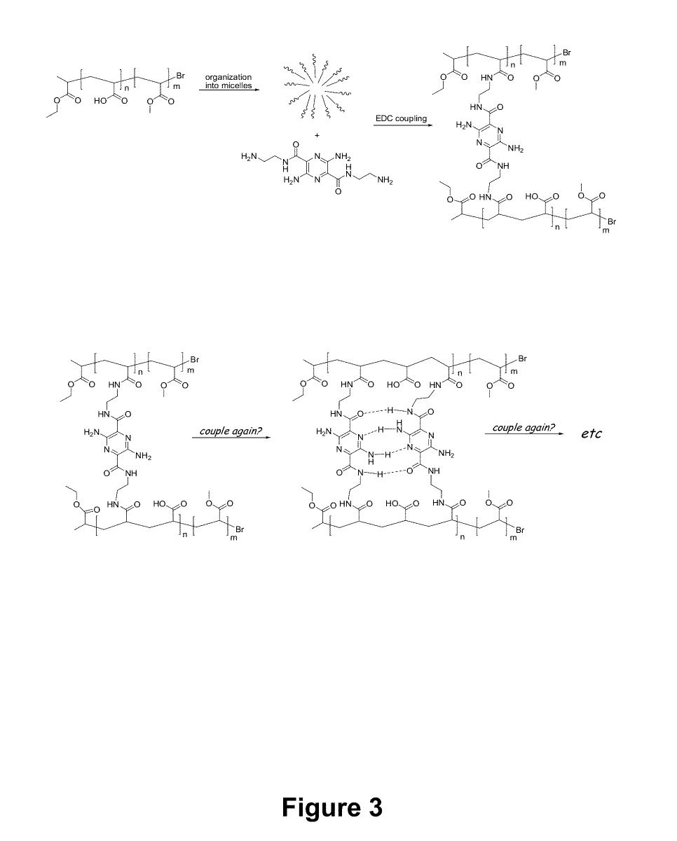

FIG. 3 provides a schematic diagram illustrating a synthetic pathway for the formation of photonic shell containing SCKs via cross linking chemistry with a linking group of the present invention.

The present photonic nanosystems and compositions thereof enable a number of potential biomedical uses.

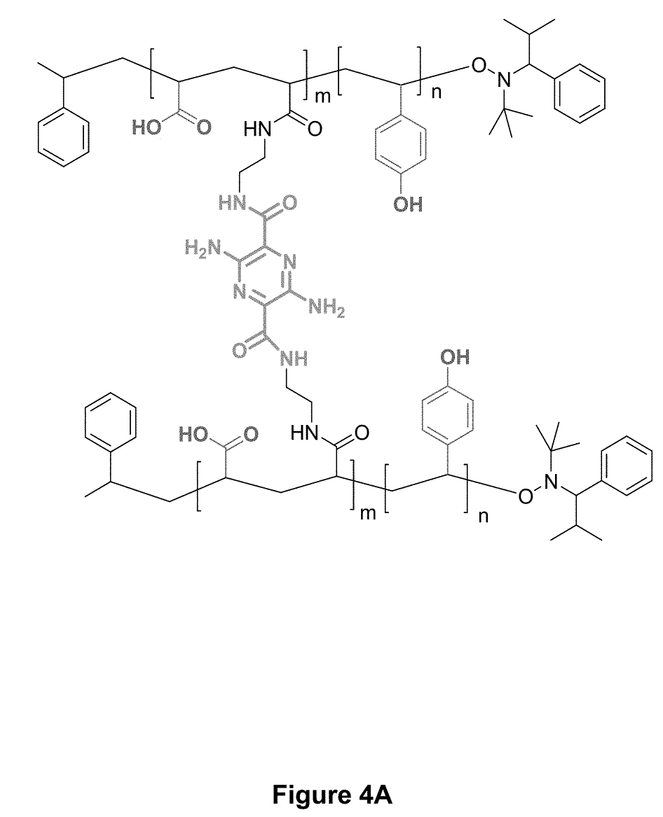

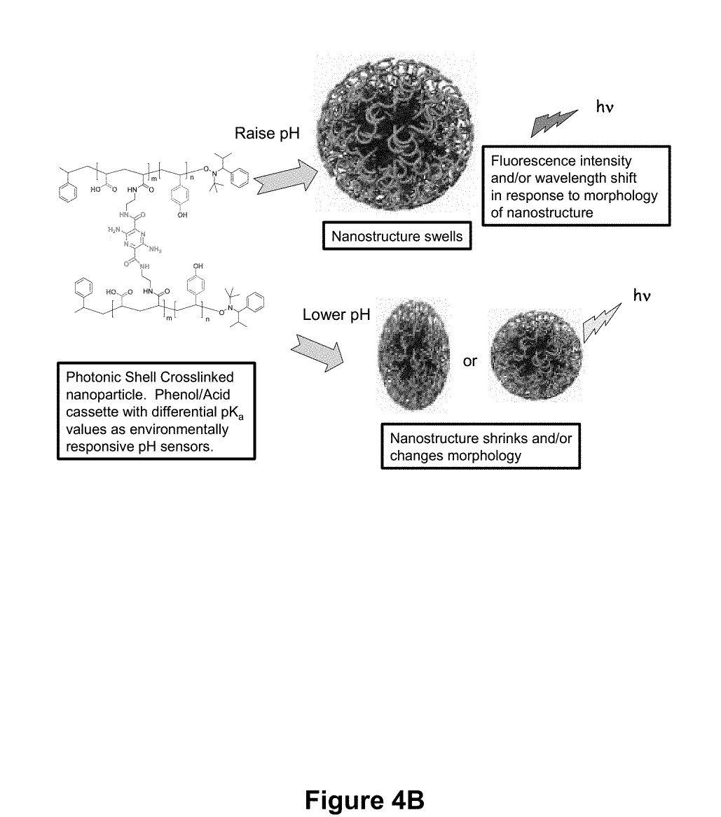

In an embodiment, photonic nanosystems and compositions thereof enable chemical and/or physiological sensors and sensing methods. In some embodiments, block copolymer micelle systems are used that respond morphologically to pH (e.g., swell at high pH and shrink at low pH) through the incorporation of functionality of differential pKa (e.g., phenol/carboxylate cassette). Photophysical consequences of these changes in morphology are manifested as “fluorescence on” at higher pH's and “fluorescence off” at lower due to proximity quenching. In an alternatively embodiment, photophysical consequences of these changes in morphology are manifested as the shifting of emission maxima as a function of nanostructure morphology enabling ratiometric pH measurement. The pyrazines are quadrupoles and display photophysical characteristics that are fairly insensitive to pH changes, thus the resulting photophysical changes will be a function of nanostructure morphology alone. The Nile Red analogues are dipoles and highly sensitive to solvent and potentially pH, thus the resulting photophysical changes are a function of both the morphology of the nanostructure and the local internal environment of the covalently linked probe. FIG. 4A illustrates an exemplary photonic shell cross-linked nanoparticle structure for this application. FIG. 4B schematically illustrates the effects of raising and/or lowering the pH on a photonic shell cross-linked nanoparticle.

In an embodiment, photonic nanosystems and compositions thereof provide optical imaging agents, including optical probes with organized photonic shell architecture. Aspects of the present invention useful for this application of the present compositions include (i) the potential for providing an increase the in vivo sensitivity of the nanostructure over that of small molecule probes; (ii) the potential ability to organize the shell-dye arrangement to increase fluorescence and/or induce useful shifts in wavelength; (iii) the potential capability to simulate a quantum dot semiconductor system with this organic nanostructure; and (iv) the potential quadrupolar nature of the pyrazines to induce two photon fluorescence for deeper tissue penetration and better spatial resolution with properties enhanced by the nanoarchitecture.

In an embodiment, photonic nanosystems and compositions thereof provide carriers and antennae for Type I Phototherapeutic Agents. In an embodiment of this aspect, the photonic shell of the present photonic nanosystems and compositions is used as an “Antenna/Transducer” for absorbing the appropriate laser irradiation and transferring it internally (via FRET) to type I phototherapeutic warheads that are either physically associated with the shell and/or core of the structures or covalently attached either through stable or photolabile bonds. The type I phototherapeutic warheads may be conjugatable derivatives of agents that decompose to cytotoxic reactive intermediates upon laser irradiation. The nanoparticle strategy allows the delivery of large doses in vivo. In addition, these nanophototherapeutics can be targeted with the appropriate exteriorly displayed ligand to the desired location (e.g. AᵥBₓ A₅B₁, Bombesin, EGF, VEGF, etc).

In an embodiment, photonic nanosystems and compositions thereof provide photoacoustic imaging and therapy agents. In an embodiment of this aspect, the photonic shell SCKs provide organic optical probes for photoacoustic imaging and therapy. The photonic shells containing many copies of longer wavelength probes (cypate analogues) may be tuned to provide the enhanced cross-sections for absorption based photoacoustic methods.

The present invention provides optical agents comprising optically functional cross-linked supramolecular structures and assemblies useful for a range of imaging, diagnostic, and therapeutic applications. Supramolecular structures and assemblies of the present invention include optically functional shell-cross-linked micelles wherein optical functionality is achieved via incorporation of one or more linking groups comprising photoactive moieties. The present invention further includes imaging, sensing and therapeutic methods using one or more optical agents of the present invention including optically functional shell cross-linked micelles. The present invention includes in situ monitoring methods, for example, wherein physical and/or structural changes in an optically functional shell-cross-linked micelle generated in response to changes in chemical environment or physiological conditions causes a measurable change in the wavelengths or intensities of emission from the micelle.

In an aspect, the present invention provides an optical agent comprising an optically functional shell-cross-linked micelle, comprising: (i) a plurality of cross-linked block copolymers, wherein each of the block copolymers comprises a hydrophilic block and a hydrophobic block; and (ii) a plurality of linking groups covalently cross linking at least a portion the hydrophilic blocks of the block copolymers, wherein at least a portion of the linking groups comprise one or more photoactive moieties, such as such as chromophores, fluorophores and/or phototherapeutic agents. The optically functional shell-cross-linked micelle has an interior hydrophobic core comprising the hydrophobic blocks of the block copolymers and a covalently cross-linked hydrophilic shell comprising the hydrophilic blocks of the block copolymers. Optionally, the extent of cross linking in the cross-linked micelle is selected over the range of 1% to 99% of the monomers of the hydrophilic blocks of the block copolymers, optionally 1% to 75% of the monomers of the hydrophilic blocks of the block copolymers, and optionally 10 to 75% of the monomers of the hydrophilic blocks of the block copolymers.

An optically functional shell-cross-linked micelle composition useful for biomedical applications, for example, can comprise block copolymers having poly(acrylic acid) polymer hydrophilic blocks, optionally having between 20-250 monomer units. In an embodiment, for example, linking groups comprising one or more photoactive moieties are bound to at least a portion of the monomers of the poly(acrylic acid) polymer block by carboxamide bonds.

In an embodiment, at least a portion of the hydrophilic blocks of the block copolymers comprise monomers bound to linking groups having the formula:

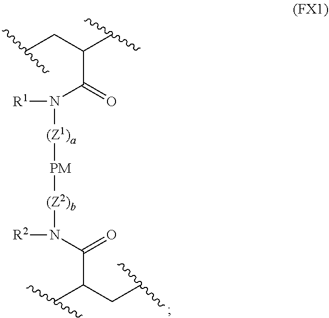

wherein PM is the linking group; wherein each of R¹ and R² is independently selected from the group consisting of —R, —COOR, —COR, —CON(R)₂, —OCON(R)₂, —N(R)₂, —SR, —SO₂R, —SOR, —OCOOR, —SO₂N(R)₂, and —OR; R is selected from the group consisting of a hydrogen, C₁-C₂₀ alkyl, C₅-C₂₀ aryl, C₁-C₂₀ carbonyl, C₂-C₂₀ alkenyl, C₂-C₂₀ alkynyl, C₅-C₂₀ alkylaryl, C₁-C₂₀ alkoxy, halo, amine, amide, hydroxyl, carboxyl, cyano, a nitrile group, an azide group, a nitro group, an acyl group, a thiol group or a natural or non-natural amino acid or fragment (e.g., side chain) thereof;

each of Z¹ and Z² is independently

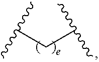

wherein one or more CH₂ groups may be replaced by NH, O, S, a carbonyl (C═O), or a sulfonyl (S═O or O═S=O); two adjacent CH₂ groups may be replaced by —CH═CH— or —C≡C—; and wherein each e is independently selected from the range of 0 to 10; and each of a and b is independently 0 or 1. As used herein, reference to a or b equal to 0 represents a formula wherein there is no Z¹ or Z² group present, respectively. As applied to formula (FX1), reference to a or b equal to 0, for example, refers to a formula wherein PM is directly bound to the adjacent nitrogen(s). As used herein, reference to e equal to 0 also represents a formula wherein there is no Z¹ and/or Z² group present. In an embodiment, each e is independently is selected from the range of 1 to 5. The present invention includes compositions comprising enantiomers, diastereomers and/or ionic forms (e.g., protonated and deprotonated forms) of formula (FX1).

In an optical agent of the invention, at least a portion of the monomers of the hydrophilic blocks of the block copolymers are bound to the linking groups by formula (FX1) and the linking group PM comprises at least one chromophore or fluorophore group capable of excitation by absorption of electromagnetic radiation having wavelengths in the visible (e.g. 400 nm to 750 nm) and/or the near infrared region (e.g., 750-1300 nm) regions of the electromagnetic spectrum. In an embodiment, for example, the linking group PM comprises a chromophore or fluorophore group selected from the group consisting of a phenylxanthene, a phenothiazine, a phenoselenazine, a cyanine, an indocyanine, a squaraine, a dipyrrolo pyrimidone, an anthraquinone, a tetracene, a quinoline, a pyrazine, an acridine, an acridone, a phenanthridine, an azo dye, a rhodamine, a phenoxazine, an azulene, an azaazulene, a triphenyl methane dye, an indole, a benzoindole, an indocarbocyanine, a Nile Red dye, a benzoindocarbocyanine, and conjugates, complexes, fragments and derivatives thereof. In an optical agent of the invention, at least a portion of the monomers of the hydrophilic blocks of the block copolymers are bound to the linking groups by formula (FX1) and PM comprises one or more pyrazine groups.

In an embodiment wherein PM is connected to the hydrophilic blocks of the copolymer via spacers, each of Z¹ and Z² is independently amide, C₁-C₁₀ alkylene, C₃-C₁₀ cycloalkylene, poly(alkylene glycol), C₂-C₁₀ alkenylene, C₃-C₁₀ cycloalkenylene, carbonyl, or C₂-C₁₀ alkynylene. In an embodiment, at least one of Z¹ and Z² is a substituent comprising —(CH₂CH₂O)_{b}— (PEG, poly(ethylene glycol)) wherein b is selected from the range of 1 to 10. In an optical agent of the invention, at least a portion of the monomers of the hydrophilic blocks of the block copolymers are bound to the linking groups by formula (FX1) and at least one of, and optionally each of, R¹ and R² is independently hydrogen, C₁-C₂₀ alkyl, C₅-C₂₀ aryl, C₁-C₂₀ acyl, C₂-C₂₀ alkenyl, C₂-C₂₀ alkynyl, C₅-C₂₀ alkylaryl, halo, amine, hydroxyl, carboxyl, C₁-C₂₀ alkoxycarbonyl or a natural or non-natural amino acid or fragment (e.g., side chain) thereof. In an optical agent of the invention, at least a portion of the monomers of the hydrophilic blocks of the block copolymers are bound to the linking groups by formula (FX1) and each of R¹ and R² is independently hydrogen, C₁-C₁₀ alkyl, C₅-C₁₀ aryl, or C₁-C₁₀ acyl.

In an embodiment, at least a portion of the monomers of the hydrophilic blocks are bound to pyrazine-based linking groups such as a pyrazine-based amino linking group. In an embodiment, for example, at least a portion of the hydrophilic blocks of the block copolymers comprise monomers bound to linking groups having the formula:

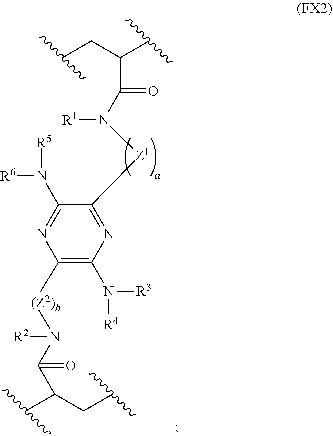

wherein each of R¹-R⁶ is independently selected from the group consisting of —R, —COOR, —COR, —CON(R)₂, —OCON(R)₂, —N(R)₂, —SR, —SO₂R, —SOR, —OCOOR, —SO₂N(R)₂, and —OR; R is selected from the group consisting of a hydrogen, C₁-C₂₀ alkyl, C₅-C₂₀ aryl, C₁-C₂₀ carbonyl, C₂-C₂₀ alkenyl, C₂-C₂₀ alkynyl, C₅-C₂₀ alkylaryl, C₁-C₂₀ alkoxy, halo, amine, amide, hydroxyl, carboxyl, cyano, a nitrile group, an azide group, a nitro group, an acyl group, a thiol group or a natural or non-natural amino acid or fragment (e.g., side chain) thereof;

each of Z¹ and Z² is independently

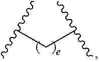

wherein one or more CH₂ groups may be replaced by NH, O, S, a carbonyl (C═O), or a sulfonyl (S═O or O═S=O); two adjacent CH₂ groups may be replaced by —CH═CH— or —C≡C—; and wherein each e is independently is selected from the range of 0 to 10; and each of a and b is independently 0 or 1. The present invention includes compositions comprising enantiomers, diastereomers, and/or ionic forms (e.g., protonated and deprotonated forms) of formula (FX2).

In an optical agent of the invention, at least a portion of the monomers of the hydrophilic blocks of the block copolymers are bound to the linking groups by formula (FX2) and each R¹-R⁶ is independently hydrogen, C₁-C₂₀ alkyl, C₅-C₂₀ aryl, C₁-C₂₀ acyl, C₂-C₂₀ alkenyl, C₂-C₂₀ alkynyl, C₅-C₂₀ alkylaryl, halo, amine, hydroxyl, carboxyl, C₁-C₂₀ alkoxycarbonyl, or a natural or non-natural amino acid or fragment (e.g., side chain) thereof In an optical agent of the invention, at least a portion of the monomers of the hydrophilic blocks of the block copolymers are bound to the linking groups by formula (FX2) and at least one of, and optionally each of, R¹-R⁶ is independently hydrogen, C₁-C₁₀ alkyl, C₅-C₁₀ aryl, or C₁-C₁₀ acyl. In an embodiment, each e is independently is selected from the range of 1 to 5. In an optical agent of the invention, at least a portion of the monomers of the hydrophilic blocks of the block copolymers are bound to the linking groups by formula (FX2) and each of Z¹ and Z² is independently amide, C₁-C₁₀ alkylene, C₃-C₁₀ cycloalkylene, poly(alkylene glycol), C₂-C₁₀ alkenylene, C₃-C₁₀ cycloalkenylene, carbonyl, or C₂-C₁₀ alkynylene. In an embodiment, at least one of Z¹ and Z² is a substituent comprising —(CH₂CH₂O)_{b}— (PEG, poly(ethylene glycol)) wherein b is selected from the range of 1 to 10.

In an embodiment, at least a portion of the monomers of the hydrophilic blocks are bound to pyrazine-based linking groups via a carboxamide bonding scheme (e.g., via amino carbonyl groups). In an embodiment, for example, at least a portion of the hydrophilic blocks of the block copolymers comprise monomers bound to linking groups having the formula:

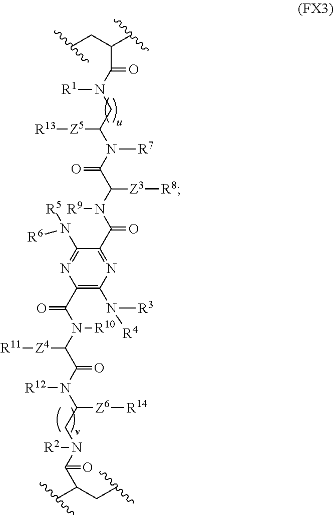

wherein each of R¹-R¹⁴ is independently selected from the group consisting of —R, —COOR, —COR, —CON(R)₂, —OCON(R)₂, —N(R)₂, —SR, —SO₂R, —SOR, —OCOOR, —SO₂N(R)₂, and —OR; R is selected from the group consisting of a hydrogen, C₁-C₂₀ alkyl, C₅-C₂₀ aryl, C₁-C₂₀ carbonyl, C₂-C₂₀ alkenyl, C₂-C₂₀ alkynyl, C₅-C₂₀ alkylaryl, C₁-C₂₀ alkoxy, halo, amine, amide, hydroxyl, carboxyl, cyano, a nitrile group, an azide group, a nitro group, an acyl group, a thiol group or a natural or non-natural amino acid or fragment (e.g., side chain) thereof; each of u and v is independently selected from the range of 0 to 10; each of Z³-Z⁶ is independently

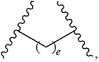

wherein one or more CH₂ groups may be replaced by NH, O, S, a carbonyl (C═O), or a sulfonyl (S═O or O=S=O); two adjacent CH₂ groups may be replaced by —CH═CH— or —C≡C—; and wherein each e is independently is selected from the range of 0 to 10. In an embodiment, e is selected from the range of 1 to 5. The present invention includes compositions comprising enantiomers, diastereomers, and/or ionic forms (e.g., protonated and deprotonated forms) of formula (FX3).

In an optical agent of the present invention having the cross linking between hydrophilic blocks of block copolymers as shown in formula (FX3) at least one of R⁸, R¹¹, R¹³, and R¹⁴ is the side chain of a basic natural or non-natural amino acid, such as at least one of R⁸, R¹¹, R¹³, and R¹⁴ is a side chain of an amino acid selected from the group consisting of arginine, lysine, histidine, ornithine, and homoarginine. In an embodiment, at least one of R⁸, R¹¹, R¹³, and R¹⁴ is selected from the group consisting of:

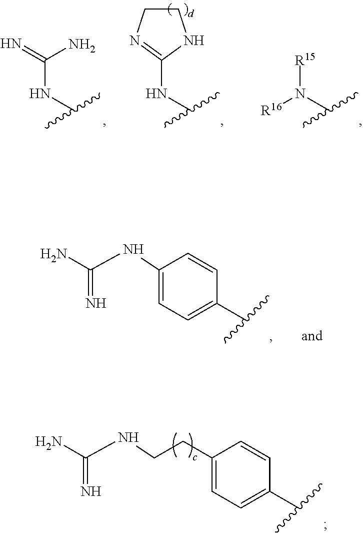

wherein d is selected from the range of 1 to 4 and wherein c is selected from the range of 1 to 7, and wherein each of wherein each of R¹⁵ and R¹⁶ is independently selected from the group consisting of —R, —COOR, —COR, —CON(R)₂, —OCON(R)₂, —N(R)₂, —SR, —SO₂R, —SOR, —OCOOR, —SO₂N(R)₂, and —OR; R is selected from the group consisting of a hydrogen, C₁-C₂₀ alkyl, C₅-C₂₀ aryl, C₁-C₂₀ carbonyl, C₂-C₂₀ alkenyl, C₂-C₂₀ alkynyl, C₀₅-C₂₀ alkylaryl, C₁-C₂₀ alkoxy, halo, amine, amide, hydroxyl, carboxyl, cyano, a nitrile group, an azide group, a nitro group, an acyl group, a thiol group or a natural or non-natural amino acid or fragment (e.g., side chain) thereof, and optionally, R¹⁵ and R¹⁶ can together form a aliphatic or aromatic ring of 4-8 carbons, optionally substituted with one or more S, C or O heteroatoms provided in the aliphatic or aromatic ring. In an embodiment, each of R¹⁵ and R¹⁶ is independently a hydrogen or C₁-C₅ alkyl.

In an optical agent of the invention, at least a portion of the monomers of the hydrophilic blocks of the block copolymers are bound to the linking groups by formula (FX3) and each R¹-R¹⁶ is independently hydrogen, C₁-C₂₀ alkyl, C₅-C₂₀ aryl, C₁-C₂₀ acyl, C₂-C₂₀ alkenyl, C₂-C₂₀ alkynyl, C₅-C₂₀ alkylaryl, halo, amine, hydroxyl, carboxyl, C₁-C₂₀ alkoxycarbonyl, or a natural or non-natural amino acid or fragment (e.g., side chain) thereof. In an optical agent of the invention, at least a portion of the monomers of the hydrophilic blocks of the block copolymers are bound to the linking groups by formula (FX3) and at least one of R¹-R¹⁶, and optionally each of R¹-R¹⁶, is independently hydrogen, C₁-C₁₀ alkyl, C₅-C₁₀ aryl, or C₁-C₁₀ acyl. In an embodiment, each e is independently is selected from the range of 1 to 5. In an optical agent of the invention, at least a portion of the monomers of the hydrophilic blocks of the block copolymers are bound to the linking groups by formula (FX3) and each of Z³-Z⁶ is independently amide, C₁-C₁₀ alkylene, C₃-C₁₀ cycloalkylene, poly(alkylene glycol), C₂-C₁₀ alkenylene, C₃-C₁₀ cycloalkenylene, carbonyl, or C₂-C₁₀ alkynylene. In an embodiment, at least one of Z³-Z⁶ is a substituent comprising —(CH₂CH₂O)_{b}— (PEG, poly(ethylene glycol)) wherein b is selected from the range of 1 to 10.

In an embodiment, at least a portion of the monomers of the hydrophilic blocks are bound to pyrazine-based linking groups having one or more guanidine or guanidine derivative moieties (e.g., the side chain of the amino acid arginine). In an embodiment, for example, at least a portion of the hydrophilic blocks of the block copolymers comprise monomers bound to linking groups having the formula:

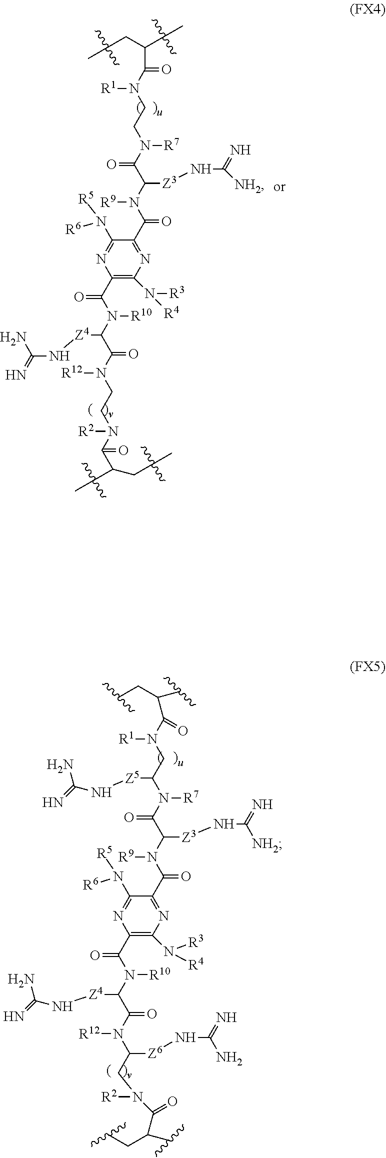

wherein R¹-R⁷, R⁹-R¹⁰, R¹², Z³, Z⁴, Z⁵, Z⁶, e, u and v are defined as described above in the context of formula (FX3). In an optical agent of the invention, at least a portion of the monomers of the hydrophilic blocks of the block copolymers are bound to the linking groups by formula (FX4) or (FX5) and each R¹-R⁷, R⁹-R¹⁰, and R¹² is independently hydrogen, C₁-C₂₀ alkyl, 05-C₂₀ aryl, C₁-C₂₀ acyl, C₂-C₂₀ alkenyl, C₂-C₂₀ alkynyl, C₅-C₂₀ alkylaryl, halo, amine, hydroxyl, carboxyl, C₁-C₂₀ alkoxycarbonyl, or a natural or non-natural amino acid or fragment (e.g., side chain) thereof. The present invention includes compositions comprising enantiomers, diastereomers, and/or ionic forms (e.g., protonated and deprotonated forms) of formula (F×4) and (F×5).

In an optical agent of the invention, at least a portion of the monomers of the hydrophilic blocks of the block copolymers are bound to the linking groups by formula (FX4) or (FX5) and at least one of R¹-R⁷, R⁹-R¹, and R¹² is hydrogen, C₁-C₁₀ alkyl, C₅-C₁₀ aryl, or C₁-C₁₀ acyl. In an embodiment, each e is independently is selected from the range of 1 to 5. In an optical agent of the invention, at least a portion of the monomers of the hydrophilic blocks of the block copolymers are bound to the linking groups by formula (FX4) or (FX5) and each of Z³-Z⁶ is independently amide, C₁-C₁₀ alkylene, C₃-C₁₀ cycloalkylene, poly(alkylene glycol), C₂-C₁₀ alkenylene, C₃-C₁₀ cycloalkenylene, carbonyl, or C₂-C₁₀ alkynylene. In an embodiment, at least one of Z³-Z⁶ is a substituent comprising —(CH₂CH₂O)_{b}— (PEG, poly(ethylene glycol)) wherein b is selected from the range of 1 to 10.

In an embodiment of this aspect, at least a portion of the hydrophilic blocks of the block copolymers comprise monomers bound to linking groups having the formula:

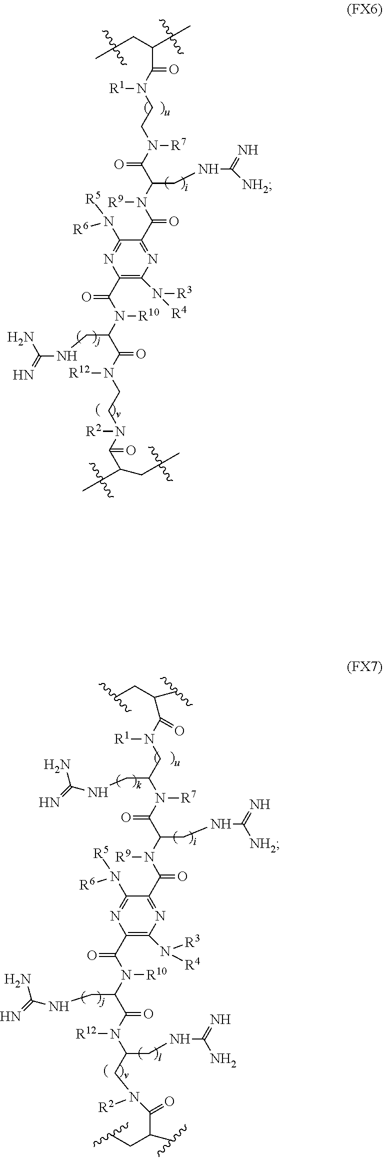

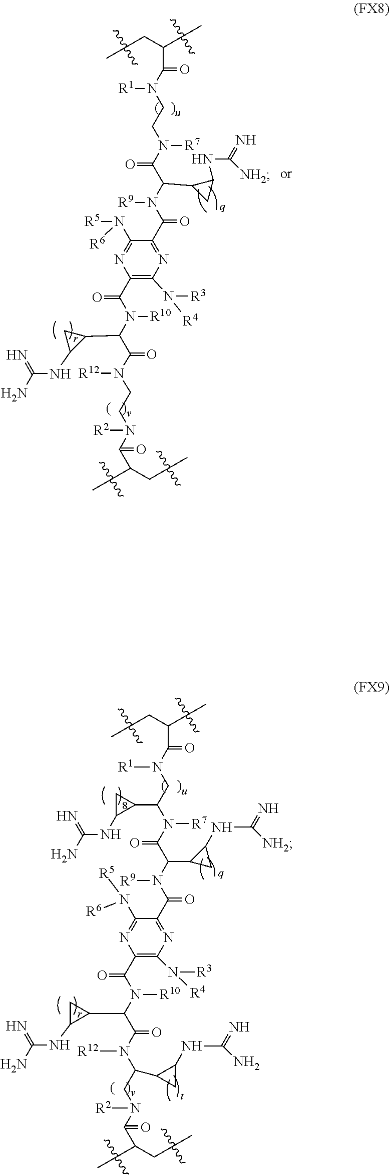

wherein R¹-R¹⁰, R¹², e, u and v are defined as described above in the context of formula (FX3), each of i, j, k and l is independently selected from the range of 0 to 9, and each of q, r, s and t is independently selected from the range of 1 to 3. The present invention includes compositions comprising enantiomers, diastereomers, and/or ionic forms (e.g., protonated and deprotonated forms) of formula (FX5)-(FX9).

In an optical agent of the invention, at least a portion of the monomers of the hydrophilic blocks of the block copolymers are bound to the linking groups by formula ((FX6), (FX7), (FX8) or (FX9) and each of R¹-R¹⁰, and R¹² is independently hydrogen, C₁-C₂₀ alkyl, C₅-C₂₀ aryl, C₁-C₂₀ acyl, C₂-C₂₀ alkenyl, C₂-C₂₀ alkynyl, C₅-C₂₀ alkylaryl, halo, amine, hydroxyl, carboxyl, C₁-C₂₀ alkoxycarbonyl, or a natural or non-natural amino acid or fragment (e.g., side chain) thereof. In an optical agent of the invention, at least a portion of the monomers of the hydrophilic blocks of the block copolymers are bound to the linking groups by formula (FX6), (FX7), (FX8) or (FX9) and at least one of R¹-R¹⁰, and R¹² is hydrogen, C₁-C₁₀ alkyl, C₅-C₁₀ aryl, or C₁-C₁₀ acyl. In an embodiment, each e is independently is selected from the range of 1 to 5.

An optically functional shell-cross-linked micelle composition of the present invention comprises block copolymers having poly(acrylic acid) polymer hydrophilic block cross-linked by one or more pyrazine photoactive moieties or conjugates or derivatives thereof. In an embodiment, for example, at least a portion of the hydrophilic blocks of the block copolymers comprise monomers bound to linking groups having the formula:

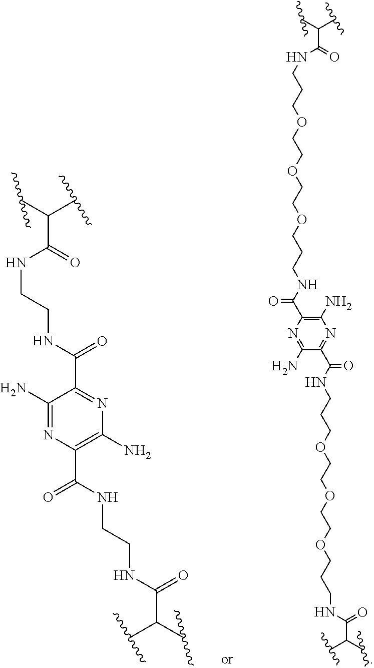 [SMILES: *C(*)C(=O)NCCNC(=O)c1nc(N)c(C(=O)NCCNC(=O)C(*)*)nc1N ; *C(*)C(=O)NCCCOCCOCCOCCCNC(=O)c1nc(N)c(C(=O)NCCCOCCOCCOCCCNC(=O)C(*)*)nc1N]

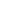

As will be understood by those having skill in the art, the present invention includes supramolecular structures and compositions cross-linked via other types of covalent bonding known in the art of synthetic organic chemistry and polymer chemistry.

An optically functional shell cross-linked micelle of the invention comprises block copolymer and linking group components having the structure:

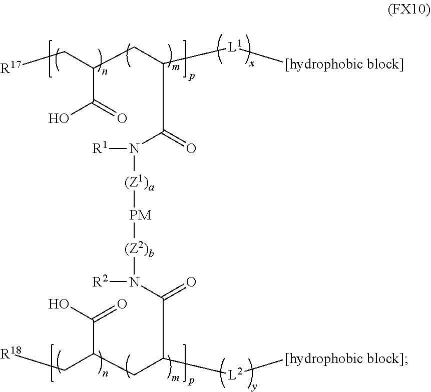

wherein PM, R¹, R², Z¹, Z², a and b are defined as described above in the context of formula (FX1); wherein p is selected from the range of 20 to 250, wherein independently for each value of p, n is independently equal to 1 or 0 and m is independently equal to 1 or 0; each of R¹⁷ and R¹⁸ is independently selected from the group consisting of —R, —COOR, —COR, —CON(R)₂, —OCON(R)₂, —N(R)₂, —SR, —SO₂R, —SOR, —OCOOR, —SO₂N(R)₂, and —OR; R is selected from the group consisting of a hydrogen, C₁-C₂₀ alkyl, C₅-C₂₀ aryl, C₁-C₂₀ carbonyl, C₂-C₂₀ alkenyl, C₂-C₂₀ alkynyl, C₅-C₂₀ alkylaryl, C₁-C₂₀ alkoxy, halo, amine, amide, hydroxyl, carboxyl, cyano, a nitrile group, an azide group, a nitro group, an acyl group, a thiol group, a natural or non-natural amino acid or fragment thereof or an additional hydrophilic block of the copolymers; each of L¹ and L² is independently

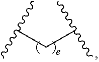

wherein one or more CH₂ groups may be replaced by NH, O, S, a carbonyl (C═O), or a sulfonyl (S═O or O=S=O); two adjacent CH₂ groups may be replaced by —CH═CH— or —C≡C—; and wherein each e is independently selected from the range of 0 to 10; each of a and b is independently 0 or 1; wherein [hydrophobic block] is a hydrophobic block of the block copolymers; wherein each of x and y is independently 0 or 1. The present invention includes compositions comprising enantiomers, diastereomers, and/or ionic forms (e.g., protonated and deprotonated forms) of formula (FX10). As used herein, reference to x or y equal to 0 represents a formula wherein there is no L¹ or L² group present, respectively. In an embodiment, each e is independently selected from the range of 1 to 5. Optionally, p is selected from the range of 50 to 200. In an optical agent of the invention, at least a portion of the block copolymer and linking group components have the structure (FX10) and PM comprises one or more pyrazine groups.

In a specific embodiment, each of R¹⁷ and R¹⁸ is independently an additional hydrophilic block of the copolymers. In a specific embodiment, each of R¹⁷ and R¹⁸ is independently a hydrophilic block selected from the group consisting of a poly(acrylic acid) polymer block, a poly(N-(acryloyloxy)succinimide) polymer block; a poly(N-acryloylmorpholine) polymer block; a poly(ethylene glycol) polymer block, poly(p-vinyl benzaldehyde) block or a poly(phenyl vinyl ketone) block. In an embodiment, each of R¹⁷ and R¹⁸ is —(CH₂CH₂O)ₕ— wherein h is selected from the range of 10 to 500.

As shown in formula (FX10), a portion of the polymer backbones of the block copolymers is shown in block parenthesis (i.e., the parenthesis with the subscript “p”) indicating repeating units of the hydrophilic block. For each repeating unit in this portion of the polymer backbone n and m can independently have values of 0 and 1, indicating that the monomers of the repeating unit may vary in this embodiment along on the polymer backbone. This structure reflects that fact that the extent and structure of cross linking between cross-linked block copolymers can vary along the polymer back bone. For example, n and m may both equal 1 for the first unit of the polymer backbone showing in formula (FX10), signifying that both cross-linked and non-cross-linked monomer groups are present in this unit, and m may equal 1 and n equal 0 in the second repeating unit of the polymer backbone signifying that only the cross-linked monomer groups is present in the second unit. Accordingly, the optical agent of formula (FX10)-(FX18) represent a class of compositions having a variable extent of cross linking, for example, an extent of cross linking ranging from 1 to 99%, optionally 1 to 75%, and optionally 20 to 75%. The hydrophilic block of the block copolymer may have any number of additional chemical domains. In an embodiment, for example, R¹⁷ and/or R¹⁸ are independently a substituent comprising —(CH₂CH₂O)_{b}— (i.e., (PEG, poly(ethylene glycol))), wherein b is selected from the range of 1 to 10.

In an embodiment, at least a portion of the monomers of the hydrophilic blocks are bound to pyrazine-based linking groups such as pyrazine-based amino linking groups. In an embodiment, for example, at least a portion of the block copolymers and linking groups of the optical agent have the formula:

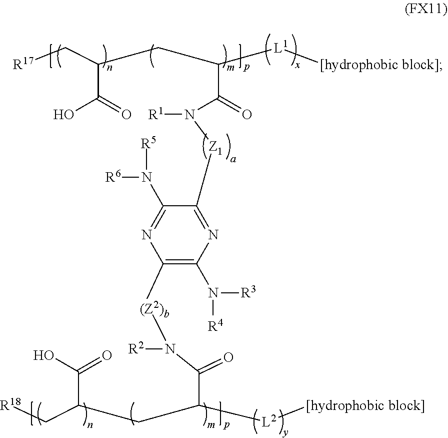

wherein R¹-R⁶, R¹⁷, R¹⁸, Z¹, Z², L¹, L², a, b, n, m, p, x and y are defined as described above in the context of formulae (FX1), (FX2), and (FX10). The present invention includes compositions comprising enantiomers, diastereomers, and/or ionic forms (e.g., protonated and deprotonated forms) of formula (FX11).

In an embodiment, for example, at least a portion of the block copolymers and linking groups of the optical agent have the formula:

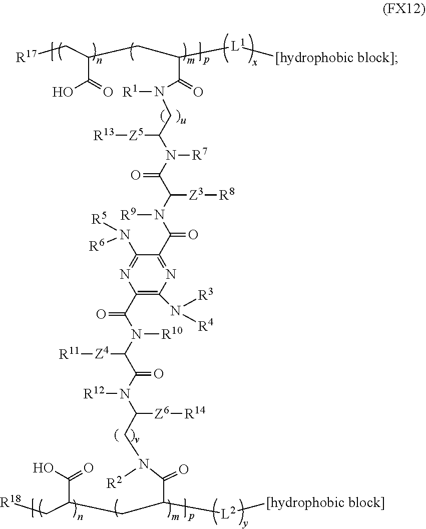

wherein R¹-R¹⁴, R¹⁷, R¹⁸, Z¹, Z², L¹, L², a, b, n, m, p, x, u, v and y are defined as described above in the context of formulae (FX1), (FX2), (FX3), (FX10) and (FX11). The present invention includes compositions comprising enantiomers, diastereomers, and/or ionic forms (e.g., protonated and deprotonated forms) of formula (FX12).

In an embodiment, at least a portion of the monomers of the hydrophilic blocks are bound to pyrazine-based linking groups having one or more guanidine or guanidine derivative moieties (e.g., side chain of the amino acid arginine). In an embodiment, for example, at least a portion of the block copolymers and linking groups of the optical agent have the formula:

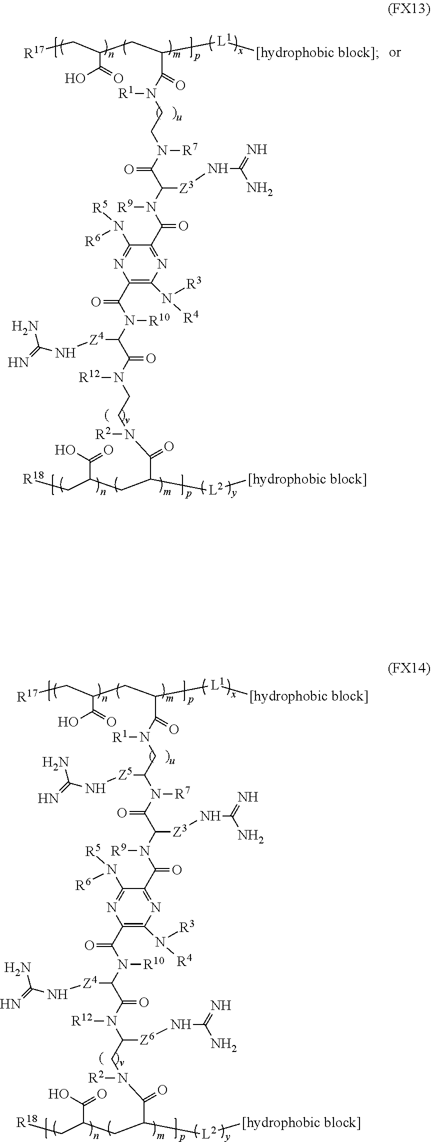

wherein R¹-R¹⁴, R¹⁷, R¹⁸, Z¹, Z², L¹, L², a, b, n, m, p, x, u, v and y are defined as described above in the context of formulae (FX1)-(FX5), (FX10), (FX11) and (FX12). The present invention includes compositions comprising enantiomers, diastereomers, and/or ionic forms (e.g., protonated and deprotonated forms) of formula (FX13) and (FX14).

In an embodiment, for example, at least a portion of the block copolymers and linking groups of the optical agent have the formula:

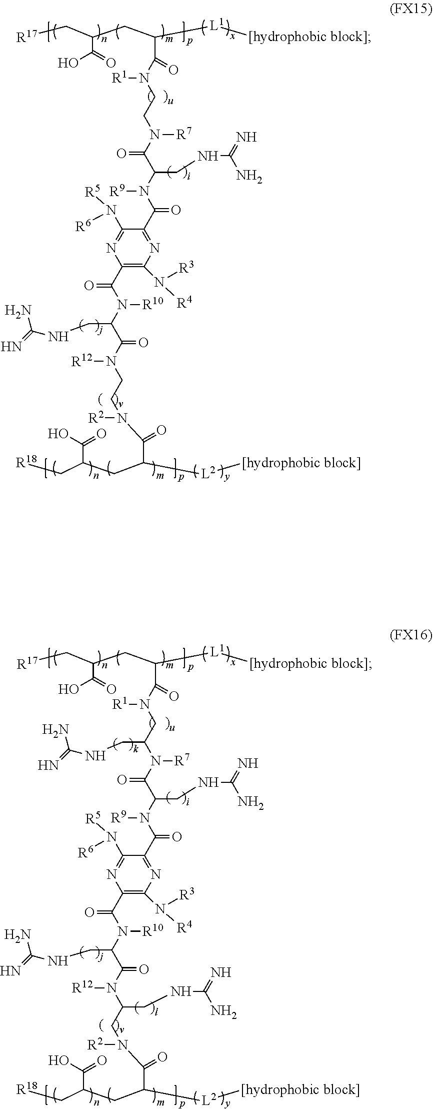

wherein R¹-R¹⁴, R¹⁷, R¹⁸, Z¹, Z², L¹, L², a, b, n, m, p, x, y, i, j, k, l, q, r, s, t, u, v, x and y are defined as described above in the context of formulae (FX1)-(FX14). The present invention includes compositions comprising enantiomers, diastereomers, and/or ionic forms (e.g., protonated and deprotonated forms) of formula (FX15)-(FX18).

In an embodiment, an optically functional shell cross-linked micelle of present invention comprises block copolymer and pyrazine linking group components having the structure:

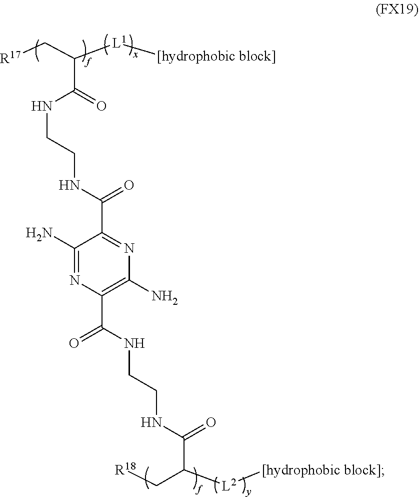

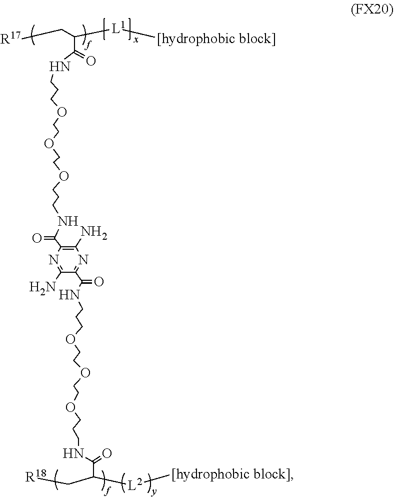

and enantiomers, diastereomers, and/or ionic forms (e.g., protonated and deprotonated forms) thereof; wherein f is selected from the range of 20 to 250; each of R¹⁷ and R¹⁸ is independently selected from the group consisting of —R, —COOR, —COR, —CON(R)₂, —OCON(R)₂, —N(R)₂, —SR, —SO₂R, —SOR, —OCOOR, —SO₂N(R)₂, and —OR; R is selected from the group consisting of a hydrogen, C₁-C₂₀ alkyl, C₅-C₂₀ aryl, C₁-C₂₀ carbonyl, C₂-C₂₀ alkenyl, C₂-C₂₀ alkynyl, C₅-C₂₀ alkylaryl, C₁-C₂₀ alkoxy, halo, amine, amide, hydroxyl, carboxyl, cyano, a nitrile group, an azide group, a nitro group, an acyl group, a thiol group, a natural or non-natural amino acid or fragment thereof or an additional hydrophilic block of the copolymers; each of L¹ and L² is independently

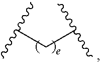

wherein one or more CH₂ groups may be replaced by NH, O, S, a carbonyl (C═O), or a sulfonyl (S═O or O=S=O); two adjacent CH₂ groups may be replaced by —CH═CH— or —C≡C—; and wherein each e is independently selected from the range of 0 to 10; each of a and b is independently 0 or 1; wherein [hydrophobic block] is a hydrophobic block of the block copolymers; wherein each of x and y is independently 0 or 1. As used herein, reference to x or y equal to 0 represents a formula wherein there is no L¹ or L² group present, respectively. In an embodiment, each e is independently selected from the range of 1 to 5. Optionally, p is selected from the range of 50 to 200.

The hydrophobic block in formula (FX10)-(FX20) (represented by [hydrophobic block]) can have a wide range of compositions depending on the desired application and use of the present optical agents. In an embodiment, the composition of [hydrophobic block] is selected from the group consisting of a poly(p-hydroxystyrene) polymer block; a polystyrene polymer block; a polyacrylate polymer block, a poly(propylene glycol) polymer block; a poly(amino acid) polymer block; a poly(ester) polymer block; a poly (ε-caprolactone) polymer block, and a phospholipid; or a copolymer thereof. In an embodiment, the [hydrophobic block] comprises monomers including one or more aryl groups, such as phenyl, phenol and/or derivative thereof. In an embodiment, the hydrophobic block has a number of monomers selected from the range of 20 to 250, optionally 210 to 250, optionally 40 to 100. In an embodiment, for example, at least a portion of the block copolymers and linking groups of the optical agent have the formula:

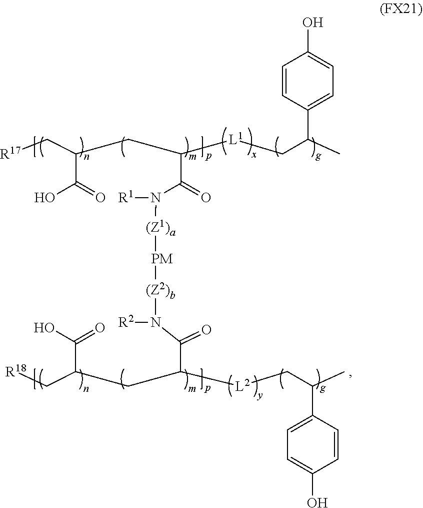

and enantiomers, diastereomers, and/or ionic forms (e.g., protonated and deprotonated forms) thereof; wherein PM, R¹, R², Z¹, Z², L¹, L², a, b, n, m, p, y, x, R¹⁷, R¹⁸, are defined as described above in the context of formula (FX1)-(FX19); wherein each g is independently selected from the range of 20 to 250.

In an embodiment, the hydrophilic groups of at least a portion of the block copolymers further comprise a poly(ethylene glycol) domain (PEG), for example a domain comprising —(CH₂CH₂O)ₕ— wherein h is selected from the range of 10 to 500, optionally 20 to 100. In an embodiment, for example, at least a portion of the block copolymers and linking groups of the optical agent have the formula:

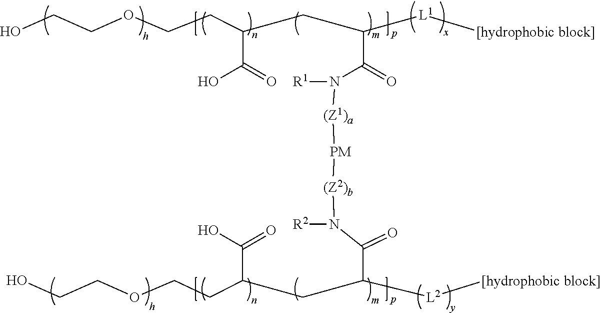

(FX22) and enantiomers, diastereomers, and/or ionic forms (e.g., protonated and deprotonated forms) thereof; wherein PM, R¹, R², Z¹, Z², L¹, L², a, b, n, m, p, y, x, are defined as described above in the context of formula (FX1)-(FX19); wherein each h is independently selected from the range of 10 to 500.

### STATEMENTS REGARDING INCORPORATION BY REFERENCE AND VARIATIONS

All references throughout this application, for example patent documents including issued or granted patents or equivalents; patent application publications; and non-patent literature documents or other source material; are hereby incorporated by reference herein in their entireties, as though individually incorporated by reference, to the extent each reference is at least partially not inconsistent with the disclosure in this application (for example, a reference that is partially inconsistent is incorporated by reference except for the partially inconsistent portion of the reference).

All patents and publications mentioned in the specification are indicative of the levels of skill of those skilled in the art to which the invention pertains. References cited herein are incorporated by reference herein in their entirety to indicate the state of the art, in some cases as of their filing date, and it is intended that this information can be employed herein, if needed, to exclude (for example, to disclaim) specific embodiments that are in the prior art. For example, when a compound is claimed, it should be understood that compounds known in the prior art, including certain compounds disclosed in the references disclosed herein (particularly in referenced patent documents), are not intended to be included in the claim.

When a group of substituents is disclosed herein, it is understood that all individual members of those groups and all subgroups, including any isomers and enantiomers of the group members, and classes of compounds that can be formed using the substituents are disclosed separately. When a compound is claimed, it should be understood that compounds known in the art including the compounds disclosed in the references disclosed herein are not intended to be included. When a Markush group or other grouping is used herein, all individual members of the group and all combinations and subcombinations possible of the group are intended to be individually included in the disclosure.

Every formulation or combination of components described or exemplified can be used to practice the invention, unless otherwise stated. Specific names of compounds are intended to be exemplary, as it is known that one of ordinary skill in the art can name the same compounds differently. When a compound is described herein such that a particular isomer or enantiomer of the compound is not specified, for example, in a formula or in a chemical name, that description is intended to include each isomers and enantiomer of the compound described individual or in any combination. One of ordinary skill in the art will appreciate that methods, device elements, starting materials, and synthetic methods other than those specifically exemplified can be employed in the practice of the invention without resort to undue experimentation. All art-known functional equivalents, of any such methods, device elements, starting materials, and synthetic methods are intended to be included in this invention. Whenever a range is given in the specification, for example, a temperature range, a time range, or a composition range, all intermediate ranges and subranges, as well as all individual values included in the ranges given are intended to be included in the disclosure.

Whenever a range is given in the specification, for example, a temperature range, a time range, or a composition or concentration range, all intermediate ranges and subranges, as well as all individual values included in the ranges given are intended to be included in the disclosure. As used herein, ranges specifically include the values provided as endpoint values of the range. For example, a range of 1 to 100 specifically includes the end point values of 1 and 100. It will be understood that any subranges or individual values in a range or subrange that are included in the description herein can be excluded from the claims herein.

As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. Any recitation herein of the term “comprising”, particularly in a description of components of a composition or in a description of elements of a device, is understood to encompass those compositions and methods consisting essentially of and consisting of the recited components or elements. The invention illustratively described herein suitably may be practiced in the absence of any element or elements, limitation or limitations which is not specifically disclosed herein.

The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

Many of the molecules disclosed herein contain one or more ionizable groups [groups from which a proton can be removed (e.g., —COOH) or added (e.g., amines) or which can be quaternized (e.g., amines)]. All possible ionic forms of such molecules and salts thereof are intended to be included individually in the disclosure herein. With regard to salts of the compounds herein, one of ordinary skill in the art can select from among a wide variety of available counterions those that are appropriate for preparation of salts of this invention for a given application. In specific applications, the selection of a given anion or cation for preparation of a salt may result in increased or decreased solubility of that salt.

Every formulation or combination of components described or exemplified herein can be used to practice the invention, unless otherwise stated.

The present compositions, preparations and formulations can be used both as a diagnostic agent as well as a photodynamic therapeutic agent concomitantly. For example, an effective amount of the present compositions, preparations and formulations in a pharmaceutically acceptable formulation is administered to a patient. Administration is followed by a procedure that combines photodiagnosis and phototherapy. For example, a composition comprising compounds for combined photodiagnosis and phototherapy is administered to a patient and its concentration, localization, or other parameters is determined at the target site of interest. More than one measurement may be taken to determine the location of the target site. The time it takes for the compound to accumulate at the target site depends upon factors such as pharmacokinetics, and may range from about thirty minutes to two days. Once the site is identified, the phototherapeutic part of the procedure may be done either immediately after determining the site or before the agent is cleared from the site. Clearance depends upon factors such as pharmacokinetics.

The present compositions, preparations and formulations can be formulated into diagnostic or therapeutic compositions for enteral, parenteral, topical, aerosol, inhalation, or cutaneous administration. Topical or cutaneous delivery of the compositions, preparations and formulations may also include aerosol formulation, creams, gels, solutions, etc. The present compositions, preparations and formulations are administered in doses effective to achieve the desired diagnostic and/or therapeutic effect. Such doses may vary widely depending upon the particular compositions employed in the composition, the organs or tissues to be examined, the equipment employed in the clinical procedure, the efficacy of the treatment achieved, and the like. These compositions, preparations and formulations contain an effective amount of the composition(s), along with conventional pharmaceutical carriers and excipients appropriate for the type of administration contemplated. These compositions, preparations and formulations may also optionally include stabilizing agents and skin penetration enhancing agents.

Methods of this invention comprise the step of administering an “effective amount” of the present diagnostic and therapeutic compositions, formulations and preparations containing the present compounds, to diagnosis, image, monitor, evaluate treat, reduce or regulate a biological condition and/or disease state in a patient. The term “effective amount,” as used herein, refers to the amount of the diagnostic and therapeutic formulation, that, when administered to the individual is effective to diagnose, image, monitor, evaluate, treat, reduce or regulate a biological condition and/or disease state. As is understood in the art, the effective amount of a given composition or formulation will depend at least in part upon, the mode of administration (e.g. intravenous, oral, topical administration), any carrier or vehicle employed, and the specific individual to whom the formulation is to be administered (age, weight, condition, sex, etc.). The dosage requirements need to achieve the “effective amount” vary with the particular formulations employed, the route of administration, and clinical objectives. Based on the results obtained in standard pharmacological test procedures, projected daily dosages of active compound can be determined as is understood in the art.

Any suitable form of administration can be employed in connection with the diagnostic and therapeutic formulations of the present invention. The diagnostic and therapeutic formulations of this invention can be administered intravenously, in oral dosage forms, intraperitoneally, subcutaneously, or intramuscularly, all using dosage forms well known to those of ordinary skill in the pharmaceutical arts.

The diagnostic and therapeutic formulations of this invention can be administered alone, but may be administered with a pharmaceutical carrier selected upon the basis of the chosen route of administration and standard pharmaceutical practice.

The diagnostic and therapeutic formulations of this invention and medicaments of this invention may further comprise one or more pharmaceutically acceptable carrier, excipient, or diluent. Such compositions and medicaments are prepared in accordance with acceptable pharmaceutical procedures, such as, for example, those described in Remingtons Pharmaceutical Sciences, 17th edition, ed. Alfonoso R. Gennaro, Mack Publishing Company, Easton, Pa. (1985), which is incorporated herein by reference in its entirety.

The invention may be further understood by the following non-limiting examples. Reference numbers given in square brackets, [ ], or parenthesis, ( ), in the following Examples refer to the numbered references listed at the end of the Example.

### Example 1: Photonic Shell-Cross-Linked Nanoparticle Probes for Optical Imaging and Monitoring

Shell-cross-linked micelles have been shown to be excellent nanostructural platforms for a variety of biomedical applications, ranging from the delivery of large payloads of chemotherapeutics and diagnostic agents to the in vivo targeting of such entities to tumors via the external multivalent presentation of tissue specific ligands. The outstanding versatility of these systems is derived from both the ease with which they are produced (by placing amphiphilic block copolymers into a solvent that is selective for solubilizing a portion of the polymer chain segments), and the final core-shell or other (multi) compartment-type morphologies. In general, for shell-cross-linked knedel-like (SCK) nanoparticles derived from amphiphilic block copolymers containing poly(acrylic acid) as the hydrophilic, cross-linkable component, non-functional diamines have been used to chemically cross-link the carboxylate-rich shells in order to generate stable discrete nanoparticles. Even in cases where the core domain is transformed from a hydrophobic block copolymer segment to a hydrophilic polymer chain or degraded into small molecule fragments through chemical excavation strategies, the covalently-cross-linked shell layer retains structural integrity, resulting in the formation of nanocage frameworks, which are able to undergo expansion and contraction under changing environmental conditions.

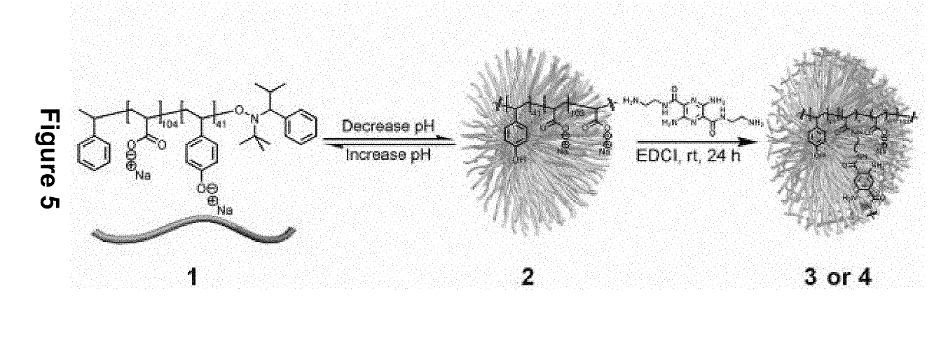

In this Example we demonstrate the use of the reversible hydrophobicity/hydrophilicity of the core domain to drive the block copolymer micelle assembly/disassembly in water without the aid of organic solvents, as a unique, green chemistry approach to the formation of SCKs. In this pursuit, we show that simple polymer nanoparticles can fashioned into sophisticated sensing devices, by bringing together the concepts of reversible hydrophobicity and nanoparticle expansion/contraction, with the use of functional cross-linkers. The functional cross-linkers provide structural integrity and optical signals to both mediate and probe the local changes within the SCKs, promoted by tuning the pH of the aqueous solution. A notable enhancement of photophysical properties for fluorophore-shell-cross-linked nanoparticles (fluorophore-SCKs), as a result of changing the pH across the physiological range. The current systems have been designed to produce high fluorescence when the shell is swollen at elevated pH and to allow for fluorescence quenching when the shell shrinks as the pH is lowered (See structures and schematic in FIG. 5). We demonstrate that the covalent attachment of fluorogenic cross-linkers within the SCK shell provides this behavior uniquely.

Photonic shell-cross-linked nanoparticles (SCKs) were prepared via cross-linking between fluorophores and micelles. These unique photonic SCKs are discussed in this Example, including their abilities to undergo pH-sensitive swelling/deswelling, which affects enhancement/quenching of the fluorescence.

The fluorophore-SCKs were assembled from the diblock copolymer precursor, poly(acrylic acid)₁₀₃-b-poly(p-hydroxystyrene)₄₁, PAA-b-PpHS, which was synthesized via nitroxide-mediated radical polymerization. Micelles were formed by first dissolving the block copolymer in water at high pH and then slowly decreasing the solution pH to 7, at which protonated PpHS block formed a hydrophobic core while maintenance of the deprotonated PAA block shaped a hydrophilic shell. FIG. 5 illustrates the preparation of micelles from poly(acrylic acid)-b-poly(p-hydroxystyrene) in water, with adjustment of the solution pH.

The resulting micelle solution 2 was incubated with 6.25 mol % or 12.5 mol % of the diamino-terminated pyrazine, relative to the acrylic acid residues, with the addition of EDCl, to afford SCK 3 or 4 having different amounts of fluorophores incorporated into the shells and, therefore, different degrees of cross-linking. The reaction mixture solutions were dialyzed against nanopure water for 4 days to remove the urea by-products and any non-attached pyrazine fluorophores. The SCK dimensions were then measured by atomic force microscopy (AFM) and transmission electron microscopy (TEM). The AFM-measured heights were observed to be 6±2 nm and 8±2 nm, and their TEM-measured diameters were 9±2 nm and 9±2 nm for SCKs 3 and 4, respectively. Dialysis of the SCK solutions against nanopure water (ca. pH 7) for 3 days and then partitioning into six vials, each containing 5 mL of 5 mM PBS at pH 4.5, 6.1, 8.0, 9.5, or 11.0, placed the SCKs into different pH environments for analysis of the effects on the SCK hydrodynamic diameters and on the fluorophore photophysical properties.

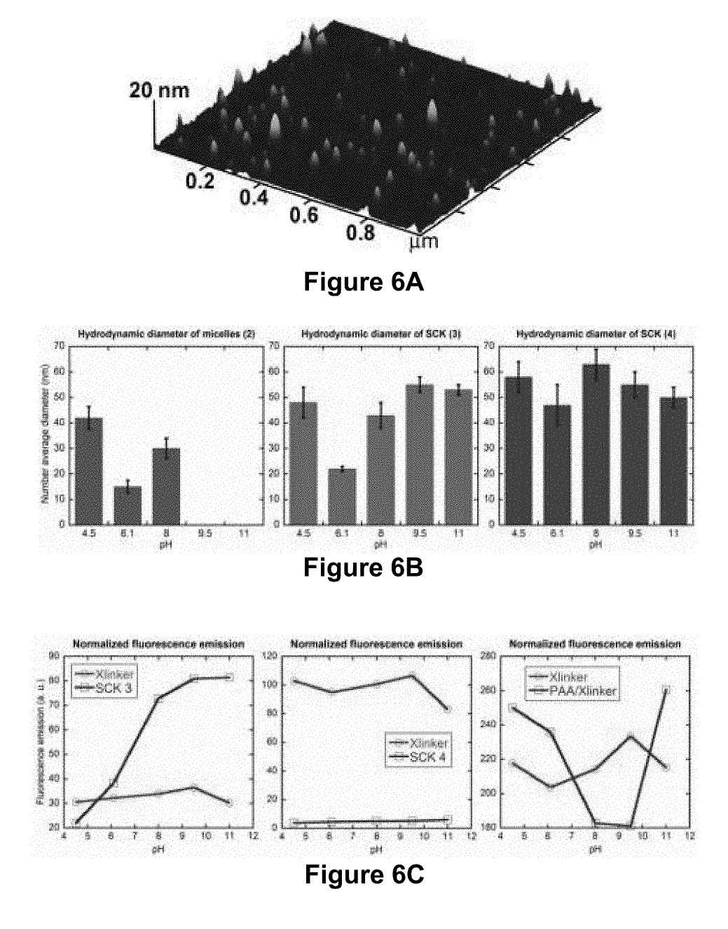

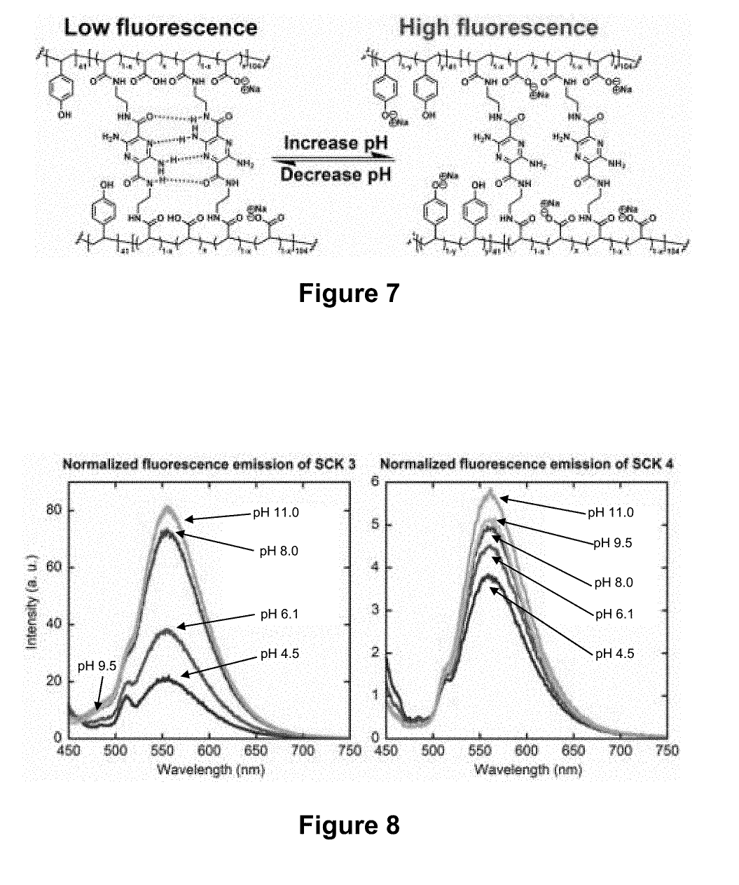

In some experiments, the resulting micelle solution was incubated with 6.25 mol % (a) or 12.5 mol % (b) of diamine-terminated pyrazine with (II) or without (I) three ethylene oxide units. The resulting solution of micelle and the cross-linker underwent covalent cross-linking by addition of EDCl, resulting in nanoparticles having hydrodynamic diameters of ca. 20 nm (as measured by DLS) and whose heights were ca. 8 nm (as measured by atomic force microscopy, AFM). FIG. 6 illustrates a representative AFM image of I-a, with an average height of 8 nm. The SCK solutions were dialyzed against 5 mM PBS at pH 7.4 for three days and then were partitioned into six vials each containing 5 mL of 5 mM PBS at pH 4.5, 6.1, 8.0, 9.5, 11.0, or 12.8.

As the SCK solution pH increases, two factors play major roles in expansion of the nanoparticles: 1) as more poly(acrylic acid) blocks become deprotonated, negatively-charged carboxylates repel PAA chains from one another within the confined SCK structure; 2) as the PpHS blocks become deprotonated at higher pH (i.e., >10), the hydrophilicity of the PpHS core increases, allowing water molecules to enter the shell-cross-linked nanoparticles. The acrylamide-pyrazine linkages are included so the composition would be able to respond to the SCKs' dual shell and core pH-driven expansion mechanisms by fluorescing upon loss of self-attractive interactions, such as hydrogen bonding, hydrophobic effects, and pi-stacking, but suffer fluorescence quenching as self-associations re-establish at lower pH values (See, FIG. 5). Due to their D₂ₕ symmetry, 2,6-diamino-2,5-diamide substituted pyrazines are quadrupolar dyes displaying photophysical characteristics that are fairly insensitive to pH changes.

Taking advantage of the SCKs' pH-responsive expansion are the diamine-terminated pyrazines, which fluoresce upon loss of intermolecular hydrogen bonding, but whose fluorescence quenches as the hydrogen bonding reestablishes. FIG. 5 illustrates the swelling/deswelling of photonic SCKs as a function of pH, where n=0 for I and n=3 for II. These pyrazine molecules are quadrupoles whose photophysical characteristics are fairly insensitive to pH changes. Thus, the resulting photophysical changes are a function of the nanostructure morphology.

Covalent cross-linking between the pyrazine units and the PAA shells, thereby, affords photonic SCKs for potential pH sensing. Covalent cross-linking between pyrazine and PAA shells constitutes photonic SCKs for potential biomedical applications. We expected to observe the photophysical consequences of the deprotonated PAA shell from pH 4.5 to 9.5 and those of the PpHS core from pH 9.5 to 11. In order to test our hypothesis, UV-vis and fluorescence measurements were collected on the resulting SCK solutions over the pH range of 4.5 to 11.0 to determine the pyrazine concentration, and then normalize the concentration relative to the fluorescence intensity values (See, FIG. 8). To demonstrate this aspect, UV-vis and fluorescence measurements were conducted on the resulting SCK solutions at different pH values, to verify the consistency in the amount of pyrazine loading in each set and to observe enhancement of photophysical properties of photonic SCKs, respectively. To observe the photophysical properties of the photonic SCKs, the data for the pyrazines within the SCK shell layers were compared between the two SCKs having different degrees of pyrazine loading and also against the pyrazine cross-linker associated physically with PAA and as a small molecule in buffered solutions.

The UV-vis and fluorescence data support the hypothesis that expansion of the fluorophore-SCKs as a function of pH provides a unique local environment to mediate the fluorescence outputs. The UV-vis measurements of 3 and 4 indicated no significant variation among data sets, confirming consistent amounts of pyrazine loading in each sample. There was an order of magnitude greater fluorescence emission intensity, however, for 3 vs. 4 (FIG. 8), suggesting that a limited amount of the fluorophore-based cross-linkers can be accommodated within the SCK shell domain while avoiding fluorescence quenching, over all of the pH values studied. Dynamic light scattering data (See, FIG. 6B) further supported this suggestion, as the variability in the SCK hydrodynamic diameter was reduced at the higher cross-linking density (12.5 mol % fluorophore for 4), whereas the lower degree of cross-linking (6.25 mol % fluorophore for 3) allowed for significant shell and core expansion with increasing pH (See, FIG. 6B). The most notable enhancement in fluorescence occurred from pH 6.1 to 8.0 (Table 1), the physiologically-relevant pH range. FIG. 6A shows a representative AFM image of a photonic SCK micelle of the present invention, having an average height of 8 nm.

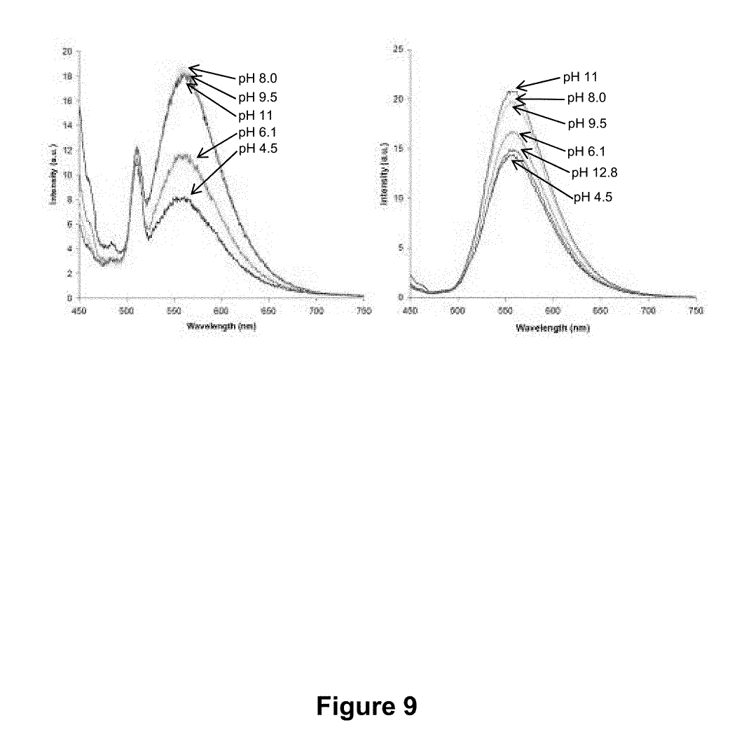

Fluorescence measurements, however, indicated significant enhancement as solution pH increased from 4.5 to 11.0, with the most notable enhancement being from 6.1 to 8.0. FIGS. 8 and 9 show fluorescence measurements of I-a, I-b, II-a, and II-b as a function of pH. All four SCK sample sets experienced the highest enhancement in fluorescence within that pH region (see, Table 1). The UV-vis and the fluorescence measurements demonstrate that the expansion of the fluorophore-SCKs at high pH disrupts the hydrogen bonding among pyrazine molecules, lowering fluorescence output. However, at pH 12.8, there was a significant drop in fluorescence (See, Table 1. I-b and II-b). In this pH region, deprotonated PpHS's quenching effect dominated, resulting in a net decrease in fluorescence.

TABLE 1
Percent increase in fluorescence as a function of pH and fluorophore
loading in SCKs I and II.

| | I | | II | |
|---|---|---|---|---|
| SCK | 12.5% | | 12.5% | |
| solution pH | xlink (a) | 25.0% xlink (b) | xlink (a) | 25.0% xlink (b) |
| pH 4.5 | 0% | 0% | 0% | 0% |
| pH 6.1 | 100% | 7% | 20% | 17% |
| pH 8.0 | 380% | 21% | 90% | 38% |
| pH 9.5 | 415% | 22% | 90% | 38% |
| pH 11.0 | 445% | 35% | 80% | 45% |
| pH 12.8 | N/A | 21% | N/A | 4% |

Only when covalently linked within the SCK shell did the pyrazine fluorophores experience an increase in fluorescence emission with increasing pH. As illustrated in FIG. 6C, the pyrazine cross-linker as a small molecule in solution or in the presence of PAA underwent no change in fluorescence intensity or gave a slight decrease in intensity on increasing from pH 4.5 to 6.1 and another decrease in intensity on increasing to pH 8.0, where it remained constant until pH 11.0 at which a slight fluorescence intensity increase was observed. In contrast, significant increases in fluorescence emission were observed for the pyrazines in 3 (Table 2), ca. 330% increase over the pH range where expansion of the shell is expected due to deprotonation of residual acrylic acid residues, and ca. 370% fluorescence increase with deprotonation of the phenolic groups and expansion of the core domain, each relative to the fluorescence intensity observed at pH 4.5. The higher degree of cross-linking and higher loading of pyrazine of 4 limited the nanostructure expansion and promoted pyrazine-pyrazine fluorescence quenching, which together reduced the observed effects on fluorescence intensity (FIG. 6C and Table 2).

TABLE 2
Normalized percent increase in fluorescence as a function of pH and
fluorophore loading in SCK.

| solution | Normalized percent increase | | | |
|---|---|---|---|---|
| pH | SCK 3^{[a]} | SCK 4^{[b]} | PAA/cross-linker^{[c]} | cross-linker^{[d]} |
| pH 4.5 | 100% | 100% | 100% | 100% |
| pH 6.1 | 180% | 120% | 90% | 100% |
| pH 8.0 | 330% | 130% | 70% | 100% |
| pH 9.5 | 370% | 140% | 70% | 110% |
| pH 11.0 | 370% | 150% | 100% | 100% |

^{[a]}6.25 mol % fluorophore loading
^{[b]}12.5 mol % fluorophore loading
^{[c]}PAA and fluorophore complex at 6.25 mol % fluorophore loading, relative to acrylic acid residues
^{[d]}fluorophore stock solution.

According, these results, demonstrated successful preparation and characterization of fluorophore-SCKs using a bifunctional optical probe molecule as a photonic linkage system for the shell-cross-linking step in SCK formation. We utilize a pH-insensitive fluorophore to generate a pH-sensing assembly through its covalent incorporation within a nanostructure derived from pH-responsive polymers. The bifunctional fluorophore could then be described as a photonic linkage system for the shell-cross-linking step in SCK formation.

Dynamic light scattering data and UV-vis/fluorescence measurements together have shown that the fluorophore-SCKs respond morphologically to pH (swell at high pH and shrink at low pH) through the incorporation of functionality of differential pKₐ (i.e. phenols/carboxylate). Photophysical consequences of these changes in morphology were manifested as “fluorescence on” at higher pH values and “fluorescence off” at lower pH values due to proximity quenching. Biomedical uses for these new photonic nanosystems include chemical/physiological sensors, among other applications.

Experimental Section

### Synthesis of poly(tert-butyl acrylate)₁₀₄ (5)

To a flame-dried 50-mL Schlenk flask equipped with a magnetic stir bar and under N₂ atmosphere, at room temperature (rt), was added 2,2,5-trimethyl-3-(1′-phenylethoxy)-4-phenyl-3-azahexane (600 mg, 1.84 mmol), 2,2,5-trimethyl-4-phenyl-3-azahexane-3-nitroxide (20.0 mg, 0.092 mmol), and tert-butyl acrylate (31.5 g, 245 mmol). The reaction flask was sealed and stirred 10 min at rt (i.e. room temperature). The reaction mixture was degassed through three cycles of freeze-pump-thaw. After the last cycle, the reaction mixture was recovered back to room temperature and stirred for 10 min before being immersed into a pre-heated oil bath at 125° C. to start the polymerization. After 72 h, ¹H NMR analysis showed 72% monomer conversion had been reached. The polymerization was quenched by quick immersion of the reaction flask into liquid N₂. The reaction mixture was dissolved in THF and precipitated into H₂O/MeOH (v:v, 1:4) three times to afford white powder, (19.3 g, 85% yield based upon monomer conversion); M_{n,GPC}=13,700 Da, PDI=1.1, DP=104.

### Synthesis of poly(tert-butylacrylate)₁₀₄-b-poly(acetoxystyrene)₄₁ (6)

To a flame-dried 50-mL Schlenk flask equipped with a magnetic stir bar and under N₂ atmosphere at room temperature, 5 (3.0 g, 0.22 mmol) and 4-acetoxystyrene (4.44 g, 27.4 mmol) were added. The reaction mixture was allowed to stir for 1 h at room temperature to obtain a homogenous solution. The reaction mixture was degassed through three cycles of freeze-pump-thaw. After the last cycle, the reaction mixture was recovered back to room temperature and stirred for 10 min before being immersed into a pre-heated oil bath at 125° C. to start the polymerization. After 6 h, 32% monomer conversion was reached, as analyzed by ¹H NMR spectroscopy. After quenching by immersion of the reaction flask into a bath of liquid N₂, THF was added to the reaction mixture and the polymer was purified by precipitating into H₂O/MeOH (v:v, 1:4) three times to afford 6 as a white powder, (3.73 g, 83% yield); M_{n,GPC}=17,400 Da, PDI=1.3, DP=41.

### Preparation of poly(tert-butyl acrylate)₁₀₄-b-poly(p-hydroxystyrene)₄₁ (7)

To a 25-mL round bottom (RB) flask, (6) (3.0 g, 0.15 mmol) and MeOH (10 mL) were added and stirred 10 min at room temperature. The cloudy mixture was heated slowly to reflux. Immediately after the solution turned clear, a sodium methoxide solution in MeOH (25 wt %) (26 mg, 0.12 mmol) was added through syringe. The reaction mixture was further allowed to heat at reflux for 4 h. After cooling down to room temperature, the reaction mixture was precipitated in water with 4% acetic acid to afford 7 as white solid (2.6 g, 95% yield). M_{n,NMR}=18,600 Da.

### Synthesis of poly(acrylic acid)₁₀₄-b-poly(p-hydroxystyrene)₄₁ (1)

To a 50 mL RB flask equipped with a stir bar, was added 7 (2.5 g, 0.13 mmol) and trifluoroacetic acid (20.2 g, 177 mmol). The reaction mixture was allowed to stir for 24 h at room temperature. Excess acid was removed under vacuum. The residue was dissolved into 10 mL of THF and purified by dialysis against nanopure water (18.0 MΩ-cm) for three days and freeze-dried to afford 1 as a white powder (1.6 g, 95% yield). M_{n,NMR}=12,000 Da

### Preparation of Micelle 2 from 1

To a 50 mL of RB flask equipped with a magnetic stir bar was added 1 (2.0 mg, 0.16 μmol) and 15 mL of nanopure water. The pH value was adjusted to 12 by adding 1.0 M NaOH solution to afford a clear solution. The micellization was initiated after decreasing the pH value to 7 by adding dropwise 1.0 M HCl. After further stirring 12 h at room temperature, the micelle solution was used directly for construction of SCK 3 and 4.

### Preparation of Shell-Cross-Linked Nanoparticles (SCK 3 or 4) from Micelle 2

To a 50 mL RB flask equipped with a magnetic stir bar was added a solution of micelles in nanopure H₂O (15.0 mL, 0.016 mmol of carboxylic acid residues). To this solution, was added a solution of 3,6-diamino-N²,N⁵-bis(2-aminoethyl)pyrazine-2,5-dicarboxamide (0.397 mg, 1.12 gmol (6.25 mol % relative to the acrylic acid residues) for 12.5% cross-linking extent; or 0.794 mg, 2.24 μmol (12.5 mol % relative to the acrylic acid residues) for 25% cross-linking extent) in 1 mL nanopure H₂O. The reaction mixture was allowed to stir for 2 h at room temperature. To this solution was added, dropwise via a syringe pump over 1 h, a solution of 1-[3′-(dimethylamino)propyl]-3-ethylcarbodiimide methiodide (EDCl, 0.849 mg, 2.86 gmol for 12.5% cross-linking extent; or 1.70 mg, 5.72 gmol for 25% cross-linking extent) in nanopure H₂O (1.0 mL) and the reaction mixture was further stirred for 16 h at room temperature. Finally, the reaction mixture was transferred to pre-soaked dialysis tubing (MWCO ca. 3,500 Da) and dialyzed against nanopure water for 3 d to remove the small molecule starting materials and by-products, and afford aqueous solutions of SCK 3 and 4. SCK solutions for DLS, UV-vis, and fluorescence studies were further partitioned into six vials each containing 5 mM PBS (with 5 mM NaCl) at pH values of 4.5, 6.1, 8.0, 9.5, and 11.

### Synthesis of 3,6-diamino-N²,N⁵-bis(2-aminoethyl)pyrazine-2,5-dicarboxamide

A mixture of sodium 3,6-diaminopyrazine-2,5-dicarboxylate (500 mg, 2.07 mmol), tert-butyl 2-aminoethylcarbamate (673 mg, 4.20 mmol), HOBt (836 mg, 5.46 mmol) and EDCl (1.05 g, 5.48 mmol) in DMF (25 mL) was allowed to stir for 16 h and was then concentrated. The residue was partitioned with 1 N NaHSO₄ (200 mL) and EtOAc (200 mL). The organic layer was separated and washed with water (200 mL×3), sat. NaHCO₃ (200 mL×3) and brine. Dried with MgSO₄, filtered and concentrated to afford the bisamide as an orange foam. 770 mg, 76% yield. ¹H NMR (300 MHz, DMSO-d₆)major conformer, δ 8.44 (t, J=5.7 Hz, 2H), 6.90 (t, J=5.7 Hz, 2H), 6.48 (br, 4H), 2.93-3.16 (m, 8H), 1.37 (s, 9H), 1.36 (s, 9H) ppm. ¹³C NMR (75 MHz, DMSO-d₆), δ 165.1, 155.5, 155.4, 146.0, 126.2, 77.7, 77.5, 45.2, 44.5, 28.2 ppm. LC-MS (15-95% gradient acetonitrile in 0.1% TFA over 10 min), single peak retention time=7.18 min on 30 mm column, (M+H)⁺=483 amu. To the product (770 mg, 1.60 mmol) in methylene chloride (100 mL), was added TFA (25 mL) and the reaction was stirred at room temperature for 2 h. The mixture was concentrated and the residue was dissolved into methanol (15 mL). Diethyl ether (200 mL) was added and the orange solid precipitate was isolated by filtration and dried at high vacuum to afford an orange powder. 627 mg, 77% yield.

¹H NMR (300 MHz, DMSO-d₆) δ 8.70 (t, J=6 Hz, 2H), 7.86 (br, 6H), 6.50 (br, 4H), 3.46-3.58 (m, 4H), 3.26-3.40 (m, 4H) ppm. ¹³C NMR (75 MHz, DMSO-d₆) δ 166.4, 146.8, 127.0, 39.4, 37.4 ppm. LC-MS (15-95% gradient acetonitrile in 0.1% TFA over 10 min), single peak retention time=2.60 min on 30 mm column, (M+H)⁺=283 amu. UV-vis (100 μM in PBS) λ_{abs}=435 nm. Fluorescence (100 nM) λₑₓ=449 nm, λₑₘ=562 nm. The product was converted to the HCl salt by coevaporation (3×100 mL) with 1 N aqueous HCl.

### Example 2: General Methods for Photonic Cross-Linker Synthesis

Analytical thin layer chromatography (TLC) was performed on Analtech 0.15 mm silica gel 60-GF₂₅₄ plates. Visualization was accomplished with exposure to UV light, exposure to Iodine or by dipping in an ethanolic PMA solution followed by heating. Solvents for extraction were HPLC or AGS grade. Chromatography was performed by the method of Still with Merck silica gel 60 (230-400 mesh) with the indicated solvent system. NMR spectra were collected on a Bruker ARX-500, or Varian Mercury-300 spectrometer. ¹H NMR spectra were reported in ppm from tetramethylsilane on the 5 scale. Data are reported as follows: Chemical shift, multiplicity (s=singlet, d=doublet, t=triplet, q=quartet, m=multiplet, b=broadened, obs=obscured), coupling constants (Hz), and assignments or relative integration. ¹³C NMR spectra were reported in ppm from the central deuterated solvent peak. Grouped shifts are provided where an ambiguity has not been resolved. Preparative reversed phased liquid chromatography runs were conducted on a low pressure system employing an AllTech Model 7125 Rheodyne Injector Valve with a 5 mL sample loop, an AllTech Model 426 pump, an ISCO UA-6 absorbance detector with built-in recorder, peak separator and type 11 optical unit, an ISCO Foxy 200 fraction collector and using Lobar LiChroprep RP-18 (40-63 μm) prepacked columns and on a Waters Autopurification System using a Waters XBrigdge Preparative C18 OBD 30×150 mm column. LCMS were run on a Shimadzu LCMS-2010A using Agilent Eclipse (XDB-C18, 4.6×30 mm, 3.5-Micron) Rapid Resolution Cartridges and Agilent Eclipse (XDB-C18 4.6×250 mm, 3.5-Micron) Columns. GCMS were run on a Varian Saturn 2000 using a DB5 capillary column (30 m×0.25 mm I.D., 1.0 μfilm thickness). MALDI mass spectra were run on a PE Biosystems Voyager System 2052. Electronic absorption spectra were measured in phosphate buffered saline using a Shimadzu UV-3101PC UV-VIS-NIR scanning spectrophotometer. Emission spectra were recorded in phosphate buffered saline using a Jobin Yvon Fluorolog-3 fluorescence spectrometer.

Photonic Cross-Linker Chemistry

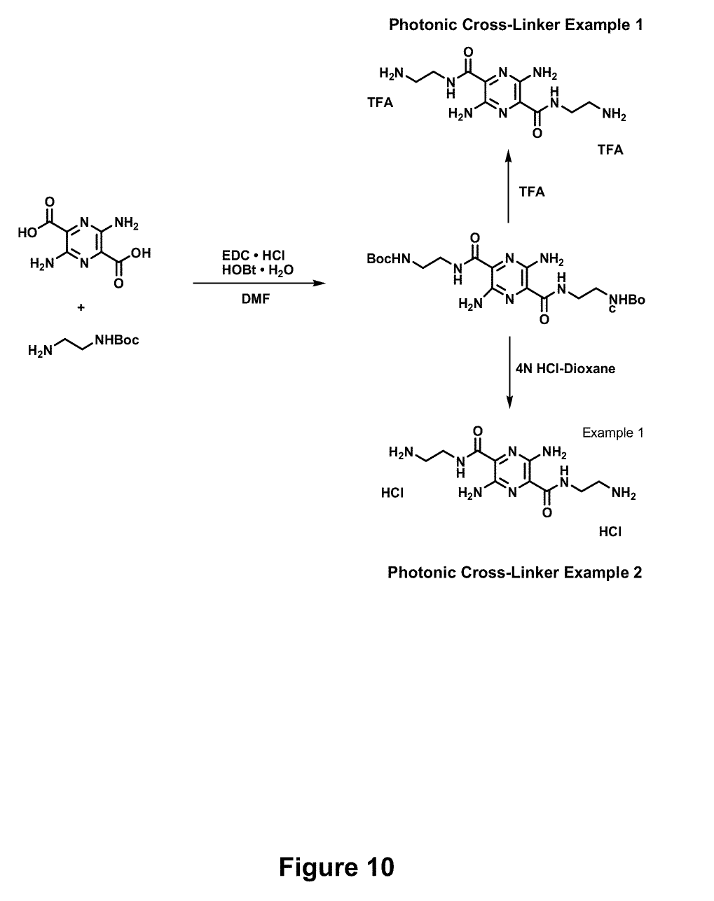

Photonic Cross-Linker Example 1: 3,6-diamino-N²,N⁵-bis(2-aminoethyl)pyrazine-2,5-dicarboxamide bis-TFA salt. FIG. 10 illustrates a synthetic pathway for production of Photonic Cross-linker Example 1.

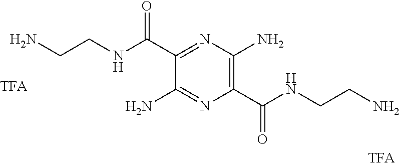 [SMILES: NCCNC(=O)c1nc(N)c(C(=O)NCCN)nc1N]

### Step 1. Synthesis of 3,6-diamino-N²,N⁵-bis[2-(tert-butoxycarbonyl)aminoethyl]pyrazine-2,5-dicarboxamide

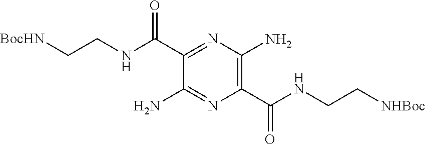 [SMILES: CC(C)(C)OC(=O)NCCNC(=O)c1nc(N)c(C(=O)NCCNC(=O)OC(C)(C)C)nc1N]

A mixture of sodium 3,6-diaminopyrazine-2,5-dicarboxylate (500 mg, 2.07 mmol), tert-butyl 2-aminoethylcarbamate (673 mg, 4.20 mmol), HOBt-H₂O (836 mg, 5.46 mmol) and EDC-HCl (1.05 g, 5.48 mmol) in DMF (25 mL) was stirred for 16 h and concentrated. The residue was partitioned with EtOAc (100 mL) and 1N NaHSO₄ (100 mL). The layers were separated and the EtOAc solution was washed with water (100 mL), saturated sodium bicarbonate (100 mL) and brine (100 mL). The EtOAc layer was dried (MgSO₄), filtered and concentrated to afford 770 mg (76% yield) of the bisamide as an orange foam: ¹H NMR (300 MHz, DMSO-d₆), major conformer, δ 8.44 (t, J=5.7 Hz, 2H), 6.90 (t, J=5.7 Hz, 2H), 6.48 (bs, 4H), 2.93-3.16 (complex m, 8H), 1.37 (s, 9H), 1.36 (s, 9H). ¹³C NMR (75 MHz, DMSO-d₆), conformational isomers δ 165.1 (s), 155.5 (bs), 155.4 (bs), 146.0 (s), 126.2 (s), 77.7 (bs), 77.5 (bs), 45.2 (bt), 44.5 (bt), 28.2 (q).

### Step 2

To the product from step 1 (770 mg, 1.60 mmol) in methylene chloride (100 mL) was added TFA (25 mL) and the reaction was stirred at room temperature for 2 h. The mixture was concentrated and the residue taken up into methanol (15 mL). Ether (200 mL) was added and the orange solid precipitate was isolated by filtration and dried at high vacuum to afford 627 mg (77% yield) of Photonic Cross-Linker Example 1 as an orange powder: ¹H NMR (300 MHz, DMSO-d₆), δ 8.70 (t, J=6 Hz, 2H), 7.86 (bs, 6H), 6.50 (bs, 4H), 3.46-3.58 (m, 4H), 3.26-3.40 (m, 4H). ¹³C NMR (75 MHz, DMSO-d₆) δ 166.4 (s), 146.8 (s), 127.0 (s), 39.4 (t), 37.4 (t). LCMS (5-95% gradient acetonitrile in 0.1% TFA over 10 min), single peak retention time=3.62 min on 30 mm column, (M+H)+=283. UV/vis (100 μM in PBS) λ_{abs}=435 nm. Fluorescence (100 nM) λₑₓ=449 nm, λₑₘ=562 nm.

Photonic Cross-Linker Example 2: 3,6-diamino-N²,N⁵-bis(2-aminoethyl)pyrazine-2,5-dicarboxamide dihydrochloride. FIG. 10 illustrates a synthetic pathway for production of Photonic Cross-linker Example 2.

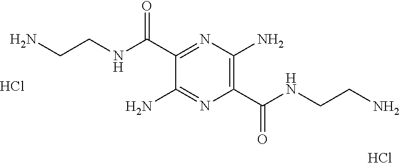 [SMILES: Cl ; NCCNC(=O)c1nc(N)c(C(=O)NCCN)nc1N ; Cl]

The product from Example 1, step 1 (351 mg, 0.73 mmol) was dissolved in 4N HCl-dioxane (35 mL) and the reaction mixture was stirred for 30 min at room temperature. The reaction was concentrated and triturated with ether (100 mL) to afford 226 mg (87% yield) of Photonic Cross-Linker Example 2 as an orange solid: MS (ESI) m/z=283 [M+H]⁺. UV/vis (100 μM in PBS) λ_{abs}=435 nm. Fluorescence (100 nM) λₑₓ=449 nm, λₑₘ=562 nm.

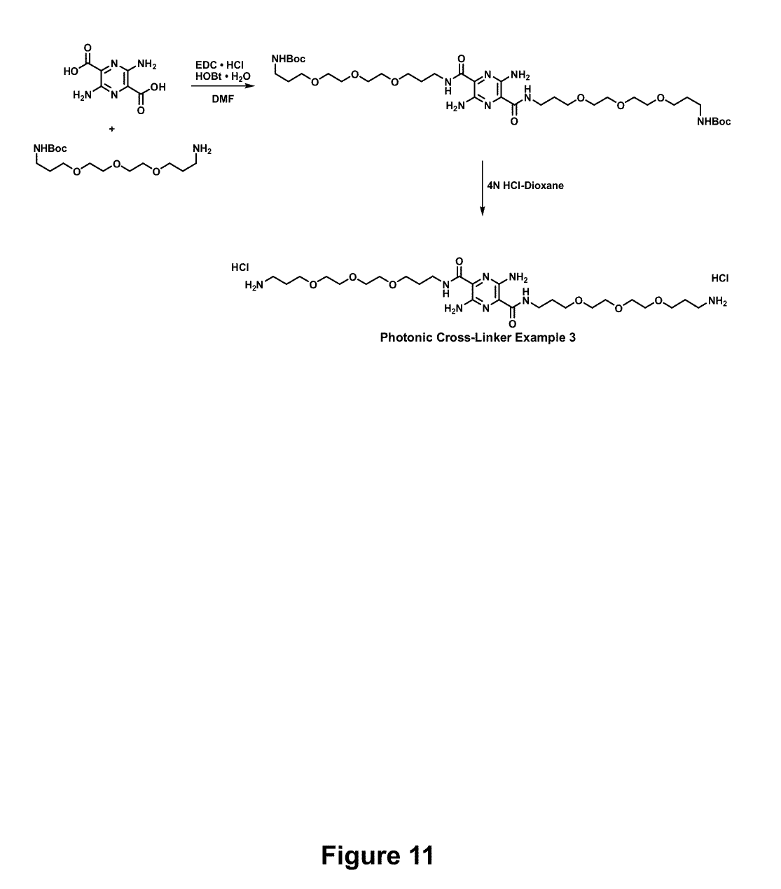

Photonic Cross-Linker Example 3: 3,6-diamino-N²,N⁵-bis(3-(2-(2-(3-aminopropoxy)ethoxy)ethoxy)propyl)pyrazine-2,5-dicarboxamide dihydrochloride. FIG. 11 illustrates a synthetic pathway for production of Photonic Cross-linker Example 3.

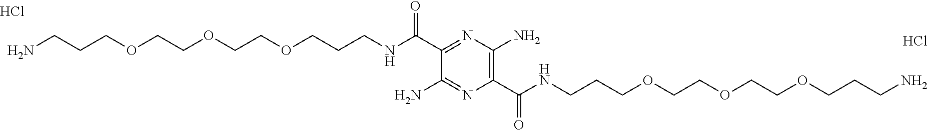 [SMILES: Cl ; NCCCOCCOCCOCCCNC(=O)c1nc(N)c(C(=O)NCCCOCCOCCOCCCN)nc1N ; Cl]

### Step 1. Synthesis of tert-butyl 1,1′-(3,6-diaminopyrazine-2,5-diyl)bis(1-oxo-6,9,12-trioxa-2-azapentadecane-15,1-diyl)dicarbamate

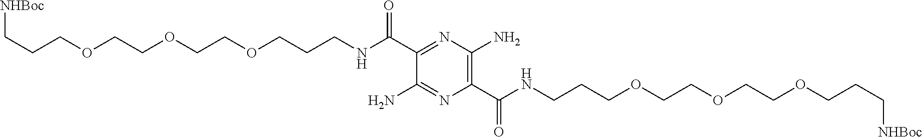 [SMILES: CC(C)(C)OC(=O)NCCCOCCOCCOCCCNC(=O)c1nc(N)c(C(=O)NCCCOCCOCCOCCCNC(=O)OC(C)(C)C)nc1N]

A mixture of 3,6-diaminopyrazine-2,5-dicarboxylic acid (0.31 g, 1.56 mmol), tert-butyl 3-(2-(2-(3-aminopropoxy)ethoxy)ethoxy)propylcarbamate (1.00 g, 3.12 mmol), EDC-HCl (0.72 g, 3.74 mmol) and HOBt (0.50, 3.74 mmol) was stirred in DMF (35 mL) for 16 hr at room temperature. Concentration and workup as in Photonic Cross-Linker Example 1 afforded the crude bis-amide which was taken on to the next step with no further purification: HRMS calcd for C₃₆H₆₆N₈O₁₂Na, 825.4692 [M+Na]⁺; found, 825.4674.

### Step 2

The crude product mixture from step 1 (˜1.20 g, 1.50 mmol) was added 4N HCl-Dioxane (10 mL) and the resulting mixture was stirred for 1 hr at room temperature. Concentration, trituration of the residue with 1:1 hexanes-ether (100 mL) and pumping at high vacuum afforded Photonic Cross-Linker Example 3 as a viscous orange oil: LCMS (5-95% gradient acetonitrile in 0.1% TFA over 10 min), single peak retention time=5.70 min on 30 mm column, [M+H]⁺=603. UV/vis (100 μM in PBS) λ_{abs}=435 nm. Fluorescence (100 nM) λₑₓ=449 nm, λₑₘ=562 nm.

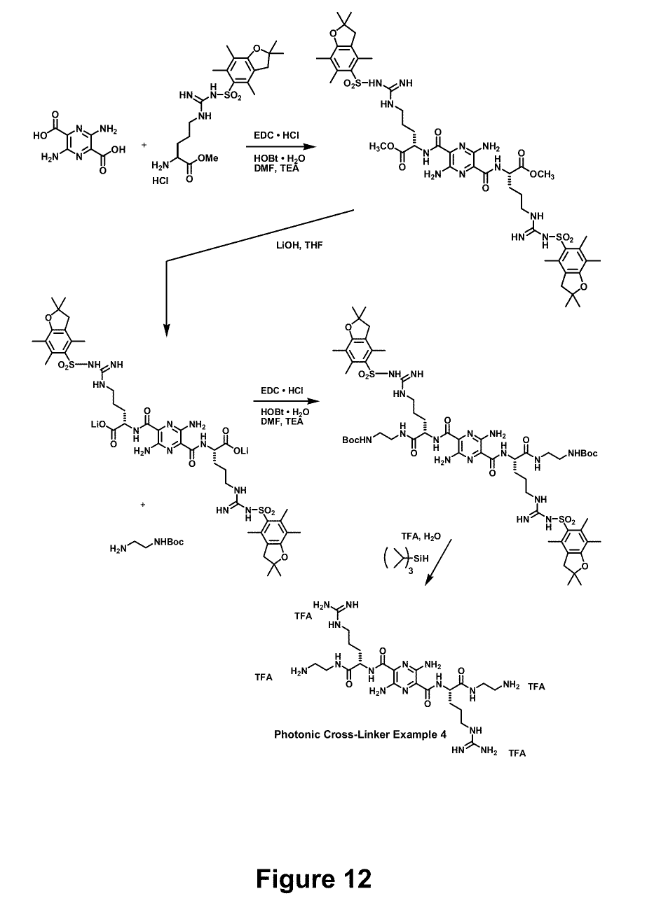

Photonic Cross-Linker Example 4: 3,6-Diamino-N2,N5-bis [N-(2-aminoethyl)-Arginine amide]-pyrazine-2,5-dicarboxamide tetra TFA salt. FIG. 12 illustrates a synthetic pathway for production of Photonic Cross-linker Example 4.

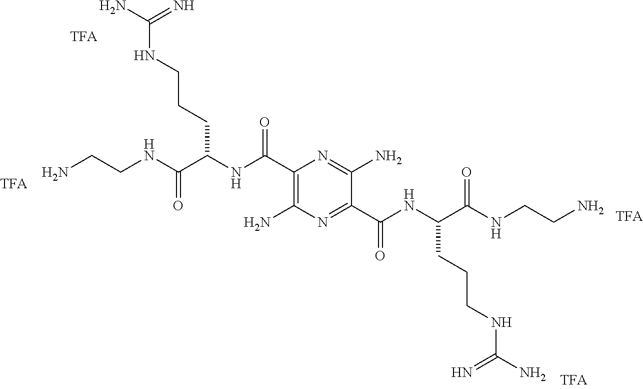 [SMILES: N=C(N)NCCC[C@H](NC(=O)c1nc(N)c(C(=O)N[C@@H](CCCNC(=N)N)C(=O)NCCN)nc1N)C(=O)NCCN]

### Step 1. Synthesis of 3,6-Diamino-N2,N5-bis (N-pbf-Arginine methyl ester)-pyrazine-2,5-dicarboxamide

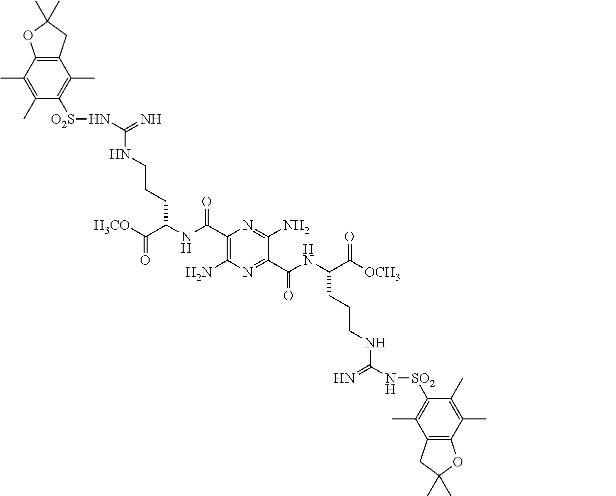

A mixture of 3,6-diaminopyrazine-2,5-dicarboxylic acid (0.90 g, 4.54 mmol), H-Arg(pbf)-OMe*HCl (4.77 g, 9.99 mmol), EDC (1.53 g, 9.99 mmol), HOBt (1.34 g, 9.99 mmol) and TEA (726 μL, 9.99 mmol) was stirred in DMF (35 mL) for 16 hr at room temperature. Concentration and workup as in Photonic Cross-Linker Example 1 followed by filtration through a plug of silica gel afforded the crude bis-amide which was taken on to the next step with no further purification.

### Step 2. Synthesis of 3,6-Diamino-N2,N5-bis (N-pbf-Arginine)-pyrazine-2,5-dicarboxamide di-lithium salt

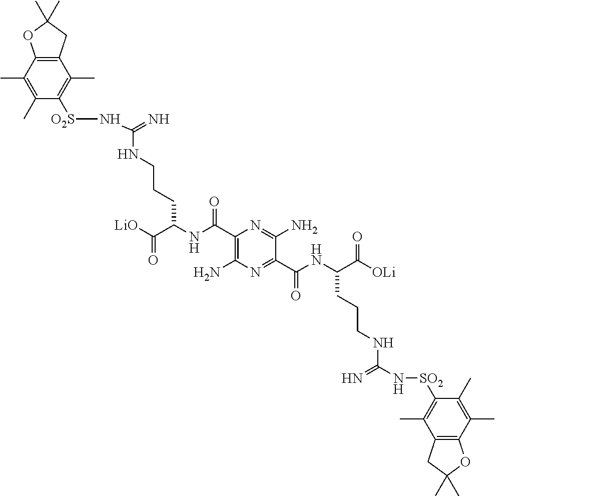

A solution of the product from Step 1 (2.40 g, 2.30 mmol) in THF (35 mL) was treated with a solution of lithium hydroxide (276 mg 11.5 mmol) in water (5.0 mL). After stirring for 1 hr at room temperature, HPLC analysis indicated reaction was complete. The reaction was quenched by the addition of dry ice and concentrated. This material was used in the next step without further purification.

### Step 3. Synthesis of 3,6-Diamino-N²,N⁵-bis [N-(2-Bocaminoethyl)-Arginine amide]-pyrazine-2,5-di-carboxamide

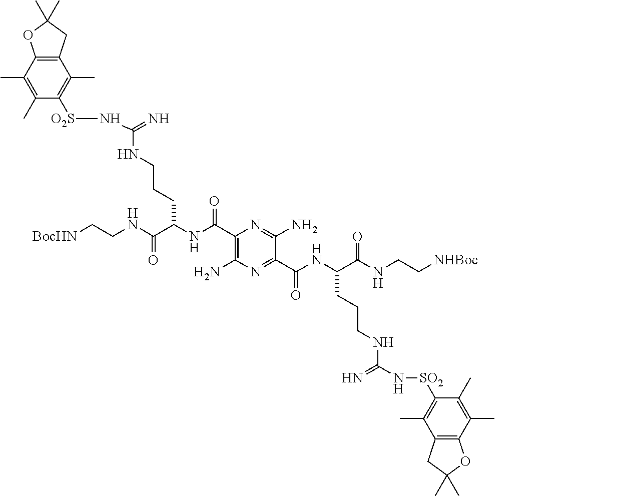

A mixture of the product from Step 2 (1.00 g, 0.97 mmol), tert-butyl 2-aminoethyl-carbamate (350 mg, 2.19 mmol), EDC-HCl (420 mg, 2.19 mmol) HOBt (290 mg, 2.15 mmol) and TEA (˜0.5 mL) in DMF (50 mL) was stirred at room temperature for 16 h. The reaction was concentrated and the residue was processed as in Photonic Cross-Linker Example 1 to afford 1.05 g of product as a red semi-solid: MS (ESI) [M+H]⁺=1300; [M+Na]⁺=1323. This material was used in the next step without further purification.

### Step 4

Synthesis of Photonic Cross-Linker Example 4. To the product from Step 3 (900 mg, 0.69 mmol) was added TFA (9.25 mL), water (25 μL), and triisopropyl silane (25, μL). The resulting mixture was stirred at room temperature for 72 h (convenience—over weekend). The reaction mixture was concentrated. The residue was purified by preparative HPLC (C18, 30×150 mm column, 5% ACN in H₂O to 95% over 12 min, 0.1% TFA) to afford 178 mg (26% yield) of Photonic Cross-Linker Example 4 as a red foam: HRMS calcd for C₂₂H₄₃N₁₆O₄, 595.3648 [M+H]⁺; found, 595.3654.

### Poly(acrylic acid)-b-poly(p-hydroxystyrene) Chemistry: Synthesis of Block Copolymers Via Nitroxide-Mediated Radical Polymerization

Synthesis of poly(tert-butylacrylate)₁₀₄ (I): In a 50-mL Schlenk flask with a magnetic stir bar, 2,2,5-trimethyl-3-(1-phenylethoxy)-4-phenyl-3-azahexane (500 mg, 1.34 mmol), 2,2,5-trimethyl-4-phenyl-3-azahexane-3-nitroxide (17.0 mg, 0.077 mmol), and tert-butyl acrylate (16.4 g, 128 mmol) were mixed together. The reaction mixture underwent three cycles of freeze-pump-thaw. The reaction was heated to 125° C. rapidly in a pre-heated oil bath. After 23 hrs, the reaction was quenched in liquid nitrogen. The reaction mixture was dissolved in THF and precipitated in 20% H₂O in MeOH three times to afford white powder, (7.17 g, 86% yield); M, =13,700 Da, PDI=1.1, DP=40, conv=61%.

Synthesis of poly(tert-butylacrylate)₁₀₄-b-poly(acetoxystyrene)₄₁ (II): In a 50-mL Schlenk flask, I (2.0 g, 0.37 mmol), 4-acetoxystyrene (5.95 g, 37 mmol), and DMF (0.5 mL) was added to obtain a homogenous mixture. The reaction mixture was heated to 125° C. in a pre-heated oil bath and heated under stirring for 23 h. The reaction was dissolved in THF and precipitated in 20% H₂O in MeOH three times to afford white powder, (5.81 g, 91% yield); Mₙ=17,402 Da, PDI=1.3, DP=70, conv=80%.

Synthesis of poly(tert-butylacrylate)₁₁₀ (Ill): In a 50-mL Schlenk flask with a magnetic stir bar, 2,2,5-trimethyl-3-(1-phenylethoxy)-4-phenyl-3-azahexane (600 mg, 1.84 mmol), 2,2,5-trimethyl-4-phenyl-3-azahexane-3-nitroxide (20.0 mg, 0.092 mmol), and tert-butyl acrylate (31.5 g, 245 mmol) were mixed together. The reaction mixture underwent three cycles of freeze-pump-thaw. The reaction was heated to 125° C. rapidly in a pre-heated oil bath. After 72 hrs, the reaction was quenched in liquid nitrogen. The reaction mixture was dissolved in THF and precipitated in 20% H₂O in MeOH three times to afford white powder, (19.33 g, 73% yield); Mₙ=14,400 Da, PDI=1.1, DP=40, conv=61%.

Synthesis of poly(tert-butylacrylate)₁₁₀-b-poly(acetoxystyrene)₂₀₇ (IV): In a 50-mL Schlenk flask, III (1.70 g, 0.108 mmol), 4-acetoxystyrene (11.87 g, 64.6 mmol), 2,2,5-trimethyl-4-phenyl-3-azahexane-3-nitroxide (1.19 mg, 5.39 μmol), and DMF (4.0 mL) was added to obtain a homogenous mixture. The reaction mixture was heated to 125° C. in a pre-heated oil bath and let stir for 8 h. The reaction was dissolved in THF and precipitated in 20% H₂O in MeOH three times to afford white powder, (3.84 g, 74% yield); M, =48,300 Da, PDI=1.3, DP=200, conv=35%.

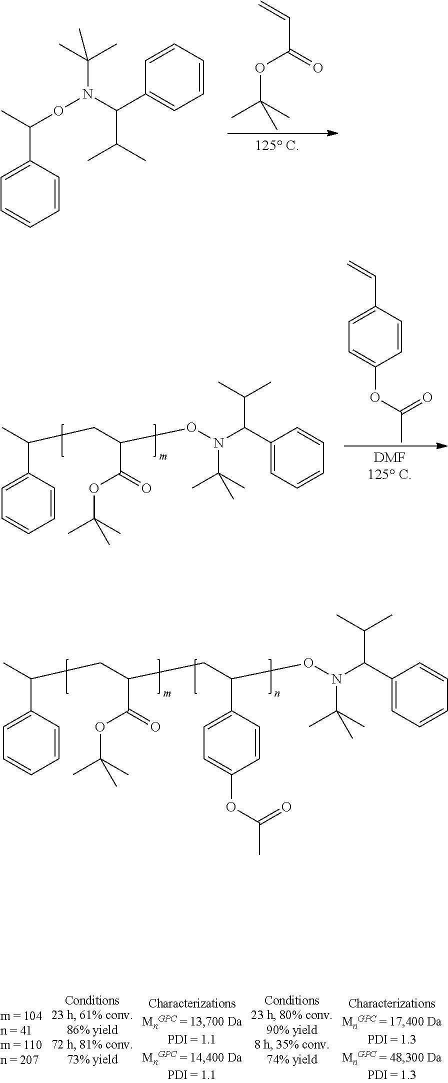

Hydrolysis of II or IV to afford poly(tert-butylacrylate)₁₀₄-b-poly(p-hydroxystyrene)₄₁ (V) or poly(tert-butylacrylate)₁₁₀-b-poly(p-hydroxystyrene)₂₀₇ (VI): In a 25-mL rb flask, II (3.0 g, 0.148 mmol) or IV (3.84 g, 0.08 mmol) and MeOH (10 mL) were added and let stirred at room temperature for 10 min. A cloudy mixture was heated slowly to reflux. Immediately after the solution cleared, sodium methoxide (25% in MeOH) (26 mg, 0.12 mmol or 76 mg, 0.35 mmol) was syringed into the reaction pot. The reaction mixture was allowed to heat at reflux for 4 h. The reaction mixture was precipitated in acetic acid (4% in water) to afford 2.6 g (95% yield), Mₙ^{NMR}=18,600 Da (V) or 3.0 g (97% yield), Mₙ^{NMR}=38,700 Da (VI).

Acidolysis of V or VI to afford poly(acrylic acid)₁₀₄-b-poly(p-hydroxystyrene)₄₁ (VII) or poly(acrylic acid)₁₁₀-b-poly(p-hydroxystyrene)₂₀₇ (VIII): In a Schlenk flask, V (2.5 g, 0.134 mmol) or VI (2.9 g, 0.075 mmol) was added with a stir bar. Excess amount of trifluoroacetic acid (20.2 g, 177 mmol) was syringed into the reaction pot to solubilize the block copolymer and let stirred for 24 h. The reaction mixture was dissolved in 10 mL of methylene chloride. Residual acid and solvent were removed in vacuum. The purification process was repeated three times. Slightly pink solution was dialyzed against nanopure water for three days and freeze-dried to afford 1.6 g or 2.4 g of the white polymer (95% or 94% yield). IR (v): 3438 (OH inter-, intramolecular H-bond), 2928 (COOH dimer), 1654 (COOH intramolecular H-bond), 1560-1384 (COOH anion), 1249 (Aryl-OH), 1172-1123 (C—OH) cm⁻¹.

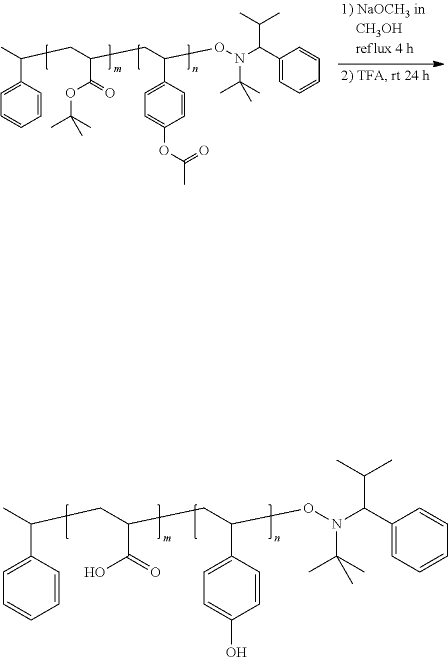

Photonic Shell Cross-Linked Nanoparticle Probe Chemistry

Preparation of micelles from VII or VIII: Micelles were prepared by first dissolving 2 mg of the block copolymer VII or VIII in 15 mL of nanopure water and stirring for 12 hrs.

Preparation of shell cross-linked nanoparticles (SCK) from micelles: Micelle solution pH was adjusted between 5 and 6. An electronic pipette was used to add 6.25 mol % or 12.5 mol % of diamine-terminated cross-linker (from stock solution with concentration 2.392 mg/mL or 6.2957 mg/mL) to the micelle solution and let stir for 3 hrs. To this reaction mixture was added dropwise, via a metering pump, a solution of 1-[3-(dimethylamino)propyl]-3-ethylcarbodiimide methiodide dissolved in nanopure water (12.5 mol % or 25.0 mol %). The reaction mixture was allowed to stir for 24 hrs at room temperature and was then transferred to presoaked dialysis membrane tube (MWCO ca. 3.5 kDa), and dialyzed against 5 mM PBS solution for three days to remove small molecules. SCK solutions for TEM and AFM studies were further dialyzed against nanopure water for three days and its pH adjusted to the desired value by addition of NaOH/HCl. SCK solutions for DLS, UV-vis, and fluorescence studies were further partitioned into six vials each containing 5 mM PBS at pH values 4.5, 6.1, 8.0, 9.5, 11.0, and 12.8.

Characterization Methods: Characterization of the polymers by gel permeation chromatography (GPC): Molecular weight and the molecular weight distribution (PDI) of the polymers I, II, III, and IV were determined by GPC. GPC was conducted on a Waters 1515 HPLC (Waters Chromatography, Inc.) equipped with a Waters 2414 differential refractometer, a PD2020 dual angle (15° and 90°) light scattering detector (Precision Detectors, Inc.), and a three column series PL gel 5 μm Mixed C, 500 Å, and 10⁴ Å, 300×7.5 mm columns (Polymer Laboratories Inc.). The system was equilibrated at 35° C. in anhydrous THF, which served as the polymer solvent and eluent with a flow rate of 1.0 mL/min. Polymer solutions were prepared at a known concentration (ca. 3 mg/mL) and an injection volume of 200 μL was used. Data collection and analysis were performed, respectively, with Precision Acquire software and Discovery 32 software (Precision Detectors, Inc.). Interdetector delay volume and the light scattering detector calibration constant were determined by calibration using a nearly monodispersed polystyrene standard (Pressure Chemical Co., Mₚ=90 kDa, M_{w}/Mₙ<1.04). The differential refractometer was calibrated with standard polystyrene reference material (SRM 706 NIST), of known specific refractive index increment dn/dc (0.184 mL/g). The dn/dc values of the analyzed polymers were then determined from the differential refractometer response.

Analysis of the SCKs or micelles by dynamic light scattering (DLS): Hydrodynamic diameters (Dz, Dn) and size distributions for the SCKs in aqueous solutions were determined by dynamic light scattering (DLS). The DLS instrumentation consisted of a Brookhaven Instruments Limited (Holtsville, N.Y.) system, including a model BI-200SM goniometer, a model BI-9000AT digital correlator, a model EMI-9865 photomultiplier, and a model 95-2 Ar ion laser (Lexel, Corp.; Farmindale, N.Y.) operated at 514.5 nm. Measurements were made at 20 (1° C. Prior to analysis, solutions were centrifuged in a model 5414 microfuge (Brinkman Instruments, Inc.; Westbury, N.Y.) for 4 min to remove dust particles. Scattered light was collected at a fixed angle of 90°. The digital correlator was operated with 522 ratio spaced channels, an initial delay of 0.1 μs, a final delay of 5.0 μs, and a duration of 15 min. A photomultiplier aperture of 200 μm was used, and the incident laser intensity was adjusted to obtain a photon counting of 200 kcps. Only measurements in which the measured and calculated baselines of the intensity autocorrelation function agreed to within 0.1% were used to calculate particle size. The calculations of the particle size distributions and distribution averages were performed with the ISDA software package (Brookhaven Instruments Company), which employed single-exponential fitting, cumulants analysis, and nonnegatively constrained least-squares particle size distribution analysis routines. A stock solution of PBS was made by dissolving 7.564 g of NaH₂PO₄, 19.681 g of Na₂HPO₄, and 11.688 g of NaCl in 4 liters of nanopure water. After complete dissolution, NaOH or HCl was added to achieve the desired pH value. The samples were filtered using 0.45 μm pore size nylon membrane filters in order to remove dust and any large, nonmicellar aggregates.

Analysis of the SCKs or micelles by atomic force microscopy (AFM): The height measurements and distributions for the SCCs were determined by tapping-mode AFM under ambient conditions in air. The AFM instrumentation consisted of a Nanoscope III BioScope system (Digital Instruments, Veeco Metrology Group; Santa Barbara, Calif.) and standard silicon tips (type, OTESPA-70; L, 160 μm; normal spring constant, 50 N/m; resonance frequency, 224-272 kHz). The sample solutions were drop (2 μL) deposited onto freshly cleaved mica and allowed to dry freely in air.

Analysis of the SCKs or micelles by transmission electron microscopy (TEM): TEM samples were diluted in water (9:1) and further diluted with a 1% phosphotungstic acid (PTA) stain (1:1). Carbon grids were prepared by a plasma treatment to increase the surface hydrophilicity. Micrographs were collected at 100,000× magnification and calibrated using a 41 nm polyacrylamide bead standard from NIST. Histograms of particle diameters were generated from the analysis of a minimum of 150 particles from at least three different micrographs.

Analysis of the SCKs by UV-vis/Fluorescence: UV-vis spectroscopy data were acquired on a Varian Cary 1E UV-vis spectrophotometer. Fluorescence spectroscopy data were acquired on a Varian Cary Eclipse Fluorescence spectrophotometer. Each sample was prepared independently from a nanoparticle stock solution at ca. 0.13 mg/mL. Sample solutions at various pH values from 4.5, 6.1, 8.0, 9.5, 11.0, and 12.8 were excited at λₑₘ=435 nm, and the fluorescence emission spectra in the range 445-800 nm were recorded.

### Example 3: Optical Results for Photon Shell Cross-Linked Nanoparticles

The optical properties of compositions of the present invention were evaluated. The optical absorption and optical fluorescence were measured as a function of pH for Photonic Cross-Linker Example 2, Photonic Cross-Linker Example 3, Shell Cross-Linked Nanoparticle Example 5, Shell Cross-Linked Nanoparticle Example 6, Shell Cross-Linked Nanoparticle Example 7, and Shell Cross-Linked Nanoparticle Example 8.

Control Experiment: Change in fluorescence output for Photonic Cross-Linker Example 2 alone (prior to cross-linking into nanoshells). As seen in FIG. 17, this molecule is relatively insensitive to pH change itself due to its quadrupolar nature; less than 10% change in fluorescence is observed in Photonic Cross-Linker Example 2 as a function of increasing pH.

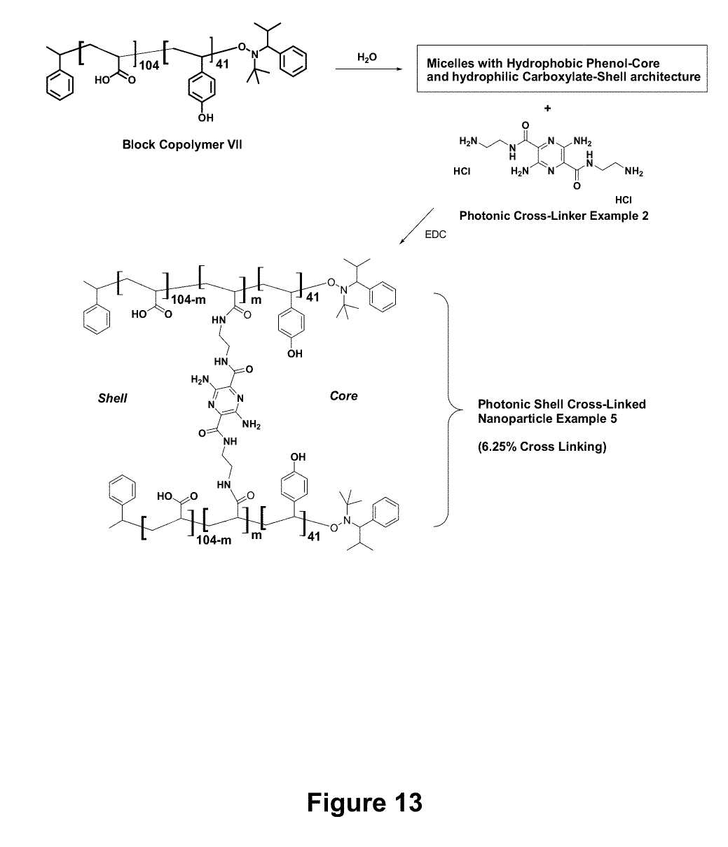

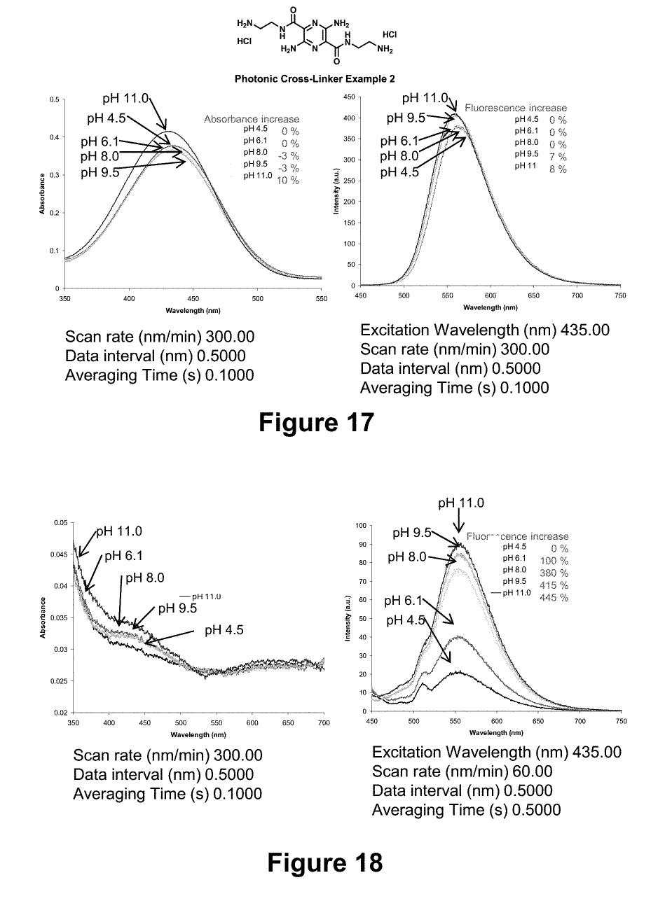

Shell Cross-Linked Nanoparticle Example 5: Optical Fluorescence Output of as a Function of pH. Block Copolymer VII was Cross-Linked with 6.25% Photonic Cross-Linker Example 2 to provide Shell Cross-Linked Nanoparticle Example 5, as illustrated in FIG. 13. As shown in FIG. 18, more than quadruple increase in fluorescence is observed in micelles cross-linked with 6.25 mol % Photonic Cross-Linker Example B as a function of increasing pH.

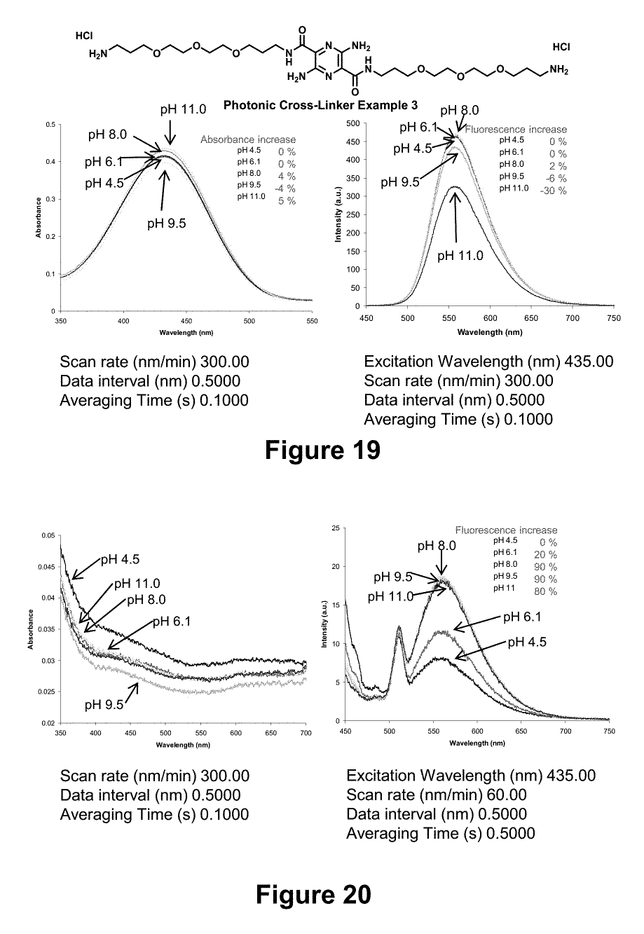

Control Experiment: Change in fluorescence output for Photonic Cross-Linker Example 3 alone (prior to cross-linking into nanoshells), was determined as a function of pH and is shown in FIG. 19. Photonic Cross-Linker Example 3, however, experienced ca. 30% decrease in fluorescence as a function of increasing pH.

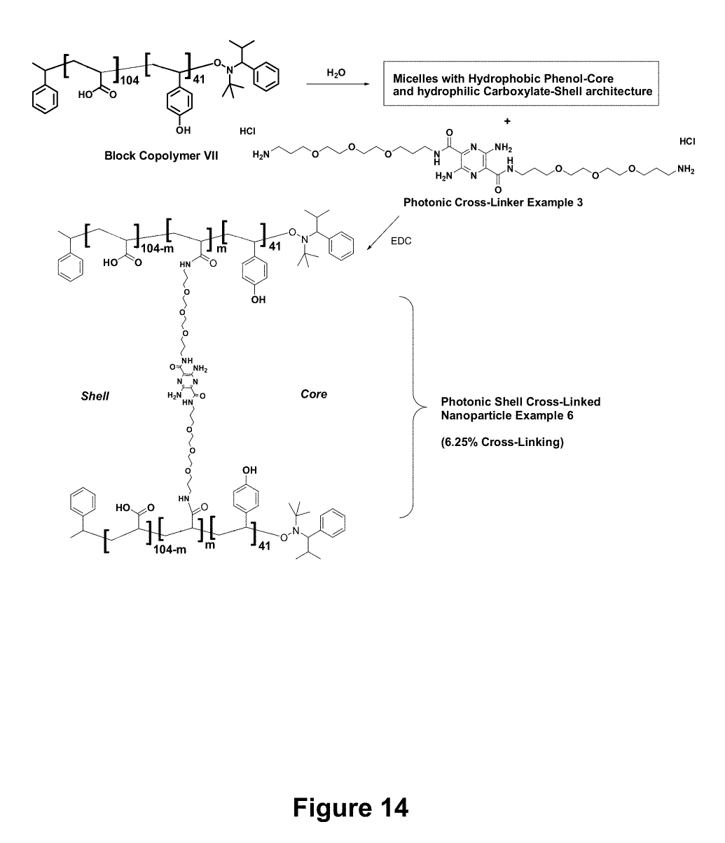

Shell Cross-Linked Nanoparticle Example 6: Optical Fluorescence Output of as a function of pH. Block Copolymer VII was Cross-Linked with 6.25% Photonic Cross-Linker Example 3 to provide Shell Cross-Linked Nanoparticle Example 6, as illustrated in FIG. 14. FIG. 20 shows up to 90% increase in fluorescence is observed in micelles cross-linked with 6.25 mol % Photonic Cross-Linker Example 3 as a function of increasing pH.

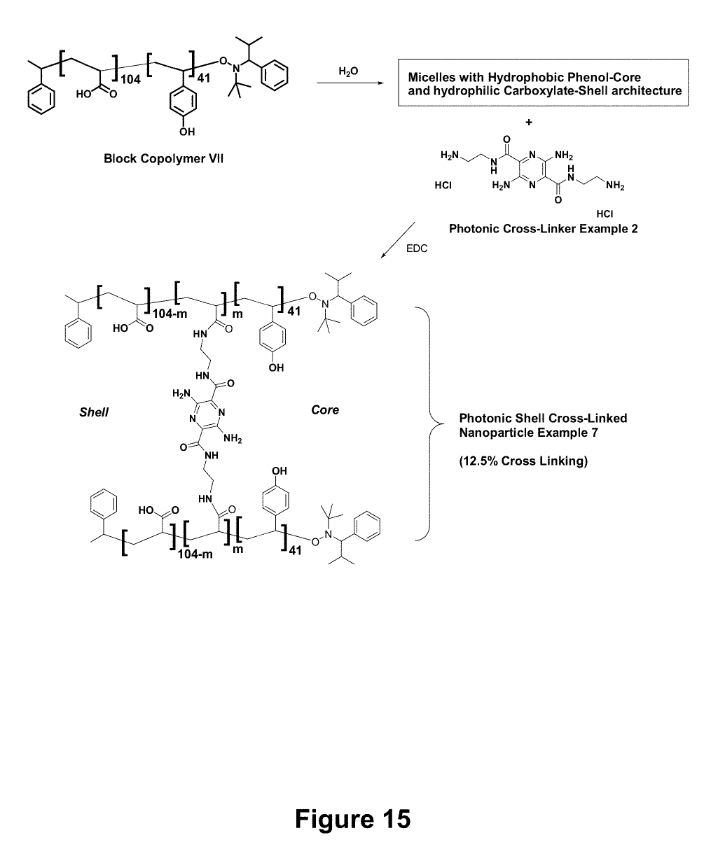

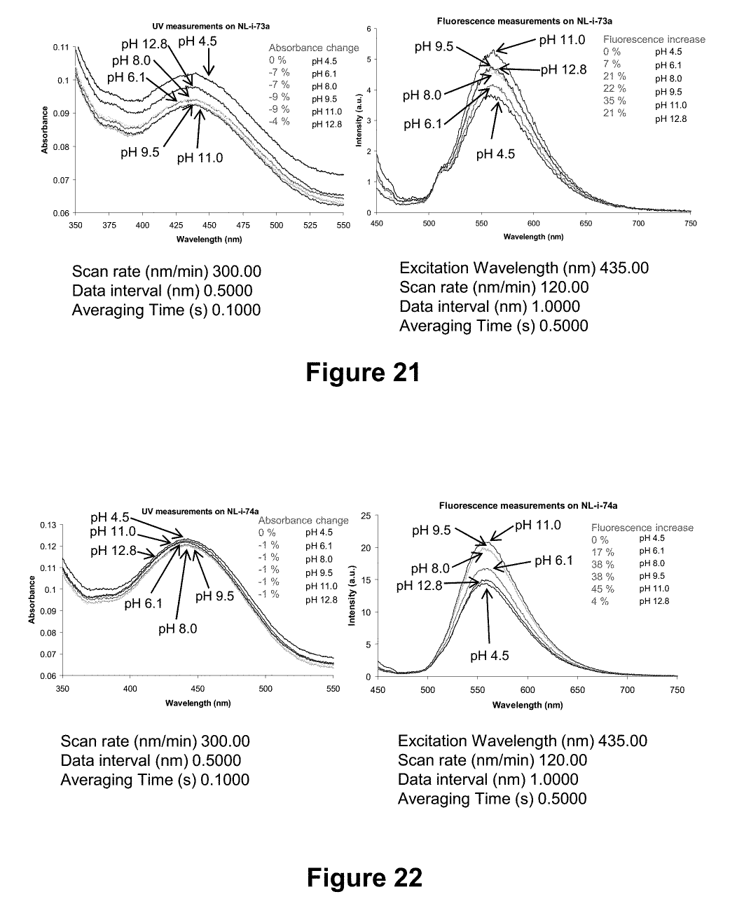

Shell Cross-Linked Nanoparticle Example 7: Optical Fluorescence Output of as a Function of pH. Block Copolymer VII was Cross-Linked with 12.5% Photonic Cross-Linker Example 2 to provide Shell Cross-Linked Nanoparticle Example 7, as illustrated in FIG. 15. FIG. 21 shows a substantial increase in fluorescence output of Shell Cross-Linked Nanoparticle Example 7 throughout physiological pH range. The increase tracks with swelling induced by deprotonation of shell carboxylic acids as pH increases from 4.5-8.0 and again as further swelling is induced by phenolate formation as pH traverses phenol pKa range from 9.5 to 11.0. Decrease of fluorescence upon further increased pH (to 12.8) may result from deprotonation of the pyrazine cross-link NH₂ groups.

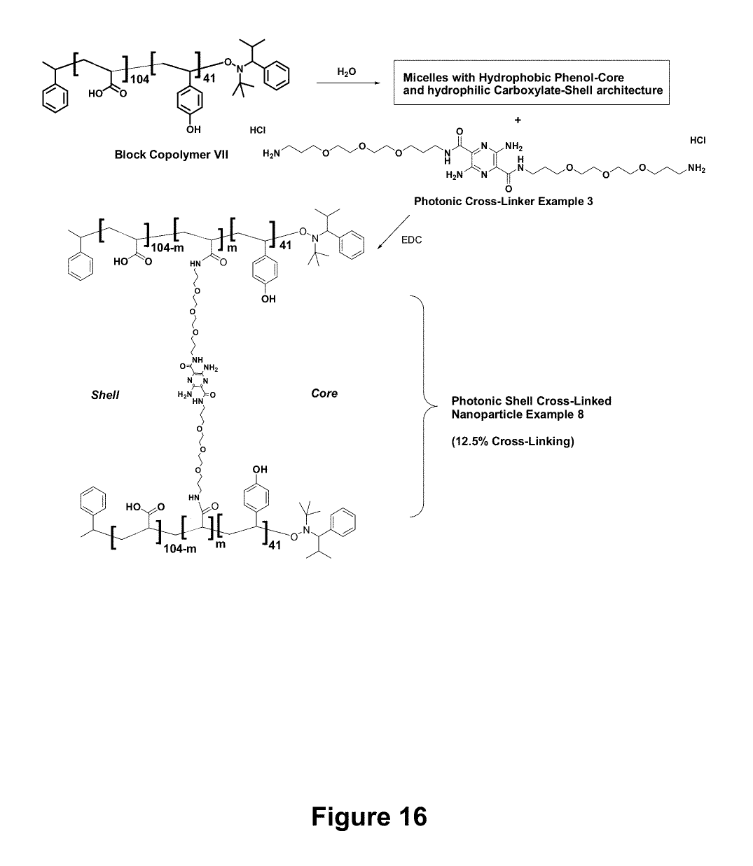

Shell Cross-Linked Nanoparticle Example 8: Optical Fluorescence Output of as a Function of pH. Block Copolymer VII was Cross-Linked with 12.5% Photonic Cross-Linker Example 3 to provide Shell Cross-Linked Nanoparticle Example 8, as illustrated in FIG. 16. As seen in FIG. 22, fluorescence output again increases through physiological pH range as in the previous example.

### Example 4: Additional Linkage Systems

Additional chemistries were explored to identify further photonic cross-linking systems.

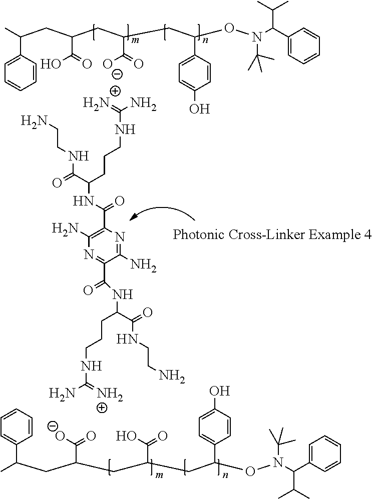

Preassociation of Photonic Cross-Linker Example 4 with Block Copolymer VII. The guanidine groups can form salt bridge coordination with the carboxylate shell region over a large pH range (˜3-12) and facilitate cross-linking. In addition the positive guanidinium charge can modulate surrounding pH and swelling characteristics of the nanoparticle.

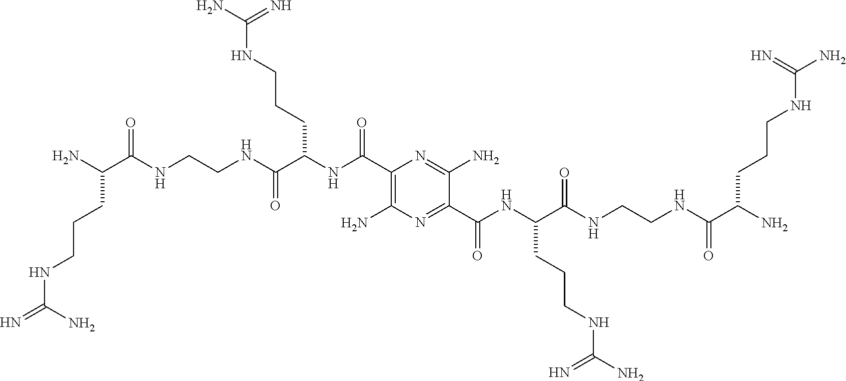 [SMILES: N=C(N)NCCC[C@H](NC(=O)c1nc(N)c(C(=O)N[C@@H](CCCNC(=N)N)C(=O)NCCNC(=O)[C@@H](N)CCCNC(=N)N)nc1N)C(=O)NCCNC(=O)[C@@H](N)CCCNC(=N)N]

Photonic Cross-Linker Example 9.

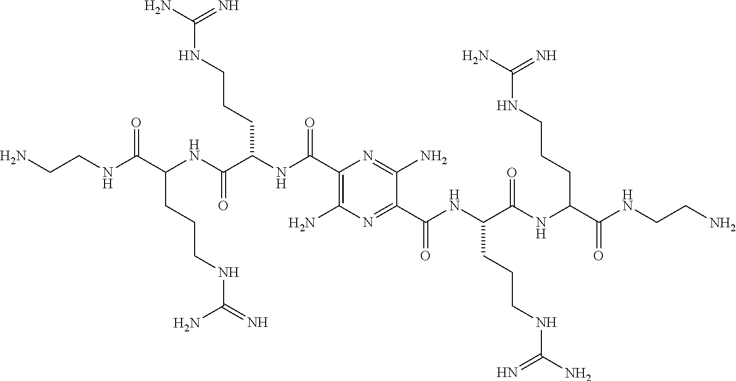 [SMILES: N=C(N)NCCCC(NC(=O)[C@H](CCCNC(=N)N)NC(=O)c1nc(N)c(C(=O)N[C@@H](CCCNC(=N)N)C(=O)NC(CCCNC(=N)N)C(=O)NCCN)nc1N)C(=O)NCCN]

Photonic Cross-Linker Example 10.

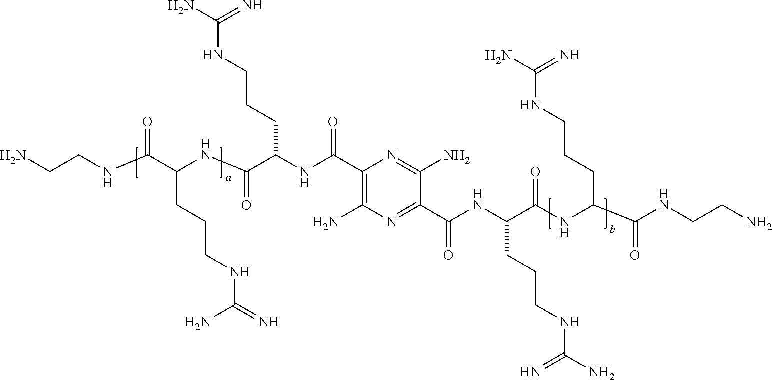

Photonic Cross-Linker Example 11: a and b can be any whole number, most preferably 1-7 to generate poly-Arg containing cross-linkers.

Photonic Cross-Linker Example 12

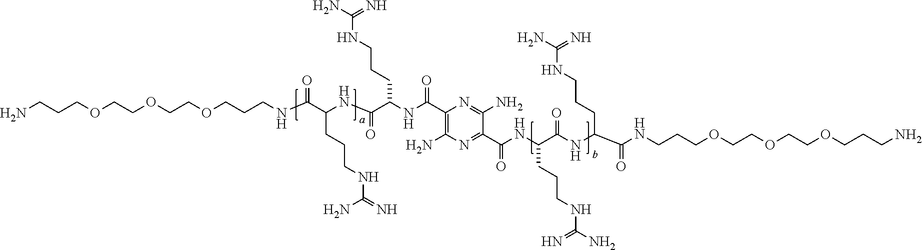

Photonic Cross-Linker Example 13.

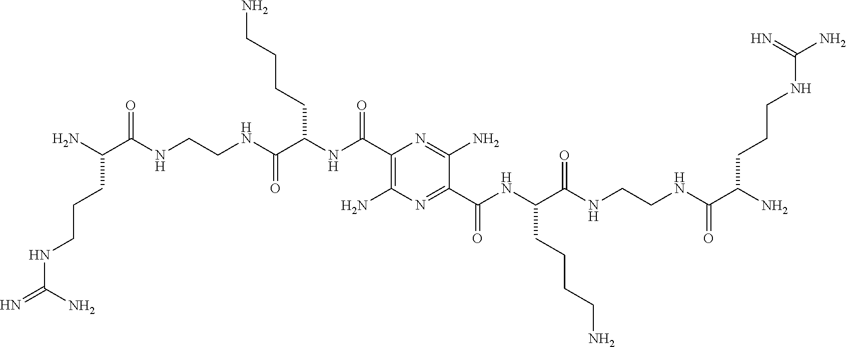 [SMILES: N=C(N)NCCC[C@H](N)C(=O)NCCNC(=O)[C@H](CCCCN)NC(=O)c1nc(N)c(C(=O)N[C@@H](CCCCN)C(=O)NCCNC(=O)[C@@H](N)CCCNC(=N)N)nc1N]

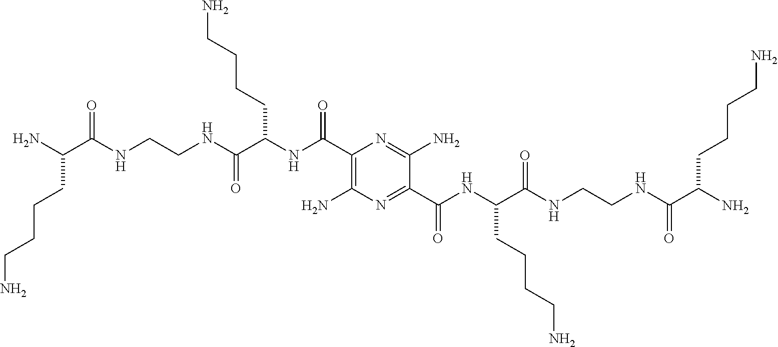 [SMILES: NCCCC[C@H](NC(=O)c1nc(N)c(C(=O)N[C@@H](CCCCN)C(=O)NCCNC(=O)[C@@H](N)CCCCN)nc1N)C(=O)NCCNC(=O)[C@@H](N)CCCCN]

Photonic Cross-Linker Example 14.

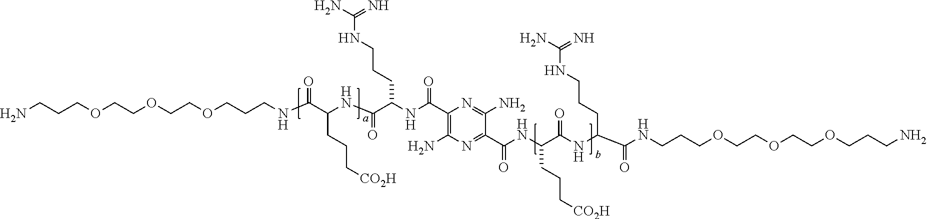

Photonic Cross-Linker Example 15.

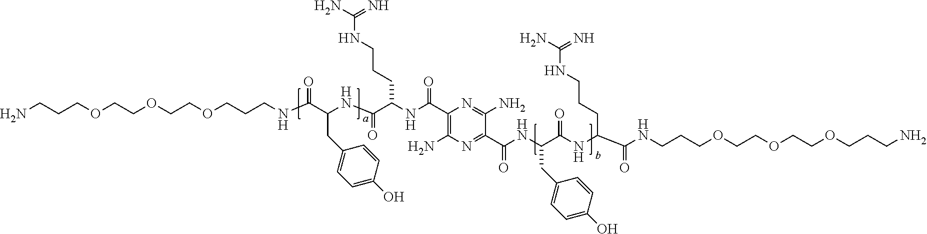

Photonic Cross-Linker Example 16 (Polyphenols to modulate pKa's).

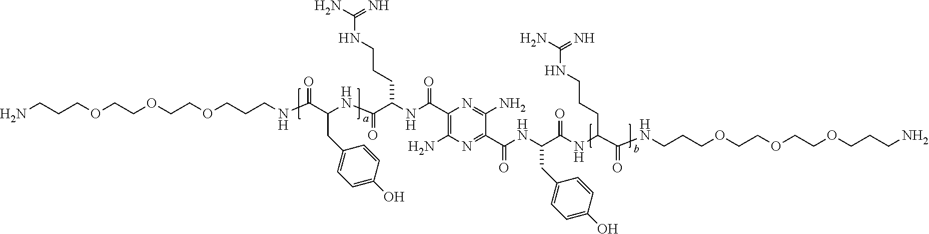

Photonic Cross-Linker Example 17 (Polyphenol and PolyArg to modulate pKa's).

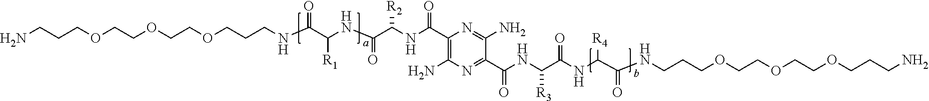

Photonic Cross-Linker General Example 18: R₁, R₂, R₃, R₄ can be ANY natural or unnatural amino acid, in repeating units defined by a and b.

### Example 5: Construction of Functionalizable, Cross-Linked Nanostructures

### Introduction

During the past decade, nanoscale micelles and vesicles assembled from amphiphilic block copolymer precursors have attracted much attention due to their promise for applications in the field of nanomedicine, ranging from controlled delivery of drugs and other diagnostic and therapeutic agents, to targeting of specific diseases and reporting of biological mechanisms via introduction of various functionalities. The thermodynamic stability of such nanoscale systems is only achieved above the critical micelle/vesicle concentration and their stability in vivo is therefore of concern. To overcome this restriction, covalent cross-linking throughout the shell/core domain of micelles or membrane domain of vesicles has been developed and demonstrated as an effective methodology for providing robust nanostructures.

In this Example, functional block copolymer systems were established based on N-acryloxysuccinimide (NAS) monomer building blocks, containing pre-installed active esters as amidation sites. A series of pyrazine-based diamino cross-linkers (Scheme 1 of this Example 5, Cross linkers 1-3) were designed for exploring the potential factors during the reaction with pyrazine acting as a monitoring probe. Furthermore, the photophysical properties of these cross-linked nanostructures were also investigated to explore their potential application for optical imaging and monitoring.

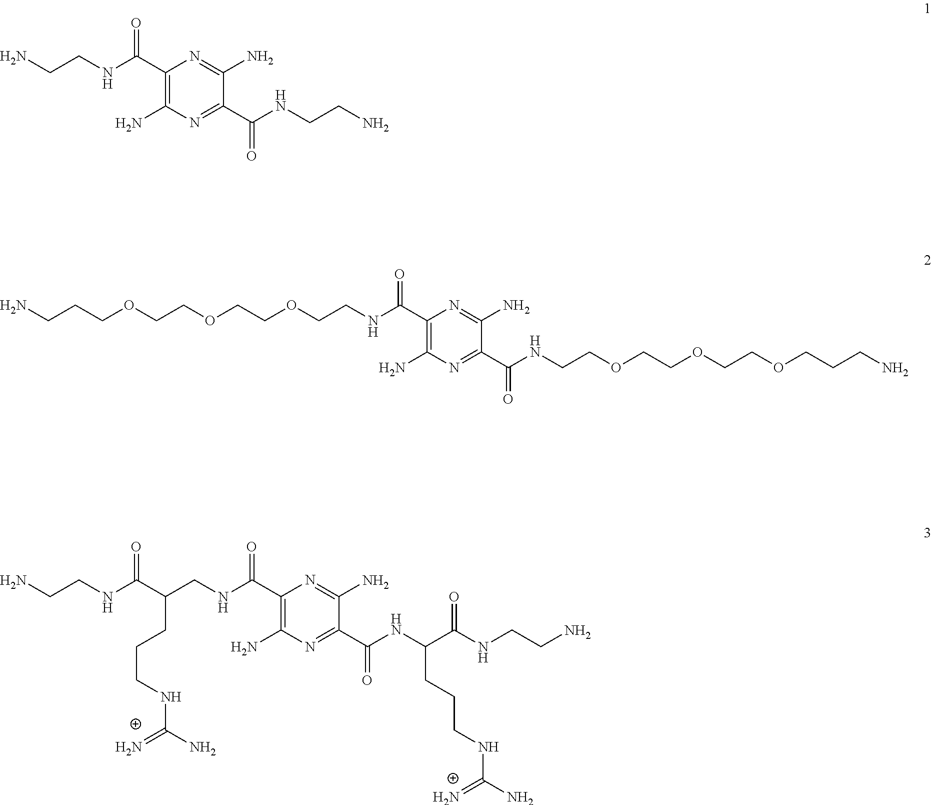 [SMILES: NCCNC(=O)c1nc(N)c(C(=O)NCCN)nc1N ; NCCCOCCOCCOCCNC(=O)c1nc(N)c(C(=O)NCCOCCOCCOCCCN)nc1N ; N=C(N)NCCCC(CNC(=O)c1nc(N)c(C(=O)NC(CCCNC(N)=[NH2+])C(=O)NCCN)nc1N)C(=O)NCCN]

Scheme 1 of Example 5:

Chemical structures of pyrazine-based diamino cross-linkers.

Results and Discussion

As depicted in Scheme 1 of Example 5, well-defined diblock copolymers PEO₄₅-b-PNAS₉₅ and PEO₄₅-b-PNAS₁₀₅ were obtained via reversible addition-fragmentation chain transfer (RAFT) polymerization,³ starting from a PEO₄₅ based macro chain transfer agent (macro-CTA). GPC analyses of these two polymers (Scheme 1 of Example 5, insertions) clearly demonstrated their monomodal molecular weight distributions, even at higher NAS monomer conversions (90% and 95% respectively). Further chain extension with styrene yielded triblock copolymers PEO₄₅-b-PNAS₉₅-b-PS₆₀ (compound 4) and PEO₄₅-b-PNAS₁₀₅-b-PS₅₀ (compound 5).

Scheme 2 of Example 5.

Preparation of amphiphilic triblock copolymers. Insertions are DMF SEC profiles for the diblock (top) and triblock (bottom) copolymers.

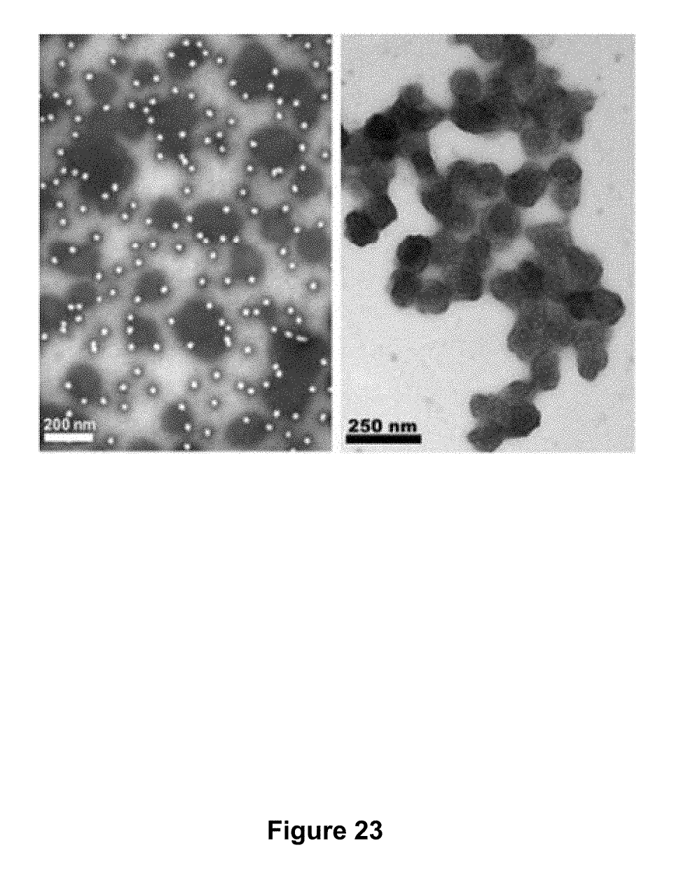

A typical self-assembly protocol was employed consisting of addition of water, a selective solvent for PEO, to the polymer precursor solution in DMF, a common solvent for all blocks. Interestingly, compound 4 provided micelles with hydrodynamic diameter of ca. 50 nm, while vesicles with hydrodynamic diameter of ca. 160 nm were generated from the assembly of compound 5 (See, FIG. 23). FIG. 23 provides TEM images of micelles (left) generated from compounds 4 of Example 5 and vesicles (right) generated from compound 5 of Example 5.

The cross-linking/functionalization efficiency for cross linkers 1 and 2 was almost identical, although the hydrophilicity of cross linker 2 was increased. A maximum of 30% actual cross-linking extent was achieved at each nominal extent (20%, 50%, and 100%, respectively). Dramatic improvement to a maximum of 60% actual cross-linking extent at each nominal extent was achieved while using cross linker 3, a cross-linker bearing positive charge. This improvement could be attributed to strong electrostatic interactions between the guanidine moieties of the bifunctional bis-arginyl-pyrazine 3, and copolymer NAS-derived carboxylates, generated by partial hydrolysis of active esters during the micellization process. The present invention includes the use of a variety of cross linking moieties having one or more natural or non-natural amino acid groups, particularly one or more basic amino acids, such as arginine, lysine, histidine, ornithine, and homoarginine. Thus, pre-coordination of cross linker 3 with the micelles/vesicles via guanidine-carboxylate complexes, resulted in a vast enhancement of inter-strand amide cross-linking reaction efficiency. The morphology of all of these nanoobjects was maintained for micelles and vesicles after cross-linking at the nominal 20% and 50% extents, while different morphologies were observed for cross-linked micelles at the nominal 100% extents.

Photophysical properties of these photonic nanoparticles and vesicles were then measured. For cross linkers 1 and 2, only the nominal 100% cross-linked nanoparticles exhibited similar UV-Vis profiles as the cross-linkers themselves while a blue shift (ca. 35 nm) was observed for the nominal 20% cross-linked micelles. For cross linker 3, blue shift (ca. 40 nm) was also observed for nominal 20% cross-linked micelles, but the nominal 50% cross-linked nanoparticles already displayed identical maximum UV-Vis absorption at 440 nm as the cross-linker. All of these nano-objects showed pH-sensitive fluorescence enhancements up to 300% in the range of pH 5.5 to 8.5. There were no obvious hydrodynamic diameter variations of these nanoparticles and vesicles in the surveyed pH range, as measured by DLS.

A novel amphiphilic triblock copolymer system having a functionalized PNAS segment was established. Further treatments of this functional polymer led to functionalized nanostructures bearing interesting stoichiometric and pH-sensitive photophyscial properties. This method also allowed for the facile quantification of actual cross-linking extents.

This Example highlights the usefulness of controlled radical polymerization of functional monomers to provide well-defined, reactive block copolymers that can be transformed into functional nanoscale objects. Employing reversible addition-fragmentation chain transfer (RAFT) polymerization, well-defined amphiphilic triblock copolymers poly(ethylene oxide)-b-poly(N-acryloxysuccinimide)-b-polystyrene (PEO-b-PNAS-b-PS) were obtained. These polymer precursors were assembled into highly functionalizable nanoparticles and nano-scale vesicles in aqueous media. After in situ cross-linking with a series of pyrazine-based diamino cross-linkers through amidation, it was revealed that the reaction efficiency varied with the composition and properties of the cross-linkers. The photophysical properties of the pyrazine fluorophore (i.e. UV absorption and fluorescence) were also found to be altered after covalent incorporation into the polymer assemblies. These results not only provided direct “visualization” of the extent of cross-linking, but also demonstrated that the photonic cross-linked nanostructures could be utilized for optical imaging and monitoring.

### REFERENCES FOR EXAMPLE 5

J. Xu, G. Sun, R. Rossin, A. Hagooly, Z. Li, K-I, Fukukawa, B. W. Messmore, D. A. Moore, M. J. Welch, C. J. Hawker, K. L. Wooley, “Labeling of polymer nanostructures for medical imaging: importance of cross-linking extent, spacer length, and charge density,” Macromolecules. 40, 2971-2973 (2007).
Q. Ma, E. E. Remsem, T. Kowalewski, J. Schaefer, K. L. Wooley, “Environmentally-responsive, entirely hydrophilic, shell-cross-linked (SCK) nanoparticles,” Nano Lett. 1, 651-655 (2001).
H. Cui, Z. Chen, S. Zhong, K. L. Wooley, D. J. Pochan, “Block copolymer assembly via kinetic control,” Science. 317, 647-650 (2007).
D. Benoit, V. Chaplinski, R. Braslau, C. J. Hawker, “Development of a universal alkoxyamine for “living” free radical polymerizations,” J. Am. Chem. Soc. 121, 3904-3920 (1999).
Joralemon, M. J.; O'Reilly, R. K.; Hawker, C. J.; Wooley K. L. J. Am. Chem. Soc. 2005, 127, 16892-16899.
Li, Yali; Sun, Guorong; Xu, Jinqi; Wooley, Karen L. Nanotechnology in Therapeutics (2007), 381-407.
Kai Qi, Qinggao Ma, Edward E. Remsen, Christopher G. Clark, Jr., Karen Wooley J. Am. Chem. Soc., 2004, 126, 6599.
Qi Zhang, Edward Remsen, Karen Wooley, J. Am. Chem. Soc. 2000, 122, 3642.
Greenspan, P; Fowler, S. D., Journal of Lipid Research 1985, 26, 781.
M. Barzoukas, M. Blanchard-Desce, J. Chem. Phys. 2000, 113, 3951.
R. K. O'Reilly, C. J. Hawker, K. L. Wooley, Chem. Soc. Rev., 2006, 35, 1068-1083.
A. Walther, A. S. Goldmann, R. S. Yelamanchili, M. Drechsler, H. Schmalz, A. Eisenberg, A. H. E. Mller, Macromolecules, 2008, 41, 3254-3260.
Z. Li, E. Kesselman, Y. Talmon, M. A. Hillmyer, T. P. Lodge, Science, 2004, 306, 98-101.
I. W. Hamley, Nanotechnology, 2003, 14, R39-R⁵⁴.
S. Liu, S. P. Armes, Angew. Chem. Int. Ed. 2002, 41, 1413-1416.
B. P. Binks, R. Murakami, S. P. Armes, S. Fujii, Angew. Chem. Int. Ed. 2005, 44, 4795-4798.
H. Huang; T. Kowalewski; E. E. Remsen; R. Gertzmann; K. L. Wooley, J. Am. Chem. Soc., 1997,119, 11653-11659.
V. L. Alexeev, A. C. Sharma, A. V. Goponenko, S. Das, I. K. Lednev, C. S. Wilcox, D. N. Finegold, S. A. Asher, Anal. Chem. 2003, 75, 2316-2323.
X. Xu, A. V. Goponenko, S. A. Asher, J. Am. Chem. Soc. 2008, 130, 3113-3119.

### Example 6: Uniform, Functionalized, Cross-Linked Multicompartment Nanostructures with Tunable Photo-Physical Properties

The development of polymeric nanostructures from block copolymer aqueous supramolecular assemblies has gained significant attention due to their diverse promising applications.[1] It has been recognized that their chemical composition and also their size and morphology each require precise tuning.[2] Benefiting from the advances of living/controlled polymerization methodologies to afford varied block copolymer structures,[3] together with extensive investigation of their aqueous assembly,[4-6] polymeric nanostructures with diverse morphologies have been established. In addition to conventional morphologies, such as spheres, cylinders and vesicles, nanostructures with novel morphologies, including bowls,[4a] discs,[4b] helices,[4c] and toroids,[4d] have been reported. Moreover, Janus,[5a] multicompartment,[5b,5c,6] onion,[5d] and large compound[5e] micelles, from higher-order inter- and/or intra-micellar phase segregation, have been created.

Multicompartment micelles (MCMs) represent intra-micellar phase-segregated block copolymer supramolecular assemblies, in which the core domains are heterogeneous and compartmentalized.[6] Utilizing ABC starlike block terpolymers, by Lodge, Hillmyer and co-workers,[5b] and ABC linear triblock copolymers, by Laschewsky et al.[5c] (in both cases, A represents the hydrophilic block segment, B and C represent incompatible hydrophobic block segments), MCMs were realized through the compartmentalization of B and C blocks during the aqueous assembly process, as visualized by cryogenic transmission electron microscopy (cryo-TEM). Later, additional MCMs were prepared by tuning of both polymeric and supramolecular parameters to manipulate the sizes, morphologies,[6a-d] internal environments of the compartmentalized cores,[6e] and stimuli-induced responses.[6f,6 g] Meanwhile, the performance of MCMs as delivery vehicles for various cargos was investigated to address their unique potential for biomedical applications.[7]

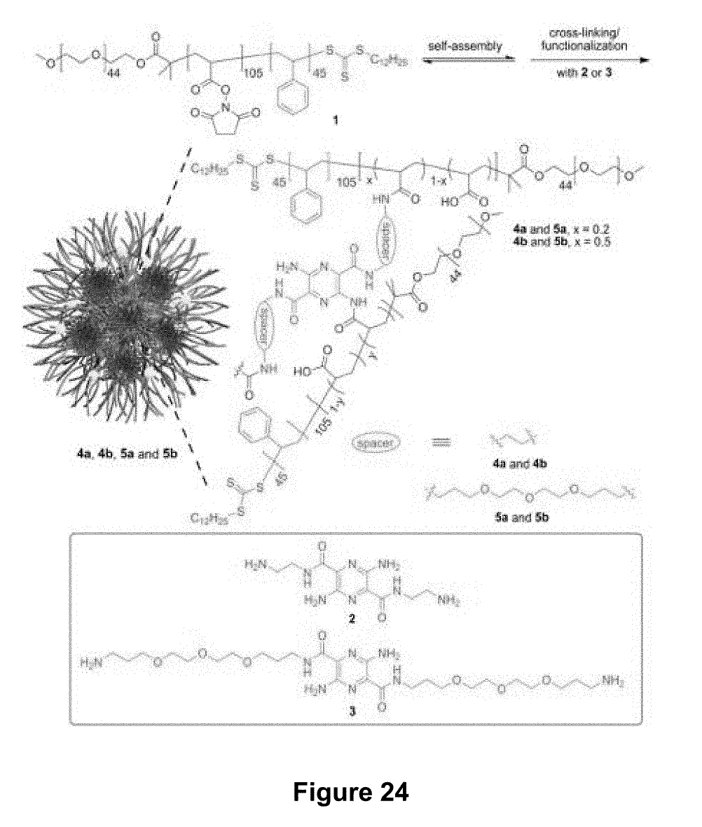

Although a variety of star terpolymer and linear block polymers have already been explored as precursors to prepare MCMs, most lacked functionalities for facile and practical chemical transformations.[6h] Herein, we report our approach for the construction of functionalized cross-linked multicompartment nanostructures (MCNs) from aqueous assembly of a linear poly(ethylene oxide)-block-poly(N-acryloxysuccinimide)-block-polystyrene (PEO-b-PNAS-b-PS), 1 (FIG. 24), amphiphilic ABC triblock copolymer, followed by cross-linking/functionalization of the MCMs with photophysically-active pyrazine-based diamino cross-linkers, 2 or 3 (FIG. 24), via well-known amidation chemistry (Scheme 1.1, FIG. 24) to establish the photonic MCNs, 4a, 4b, 5a, and 5b (FIG. 24), respectively. These functionalized MCNs were found to exhibit unique fluorescence emission characteristics.

ABC linear triblock copolymers have been shown to undergo greater variability in their assembly behaviors, in comparison to diblock copolymers.[4b-d,5c,6c-h,8] The particular PEO-b-PNAS-b-PS composition and sequence were selected to provide for a hydrophilic PEO end segment for water dispersibility, a central PNAS segment for reactivity, and a terminal hydrophobic and glassy PS segment to provide for nucleation of micellar assemblies in water and provide ability to trap initial MCM morphologies kinetically. The reactive activated ester functionalities enable further chemical modifications to improve the structural stability and expand the application scope.

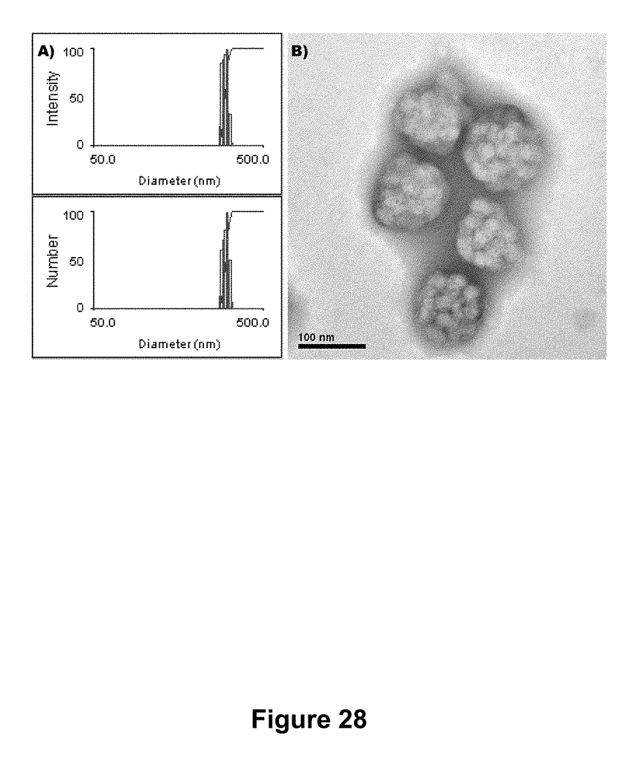

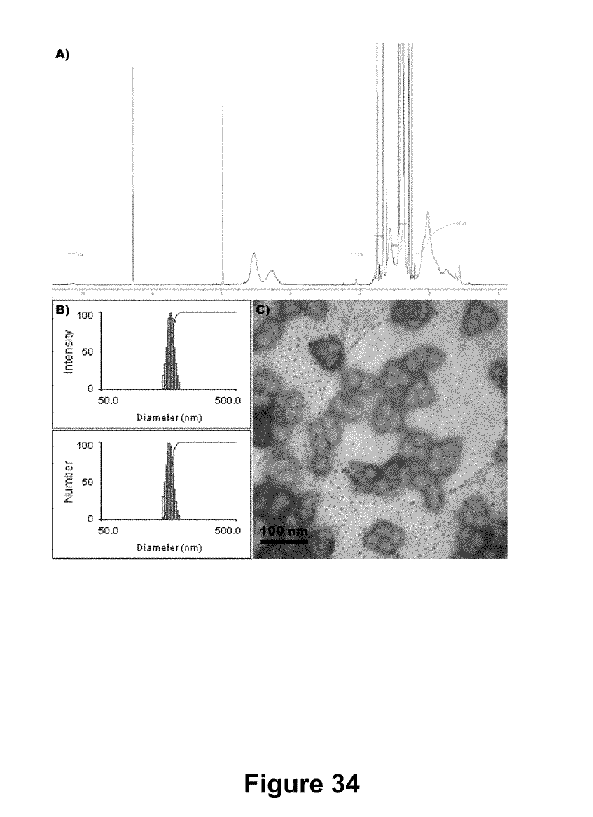

The well-defined PEO-b-PNAS-b-PS triblock copolymer precursor 1 (FIG. 24) used in this study (PEO₄₅-b-PNAS₁₀₅-b-PS₄₅, Mₙ^{NMR}=24,800 Da, PDI=1.2) was prepared by reversible addition-fragmentation chain transfer (RAFT) polymerization[3c] as reported elsewhere.[9] The aqueous assembly of 1 (FIG. 24) was carried out when the polymers were freshly prepared by introducing water (a selective solvent for the PEO block) to solutions of the triblock copolymer in N,N-dimethylformamide, DMF (a good solvent for all three blocks). [Footnote 10: The hydrolysis of NAS dramatically affected the self-assembly behavior of the triblock copolymer precursors. Uniform MCMs with smaller size (Dₕ=160±15 nm, FIG. 34, Panel B) and less number of compartments (FIG. 34, Panel C) were achieved through the assembly of PEO₄₅-b-P(NAS₉₅-co-AA₁₀)-b-PS₄₅ (FIG. 34, Panel A) precursors.] The nano-scale MCM assemblies in H₂O/DMF (v:v=1:1) were characterized immediately by dynamic light scattering (DLS, FIG. 28, Panel A) and TEM (FIG. 28, Panel B). The DLS results confirmed that uniform nanostructures (PDI<0.1, after cumulative analyses) with hydrodynamic diameter (Dₕ) of 300±20 nm were obtained.

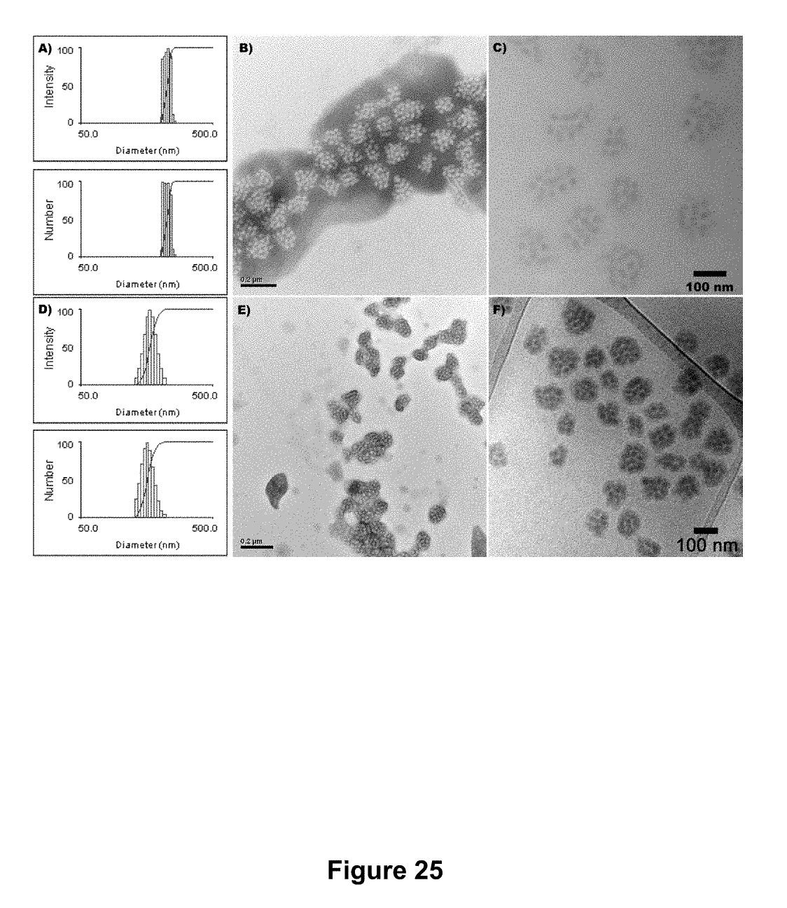

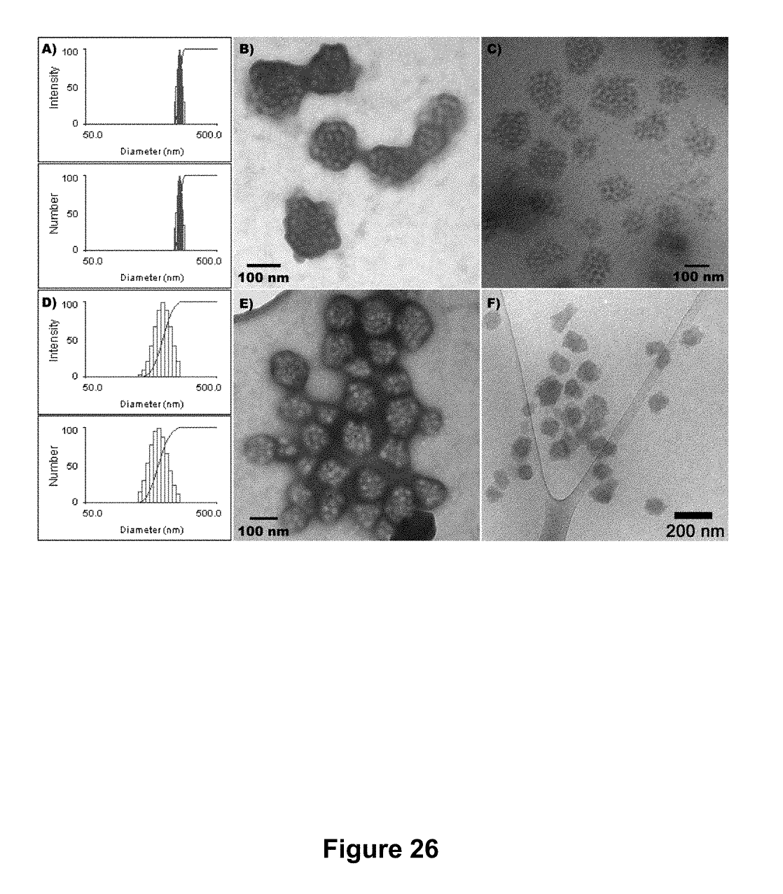

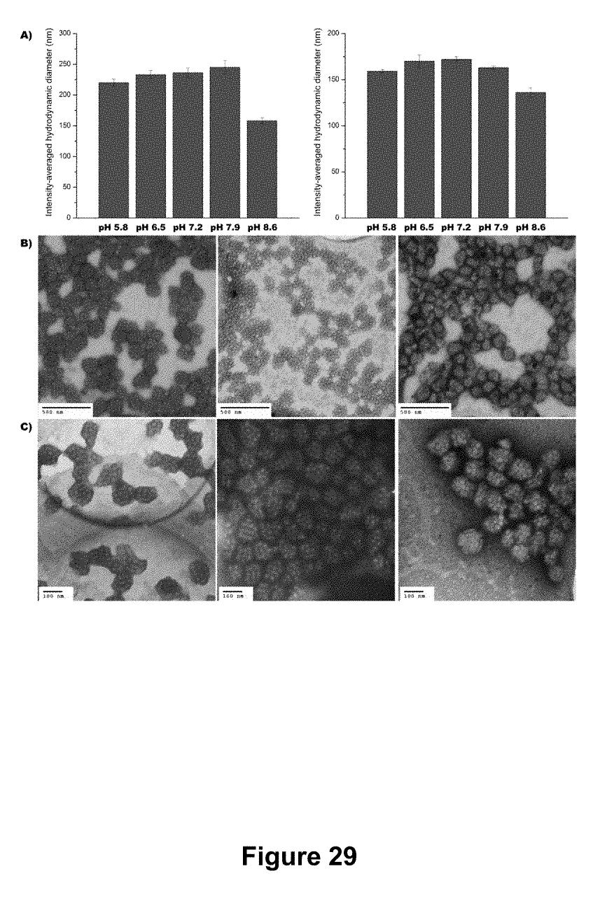

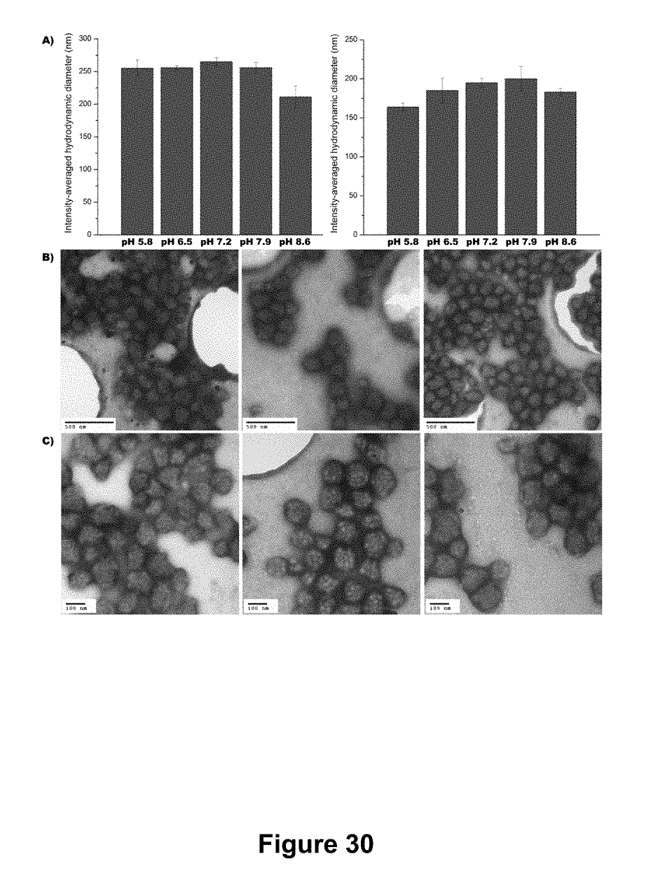

Covalent cross-linking and functionalization of the MCMs were accomplished by a “one-pot” approach, utilizing cross-linkers 2 or 3 (FIG. 24), designed to also determine the incorporation/cross-linking efficiency[9] and to enable unique pH-driven photo-physical property responses.[12] Compared with the MCM precursors, the hydrodynamic diameters of cross-linked MCNs with cross-linker 2 (FIG. 24) decreased, as confirmed by DLS (FIG. 25, Panels A and D, also see FIG. 29, Panel A). The observed shrinkage effect correlated with the cross-linking extents, i.e., as the extents of pyrazine incorporation increased from 0% to 9% to 17%, the corresponding Dₕ decreased from 300±20 nm to 225±25 nm to 165±30 nm. It also was found that the work-up procedure affected the final size for the MCNs with 9% of cross-linking (FIG. 29, Panel A, left). Although the cross-linked MCNs retained similar size of about 220 nm over a pH range of 5.8 to 7.9, with further increase of the pH value to 8.6 the hydrodynamic diameter decreased to about 160 nm. This reduction was tightly associated with the cross-linking extents, at higher degrees of cross-linking, the pH-responsive shrinkage was diminished (FIG. 29, Panel A, right). These trends were also observed for cross-linker 3 of FIG. 24 (FIG. 26, Panels A and D, and FIG. 30, Panel A). Interestingly, the incorporation efficiency of 3 (about 60%) was higher than that of 2 (about 40%) for MCNs at both examined cross-linking extents, which was different from the constant relative incorporation of each cross-linker within core-shell micelle systems studied previously.[9]

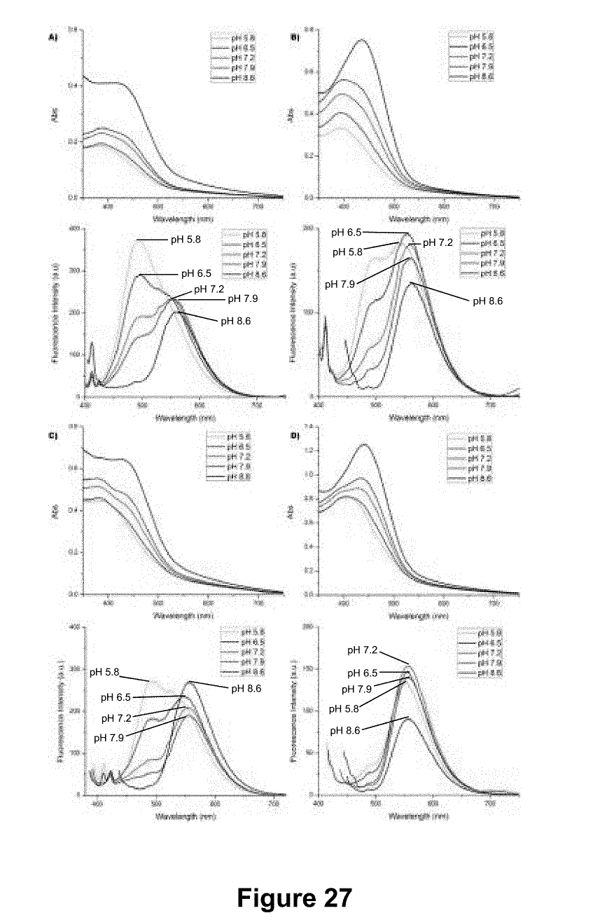

TEM and cryo-TEM imaging (middle and right column in FIGS. 26 and 27, respectively) of cross-linked MCNs gave diameters that were in agreement with the DLS results and provided more structural information (also see FIGS. 29 and 30 for additional TEM images at other pH values). Comparison of MCM and MCN microscopy images (FIG. 28 vs. 29) demonstrates maintenance of the internal segregated architecture and enhanced compartmentalization after cross-linking. However, different packing patterns of the compartments occurred with different cross-linking extents. As depicted by the cryo-TEM micrographs in FIGS. 26 and 27, noticeably different local environments around the compartments were detected. With the increased level of functionalization, more pyrazine moieties were introduced into MCNs, and these different compositions, combined with variable degrees of phase segregation can lead to the observed differences in the images.

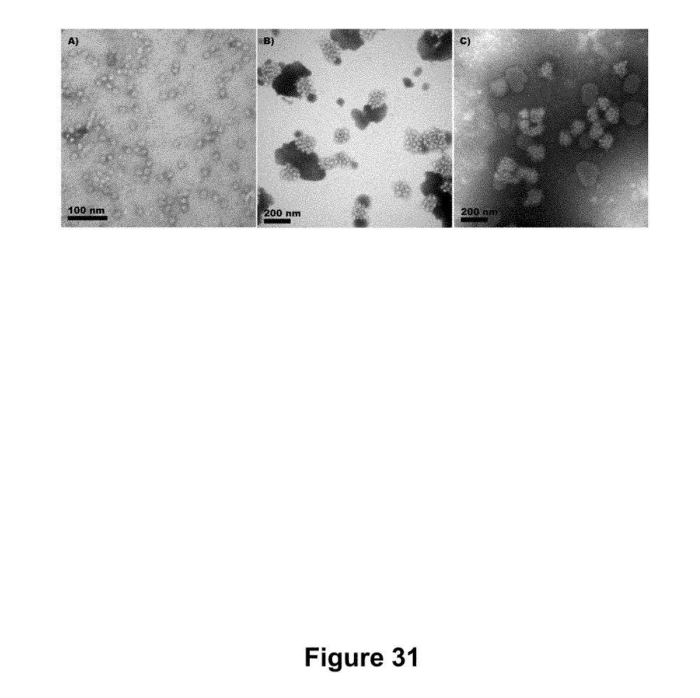

The significant increase of MCN structural stability after cross-linking was verified by comparing morphology of the pre-established MCMs and MCNs cross-linked with 2 (4a and 4b) in mixed organic/aqueous media (DMF/H₂O) over storage times at room temperature. The disassembly of MCMs (without any covalent stabilization) into discrete micellar forms ultimately occurred over long storage times (9 month in this study, FIG. 31, Panel A). And for the cross-linked MCNs (4a-b), no appreciable morphology variations were noticed (FIG. 31, Panels B and C), even at lower degree of cross-linking extents (4a, maximum cross-linking extents less than 10%).

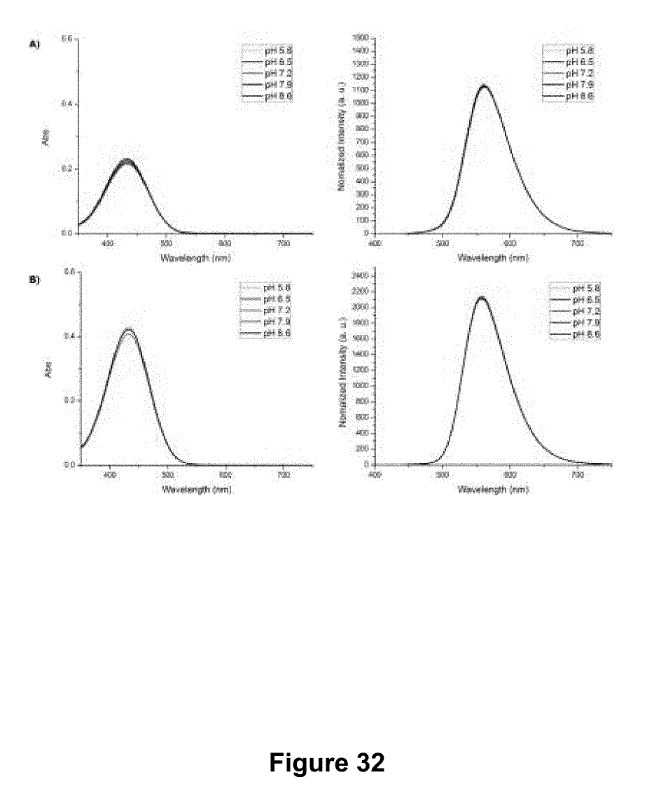

Finally, the photo-physical properties of these fluorogenic MCNs were studied. For 2 and 3 small molecules at the surveyed pH values, no apparent UV-Vis absorbance and fluorescence emission spectra variation was detected (FIG. 32), which indicated their intrinsic non-pH-responsive properties. As 2 and 3 were incorporated into MCNs through covalent functionalization, the UV-Vis maximum absorbance peaks were blue shifted from 433 nm to about 390 nm at pH 5.8. With increase of external pH values, the 433 nm peak started appearing along the UV-Vis profile and, eventually became the equivalent or even dominant absorbance peak, depending upon the incorporation extents (FIG. 27, panels A-D, top). More interestingly, the fluorescence emission at corresponding pH values also experienced such a tendency (FIG. 27, panels A-D, bottom).

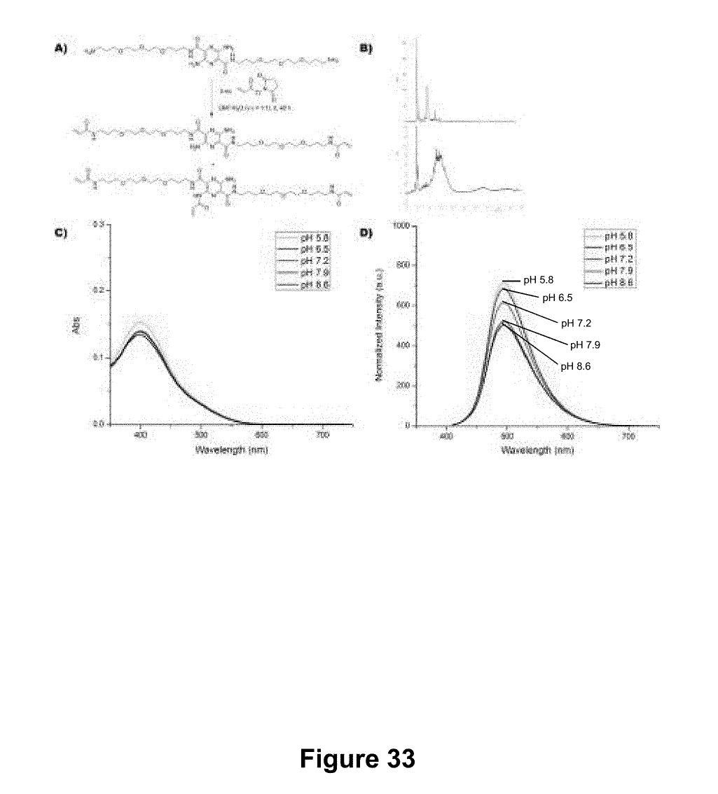

We synthesized the tri-acylated derivative of 3 (FIG. 33, Panel A) and studied its photo-physical properties with the corresponding pH value range. The blue-shifts of both the UV-Vis maximum absorbance peak (from 433 nm to 400 nm, FIG. 33, Panel B) and fluorescence emission peak (from 560 nm to 495 nm, FIG. 33, Panel C) were noticed, which was consistent with an early literature report.[11] In addition, pH-responsive fluorescence intensity decreases were observed, in response to the increasing of pH from 5.8 to 8.6. This control experiment demonstrated that the pH-sensitive MCN photo-physical response originated from the acylation of pyrazine aromatic amine. The unique environment within the MCMs seemed to promote acylation of the aromatic amines, whereas previous work with spherical core-shell micelles observed primarily reaction of the aliphatic amines of 2 and 3.[9,12] However, other factors including the photon re-absorption and subsequent photon re-emission, the twisted intramolecular charge-transfer,[13] as well as the ionic strength of the media, should also be taken into account.

In summary, uniform multicompartment nanostructures bearing NHS active ester functionalities have been prepared from self-assembly of linear triblock copolymer PEO₄₅-b-PNAS₁₀₅-b-PS₄₅. The active ester functionalities were demonstrated to allow for modifications through facile and practical chemistry, including cross-linking and functionalizing with pyrazine-based cross-linkers to achieve enhanced stability and to enable pH-sensitive photo-physical responses. It is expected the above unique properties of these MCNs will make them promising materials for fundamental study in biotechnology as well as practical applications.

### Experimental

Materials

The mono-methoxy terminated mono-hydroxy poly(ethylene glycol) (mPEG2k, MW=2,000 Da, PDI=1.06) was purchased from Intezyne Technologies and was used for the synthesis of macro-CTA without further purification. The PEO-b-PNAS-b-PS triblock copolymer (vide infra) and the cross-linkers 2 and 3 of FIG. 24 were synthesized according to previous reports.[9,12] Other chemicals were purchased from Aldrich and Acrose were used without further purification unless otherwise noted. Prior to use, N-acryloxysuccinimide (Acrose, 99%) was recrystallized from dry ethyl acetate and stored under argon. Styrene (Aldrich, 99%) was distilled over calcium hydride and stored under N₂. The Supor 25 mm 0.1 μm Spectra/Por Membrane tubes (molecular weight cut-off (MWCO) 6-8 kDa), used for dialysis, were purchased from Spectrum Medical Industries Inc. Nanopure water (18 mΩ·cm) was acquired by means of a Milli-Q water filtration system (Millipore Corp.).

Measurements

¹H and ¹³C NMR spectra were recorded on a Varian 600 MHz spectrometer interfaced to a UNIX computer using Mercury software. Chemical shifts are referred to the solvent proton resonance.

The molecular weight distribution was determined by Gel Permeation Chromatography (GPC). The N,N-dimethylformamide (DMF) GPC was conducted on a Waters Chromatography Inc. system equipped with an isocratic pump model 1515, a differential refractometer model 2414, and a two-column set of Styragel HR 4 and HR 4E 5 μm DMF 7.8×300 mm columns. The system was equilibrated at 70° C. in pre-filtered DMF containing 0.05 M LiBr, which served as polymer solvent and eluent (flow rate set to 1.00 mL/min). Polymer solutions were prepared at a concentration of ca. 3 mg/mL and an injection volume of 200 μL was used. Data collection and analysis was performed with Empower Pro software (Waters Inc.). The system was calibrated with poly(ethylene glycol) standards (Polymer Laboratories) ranging from 615 to 442,800 Da.

Transmission Electron Microscopy (TEM) bright-field imaging was conducted on a Hitachi H-7500 microscope, operating at 80 kV. The samples were prepared as following: 4 μL of the dilute solution (with a polymer concentration of ca. 0.2-0.5 mg/mL) was deposited onto a carbon-coated copper grid, which was pre-treated with absolute ethanol to increase the surface hydrophilicity. After 5 min, the excess of the solution was quickly wicked away by a piece of filter paper. The samples were then negatively stained by placing 4 μL of 1 wt % phosphotungstic acid (PTA) aqueous solution on the top. After 1 min, the excess PTA solution was quickly wicked away by a piece of filter paper and the samples were left to dry under room temperature overnight.

Cryogenic Transmission Electron Microscopy (Cryo-TEM) imaging was performed on a JEOL 1230 microscope, operating at 80 kV. A small droplet of the solution (5-10 μL) was placed on a holey carbon film supported on a TEM copper grid within a controlled environment vitrification system (Gatan Inc.). The specimen was blotted and plunged into a liquid ethane reservoir cooled by liquid N₂. The vitrified samples were transferred to a Gatan 626 cryo-holder and cryo-transfer stage cooled by N₂. During observation of the vitrified samples, the cryo-holder temperature was maintained below −170° C. to prevent sublimation of vitreous water.

Hydrodynamic diameters (Dₕ) and size distributions for the nanostructures in aqueous solutions were determined by dynamic light scattering (DLS). The DLS instrumentation consisted of a Brookhaven Instruments Limited system, including a model BI-200SM goniometer, a model BI-9000AT digital correlator, a model EMI-9865 photomultiplier, and a model 95-2 Ar ion laser (Lexel Corp.) operated at 514.5 nm. Measurements were made at 25±1° C. Scattered light was collected at a fixed angle of 90°. The digital correlator was operated with 522 ratio spaced channels, and initial delay of 5 μs, a final delay of 100 ms, and a duration of 6 minutes. A photomultiplier aperture of 100 μm was used, and the incident laser intensity was adjusted to obtain a photon counting of between 200 and 300 kcps. Only measurements in which the measured and calculated baselines of the intensity autocorrelation function agreed to within 0.1% were used to calculate particle size. The calculations of the particle size distributions and distribution averages were performed with the ISDA software package (Brookhaven Instruments Company), which employed single-exponential fitting, cumulants analysis, and CONTIN particle size distribution analysis routines. All determinations were repeated 5 times.

The UV-vis absorption spectra of MCNs were collected at room temperature using a Varian Cary 100 Bio UV-visible spectrophotometer and plastic cuvettes with 10 mm of light path. For each MCN absorption spectroscopy measurement, the corresponding buffer solution (5 mM with 5 mM of NaCl) outside the dialysis tubing was used as control.

The fluorescence spectra of MCNs were obtained at room temperature using a Varian Cary Eclipse fluorescence spectrophotometer. All fluorescence spectra from MCN solutions were measured at optical densities at the excitation wavelength. If not specially mentioned otherwise, an excitation wavelength of the observed maximum absorption peak was used. Each fluorescence spectrum was normalized with respect to the absorbed light intensity at the excitation wavelength.

Synthesis of PEO₄₅-b-PNAS₁₀₅

To a 25 mL Schlenk flask equipped with a magnetic stir bar dried with flame under N₂ atmosphere, was added the mPEG2k macro-CTA (0.24 g, 0.10 mmol) and 1,4-dioxane (10 mL). The reaction mixture was stirred 0.5 h at room temperature to obtain a homogeneous solution. To this solution was added NAS (1.9 g, 11 mmol) and AIBN (0.9 mg, 6 μmol). The reaction flask was sealed and stirred 10 min at room temperature. The reaction mixture was degassed through several cycles of freeze-pump-thaw. After the last cycle, the reaction mixture was stirred for 10 min at room temperature before being immersed into a pre-heated oil bath at 60° C. to start the polymerization. After 105 min, the monomer conversion reached ca. 95% by analyzing aliquots collected through 1H-NMR spectroscopy. The polymerization was quenched by cooling the reaction flask with liquid N₂. The solution was diluted with 20 mL of DMSO and precipitated into 600 mL of cold diethyl ether at 0° C. three times. The precipitants were collected, washed with 100 mL of cold ether, and dried under vacuum overnight to afford the PEO₄₅-b-PNAS₁₀₅ block copolymer precursor as a yellow solid (1.4 g, 68% yield based upon monomer conversion). 1H NMR (600 MHz, DMSO-d₆, ppm): δ 0.81 (t, J=6 Hz, 3H, dodecyl CH₃), 1.09 (br, 5H, CH₃ and dodecyl CH₂), 1.20 (br, 19H, CH₃ and dodecyl CH₂s), 1.30 (br, 2H, dodecyl CH₂), 1.60 (t, J=6 Hz, 2H, dodecyl CH₂), 2.01 (br, PNAS backbone protons), 2.75 (NAS CH₂CH₂s), 3.09 (br, PNAS backbone protons), 3.20 (s, mPEG terminal OCH₃), 3.47 (m, OCH₂CH₂O from the PEG backbone), 4.07 (br, 2H from the PEO backbone terminus connected to the ester linkage); 13C NMR (150 MHz, DMSO-d₆, ppm): δ 25.2, 41.2, 69.8, 172.8. PDI=1.3 (DMF GPC).

Synthesis of PEO₄₅-b-PNAS₁₀₅-b-PS₄₅ (1)

To a 25 mL Schlenk flask equipped with a magnetic stir bar dried with flame under N₂ atmosphere, was added the PEO₄₅-b-PNAS₁₀₅ macro-CTA (1.1 g, 55 μmol), 1,4-dioxane (5.0 mL), and DMF (5.0 mL). The reaction mixture was stirred 0.5 h at room temperature to obtain a homogeneous solution. To this solution was added styrene (2.2 g, 21 mmol) and AIBN (0.49 mg, 3.0 μmol). The reaction flask was sealed and stirred 10 min at room temperature. The reaction mixture was degassed through several cycles of freeze-pump-thaw. After the last cycle, the reaction mixture was stirred for 10 min at room temperature before being immersed into a pre-heated oil bath at 58° C. to start the polymerization. After 14.5 h, the monomer conversion reached ca. 13% by analyzing aliquots collected through 1H-NMR spectroscopy. The polymerization was quenched by cooling the reaction flask with liquid N₂. The polymer was purified by precipitation into 500 mL of cold diethyl ether at 0° C. three times. The precipitants were collected and dried under vacuum overnight to afford the block copolymer precursor as a yellow solid (1.0 g, 70% yield based upon monomer conversion). 1H NMR (600 MHz, CD₂Cl₂, ppm): δ 0.81 (br, dodecyl CH₃), 1.10-2.40 (br, dodecyl Hs, PNAS, and PS backbone protons), 2.75 (NAS CH₂CH₂s), 3.15 (br, PNAS backbone protons), 3.28 (s, mPEG terminal OCH₃), 3.60 (m, OCH₂CH₂O from the PEG backbone), 6.20-7.30 (br, Ar Hs); 13C NMR (150 MHz, DMSO-d₆, ppm): δ 25.2, 41.6, 69.8, 125.7, 128.0, 145.2, 172.8. PDI=1.2 (DMF GPC).

General Procedure for Self-Assembly of 1

To a solution of PEO₄₅-b-PNAS₁₀₅-b-PS₄₅ block copolymer in DMF (ca. 1.0 mg/mL), was added dropwise an equal volume of nano-pure H₂O within 2 h via a syringe pump at a rate of 15.0 mL/h. The mixture was further stirred for 1 h at room temperature before using for characterizations and cross-linking/functionalization reactions.

General Procedure for Cross-Linking/Functionalization of PEO₄₅-b-PNAS₁₀₅-b-PS₄₅ Multicompartment Micelles (MCMs)

To a solution of PEO₄₅-b-PNAS₁₀₅-b-PS₄₅ MCMs (30.0 mg of block copolymer precursor, 127 μmol of NAS residues) in 60.0 mL of DMF/H₂O (v:v=1:1) at room temperature, was added dropwise over 10 min, a solution of cross-linker 2 or 3 of FIG. 24 (12.7 μmol for nominal 20% of cross-linking and 31.8 μmol for nominal 50% of cross-linking, respectively) in nano-pure H₂O. The reaction mixture was allowed to stir for 48 h at room temperature in the absence of light. The reaction mixture was then divided into five portions (ca. 13 mL for each) and transferred into pre-soaked dialysis tubing (MWCO 6,000-8,000 Da) and dialyzed against 5.0 mM buffer solutions (with 5.0 mM NaCl) at pH 5.8, 6.5, 7.2, 7.9, and 8.6, respectively, for 7 days to remove DMF, un-reacted cross-linker, and the small molecule by-products to afford an aqueous solution of cross-linked/functionalized multicompartment nanostructures (MCNs).

Acylation of 3

To a solution of 3 (25.2 mg, 0.15 mmol) in 4 mL of H₂O at room temperature, was added dropwise over 5 min, a solution of NAS (127 mg, 0.75 mmol) in 4 mL of DMF. The reaction mixture was allowed to stir for 48 h at room temperature in the absence of light. The solvent was removed under vacuum. The residues were re-suspending into 5 mL of CH₂Cl₂ and precipitating into 35 mL of dry diethyl ether. The solid product was collected by centrifugation and re-dissolved into 30 mL of nano-pure water. The solution was passed through a 5 μm syringe filter to afford an aqueous stock solution of acylated 3. Before photo-physical measurements, the stock solution was diluted (v:v=1:5) with 5.0 mM buffer solution (with 5.0 mM NaCl) at pH 5.8, 6.5 7.2, 7.9, and 8.6, respectively.

### REFERENCES FOR EXAMPLE 6

[1] (a) Block Copolymers in Nanoscience; Lazzari, M., Liu, G., Lecommandoux, S., Eds.; Wiley-VCH: Weinheim, 2006. (b) Ruzette, A. V.; Leibler, L. Nat. Mater. 2005, 4, 19. (c) Nishiyama, N.; Kataoka, K. Pharmacol. Ther. 2006, 112, 630. (d) Olson, D. A.; Chen, L.; Hillmyer, M. A. Chem. Mater. 2008, 20, 869. (e) Hamley, I. W. Prog. Polym. Sci. 2009, 34, 1161.
[2] (a) Hawker, C. J.; Wooley, K. L. Science 2005, 309, 1200. (b) Champion, J. A.; Katare, Y. K.; Mitragotri, S. J. Controlled Release 2007, 121, 3.
[3] (a) Tsarevsky, N. V.; Matyjaszewski, K. Chem. Rev. 2007, 107, 2270. (b) Sciannamea, V.; Jerome, R.; Detrembleur, C. Chem. Rev. 2008, 108, 1104. (c) Boyer, C.; Bulmus, V.; Davis, T. P.; Ladmiral, V.; Liu, J.; Perrier, S. Chem. Rev. 2009, 109, 5402.
[4] (a) Liu, X.; Kim, J.-S.; Wu, J.; Eisenberg, A. Macromolecules 2005, 38, 6749. (b) Cui, H.; Chen, Z.; Zhong, S.; Wooley, K. L.; Pochan, D. J. Science 2007, 317, 647. (c) Dupont, J.; Liu, G.; Niihara, K.; Kimoto, R.; Jinnai, H. Angew. Chem., Int. Ed. 2009, 48, 6144. (d) Chen, Z.; Cui, H.; Hales, K.; Li, Z.; Qi, K.; Pochan, D. J.; Wooley, K. L. J. Am. Chem. Soc. 2005, 127, 8592.
[5] (a) Erhardt, R.; Zhang, M.; Boker, A.; Zettl, H.; Abetz, C.; Frederik, P.; Krausch, G.; Abetz, V.; Muller, A. H. E. J. Am. Chem. Soc. 2003, 125, 3260. (b) Li, Z.; Kesselman, E.; Talmon, Y.; Hillmyer, M. A.; Lodge, T. P. Science 2004, 306, 98. (c) Kubowicz, S.; Baussard, J.-F.; Lutz, J.-F.; Thuenemann, A. F.; von Berlepsch, H.; Laschewsky, A. Angew. Chem., Int. Ed. 2005, 44, 5262. (d) Talingting, M. R.; Munk, P.; Webber, S. E.; Tuzar, Z. Macromolecules 1999, 32, 1593. (e) Shen, H.; Zhang, L.; Eisenberg, A. J. Am. Chem. Soc. 1999, 121, 2728.
[6] (a) Li, Z.; Hillmyer, M. A.; Lodge, T. P. Langmuir 2006, 22, 9409. (b) Liu, C.; Hillmyer, M. A.; Lodge, T. P. Langmuir 2008, 24, 12001. (c) Walther, A.; Mueller, A. H. E. Chem. Commun. 2009, 1127. (d) Fang, B.; Walther, A.; Wolf, A.; Xu, Y.; Yuan, J.; Mueller, A. H. E. Angew. Chem., Int. Ed. 2009, 48, 2877. (e) von Berlepsch, H.; Boettcher, C.; Skrabania, K.; Laschewsky, A. Chem. Commun. 2009, 2290. (f) Schacher, F.; Betthausen, E.; Walther, A.; Schmalz, H.; Pergushov, D. V.; Mueller, A. H. E. ACS Nano 2009, 3, 2095. (g) Uchman, M.; Stepanek, M.; Prochazka, K.; Mountrichas, G.; Pispas, S.; Voets, I. K.; Walther, A. Macromolecules 2009, 42, 5605. (h) Schacher, F.; Walther, A.; Ruppel, M.; Drechsler, M.; Mueller, A. H. E. Macromolecules 2009, 42, 3540.
[7] Lodge, T. P.; Rasdal, A.; Li, Z.; Hillmyer, M. A. J. Am. Chem. Soc. 2005, 127, 17608.
[8] (a) Reynhout, I. C.; Cornelissen, J.; Nolte, R. J. M. J. Am. Chem. Soc. 2007, 129, 2327. (b) Hu, J.; Liu, G.; Nijkang, G. J. Am. Chem. Soc. 2008, 130, 3236.
[9] Sun, G.; Lee, N. S.; Neumann, W. L.; Freskos, J. N.; Shieh, J. J.; Dorshow, R. B.; Wooley, K. L. Soft Matter 2009, 5, 3422.
[10] The hydrolysis of NAS dramatically affected the self-assembly behavior of the triblock copolymer precursors. Uniform MCMs with smaller size (Dₕ=160±15 nm, FIG. 34, Panel B) and less number of compartments (FIG. 34, Panel C) were achieved through the assembly of PEO₄₅-b-P(NAS₉₅-co-AA₁₀)-b-PS₄₅ (FIG. 34, Panel A) precursors.
[11] Shirai, K.; Yanagisawa, A.; Takahashi, H.; Fukunishi, K.; Matsuoka, M. Dyes Pigm. 1998, 39, 49.
[12] Lee, N. S.; Sun, G.; Neumann, W. L.; Freskos, J. N.; Shieh, J. J.; Dorshow, R. B.; Wooley, K. L. Adv. Mater. 2009, 21, 1344.
[13] (a) LaFemina, J. P.; Schenter, G. K. J. Chem. Phys. 1991, 94, 7558. (b) Gomez, I.; Reguero, M.; Boggio-Pasqua, M.; Robb, M. A. J. Am. Chem. Soc. 2005, 127, 7119. (c) Cogan, S.; Zilberg, S.; Haas, Y. J. Am. Chem. Soc. 2006, 128, 3335.

### Example 7: Controlling Fluorescence Emission Wavelength of Photonic SCKs Through Chemical Manipulation of Shell Cross-Linking Reactions

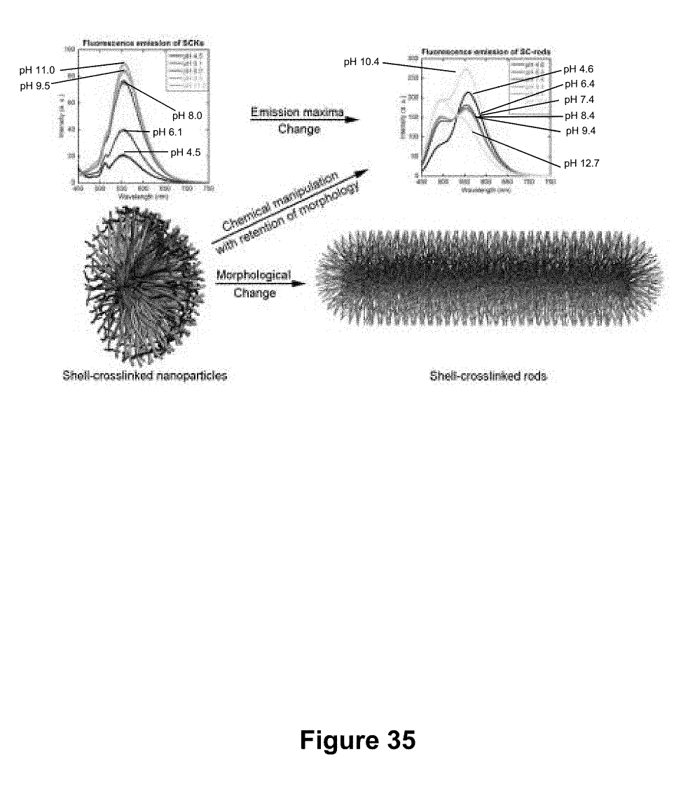

Towards the goal of developing biophotonic embedded therapeutics for optical imaging and monitoring, we have striven to understand and control the photophysical properties of various photonic nanostructures. These nanostructures represent a stable template onto which targeting peptides can be conjugated and drug molecules sequestered. In preparation of well-defined, discrete shell-cross-linked nanoparticles (SCKs), the shell cross-linking reaction between the shell moiety and the cross-linker is the key step that ensures the integrity of the nanostructures in a wide variety of conditions in vivo (pH, ionic strength, dilution, etc.). We have previously utilized cross-linking chromophores in this key step to impart pH-responsive enhancements of fluorescence emissions in the resulting fluorophore-SCKs for pH-sensing applications.[1,2] Through investigation of photonic shell-cross-linked rods (SC-rods), more recently, we first encountered and were intrigued by the blue shift (by ca. 60 nm) in fluorescence emission (FIG. 35). We decided to further study the mechanism of the blue shift towards development of a nanomaterial that response to its local pH environment by a manner that may lead to a selective signal change with predictive ratios that allow for direct determination of pH.

We initially hypothesized that the dual-peak emission of SC-rods was a result of cross-linking chromophores being exposed to two distinct environments within the nanostructure framework—namely, that of low interfacial curvature in the middle section and higher interfacial curvature on two end-caps of the rods. Our second hypothesis was that the aromatic amines in the cross-linking chromophore were involved in the cross-linking reaction thereby changing its electronic nature and the corresponding emission profile. In this part of the report, our recent effort to address this issue through chemical modifications of the shell cross-linking reactions is highlighted.

A. Controlling Photophysical Properties of Shell-Cross-Linked Rods

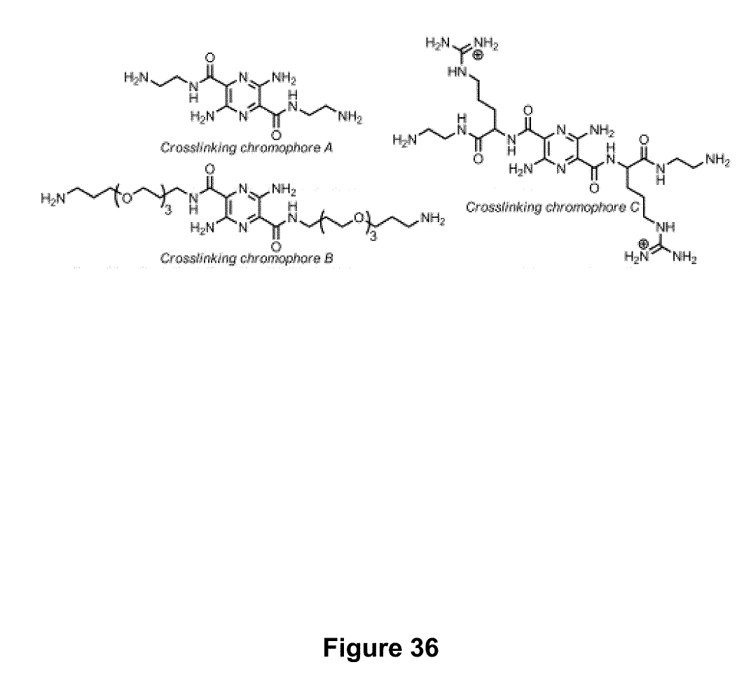

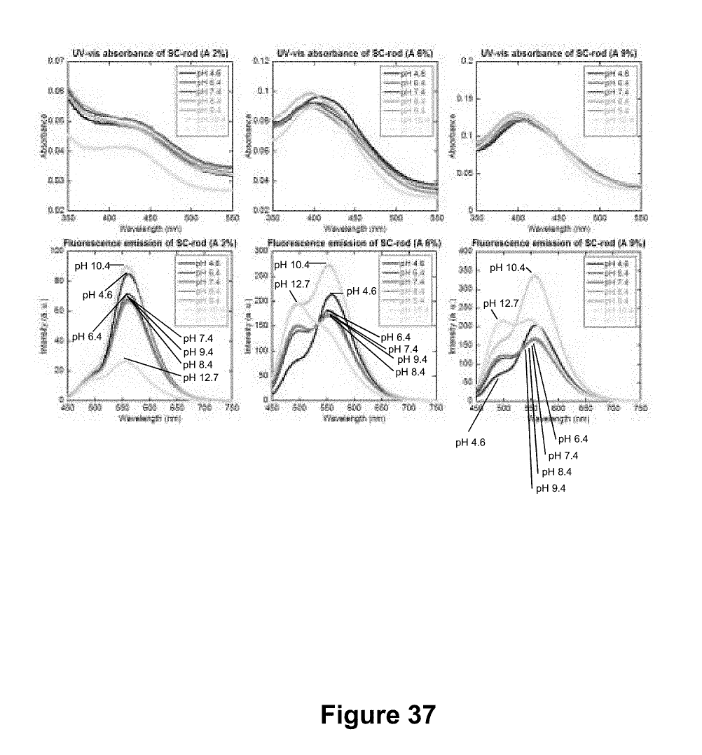

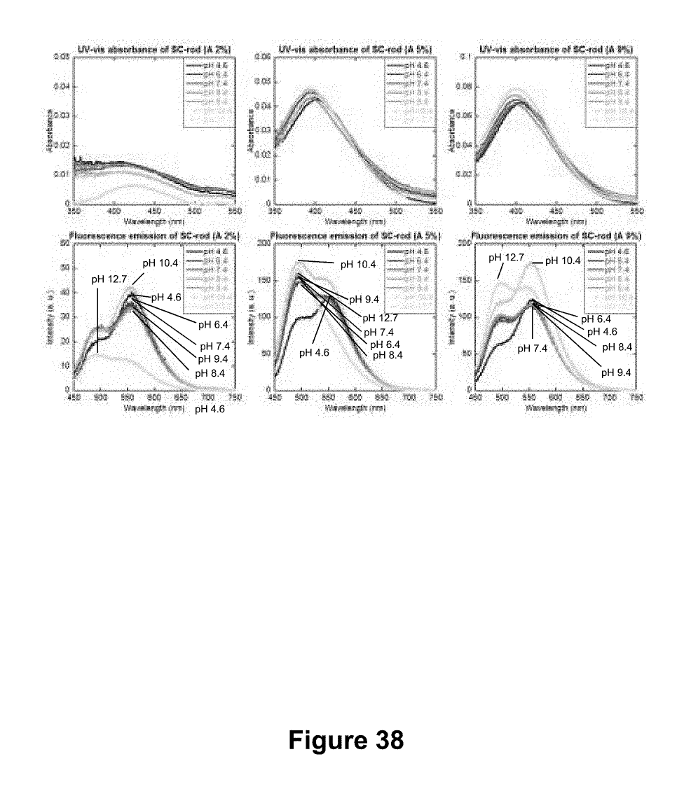

Shell-cross-linked rods in this study are comprised of poly(acrylic acid)₁₄₀-block-poly(p-hydroxystyrene)₅₀ (PAA₁₄₀-b-PpHS₅₀) as a nanoscale template and cross-linking chromophore A, B or C (FIG. 36) as a cross-linker and as an optical handle. Shell cross-linking reactions are condensation reactions between diamines and the poly(acrylic acid) shell moiety of the nanostructures in the presence of water-soluble carbodiimide, EDCl. Typically, 1:1 molar ratio or slight excess of carbodiimide to amines is added to the reaction mixture in order to form sufficient amount of activated ester for intramicellar cross-linking reactions while avoiding intermicellar reactions. In order to assess the extent to which aromatic amines participated in the cross-linking reaction, the amount of cross-linking chromophore loading (2%, 6% or 9% cross-linking density) as well as EDCl loading (stoichiometric or 2 molar excess) were varied (FIGS. 37 and 38). Cross-linking chromophore A shell-cross-linked rods (SC-rod A) already display dual-peak emission (FIG. 37). When the amount of EDCl added is doubled, blue-shifted emission peak becomes greater while the original emission peak diminishes. This is most obvious for the 6% cross-linked rods (SC-rod A 6%).

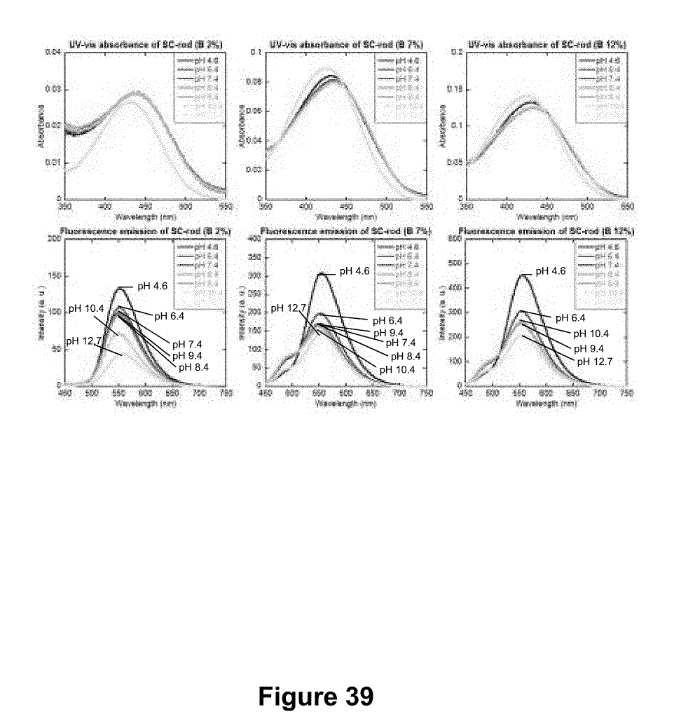

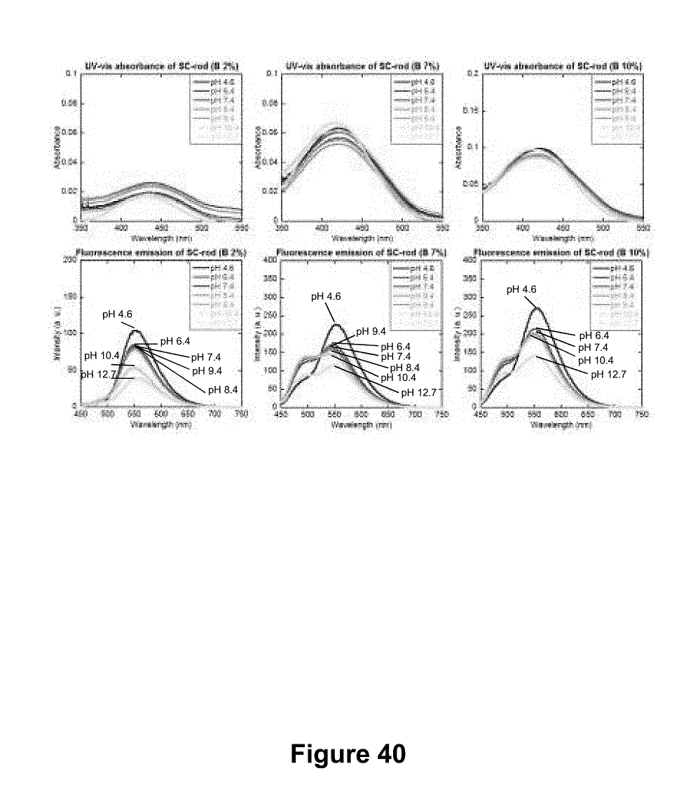

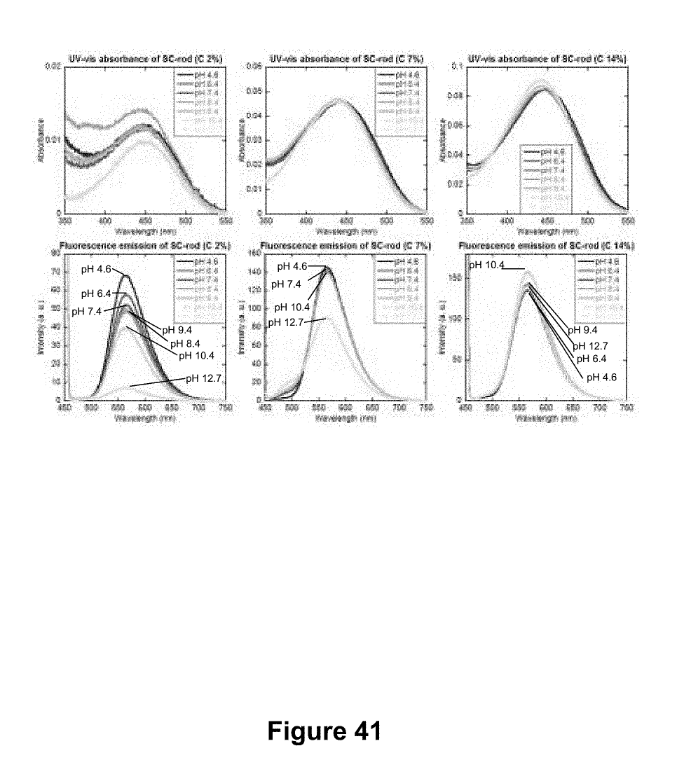

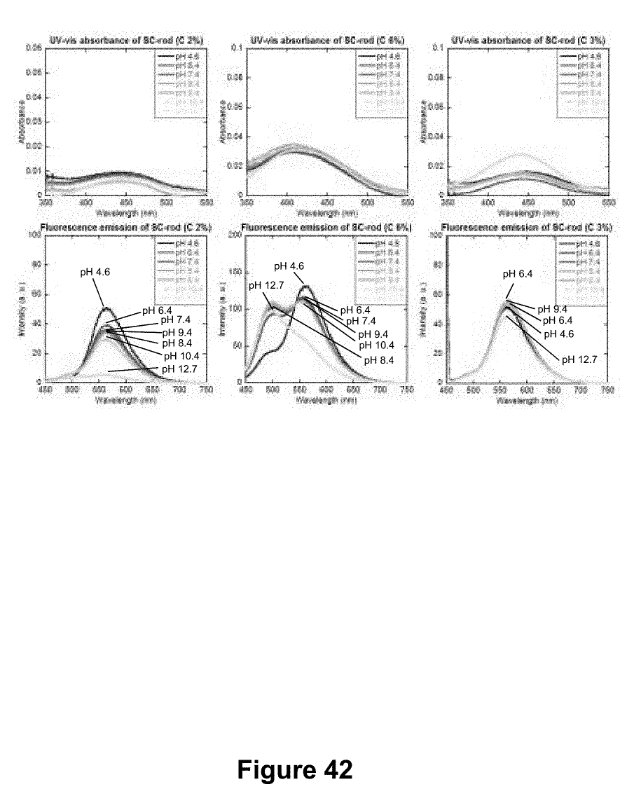

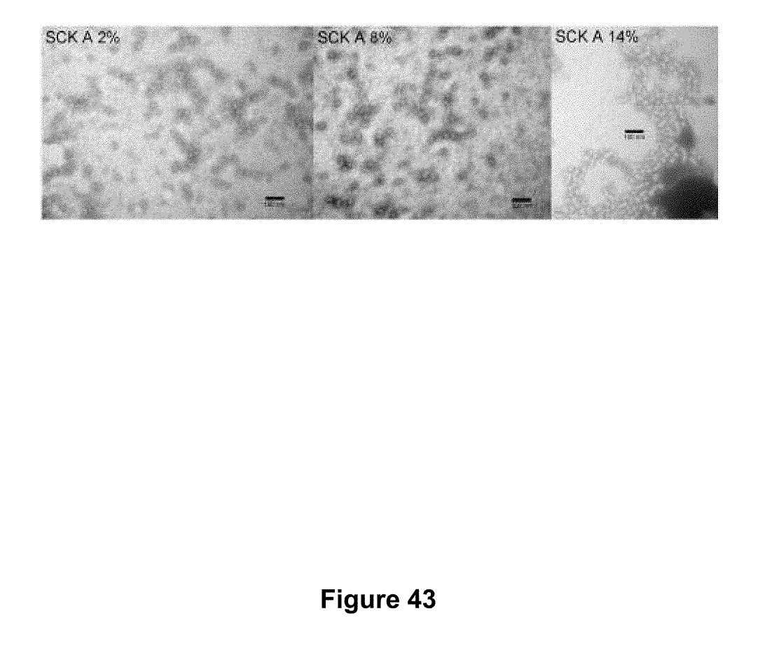

SC-rod B's show a similar trend except the blue-shifted emission peak never overwhelmingly dominates the original emission peak (FIGS. 39 and 40). In both cases, 6% cross-linked rods undergo the greatest shift in emission wavelength, suggesting that as the cross-linking density increases to 9%, or as the available acrylic acid residues to amine ratio decreases, the reaction between acrylic acids and the aromatic amines becomes less favorable. This phenomenon becomes amplified in SC-rod C series, where only 7% cross-linked rods undergo any appreciable amount of blue-shift (FIGS. 41 and 42). FIG. 43 provides transmission electron micrograph (TEM) images of SCK A series with 50 molar excess EDCl at cross-linking percentages of 2%, 8%, and 14% where the black bar in each image represents a length of 100 nanometers.

B. Controlling Photophysical Properties of Shell-Cross-Linked Nanoparticles

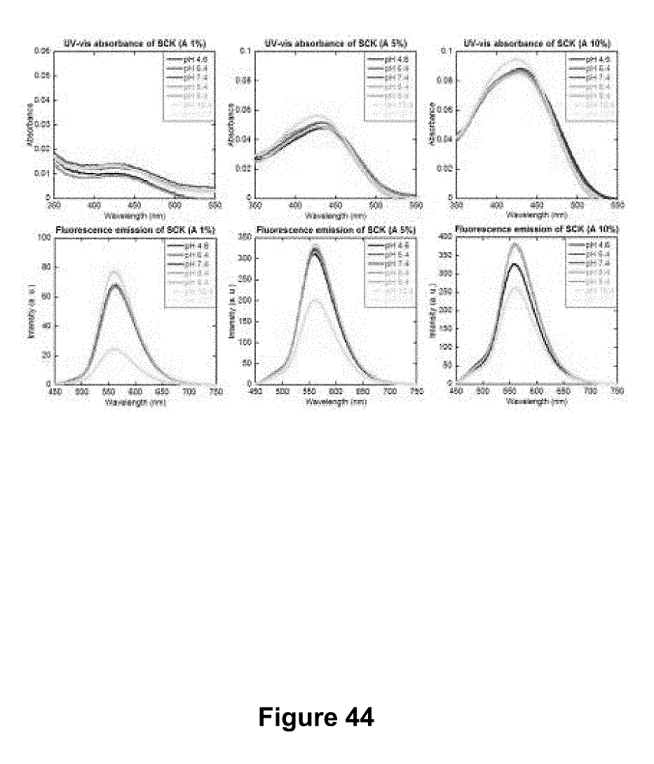

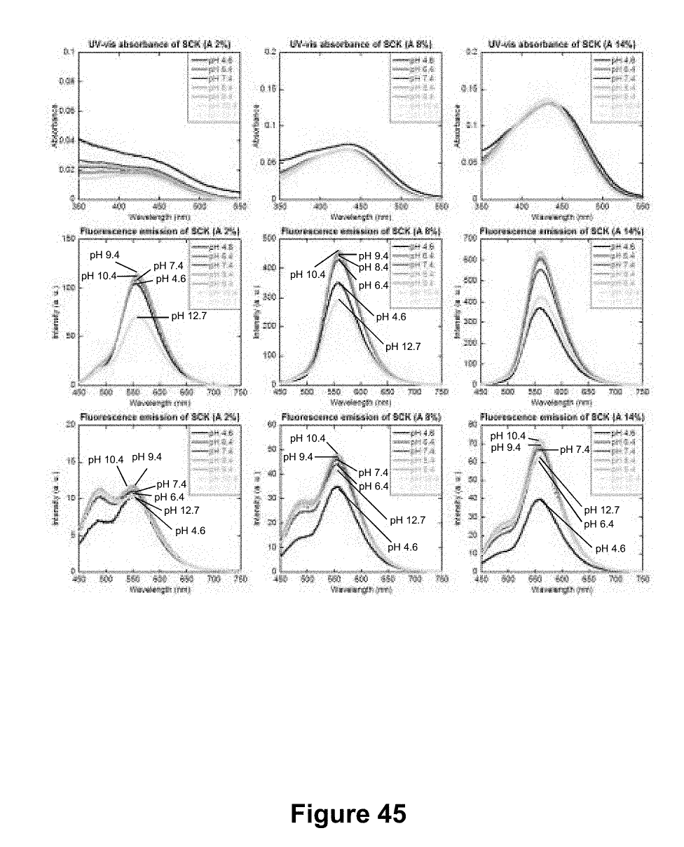

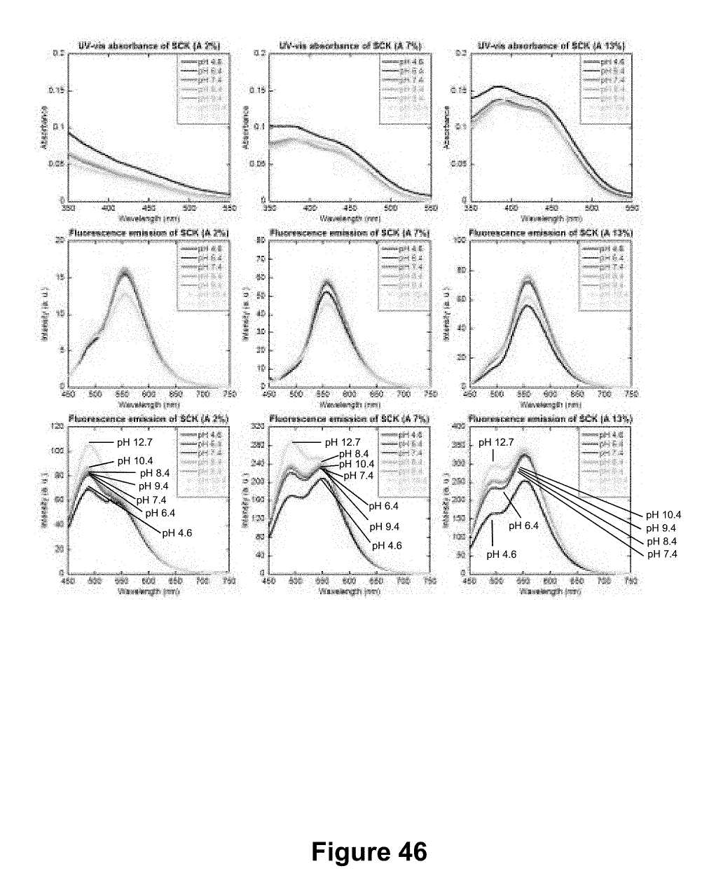

Having observed that the addition of excess EDCl in shell cross-linking reactions allowed SC-rods to blue shift to a greater extent by encouraging aromatic amines to react with acrylic acid residues, we then conducted similar experiments on photonic SCK spheres, where no blue shift was previously observed, to determine whether it would be possible to impart blue shift in fluorescence emission. In this set of experiments, we added to the spherical micelle solution, 2, 35 and 70 molar excess of EDCl, where 70 molar excess EDCl essentially activates all available carboxylic acid residues. As the EDCl loading increases, the degree to which fluorescence emission blue-shifts becomes greater. The second highest cross-linker loading still undergoes the greatest blue shift (FIGS. 44, 45 and 46). In essence, by manipulating the cross-linking reaction conditions with retention of morphology, we were able to achieve the photophysical consequences within a spherical framework that had been previously exclusive to multi-compartment nanostructures and shell-cross-linked rods.

C. Controlling Photophysical Properties of SCKs by “Cross-Linking Again”

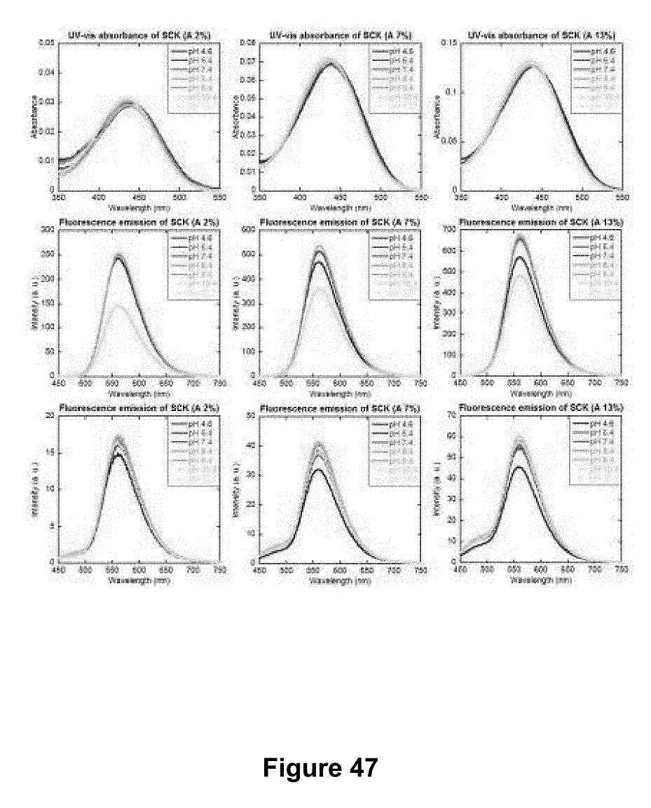

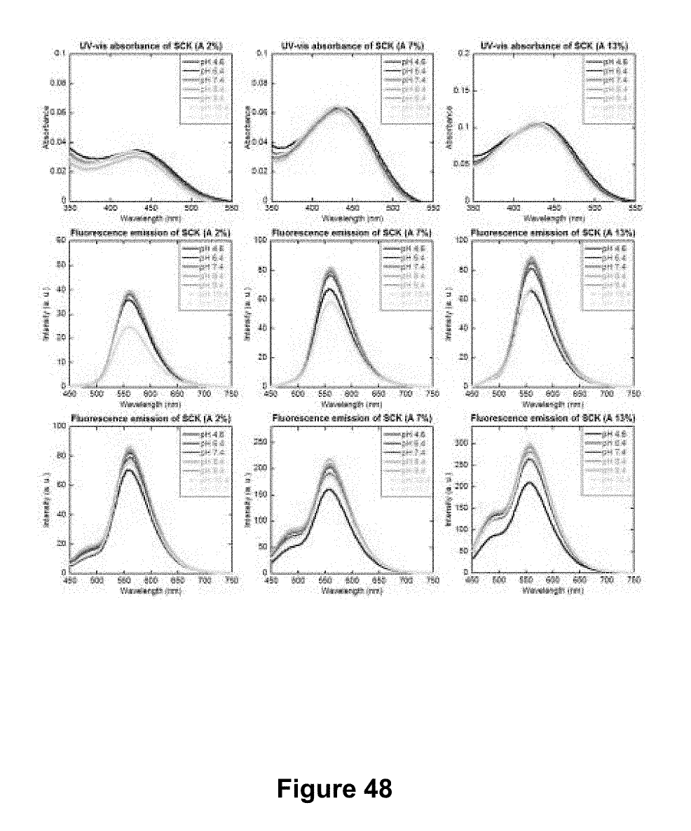

The above data indicate that unreacted aromatic amines of the cross-linking chromophore remained available for further reactions with the acids after a shell cross-linking reaction. Here, we have applied two back-to-back cross-linking reactions with a fixed amount of EDCl. We first prepared a batch of SCK A series with stoichiometric amount of EDCl and purified by dialysis to remove free cross-linking chromophores and urea by-products. To the purified batch was added an additional stoichiometric amount of EDCl to allow for reactions between unreacted amines and residual PAA units. Fluorescence emission spectra show increase in blue-shifted emission peak after the second cross-linking reaction (FIGS. 47 and 48).

The process of installing a diagnostic tool onto the nanostructure has led to several important fundamental findings that will yield a more sophisticated diagnostic therapeutic nanomaterial. We have used cross-linking chromophores to not only measure the cross-linking density (or incorporation efficiency)[3] of the resulting nanostructures, but also to take full advantage of its structural compatibility to our nanostructure and fine tune their photophysical properties. These findings will play a vital role in future developments of biophotonic embedded therapeutics.

### REFERENCES FOR EXAMPLE 7

[1] N. S. Lee, G. Sun, W. L. Neumann, J. N. Freskos, J. J. Shieh, R. B. Dorshow, K. L. Wooley, Adv. Mater, 2009, 20, 1-5.
[2] Neumann, W. L.; Rajagopalan, R.; Dorshow, R. B.; Shieh, J. J.; Freskos, J. N.; Lee, N. S.; Wooley, K. L. U. S. Provisional Patent No. 60/986,171, filed Nov. 7, 2007.
[3] G. Sun, N. S. Lee, W. L. Neumann, J. N. Freskos, J. J. Shieh, R. B. Dorshow, K. L. Wooley, Soft Matter, 2009, 5, 3422-3429.

### Example 8: Uniform, Functionalized, Cross-Linked Multicompartment Nanostructure Bioconjugates Providing Targeting Functionality

Nanoscopic drug-delivery vehicles takes advantage of (i) internal capacity of the core, maintaining a protected nanoscopic vessel-like environments for the packaging and protection of therapeutic agents and (ii) surface multi-functionality through high surface area-to-volume ratios to increase availability and multivalency of targeting ligands. This unique feature is in addition to the passive targeting (i.e., re-direction of biodistribution of the guest molecules through diffusion into leaky vasculatures of tumors). Therefore, preparation of nanoscopic diagnostic-therapeutic materials requires development of an orthogonal conjugation chemistry that is applicable to a peptide and a nanostructure of interest and tolerant towards functional groups present within the system. Installation of thiol groups at the terminus of poly(ethylene oxide) (PEO) with a number-average molecular weight of 3,000 Daltons that is grafted onto the polymer backbone addresses this issue and further makes it possible to present the targeting peptides onto the outer-most corona region of the nanostructure while a PEO of 2 kDa shapes the inner corona. Any thiol-reactive groups can be covalently attached to the end of the PEO graft (e.g., maleimide or bromoacetyl). We have studied conjugation chemistry between thiols and maleimides or bromoacetyl groups, but presumably any haloacetyl groups can also be used through the same chemistry among others (for example, thiol-thiol, thiol-aziridine, thiol-acryloyl, etc.).

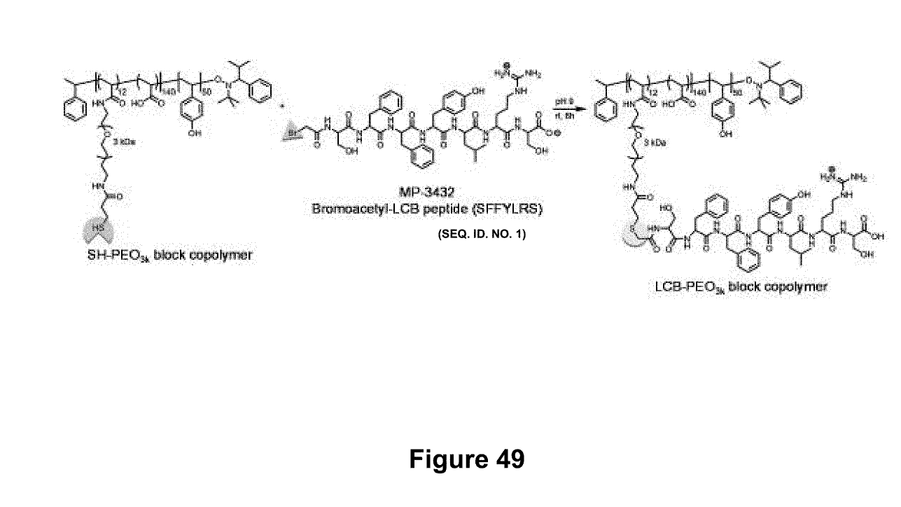

FIG. 49 provides a conjugation reaction scheme for conjugation of an SH-PEO₃ₖ block copolymer with the LCB peptide Ser-Phe-Phe-Tyr-Leu-Arg-Ser (SEQ. ID. NO. 1) for providing targeting functionality to block copolymers. The conjugation of the LCB-peptide was carried out using the thiol-bromoacetyl reaction scheme shown in FIG. 49. The reaction was carried out at pH 9 for 3 hours under nitrogen. The solution pH was then adjusted to 6 and maleimidobutyric acid was added to react with any residual thiol groups. The reaction mixture was purified by dialysis against 5 mM PBS and lyophilized to afford the LCB-PEO₃ₖ block copolymer. A separate batch of mPEO₂ₖ block copolymer was synthesized to be co-assembled with the LCB-PEO₃ₖ block copolymer.

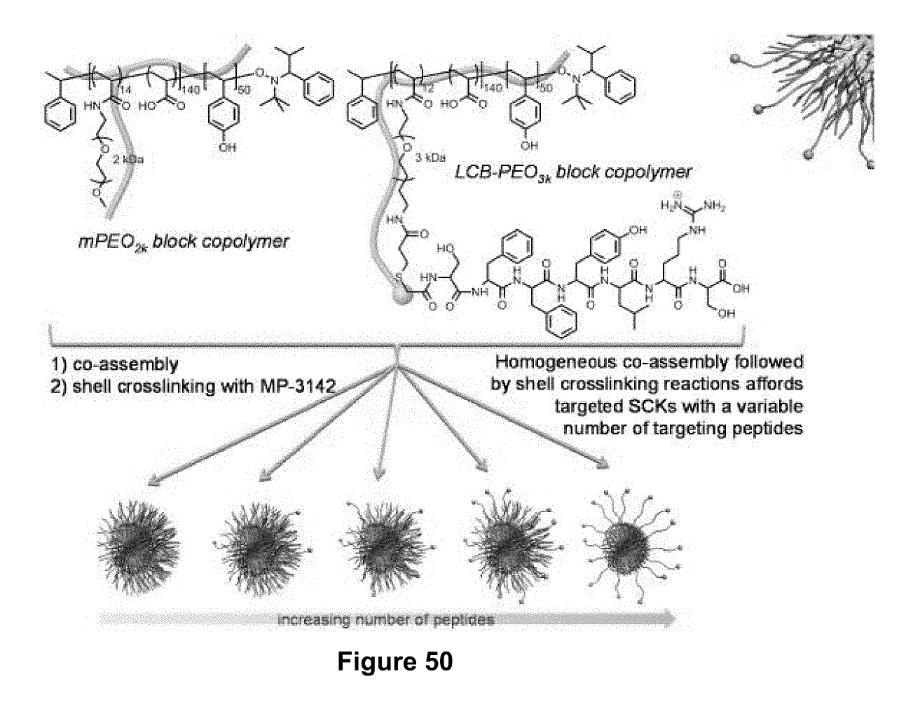

FIG. 50 provides a co-assembly reaction scheme for co-assembly of LCB-PEO₃ₖ/mPEO₂ₖ block copolymers, wherein the LCB peptide is Ser-Phe-Phe-Tyr-Leu-Arg-Ser (SEQ. ID. NO. 1), for providing targeting functionality to block copolymers. First, mPEO₂ₖ block copolymers and the LCB-PEO₃ₖ block copolymers of FIG. 49 were co-assembled. Second, the mPEO₂ₖ block copolymers were cross-linked using cross-linker MP-3142 of FIG. 2. The homogeneous co-assembly followed by shell cross-linking reactions affords targeted SCKs with a variable number of targeting peptides.

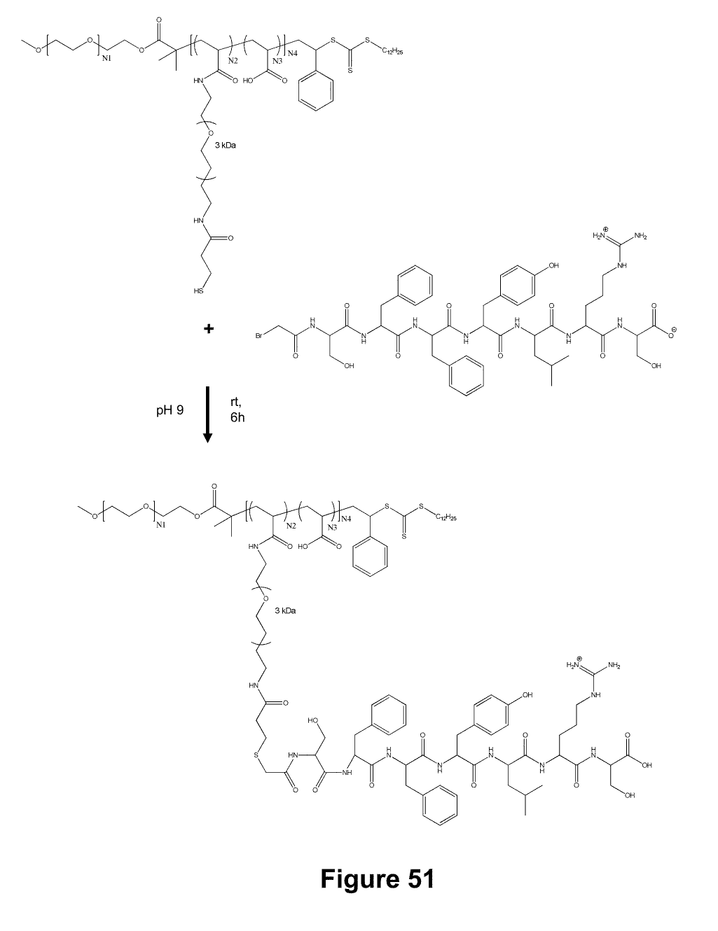

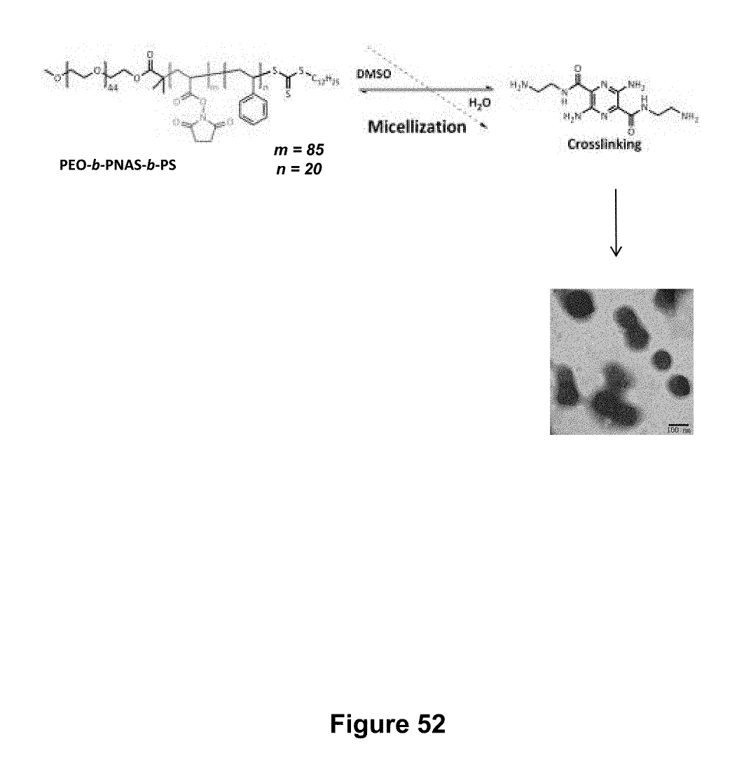

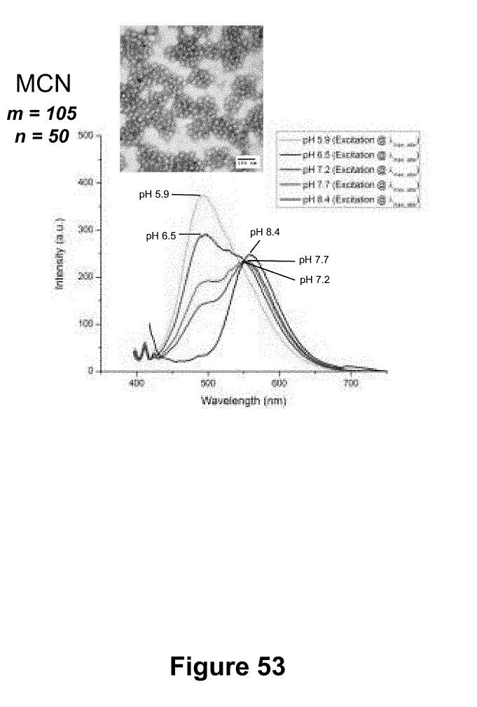

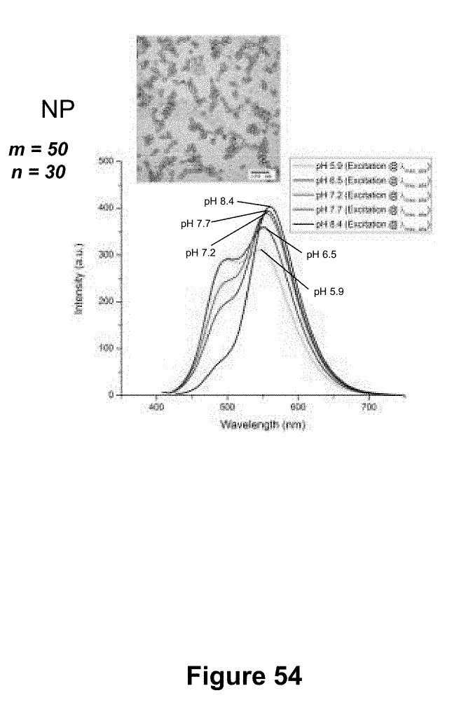

FIG. 51 provides a conjugation reaction scheme for conjugation of a PEO₄₅-b-PNAS₁₀₅ block copolymer with the LCB peptide Ser-Phe-Phe-Tyr-Leu-Arg-Ser (SEQ. ID. NO. 1) for providing targeting functionality to block copolymers. The conjugation reaction was carried out at pH 9 at room temperature for 6 hours. This bioconjugate can be further co-assembled with PEO₄₅-b-PNAS₁₀₅ block copolymers and cross-linked to yield an SCKs with a variable number of targeting peptides.

As used herein, “targeting ligand” (abbreviated as Bm) refers to a chemical group and/or substituent having functionality for targeting the functionalized, cross-linked compounds described herein—for example functionalized, cross-linked compounds comprising the triblock copolymer of (FX23)—to an anatomical and/or physiological site of a patient, such as a selected cell, tissue or organ. For some embodiments, a targeting ligand is characterized as a ligand that selectively or preferentially binds to a specific biological site(s) (e.g., enzymes, receptors, etc.) and/or biological surface(s) (e.g., membranes, fibrous networks, etc.). In an embodiment, the invention provides a functionalized, cross-linked compound described herein—for example functionalized, cross-linked compounds comprising the triblock copolymer of (FX23)—, wherein Bm is an amino acid, or a polypeptide comprising 2 to 30 amino acid units. In an embodiment, the invention provides a functionalized, cross-linked compound described herein—for example functionalized, cross-linked compounds comprising the triblock copolymer of (FX23)—, wherein Bm is a mono- or polysaccharide comprising 1 to 50 carbohydrate units. In an embodiment, the invention provides a functionalized, cross-linked compound described herein—for example functionalized, cross-linked compounds comprising the triblock copolymer of (FX23)—, wherein Bm is a mono-, oligo- or poly-nucleotide comprising 1 to 50 nucleic acid units. In an embodiment, the invention provides a functionalized, cross-linked compound described herein—for example functionalized, cross-linked compounds comprising the triblock copolymer of (FX23)—, wherein Bm is a protein, an enzyme, a carbohydrate, a peptidomimetic, a glycomimetic, a glycopeptide, a glycoprotein, a lipid, an antibody (polyclonal or monoclonal), or fragment thereof. In an embodiment, the invention provides a functionalized, cross-linked compound described herein—for example functionalized, cross-linked compounds comprising the triblock copolymer of (FX23)—, wherein Bm is an aptamer. In an embodiment, the invention provides a functionalized, cross-linked compound described herein—for example functionalized, cross-linked compounds comprising the triblock copolymer of (FX23)—, wherein Bm is a drug, a hormone, steroid or a receptor. In some embodiments, each occurrence of Bm in the compounds described herein—for example functionalized, cross-linked compounds comprising the triblock copolymer of (FX23)—is independently a monoclonal antibody, a polyclonal antibody, a metal complex, an albumin, or an inclusion compound such as a cyclodextrin. In some embodiments, each occurrence of Bm in the compounds described herein—for example functionalized, cross-linked compounds comprising the triblock copolymer of (FX23)—is independently integrin, selectin, vascular endothelial growth factor, fibrin, tissue plasminogen, thrombin, LDL, HDL, Sialyl LewisX or a mimic thereof, or an atherosclerotic plaque binding molecule. Throughout the present description, the term “biomolecule” can be a targeting ligand (Bm). In an embodiment, the invention provides a functionalized, cross-linked compound described herein—for example functionalized, cross-linked compounds comprising the triblock copolymer of (FX23)—, wherein Bm is a polysaccharide comprising 2 to 50 furanose or pyranose units.

In the functionalized, cross-linked compound described herein—for example functionalized, cross-linked compounds comprising the triblock copolymer of (FX23)—, Bm is a targeting ligand, optionally providing molecular recognition functionality. In some embodiments, the targeting ligand is a particular region of the compound that is recognized by, and binds to, a target site on an organ, tissue, tumor or cell. Targeting ligands are often, but not always, associated with biomolecules or fragments thereof which include, but are not limited to, hormones, amino acids, peptides, peptidomimetics, proteins, nucleosides, nucleotides, nucleic acids, enzymes, carbohydrates, glycomimetics, lipids, albumins, mono- and polyclonal antibodies, receptors, inclusion compounds such as cyclodextrins, and receptor binding molecules. Targeting ligands for use in the invention can also include synthetic polymers. Examples of synthetic polymers that are useful for targeting ligands include polyaminoacids, polyols, polyamines, polyacids, oligonucleotides, aborols, dendrimers, and aptamers. Still other examples of useful targeting ligands can include integrin, selectin, vascular endothelial growth factor, fibrin, tissue plasminogen activator, thrombin, LDL, HDL, Sialyl LewisX and its mimics, and atherosclerotic plaque binding molecules.

Specific examples of targeting ligands include, but are not limited to: steroid hormones for the treatment of breast and prostate lesions; whole or fragmented somatostatin, bombesin, and neurotensin receptor binding molecules for the treatment of neuroendocrine tumors; whole or fragmented cholecystekinin receptor binding molecules for the treatment of lung cancer; whole or fragmented heat sensitive bacterioendotoxin (ST) receptor and carcinoembryonic antigen (CEA) binding molecules for the treatment of colorectal cancer; dihydroxyindolecarboxylic acid and other melanin producing biosynthetic intermediates for the treatment of melanoma; whole or fragmented integrin receptor and atherosclerotic plaque binding molecules for the treatment of vascular diseases; and whole or fragmented amyloid plaque binding molecules for the treatment of brain lesions. In some embodiments, Bm, if present, is selected from heat-sensitive bacterioendotoxin receptor binding peptide, carcinoembryonic antigen antibody (anti-CEA), bombesin receptor binding peptide, neurotensin receptor binding peptide, cholecystekinin receptor binding peptide, somastatin receptor binding peptide, ST receptor binding peptide, neurotensin receptor binding peptide, leukemia binding peptides, folate receptor binding agents, steroid receptor binding peptide, carbohydrate receptor binding peptide or estrogen. In another embodiment Bm, if present, is a ST enterotoxin or fragment thereof. In some embodiments, Bm, if present, is selected from octreotide and octreotate peptides. In another embodiment Bm, if present, is a synthetic polymer. Examples of synthetic polymers useful for some applications include polyaminoacids, polyols, polyamines, polyacids, oligonucleotides, aborols, dendrimers, and aptamers. In an embodiment, Bm, if present, is an antibody or an antibody fragment, such as an antibody F_{ab} fragment, an antibody F_{(ab2)′} fragment, and an antibody F_{c} fragment. Examples of specific peptide targeting ligands are described in WO/2008/108941.

### Example 9: Tunable Dual-Emitting Shell-Cross-Linked Nano-Objects as Single-Component Ratiometric pH-Sensing Materials

Dual-emitting nano-objects that can sense changes in the environmental pH are designed based on shell-cross-linked micelles assembled from amphiphilic block copolymers and cross-linked with pH-insensitive chromophores. The ratio of fluorescence intensity at 496 nm over that of 560 nm is dependent upon the solution pH. The chromophoric cross-linkers are tetra-functionalized pyrazine molecules that bear a set of terminal aliphatic amine groups and a set of anilino amine groups, which demonstrate morphology-dependent reactivities towards the poly(acrylic acid) shell domain of the nano-objects. The extent to which the anilino amine groups react with the nano-object shell is shown to affect the hypsochromic shift (blue-shift). Disclosed herein are observations on the pH-sensitive dual-emission photophysical properties of rod-shaped or spherical nano-objects, whose shell domains offer two distinct platforms for amidation reactions to occur—through formation of activated esters upon addition of carbodiimide or pre-installation of activated ester groups. Physical manipulations (changes in morphology or particle dimensions) or chemical manipulations of the cross-linking reaction (the order of installation of activated esters) lead to fine tuning of dual-emission over ca. 60 nm in a physiologically relevant pH range. Rod-shaped shell-cross-linked nanostructures with poly(p-hydroxystyrene) core show blue-shift as a function of increasing pH while spherical shell-cross-linked nanostructures with polystyrene core and poly(ethylene oxide) corona exhibit blue-shift as a function of decreasing pH.

### 1. Introduction

Stimuli-sensitive materials that respond to changes in various biologically-relevant events with dual-emitting fluorescence as the output signals have been celebrated as a potential non-invasive diagnostic tool for various diseases. The types of stimuli of interest have naturally been associated with or caused by the characteristics of diseased cells, such as decreased pH [1-12], increased concentration of O₂ [13], presence of heavy-metal ions [14] or concentration of ATP [15] or proteins such as avidin [16] or RNase [17]. Ratiometric sensing based on a dual-emission profile is superior to single-emission, as the output is independent of sensor concentration and absolute fluorescence emission intensity. Single-component materials whose dual-emissions span over ca. 70 nm have been fabricated: For instance, Fraser and co-workers recently reported a ratiometric O₂ sensing film, prepared from iodide-substituted difluoroboron dibenzoylmethane-poly(lactic acid), which emitted fluorescence at 450 nm and 525 nm for tumor hypoxia imaging with a I₄₅₀/I₅₂₅ ratio ranging from ca. 0.22 to 0.41 [13]. For in vivo pH sensing, much of the recent developments have relied on the intrinsic pH-responsiveness of small molecule probes: The commercially-available carboxyseminaphthofluorescein (cSNARF®-1), for example, is a modified fluorescein molecule, whose emission spectrum undergoes a pH-dependent wavelength shift. The compound is usually excited between 488 nm and 530 nm while monitoring the fluorescence emission at two wavelengths, 580 nm and 640 nm, respectively, with I₅₈₀/I₆₄₀ ratios easily reaching values greater than thirty [18]. Drastic improvements on quantum yield of the chromophore have been realized by Burgess and co-workers through synthesis of a pH probe equipped with two xanthene (the fluorescent core of fluorescein) donors and one boron-dipyrromethene (BODIPY) acceptor with I₆₀₀/I₅₂₅ between one and five [5]. To minimize probe-protein interactions in vivo, small molecule chromophores have been encapsulated within the cavities of L-α-phosphatidylcholine-based liposomes, while maintaining pH responsiveness by providing minimal hindrance for the movement of protons across the liposome [19].

While great advances have been achieved in the synthesis and utilization of small molecule probes for detecting pH, it is of wide interest to design dual-emitting macromolecular/supramolecular probes that are water soluble and able to sequester guest molecules for coincident imaging and treatment of diseases. Jin and co-workers recently reported fluorescein isothiocyanate-coated quantum dots, having a hydrodynamic diameter of 7 nm, with an I₆₀₀/I₅₁₅ dual emission ratio ranging from fourteen to four from pH 6 to 8 [7]. Peng and Wolfbeis reported the preparation of a polyurethane-based nanogel loaded with the pH indicator bromothymol blue and the fluorophores Coumarin 6 and Nile Red as a two-component system that underwent Förster resonance energy transfer (FRET) in response to a pH change [10].

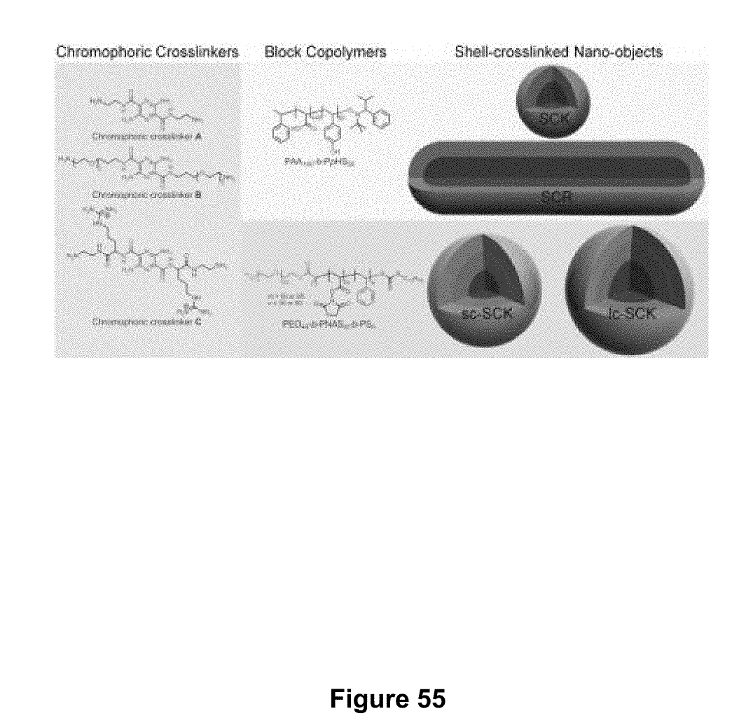

In designing a single-component, dual-emitting, pH-responsive nanoscopic probe, which is based upon pH-insensitive small molecule chromophores, shell-cross-linked knedel-like nanoparticles (SCKs) have emerged as an interesting nanotechnology platform. SCKs are well-defined, discrete macromolecular assemblies with unique covalently stabilized core-shell morphology and serve as a robust template onto which orthogonal chemical reactions can take place. In preparation of SCKs, condensation reactions between the shell of amphiphilic block copolymer micelles and cross-linkers is the key step that ensures maintenance of the integrity of the final nanostructures under a wide variety of conditions (pH, ionic strength, dilution, etc.). We have previously utilized pH-insensitive pyrazine-based chromophoric cross-linkers in this critical step to impart pH-responsive enhancement of single-wavelength fluorescence emission intensities in the resulting fluorophore-SCKs for pH-sensing applications [20]. Building upon our past advance, we envisioned a single-component, dual-emitting analogue as a powerful alternative to sense the pH, while utilizing the core/shell nature for loading of guest molecules [21-23] and attaching targeting ligands for active-targeted delivery [24-27]. Here we disclose preparation of pH-responsive, dual-emitting, single-component shell-cross-linked nano-objects and observation of their pH sensitive photophysical properties as a function of physical parameters (morphology or core/shell dimensions) or chemical parameters (stoichiometry of reagents added or pre-installation of reactive groups) using pH-insensitive chromophoric cross-linkers, some of which are shown in FIG. 55. Two morphologies were utilized in this study: shell-cross-linked rod-shaped nanostructures (SCRs) and spherical nanoparticles (SCKs). SCRs were self-assembled from poly(acrylic acid)-b-poly(p-hydroxystyrene) (PAA₁₄₀-b-PpHS₅₀) [28] and SCKs were self-assembled from either the same parent diblock copolymer, PAA₁₄₀-b-PpHS₅₀, or poly(ethylene oxide)-b-poly(N-acryloxysuccinimide)-b-polystyrene (PEO₄₅-b-PNAS₅₀-b-PS₃₀) or PEO₄₅-b-PNAS₉₅-b-PS₆₀ [29]. Surprisingly opposite behavior for SCRs vs. SCKs were observed: SCRs exhibited increasing intensities of hypsochromic shifts (blue-shift) as a function of increasing solution pH, whereas SCKs showed the same effect as a function of decreasing pH, over a solution pH range of 4.6 to 8.6. Both systems present themselves as promising dual-emitting ratiometric pH sensing materials.

### 2. Experimental

2.1 Materials

The universal alkoxyamine initiator 2,2,5-trimethyl-3-(1′-phenylethoxy)-4-phenyl-3-azahexane was obtained from Sigma-Aldrich. The corresponding nitroxide 2,2,5-trimethyl-4-phenyl-3-azahexane-3-nitroxide was synthesized according to the literature method [30]. Prior to use, N-acryloxysuccinimide, purchased from Acros (99%), was recrystallized from dry ethyl acetate and stored under argon. The mono-methoxy terminated mono-hydroxy poly(ethylene glycol) (mPEG, MW=2,000 Da, PDI=1.06) was purchased from Intezyne Technologies and was used for the synthesis of macro chain transfer agent (macro-CTA) without further purification. The mPEG2k macro-CTA and PEO₄₅-b-PNAS₉₅-b-PS₆₀ were synthesized according to previous reports [29]. All other chemicals and reagents were obtained from Aldrich and used as received, unless described otherwise. tert-Butyl acrylate (tBA) and 4-acetoxystyrene (AS) were filtered through a plug of aluminum oxide to remove the inhibitor. All reactions were performed under N₂, unless noted otherwise.

2.2 Instrumental

¹H NMR and ¹³C NMR spectra were recorded at 500 MHz and 125 MHz, respectively, as solutions with the solvent proton or carbon signal as a standard. UV-Vis spectra were collected at ambient temperature in the region of 200-800 nm, using a Varian Cary 100 Bio UV-visible spectrophotometer. The fluorescence spectra were obtained at room temperature using a Varian Cary Eclipse fluorescence spectrophotometer. An excitation wavelength of the observed maximum absorption peak was used unless otherwise noted. Each fluorescence spectrum was normalized with respect to the absorbance at the excitation wavelength. The molar extinction coefficient (E) of chromophoric cross-linkers (ε_{A}=5163, ε_{B}=5772, ε_{C}=3463 M⁻¹·cm⁻¹ at 441 nm) was determined by a calibration curve in 5 mM PBS. The chromophoric cross-linker concentrations in the nano-objects were determined by UV-vis spectroscopy. IR spectra of neat films on NaCl plates were recorded using a Shimadzu Prestige21 IR spectrometer.

Gel permeation chromatography (GPC) was conducted on a Waters 1515 HPLC (Waters Chromatography, Inc.) equipped with a Waters 2414 differential refractometer, a PD2020 dual-angle (15° and 900) light scattering detector (Precision Detectors, Inc.), and a three-column series PL gel 5 μm Mixed C, 500 Å, and 10⁴ Å, 300×7.5 mm columns (Polymer Laboratories, Inc.). The system was equilibrated at 35° C. in anhydrous THF, which served as the polymer solvent and eluent with a flow rate of 1.0 mL/min. Polymer solutions were prepared at a known concentration (ca. 4-5 mg/mL) and an injection volume of 200 μL was used. Data collection and analysis were performed, respectively, with Precision Acquire software and Discovery 32 software (Precision Detectors, Inc.). Interdetector delay volume and the light scattering detector calibration constant were determined by calibration using a nearly monodispersed polystyrene standard (Pressure Chemical Co., M_{P}=90 kDa, M_{w}/Mₙ<1.04). The differential refractometer was calibrated with standard polystyrene reference material (SRM 706 NIST), of known specific refractive index increment dn/dc (0.184 mL/g). The dn/dc values of the analyzed polymers were then determined from the differential refractometer response.

The N,N-dimethylformamide (DMF) GPC was conducted on a Waters Chromatography Inc. (Milford, Mass.) system equipped with an isocratic pump model 1515, a differential refractometer model 2414, and a two-column set of Styragel HR 4 and HR 4E 5 μm DMF 7.8×300 mm columns. The system was equilibrated at 70° C. in pre-filtered DMF containing 0.05 M LiBr, which served as polymer solvent and eluent (flow rate set to 1.00 mL/min). Polymer solutions were prepared at a concentration of ca. 3 mg/mL and an injection volume of 200 μL was used. Data collection and analysis were performed with Empower Pro software (Waters Inc.). The system was calibrated with poly(ethylene glycol) standards (Polymer Laboratories) ranging from 615 to 442,800 Da.

Dynamic light scattering measurements were conducted with a Brookhaven Instruments, Co. (Holtsville, N.Y.) DLS system equipped with a model BI-200SM goniometer, BI-9000AT digital correlator, and a model EMI-9865 photomultiplier, and a model Innova 300 Ar ion laser operated at 514.5 nm (Coherent Inc., Santa Clara, Calif.). Measurements were made at 25±1° C. Prior to analysis, solutions were filtered through a 0.45 μm Millex®-GV PVDF membrane filter (Millipore Corp., Medford, Mass.) to remove dust particles. Scattered light was collected at a fixed angle of 90°. The digital correlator was operated with 522 ratio spaced channels, and initial delay of 5 μs, a final delay of 50 ms, and a duration of 8 minutes. A photomultiplier aperture of 400 μm was used, and the incident laser intensity was adjusted to obtain a photon counting of between 200 and 300 kcps. The calculations of the particle size distributions and distribution averages were performed with the ISDA software package (Brookhaven Instruments Company), which employed single-exponential fitting, Cumulants analysis, and CONTIN particle size distribution analysis routines. All determinations were average values from ten measurements. Alternatively, DLS measurements were also conducted using Delsa Nano C from Beckman Coulter, Inc. (Fullerton, Calif.) equipped with a laser diode operating at 658 nm. Size measurements were made in nanopure water. Scattered light was detected at 15° angle and analyzed using a log correlator over 70 accumulations for a 0.5 mL of sample in a glass size cell (0.9 mL capacity). The photomultiplier aperture and the attenuator were automatically adjusted to obtain a photon counting rate of ca. 10 kcps. The calculation of the particle size distribution and distribution averages was performed using CONTIN particle size distribution analysis routines using Delsa Nano 2.31 software. The peak average of histograms from intensity, volume and number distributions out of 70 accumulations were reported as the average diameter of the particles.

Transmission electron microscopy (TEM) bright-field imaging was conducted on a Hitachi H-7500 microscope, operating at 80 kV. The samples were prepared as follows: 4 μL of the dilute solution (with a polymer concentration of ca. 0.2-0.5 mg/mL) was deposited onto a carbon-coated copper grid, which was pre-treated with absolute ethanol to increase the surface hydrophilicity. After 5 min, the excess of the solution was quickly wicked away by a piece of filter paper. The samples were then negatively stained with 4 μL of 1 wt % phosphotungstic acid (PTA) aqueous solution. After 1 min, the excess PTA solution was quickly wicked away by a piece of filter paper and the samples were left to dry under ambient conditions overnight.

2.3 Synthesis of Chromophoric Cross-Linkers

### 2.3.1. Synthesis of chromophoric cross-linker A (3,6-diamino-N²,N⁵-bis(2-aminoethyl)pyrazine-2,5-dicarboxamide)

A mixture of sodium 3,6-diaminopyrazine-2,5-dicarboxylate (500 mg, 2.07 mmol), tert-butyl 2-aminoethylcarbamate (673 mg, 4.20 mmol), HOBt (836 mg, 5.46 mmol) and EDCl (1.05 g, 5.48 mmol) in DMF (25 mL) was allowed to stir for 16 h and was then concentrated. The residue was partitioned with 1 N NaHSO₄ (200 mL) and EtOAc (200 mL). The organic layer was separated and washed with water (200 mL×3), saturated NaHCO₃ (200 mL×3), and brine. It was then dried with MgSO₄, filtered, and concentrated to afford the bisamide as an orange foam. 770 mg, 76% yield. ¹H NMR (300 MHz, DMSO-d₆, 8): major conformer, 8.44 (t, J=5.7 Hz, 2H), 6.90 (t, J=5.7 Hz, 2H), 6.48 (br, 4H), 2.93-3.16 (m, 8H), 1.37 (s, 9H), 1.36 (s, 9H). ¹³C NMR (75 MHz, DMSO-d₆, 8): 165.1, 155.5, 155.4, 146.0, 126.2, 77.7, 77.5, 45.2, 44.5, 28.2. LC-MS (15-95% gradient acetonitrile in 0.1% TFA over 10 min), single peak retention time=7.18 min on 30 mm column, (M+H)⁺=483 amu. TFA (25 mL) was added to the product (770 mg, 1.60 mmol) in methylene chloride (100 mL), and the reaction was stirred at room temperature for 2 h. The mixture was concentrated and the residue was dissolved into methanol (15 mL). Diethyl ether (200 mL) was added and the orange solid precipitate was isolated by filtration and dried in high vacuum to afford an orange powder. 627 mg, 77% yield. IR (NaCl): 2951, 2928, 1811, 1759, 1233, 1090, 1067, 864, 831, 775 cm⁻¹. ¹H NMR (300 MHz, DMSO-d₆, 6): 8.70 (t, J=6 Hz, 2H), 7.86 (br, 6H), 6.50 (br, 4H), 3.46-3.58 (m, 4H), 3.26-3.40 (m, 4H); ¹³C NMR (75 MHz, DMSO-d₆, 6): 166.4, 146.8, 127.0, 39.4, 37.4. LC-MS (15-95% gradient acetonitrile in 0.1% TFA over 10 min), single peak retention time=2.60 min on 30 mm column, (M+H)⁺=283 amu. UV-vis (100 mM in PBS): λ_{abs}=435 nm. Fluorescence (100 nM): λₑₓ=449 nm, λₑₘ=562 nm. The product was converted to the HCl salt by co-evaporation (3×100 mL) with 1N aqueous HCl.

### 2.3.2. Synthesis of chromophoric cross-linker B (3,6-diamino-N²,N⁵-bis(3-(2-(2-(3-aminopropoxy)ethoxy)ethoxy)propyl) pyrazine-2,5-dicarboxamide dihydrochloride)

Step 1.

Synthesis of tert-butyl 1,1′-(3,6-diaminopyrazine-2,5-diyl)bis(1-oxo-6,9,12-trioxa-2-azapentadecane-15,1-diyl)dicarbamate: A mixture of 3,6-diaminopyrazine-2,5-dicarboxylic acid (0.31 g, 1.56 mmol), tert-butyl 3-(2-(2-(3-aminopropoxy)ethoxy)ethoxy) propyl carbamate (1.00 g, 3.12 mmol), EDC.HCl (0.72 g, 3.74 mmol) and HOBt (0.50, 3.74 mmol) was stirred in DMF (35 mL) for 16 hr at room temperature. The residue was partitioned with EtOAc (100 mL) and saturated sodium bicarbonate (100 mL). The layers were separated and the EtOAc solution was washed with 5% aq. Citric acid (100 mL) and brine (100 mL). The EtOAc layer was dried (MgSO₄), filtered and concentrated to afford 1.2 g (48% yield) of the bisamide as an orange oil. The crude bis-amide was taken on to the next step with no further purification: HRMS calcd for C₃₆H₆₆N₈O₁₂Na, [M+Na]⁺=825.4692 g/mol; Observed, 825.4674 g/mol.

Step 2.

To the crude product mixture from step 1 (˜1.20 g, 1.50 mmol) was added 4N HCl-Dioxane (10 mL) and the resulting mixture was stirred for 1 hr at room temperature. Concentration, in vacuo and pumping at high vacuum afforded 910 mg (90% yield) product as a viscous red oil: IR (NaCl): 2957, 2940, 1809, 1751, 1231, 1098, 1070, 866, 833, 775 cm⁻¹. LCMS (5-95% gradient acetonitrile in 0.1% TFA over 10 min), single peak retention time=5.70 min on 30 mm column, HRMS calcd. for C₇₂H₁₃₆N₁₀o₃₂ [M+H]⁺=603.3824 g/mol. Observed M+H=603.3823 g/mol. UV/vis (100 μM in PBS) λ_{abs}=435 nm. Fluorescence (100 nM) λₑₓ=449 nm, λₑₘ=562 nm.

### 2.3.3. Synthesis of chromophoric cross-linker C (3,6-Diamino-N2,N5-bis [N-(2-aminoethyl)-Arginine amide]-pyrazine-2,5-dicarboxamide tetra TFA salt)

Step 1.

Synthesis of 3,6-Diamino-N2,N5-bis (N-pbf-Arginine methyl ester)-pyrazine-2,5-dicarboxamide: A mixture of 3,6-diaminopyrazine-2,5-dicarboxylic acid (0.90 g, 4.54 mmol), H-Arg(pbf)-OMe.HCl (4.77 g, 9.99 mmol), EDC (1.53 g, 9.99 mmol), HOBt (1.34 g, 9.99 mmol) and TEA (726 μL, 9.99 mmol) was stirred in DMF (35 mL) for 6 hr at room temperature. The reaction was concentrated in vacuo and partitioned between 125 ml EA and 100 ml saturated sodium bicarbonate. The organics were washed with 10% NaHSO₄, brine, dried and concentrated to ½ volume and filtered through a plug of silica gel and the filtrate was concentrated to afford 2.4 g of a red oil-glass. The crude bis-amide was taken on to the next step with no further purification.

Step 2.

Synthesis of 3,6-Diamino-N2,N5-bis (N-pbf-arginine)-pyrazine-2,5-dicarboxamide di-lithium salt: A solution of the product from Step 1 (2.40 g, 2.30 mmol) in THF (35 mL) was treated with a solution of lithium hydroxide (276 mg 11.5 mmol) in water (5.0 mL). After stirring for 1 hr at room temperature, HPLC analysis indicated reaction was complete. The reaction was quenched by the addition of dry ice and concentrated. This material was used in the next step without further purification.

Step 3. Synthesis of 3,6-Diamino-N²,N⁵-bis [N-(2-boc-aminoethyl)-arginine amide]-pyrazine-2,5-di-carboxamide: A mixture of the product from Step 2 (1.00 g, 0.97 mmol), tert-butyl 2-aminoethyl-carbamate (350 mg, 2.19 mmol), EDC.HCl (420 mg, 2.19 mmol) HOBt (290 mg, 2.15 mmol) and TEA (0.5 mL) in DMF (50 mL) was stirred at room temperature for 16 h. The reaction was concentrated and the residue was partitioned between 100 ml Ethyl Acetate and 100 ml saturated sodium bicarbonate. The organics were washed with 10 aqueous KHSO₄, brine, and concentrated in vacuo and vacuum dried to afford 905 g (71% yield) of product as a red semi-solid: MS (ESI) [M+H]⁺=1300 g/mol; [M+Na]⁺=1323 g/mol. This material was used in the next step without further purification.

Step 4.

To the product from Step 3 (900 mg, 0.69 mmol) was added TFA (9.25 mL), water (25 μL), and triisopropyl silane (25, μL). The resulting mixture was stirred at room temperature for 72 h (convenience—over weekend). The reaction mixture was concentrated in vacuo. The residue was purified by preparative HPLC (C18, 30×150 mm column, 5% ACN in H₂O to 95% over 12 min, 0.1% TFA) to afford 178 mg (26% yield) of the product as a red foam: IR (NaCl): 2957, 2934, 1811, 1749, 1233, 1094, 1067, 864, 831, 777 cm⁻¹. HRMS calcd for C₂₂H₄₃N₁₆O₄, Theoretical M+H=595.3648 g/mol; observed M+H=595.3654 g/mol.

2.4. Preparation of Shell-Cross-Linked Nano-Objects

2.4.1. Preparation of Rod-Shaped Micelles (1):

To a 100-mL RB flask equipped with a magnetic stir bar was added PAA₁₄₀-b-PpHS₅₀ (93 mg, 5.7 μmol) and nanopure water (91 mL) to achieve a polymer concentration of ca. 1.0 mg/mL. The mixture was allowed to stir at rt for 2 h. An aliquot of the solution (25 mL) was added to a 100-mL RB flask and diluted with nanopure water (60 mL) to achieve a final polymer concentration of ca. 0.3 mg/mL. The solution was allowed to stir at rt overnight.

2.4.2. Preparation of Spherical Micelles (2):

To a 100-mL RB flask equipped with a magnetic stir bar was added 50 mL of PAA₁₄₀-b-PpHS₅₀ (15 mg, 0.9 μmol). The pH value was adjusted to ca. 12 by adding a pellet of NaOH to afford a clear solution. The micellization was initiated by decreasing the solution pH value to ca. 7 by adding dropwise HCl. The micelle solution was allowed to stir at rt for 12 h. Hₐᵥ=5±2 nm (AFM); Dₐᵥ=16±3 nm (TEM); Dₕ as measured by DLS was pH dependent-see reference 31 for the data.

2.4.3. Preparation of SCR-As:

To a 50-mL round bottom flask equipped with a magnetic stir bar was added a solution of 1 in nanopure H₂O (28 mL or 21 mL, 72 μmol or 44 μmol of carboxylic acid residues). To this solution, was added a solution of A (0.20 mg, 0.57 μmol (0.79 mol % relative to the acrylic acid residues) for 2% cross-linking extent; 1.0 mg, 2.8 μmol (3.9 mol % relative to the acrylic acid residues) for 6% cross-linking extent; or 2.0 mg, 5.6 μmol (7.9 mol % relative to the acrylic acid residues) for 10% cross-linking extent). The reaction mixture was allowed to stir at rt for 2 h. To this solution was added, dropwise via a syringe pump over 1 h, a solution of 1-[3′-(dimethylamino)propyl]-3-ethylcarbodiimide methiodide (EDCl): 0.40 mg, 1.4 μmol (stoichiometric) for 2% cross-linking extent; 2.1 mg, 7.2 μmol (stoichiometric) for 6% cross-linking extent; 4.3 mg, 14 μmol (stoichiometric) for 10% cross-linking extent; 0.52 mg, 1.8 μmol (2 molar excess) for 2% cross-linking extent; 2.6 mg, 8.8 μmol (2 molar excess) for 5% cross-linking extent; or 5.2 mg, 18 μmol (2 molar excess) for 9% cross-linking extent and the reaction mixture was further stirred at rt for 16 h. Finally, the reaction mixture was transferred to pre-soaked dialysis tubing (MWCO ca. 3,500 Da) and dialyzed against 5 mM PBS (5 mM NaCl, pH 7.4) for a day then nanopure water for another day to remove the non-attached cross-linker, excess small molecule starting materials and by-products, and afford aqueous solutions of shell-cross-linked cylinder, SCR-A2%, SCR-A6%, SCR-A10%, SCR-A2%, SCR-A5% or SCR-A9% (final polymer concentration: 0.30 mg/mL, 0.30 mg/mL or 0.28 mg/mL for stoichiometric addition of EDCl and 0.22 mg/mL, 0.23 mg/mL or 0.23 mg/mL for 2 molar excess of EDCl, respectively—where in each case, the % cross-linking was determined by UV-vis spectroscopic measurement of the amount of cross-linker remaining after purification). SCR solutions for UV-vis, and fluorescence studies were further partitioned into four vials each containing 5 mM PBS (with 5 mM NaCl) at pH values of 4.6, 6.4, 7.4 and 8.4. SCRs measured 23±2 nm in width and 100 nm to a micron length, by TEM.

2.4.4. Preparation of SCR-Bs:

To a 50-mL round bottom flask equipped with a magnetic stir bar was added a solution of 1 in nanopure H₂O (22 mL or 21 mL, 57 μmol or 44 μmol of carboxylic acid residues). To this solution, was added a solution of B (0.30 mg, 0.45 μmol (0.79 mol % relative to the acrylic acid residues) for 2% cross-linking extent; 1.5 mg, 2.3 μmol (3.9 mol % relative to the acrylic acid residues) for 7% cross-linking extent; or 3.0 mg, 4.4 μmol (7.9 mol % relative to the acrylic acid residues) for 12% cross-linking extent). The reaction mixture was allowed to stir at rt for 2 h. To this solution was added, dropwise via a syringe pump over 1 h, a solution of 1-[3′-(dimethylamino)propyl]-3-ethylcarbodiimide methiodide (EDCl): 0.34 mg, 1.1 μmol (stoichiometric) for 2% cross-linking extent; 1.7 mg, 5.7 μmol (stoichiometric) for 7% cross-linking extent; 3.4 mg, 11 mol (stoichiometric) for 12% cross-linking extent); 0.52 mg, 1.8 μmol (2 molar excess) for 2% cross-linking extent; 2.6 mg, 8.8 μmol (2 molar excess) for 7% cross-linking extent or 5.2 mg, 18 μmol (2 molar excess) for 10% cross-linking extent and the reaction mixture was further stirred at rt for 16 h. Finally, the reaction mixture was transferred to pre-soaked dialysis tubing (MWCO ca. 3,500 Da) and dialyzed against 5 mM PBS (5 mM NaCl, pH 7.4) for a day then nanopure water for another day to remove the non-attached cross-linker, excess small molecule starting materials and by-products, and afford aqueous solutions of shell-cross-linked cylinder, SCR-B₂%, SCR-B₇%, SCR-B₁₂%, SCR-B₂%, SCR-B₇% or SCR-B₁₀% (final polymer concentration: 0.30 mg/mL, 0.29 mg/mL or 0.28 mg/mL for stoichiometric addition of EDCl and 0.23 mg/mL, 0.23 mg/mL or 0.23 mg/mL for 2 molar excess amount of EDCl, respectively—where in each case, the % cross-linking was determined by UV-vis spectroscopic measurement of the amount of cross-linker remaining after purification). SCR solutions for UV-vis, and fluorescence studies were further partitioned into four vials each containing 5 mM PBS (with 5 mM NaCl) at pH values of 4.6, 6.4, 7.4 and 8.4. SCRs measured 23±2 nm in width and 100 nm to a micron length, by TEM.

2.4.5. Preparation of SCR-Cs:

To a 50-mL round bottom flask equipped with a magnetic stir bar was added a solution of 1 in nanopure H₂O (28 mL or 21 mL, 72 mol or 44 μmol of carboxylic acid residues). To this solution, was added a solution of C (0.34 mg, 0.56 μmol (0.79 mol % relative to the acrylic acid residues) for 2% cross-linking extent; 1.7 mg, 2.8 μmol (3.9 mol % relative to the acrylic acid residues) for 7% cross-linking extent; or 3.4 mg, 5.6 μmol (7.9 mol % relative to the acrylic acid residues) for 14% cross-linking extent). The reaction mixture was allowed to stir at rt for 2 h. To this solution was added, dropwise via a syringe pump over 1 h, a solution of 1-[3′-(dimethylamino)propyl]-3-ethylcarbodiimide methiodide (EDCl): 0.42 mg, 1.4 μmol (stoichiometric) for 2% cross-linking extent; 2.1 mg, 7.2 μmol (stoichiometric) for 7% cross-linking extent; 4.3 mg, 14 μmol (stoichiometric) for 14% cross-linking extent; 0.52 mg, 1.8 μmol (2 molar excess) for 2% cross-linking extent; 2.6 mg, 8.8 μmol (2 molar excess) for 6% cross-linking extent or 5.2 mg, 17 μmol (2 molar excess) for 3% cross-linking extent) and the reaction mixture was further stirred at rt for 16 h. Finally, the reaction mixture was transferred to pre-soaked dialysis tubing (MWCO ca. 3,500 Da) and dialyzed against 5 mM PBS (5 mM NaCl, pH 7.4) for a day then nanopure water for another day to remove the non-attached cross-linker, excess small molecule starting materials and by-products, and afford aqueous solutions of shell-cross-linked cylinder, SCR-C2%, SCR-C7%, SCR-C14%, SCR-C2%, SCR-C6% or SCR-C3% (final polymer concentration: 0.29 mg/mL, 0.28 mg/mL or 0.27 mg/mL for stoichiometric addition of EDCl and 0.23 mg/mL, 0.23 mg/mL or 0.22 mg/mL for 2 molar excess amount of EDCl, respectively—where in each case, the % cross-linking was determined by UV-vis spectroscopic measurement of the amount of cross-linker remaining after purification). SCR solutions for UV-vis, and fluorescence studies were further partitioned into four vials each containing 5 mM PBS (with 5 mM NaCl) at pH values of 4.6, 6.4, 7.4 and 8.4. SCRs measured 23±2 nm in width and 100 nm to a micron length, by TEM.

2.4.6. Preparation of SCK-As:

To a 50-mL round bottom flask equipped with a magnetic stir bar was added a solution of 2 in nanopure H₂O (25 mL or 28 mL, 68 μmol or 72 tpmol of carboxylic acid residues). To this solution, was added a solution of A (0.19 mg, 0.54 μmol (0.79 mol % relative to the acrylic acid residues) for 2% cross-linking extent; 1.0 mg, 2.7 μmol (3.9 mol % relative to the acrylic acid residues) for 7% cross-linking extent; or 1.9 mg, 5.4 μmol (7.9 mol % relative to the acrylic acid residues) for 13% cross-linking extent). The reaction mixture was allowed to stir at rt for 2 h. To this solution was added, dropwise via a syringe pump over 1 h, a solution of 1-[3′-(dimethylamino)propyl]-3-ethylcarbodiimide methiodide (EDCl): 0.41 mg, 1.4 μmol (stoichiometric) for 2% cross-linking extent; 2.0 mg, 6.8 μmol (stoichiometric) for 7% cross-linking extent; 4.1 mg, 14 mol (stoichiometric) for 13% cross-linking extent; 15 mg, 50 μmol (36 molar excess) for 2% cross-linking extent; 15 mg, 50 μmol (36 molar excess) for 8% cross-linking extent; 15 mg, 50 μmol (35 molar excess) for 14% cross-linking extent; 30 mg, 100 μmol (75 molar excess) for 2% cross-linking extent; 30 mg, 100 μmol (75 molar excess) for 7% cross-linking extent; or 30 mg, 100 μmol (75 molar excess) for 13% cross-linking extent and the reaction mixture was further stirred at rt for 16 h. Finally, the reaction mixture was transferred to pre-soaked dialysis tubing (MWCO ca. 3,500 Da) and dialyzed against 5 mM PBS (5 mM NaCl, pH 7.4) for a day then nanopure water for another day to remove the non-attached cross-linker, excess small molecule starting materials and by-products, and afford aqueous solutions of shell-cross-linked spherical nanoparticles, SCK-A2%, SCK-A7%, SCK-A13%, SCK-A2%, SCK-A8%, SCK-A14%, SCK-A2%, SCK-A7% or SCK-A13% (final polymer concentration: 0.25 mg/mL, 0.24 mg/mL or 0.24 mg/mL for stoichiometric addition of EDCl and 0.26 mg/mL, 0.26 mg/mL or 0.26 mg/mL for 35 molar excess amount of EDCl and 0.27 mg/mL, 0.27 mg/mL or 0.26 mg/mL for 75 molar excess amount EDCl, respectively—where in each case, the % cross-linking was determined by UV-vis spectroscopic measurement of the amount of cross-linker remaining after purification). SCK solutions for UV-vis, and fluorescence studies were further partitioned into four vials each containing 5 mM PBS (with 5 mM NaCl) at pH values of 4.6, 6.4, 7.4 and 8.4. SCKs measured 27±3 nm by number-average distribution dynamic light scattering measurements and 23±2 nm in diameter, by TEM.

2.4.7. Preparation of SCK-As with Two Sequential Addition of Stoichiometric Amount of EDCl (SCK-A′):

To a 50-mL round bottom flask equipped with a magnetic stir bar was added a solution of SCK-A2%, SCK-A7% or SCK-A13% in nanopure H₂O (28 mL, 64 μmol of combined carboxylic acid and amide residues). To this solution was added, dropwise via a syringe pump over 1 h, a solution of 1-[3′-(dimethylamino)propyl]-3-ethylcarbodiimide methiodide (EDCl): 0.38 mg, 1.3 μmol (stoichiometric) for 2% cross-linking extent; 1.9 mg, 6.4 μmol (stoichiometric) for 7% cross-linking extent or 3.8 mg, 13 μmol (stoichiometric) for 13% cross-linking extent. Finally, the reaction mixture was transferred to pre-soaked dialysis tubing (MWCO ca. 3,500 Da) and dialyzed against 5 mM PBS (5 mM NaCl, pH 7.4) for a day then nanopure water for another day to remove excess small molecule starting materials and by-products, and afford aqueous solutions of shell-cross-linked spherical nanoparticles, SCK-A′2%, SCK-A′7% or SCK-A′13% (final polymer concentration: 0.26 mg/mL, 0.26 mg/mL or 0.25 mg/mL, respectively—where in each case, the % cross-linking was determined by UV-vis spectroscopic measurement of the amount of cross-linker remaining after purification). SCR solutions for UV-vis, and fluorescence studies were further partitioned into four vials each containing 5 mM PBS (with 5 mM NaCl) at pH values of 4.6, 6.4, 7.4 and 8.4. SCKs measured 27±3 nm by number-average distribution dynamic light scattering measurements and 23±2 nm in diameter, by TEM.

2.4.8. Synthesis of PEO₄₅-b-PNAS₅₀:

To a 25-mL Schlenk flask equipped with a magnetic stir bar dried with flame under N₂ atmosphere, was added the mPEG2k macro-CTA (0.19 g, 79 μmol) and 1,4-dioxane (5 mL). The reaction mixture was stirred 0.5 h at rt to obtain a homogeneous solution. To this solution was added NAS (0.8 g, 4.7 mmol) and AIBN (0.76 mg, 4.7 μmol). The reaction flask was sealed and allowed to stir 10 min at rt. The reaction mixture was degassed through several cycles of freeze-pump-thaw. After the last cycle, the reaction mixture was allowed to stir for 10 min at rt before being immersed into a pre-heated oil bath at 55° C. to start the polymerization. After 210 min, the monomer conversion reached ca. 75% by analyzing aliquots collected through ¹H NMR spectroscopy. The polymerization was quenched by cooling the reaction flask with liquid N₂. The polymer was purified by precipitation into 500 mL of cold diethyl ether at 0° C. three times. The precipitants were collected, washed with 100 mL of cold ether, and dried under vacuum overnight to afford the PEO₄₅-b-PNAS₅₀ block copolymer precursor as a yellow solid (0.68 g, 85% yield based upon monomer conversion). ¹H NMR (500 MHz, DMSO-d₆, ppm): δ 0.81 (t, J=6 Hz, 3H, dodecyl CH₃), 1.09 (br, 5H, CH₃ and dodecyl CH₂), 1.20 (br, 19H, CH₃ and dodecyl CH₂s), 1.30 (br, 2H, dodecyl CH₂), 1.60 (t, J=6 Hz, 2H, dodecyl CH₂), 2.01 (br, PNAS backbone protons), 2.75 (NAS CH₂CH₂s), 3.09 (br, PNAS backbone protons), 3.20 (s, mPEG terminal OCH₃), 3.47 (m, OCH₂CH₂OO from the PEG backbone), 4.07 (br, 2H from the PEO backbone terminus connected to the ester linkage); ¹³C NMR (125 MHz, DMSO-d₆, ppm): δ 25.2, 41.2, 69.8, 172.8. Mₙ^{NMR}=10,800 Da, PDI=1.2 (DMF GPC).

2.4.9. Synthesis of PEO₄₅-b-PNAS₅₀-b-PS₃₀:

To a 10-mL Schlenk flask equipped with a magnetic stir bar dried with flame under N₂ atmosphere, was added the PEO₄₅-b-PNAS₅₀ macro-CTA (0.5 g, 46 μmol), 1,4-dioxane (2.0 mL), and DMF (2.0 mL). The reaction mixture was allowed to stir for 0.5 h at rt to obtain a homogeneous solution. To this solution was added styrene (0.78 g, 7.5 mmol) and AIBN (0.41 mg, 2.5 μmol). The reaction flask was sealed and allowed to stir for 10 min at rt. The reaction mixture was degassed through several cycles of freeze-pump-thaw. After the last cycle, the reaction mixture was allowed to stir for 10 min at rt before being immersed into a pre-heated oil bath at 58° C. to start the polymerization. After 12.5 h, the monomer conversion reached ca. 18% by analyzing aliquots collected through ¹H NMR spectroscopy. The polymerization was quenched by cooling the reaction flask with liquid N₂. The polymer was purified by precipitation into 500 mL of cold diethyl ether at 0° C. three times. The precipitants were collected and dried under vacuum overnight to afford the block copolymer precursor as a yellow solid (0.55 g, 85% yield based upon monomer conversion). ¹H NMR (500 MHz, DMSO-d₆, ppm): δ 0.81 (br, dodecyl CH₃), 1.10-2.40 (br, dodecyl Hs, PNAS, and PS backbone protons), 2.75 (NAS CH₂CH₂s), 3.15 (br, PNAS backbone protons), 3.28 (s, mPEG terminal OCH₃), 3.60 (m, OCH₂CH₂O from the PEG backbone), 6.20-7.30 (br, Ar Hs); ¹³C NMR (125 MHz, DMSO-d₆, ppm): δ 25.2, 41.6, 69.8, 125.7, 128.0, 145.2, 172.8. Mₙ^{NMR}=13,900 Da, PDI=1.2 (DMF GPC).

2.4.10. General Procedure for Self-Assembly of PEO-b-PNAS-b-PS Block Copolymers:

To a solution of PEO-b-PNAS-b-PS block copolymer in DMF (ca. 1.0 mg/mL), was added dropwise an equal volume of nanopure H₂O within 2 h via a syringe pump at a rate of 15.0 mL/h. The mixture was further allowed to stir for 1 h at rt before used for cross-linking/functionalization reactions.

2.4.11. General procedure for cross-linking/functionalization of PEO-b-PNAS-b-PS micelles: To a solution of PEO-b-PNAS-b-PS micelles in DMF/H₂O (v:v=1:1) at rt, was added dropwise over 10 min, a solution of cross-linker A or B (0.1 eq., relative to the amounts of NAS residues, for nominal 20% of cross-linking) in nanopure water. The reaction mixture was allowed to stir for 48 h at rt in the absence of light. The reaction mixture was then divided into five portions (ca. 13 mL each) and transferred into pre-soaked dialysis tubing (MWCO 3,500 Da) and dialyzed against 5.0 mM buffer solutions (with 5.0 mM NaCl) at pH 5.8, 6.5, 7.2, 7.9, and 8.6, respectively, for 7 days to remove DMF, unreacted cross-linkers, and the small molecule by-products to afford an aqueous solution of sc-SCK-A and B (from PEO₄₅-b-PNAS₅₀-b-PS₃₀ block copolymer precursors) and Ic-SCK-A and B (from PEO₄₅-b-PNAS₉₅-b-PS₆₀ block copolymer precursors), respectively.

### 3. Results and Discussion

3.1. Photophysical Properties of SCRs

Block copolymers having two different compositions and three different block lengths were utilized to give rise to SCRs and SCKs with unique morphological and chemical properties. The pH-responsive diblock copolymer, PAA₁₄₀-b-PpHS₅₀, was used to create SCR precursors. Three chromophoric cross-linkers were then utilized in varying amounts to prepare SCR-A, SCR-B or SCR-C. Similarly, the spherical structural analog was created from the same block copolymer and subsequently shell cross-linked with the chromophoric cross-linker A to yield a set of SCK-As having different cross-linking extents. SCKs self-assembled from PEO₄₅-b-PNAS₅₀-b-PS₃₀ and cross-linked with A or B gave rise to small core-SCK-A (sc-SCK-A) or sc-SCK-B. Likewise, PEO₄₅-b-PNAS₉₅-b-PS₆₀ afforded large core-SCKs (Ic-SCK-A or Ic-SCK-B). These sets of nano-objects allowed for observation of pH-responsive photophysical properties due to the changes in morphology, spherical particle size or regioselective reactions within the shell region.

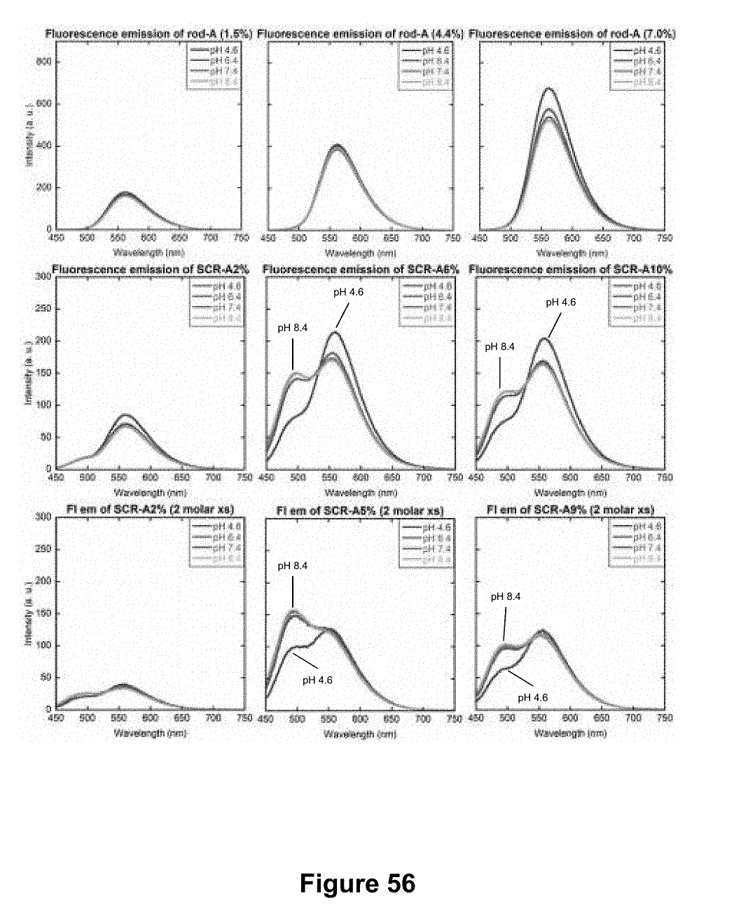

With the PAA₁₄₀-b-PpHS₅₀ block copolymer system, the shell cross-linking reactions involved condensation reactions between diamines of the chromophore and PAAs of the nanostructures in the presence of water-soluble carbodiimide, 1-[3′-(dimethylamino)propyl]-3-ethylcarbodiimide methiodide (EDCl). Typically, a 1:1 molar ratio or slight excess of carbodiimide to amines was added to the reaction mixture in order to form sufficient amounts of activated intermediates for intramicellar cross-linking reactions while avoiding intermicellar reactions. The chromophoric cross-linkers were based on a tetra-substituted pyrazine ring structure that shows a strong yellowish-green fluorescence in solution. While acylation of the terminal primary amines does not affect the emission wavelength, acylation of the anilino amine groups have been reported to cause blue-shifts in the fluorescence emission by ca. 50 nm, due to decrease of the donor property of the amino groups [32]. The chromophoric cross-linkers bear two terminal amine groups that are more reactive towards amidation of the PAAs than are the anilino amine groups on the pyrazine ring. We hypothesized that the degree to which the amine groups would undergo reaction with the PAAs could be controlled by the amount of EDCl added to the reaction mixture during the shell cross-linking reaction. In order to assess the extent to which aromatic amines participated in the cross-linking reaction, the amounts of cross-linking chromophore loaded (2, 6 or 9% cross-linking density) as well as the EDCl loaded (stoichiometric or 2 molar excess, relative to the aliphatic amines of the cross-linker) were varied, as shown in FIG. 56. Physical mixtures of A and rod-shaped block copolymer micelles resulted in a single fluorescence emission, as shown in FIG. 2, upper row; only when EDCl was added to the mixture did dual-emission arise from the resulting SCRs, as shown in FIG. 56, middle and lower rows.

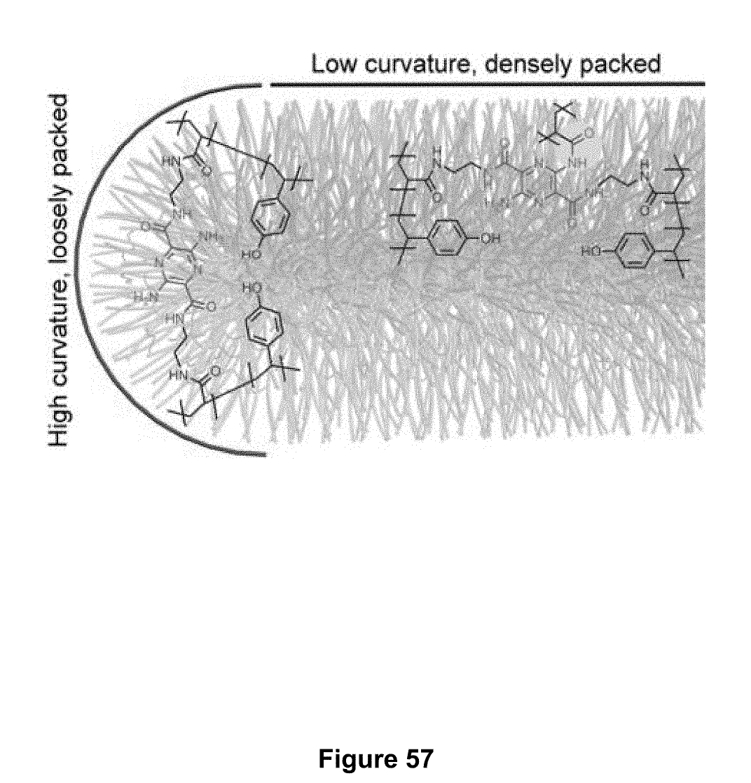

The addition of stoichiometric amounts of EDCl to solutions of rod-shaped micelles with A displayed a significant amount of blue-shift. Such unique behavior (in comparison to their spherical structural analogs under identical reaction conditions, vide infra) is attributed to the linear section of the rods, which consists of densely packed polymer chains, as shown in FIG. 57. In contrast, the spherical assemblies and the rod end caps have higher curvature, which reduces the density of chain packing. From a previous literature report [32], acylation of both anilino amines (corresponding to ca. 100 nm blue-shift) of A is not very likely under such mild reaction conditions. Therefore, mono-anilino acylation (corresponding to ca. 53 nm blue-shift) is proposed to occur throughout the studies presented here. Addition of 2 molar excess amount of EDCl to the reaction mixture resulted in greater intensities of the blue-shifted fluorescence emission for all SCR samples, further confirming the hypothesis that the extent to which anilino amine groups participated in the shell cross-linking reaction determined the degree of pyrazine units that experienced the blue-shift, exhibited by the resulting nanostructures. This finding represents a unique ability for the rod-shaped block copolymer micelles to create a local environment that facilitates enhanced cross coupling reactions between the polymer chains and cross-linkers.

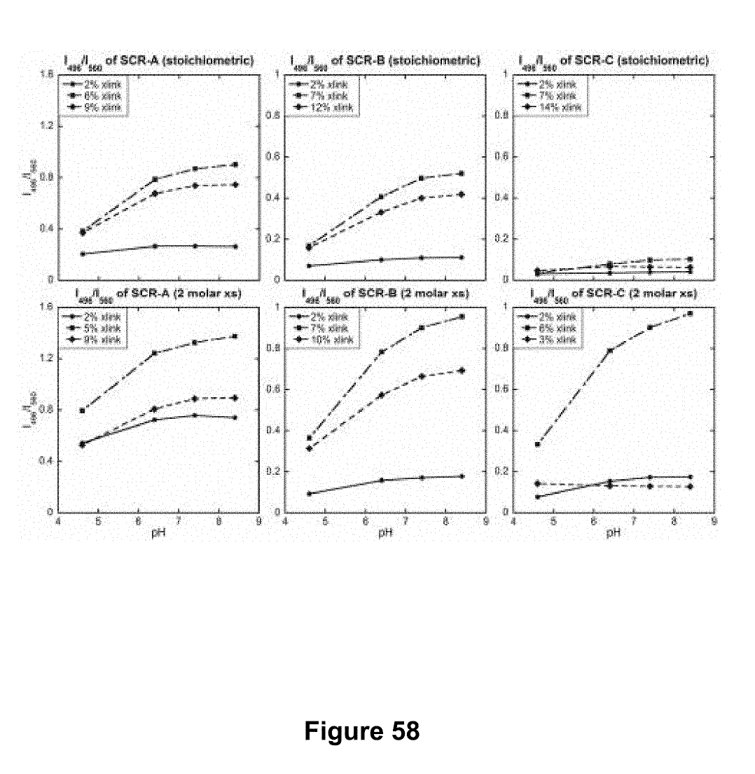

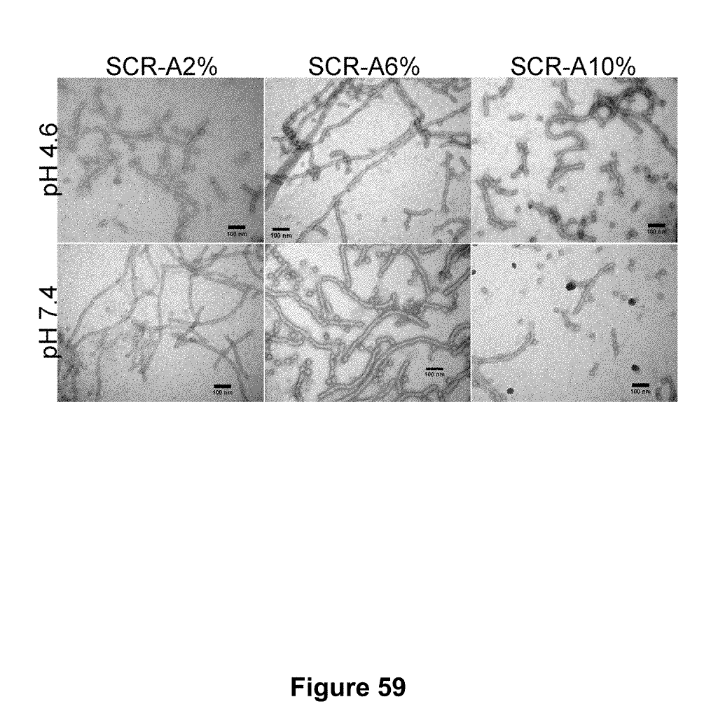

We then performed a similar set of studies with chromophoric cross-linkers A, B and C, to observe that each exhibited increasing blue-shifted fluorescence emission intensity with increasing amounts of EDCl activator, and further extended the studies to allow for observation of their pH-responsive ratiometric dual-emission when incorporated into the SCR nanostructures, as shown in FIG. 58. With addition of a stoichiometric amount of EDCl, the SCR-A series displayed a moderate pH-responsiveness (I₄₉₆/I₅₆₀ ranging from 0.2 to 0.9). SCR-A series with addition of 2 molar excess EDCl exhibited an increase in absolute blue-shift and pH-sensitivity (I₄₉₆/I₅₆₀ ranging from 0.5 to 1.4). Likewise, the SCK-B series showed I₄₉₆/I₅₆₀ that ranged from 0 to 0.5, at a stoichiometric amount of EDCl, and increased to 0.1 to 1, at 2 molar excess of EDCl. Most interestingly, the SCR-C series demonstrated an absence of appreciable pH-sensitivity at a stoichiometric amount of EDCl. However, SCR-C6% exhibited a remarkable pH-sensitivity upon addition of a 2 molar excess amount of EDCl (I₄₉₆/I₅₆₀ ranging from 0.3 to 1). In all cases, 5% to 7% cross-linked SCRs displayed the highest absolute blue-shift while 9% to 14% cross-linked SCRs suffered from self-quenching. The SCR series was also characterized by transmission electron microscopy (TEM), which revealed no apparent changes in morphology as a function of shell cross-linking density or solution pH value, as shown in FIG. 59.

3.2. Photophysical Properties of SCKs

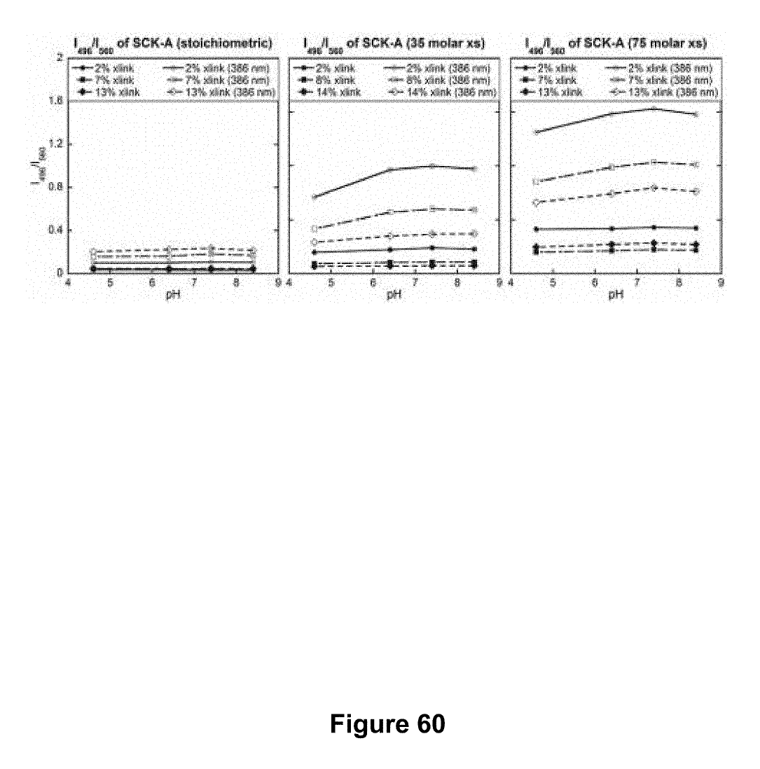

Similar experiments were conducted on SCKs, for which no blue-shift was previously observed, to develop a better understanding of the chemistry involved and the influence of block copolymer morphology, by attempting to impart a blue-shift in the fluorescence emission. In this set of experiments, 35 or 70 molar excesses of EDCl were added to the spherical micelle solutions, where 70 molar excess EDCl, relative to the amount of cross-linker aliphatic amines, was sufficient to activate essentially all carboxylic acid residues on the PAA chains. As the EDCl loading increased, the intensity of blue-shifted fluorescence emission became greater. The second highest cross-linker loading underwent the greatest relative amount of blue-shifted fluorescence, as shown in FIG. 60. In essence, we were able to achieve the photophysical consequences that were exclusive to shell-cross-linked rods within a spherical framework by manipulating the cross-linking reaction conditions with retention of morphology.

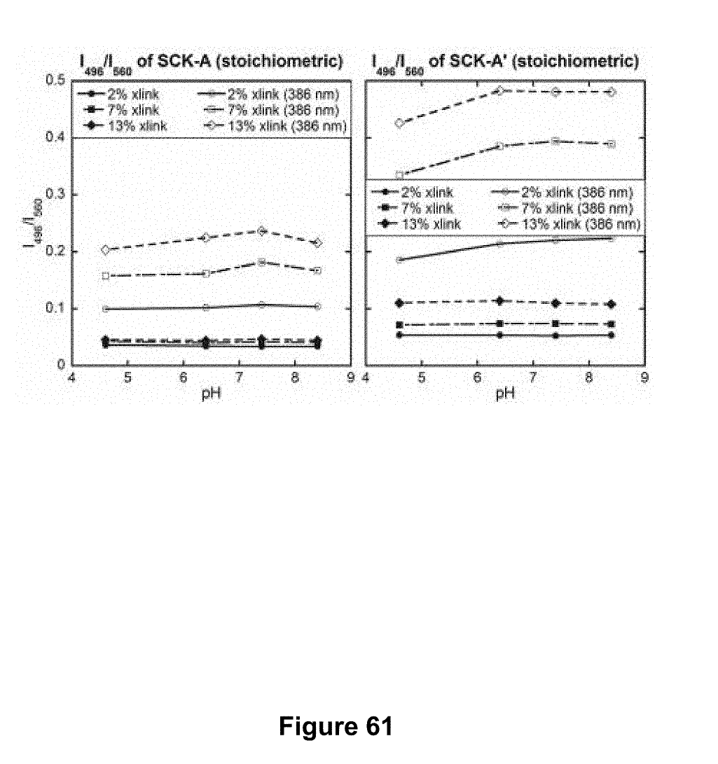

The data presented in this Example indicate that the less reactive aromatic amines of the cross-linking chromophore were available for reactions with the acids after a shell cross-linking reaction with the aliphatic amines. Therefore, we applied sequential cross-linking reactions twice, each with a fixed amount of EDCl, with the intention of first cross-linking the structure and then imparting the blue-shifted fluorescence emission. We prepared a batch of SCK-A series with a stoichiometric amount of EDCl and purified the sample by dialysis to remove free cross-linking chromophores and urea by-products. To the purified batch was added an additional stoichiometric amount of EDCl to allow for reactions between unreacted amines and residual PAA units. The fluorescence emission spectra, collected as a function of pH, showed increased intensities for the blue-shifted emission after the second cross-linking reaction, and are provided in FIG. 61.

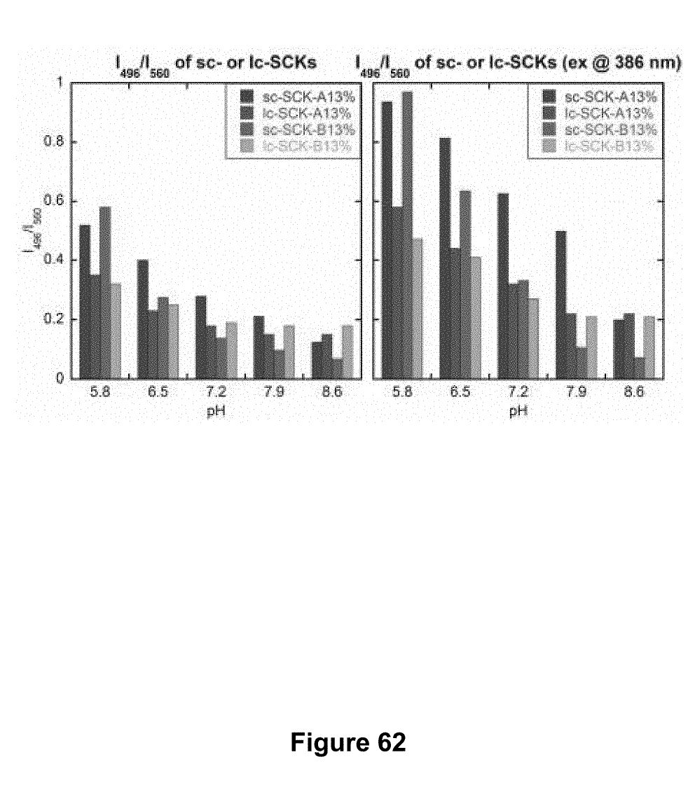

The pH-responsiveness of the ratiometric dual-emission of the SCKs, however, diminished in comparison to the SCRs. The degree to which the particles exhibited blue-shifted fluorescence emission was dependent upon the amount of EDCl added during the shell cross-linking reaction whether at once (as shown in FIG. 60, left vs. middle vs. right plots) or consecutively in two batches (as shown in FIG. 61, left vs. right plots), but unlike the rod-shaped isomers, these spherical analogs exhibited no pH-responsive behavior, giving no appreciable 496 nm fluorescence emission intensity enhancement. We then utilized a unique triblock terpolymer system (PEO₄₅-b-PNAS₅₀-b-PS₃₀ or PEO₄₅-b-PNAS₉₅-b-PS₆₀) that was recently developed to give rise to SCKs with activated esters pre-installed within the shell domain [33]. Addition of chromophoric cross-linkers to these SCK solutions resulted in direct formation of covalent bonds between the cross-linkers and the shell domain. The resulting photophysical properties revealed opposite pH-responsive dual-emission profiles, as shown in FIG. 62, than those observed for the EDCl activated rods (vide infra). In addition to having the pre-activated esters, these SCKs had a PEO corona and PS core, each differing compositionally from the EDCl-activated SCRs and SCKs.

3.3. Photophysical Properties of SCKs with Pre-Installation of Activated Esters

With the demonstration of morphological effect (i.e., cylinders vs. spheres) on the photophysical properties of the photonic nanostructures, we continued to investigate other critical parameters of the nanoscale materials, namely, the shell composition and size of nanoparticles. As described above, the packing mode of the chromophoric cross-linkers throughout the hydrophilic domains of rod-shaped nanostructures played an important role in the fluorescence emission outputs. It can be speculated that, for spherical nanoparticles with a core-shell morphology, changes in the volumetric ratio between the hydrophilic shell (in which the chromophoric cross-linkers were accommodated) and the hydrophobic core domains could induce significant effects on tuning of their photophysical properties.

Two kinds of SCK nanoparticles, i.e., SCKs with relatively smaller and larger core domains (sc-SCK and Ic-SCK, respectively), were prepared from aqueous self-assembly of PEO₄₅-b-PNAS₅₀-b-PS₃₀ and PEO₄₅-b-PNAS₉₅-b-PS₆₀ triblock terpolymer precursors, respectively, followed by cross-linking of the corresponding micelles with A or B at 13% of cross-linking extents, through amidation chemistry to afford sc-SCK-A13%, and sc-SCK-B13% or Ic-SCK-A13%, and Ic-SCK-B13%. Interestingly, although the repeat units of these two triblock terpolymers were different, we did not notice dramatic difference in the overall hydrodynamic diameter, as measured by DLS, for a majority of the constructed SCKs over the surveyed pH range (see Electronic Supplementary Information). The structure analysis revealed that the sc-SCK had a relatively thicker PNAS domain, in comparison with Ic-SCK, therefore, the chromophoric cross-linker was applied to a local environment that had more active esters during the amidation and the acylation of aromatic amines consequentially increased. Other factors, such as the steric packing modes of PNAS in the micelles with smaller core domains during cross-linking, and the resulting effects onto A and B ring moieties, after being incorporated into sc-SCKs, should also been taken into account. Ultimately, the residual NAS units underwent hydrolysis to afford spherical SCKs having PS cores and pyrazine-cross-linked PAA-based shells with PEO corona.

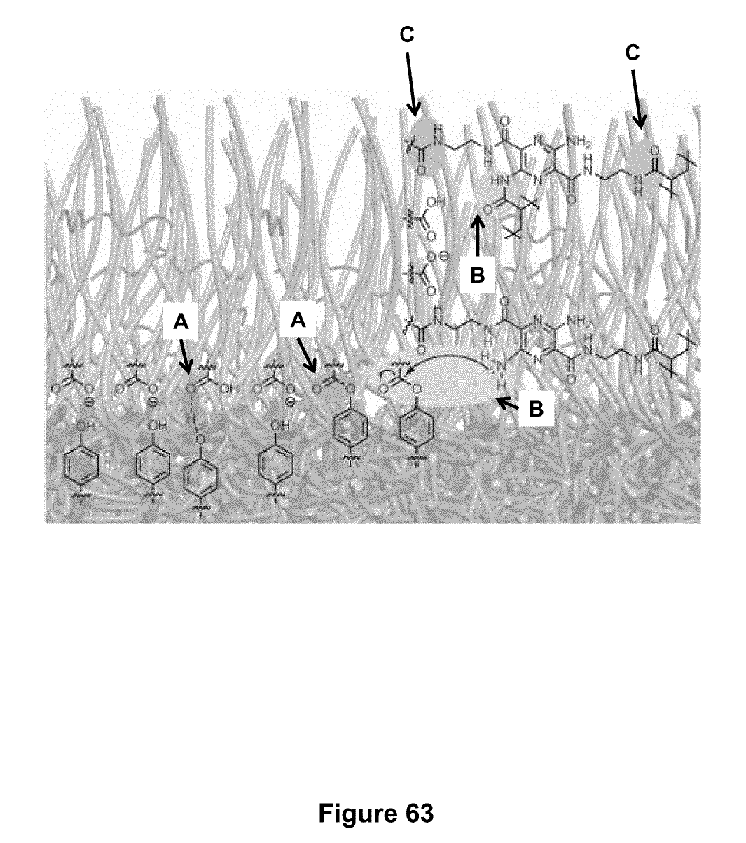

The increase in the fluorescence emission intensity at 496 nm relative to that at 560 nm with decreasing pH, opposite to the behavior observed for SCRs derived from EDCl-activated A or B cross-linking of PAA-b-PpHS micelles, is highly interesting. Because the sc-SCKs, Ic-SCKs and SCRs give opposite pH responses, whereas the SCKs give no response, at the moment, we can only speculate that combinations of morphological differences and compositional variations between the core and corona chemistries may each play roles. Due to the low curvature and dense packing of polymer chains in the rod structures, it is expected that there would be regions near the interface that provide opportunities for significant interaction between the PpHS and PAA domains. Micellization conditions that favor formation of the rods may allow for intimate exposure of zones of shell carboxylates with zones of core phenols, possibly aided by hydrogen bonding interactions. When these types of micelles are exposed to EDCl and the chromophoric cross-linkers, standard shell cross-linking can proceed, but ester formation may also occur. Due to the close interfacial exposure of segments of the two domains in the rods, some reaction takes place on the aryl amines. Thus, both the normal 560 nm and the blue-shifted 496 nm fluorescence are observed. Also, as mentioned, there may be a significant presence of phenyl esters generated from the EDCl treatment. Closely spaced cross-linked pyrazines could then intercept the phenyl esters to form the arylamino amide derivative, as shown in FIG. 63, which could be promoted at elevated pH values, giving enhanced formation of the 496 nm-emitting chromophore. Since the PEO-b-PNAS-b-PS preformed activated ester terpolymers have no phenolic groups, this type of morphology-driven aryl amide formation pathway is not available. Finally, the fact that the I₄₉₆/I₅₆₀ ratio drops with increasing pH with the Ic- and sc-SCKs (sc-SCK-A and sc-SCK-B) further supports the proposition that the phenyl esters are necessary for the increase in the blue-shifted fluorescence (FIG. 8). These SCKs have some 496 nm fluorescence due to non-selective cross-linking just as in the case of the rods. But, when the pH is increased in these systems there are no phenyl esters to further acylate the aminopyrazine groups. In this case, the existing pyrazine-arylamides that formed on EDCl treatment are probably hydrolyzed back to the desired difunctional pyrazine cross-linkers. Thus the 496 fluorescence is lost in favor of 560 nm and thus the I₄₉₆/I₅₆₀ ratio drops.

### CONCLUSIONS

In the process of installing a chromophoric cross-linker into block copolymer nanostructures, we have made several important fundamental findings that may lead to the creation of responsive diagnostic nanomaterials. Utilization of a single parent diblock copolymer of acrylic acid and para-hydroxystyrene to create two structural isomers has allowed for studies of photophysical properties that were strictly due to the changes manifested by two different morphologies (rods vs. spheres). Rods, having more densely packed regions with a low interfacial curvature, provided unique shell domains, rich with high local concentrations of carboxylic acids for the cross-linkers to reside, while also having the possibility of formation of phenyl esters at the core-shell interface, both of which contributed towards formation of arylamide that was responsible for blue-shifted fluorescence emission. The extent to which the blue-shifting occurred was fine-tuned by the addition of varying amounts of activating carbodiimide during the shell cross-linking reaction. These nanorods underwent ratiometric pH-sensing, exhibiting increases in blue-shifted fluorescence emission with increasing pH over the range from 4.6 to 8.6, whereas the analogous spherical structures gave almost no pH response. Spherical nanoparticles derived from a different parent triblock terpolymer, having a terminal poly(ethylene oxide) chain segment, activated esters along the poly(acrylic acid) segment and polystyrene block, demonstrated opposite pH-responsiveness in photophysical properties than did the rods, presumably due to combined effects from the lack of reactive groups at the core/shell interface and differences in morphology. It is interesting that the rod-shaped nanostructures exhibited blue-shifted fluorescence emission in high pH solutions while the spherical nanoparticles showed similar behavior in low pH solutions. By having dual fluorescence emission, direct measurement of pH may be possible without the need for an internal standard or potential complications from fluorescence quenching. Given the exciting field of shape-dependent cell internalization research [26, 34, 35], these findings should provide further insight into designing future diagnostic tools, including diagnostic embedded therapeutics.

### REFERENCES FOR EXAMPLE 9

1. S. Dong, H. Ma, X. Li, M. Sun and X. Duan, Anal. Lett., 2004, 37, 2937-2948.
2. C.-G. Niu, X.-Q. Gui, G.-M. Zeng, A.-L. Guan, P.-F. Gao and P.-Z. Qin, Anal. Bioanal. Chem., 2005, 383, 349-357.
3. H. Sun, A. M. Scharff-Poulsen, H. Gu and K. Almdal, Chem. Mater., 2006, 18, 3381-3384.
4. E. Allard and C. Larpent, J. Polym. Sci., Part A: Polym. Chem., 2008, 46, 6206-6213.
5. J. Han, A. Loudet, R. Barhoumi, R. C. Burghardt and K. Burgess, J. Am. Chem. Soc., 2009, 131, 1642-1643.
6. Y.-L. Chiu, S.-A. Chen, J.-H. Chen, K.-J. Chen, H.-L. Chen and H.-W. Sung, ACS Nano, 2010, 4, 7467-7474.
7. T. Jin, A. Sasaki, M. Kinjo and J. Miyazaki, Chem. Commun., 2010, 46, 2408-2410.
8. M. Lee, N. G. Gubernator, D. Sulzer and D. Sames, J. Am. Chem. Soc., 2010, 132, 8828-8830.
9. J. Lei, L. Wang and J. Zhang, Chem. Commun., 2010, 46, 8445-8447.
10. H.-S. Peng, J. A. Stolwijk, L.-N. Sun, J. Wegener and O. S. Wolfbeis, Angew. Chem. Int. Ed., 2010, 49, 4246-4249.
11. Y.-H. Chan, C. Wu, F. Ye, Y. Jin, P. B. Smith and D. T. Chiu, Anal. Chem., 2011, 83, 1448-1455.
12. S. Kim, H. E. Pudavar and P. N. Prasad, Chem. Commun., 2006, 2071-2073.
13. G. Zhang, G. M. Palmer, M. W. Dewhirst and C. L. Fraser, Nature Mater., 2009, 8, 747-751.
14. Y. Song, X. Cao, Y. Guo, P. Chen, Q. Zhao and G. Shen, Chem. Mater., 2009, 21, 68-77.
15. Y. Kurishita, T. Kohira, A. Ojida and I. Hamachi, J. Am. Chem. Soc., 2010, 132, 13290-13299.
16. H.-J. Kim, J. Lee, T.-H. Kim, T.-S. Lee and J. Kim, Adv. Mater., 2008, 20, 1117-1121.
17. K. Honda, E. Nakata, A. Ojida and I. Hamachi, Chem. Commun., 2006, 4024-4026.
18. J. E. Whitaker, R. P. Haugland and F. G. Prendergast, Ana. Biochem., 1991, 194, 330-344.
19. Y.-C. Chen, A. Ostafin and H. Mizukami, Nanotechnology, 2010, 21,215503.215501-215510.
20. N. S. Lee, G. Sun, W. L. Neumann, J. N. Freskos, J. J. Shieh, R. B. Dorshow and K. L. Wooley, Adv. Mater., 2009, 20, 1344-1348.
21. A. M. Nyström, Z. Xu, J. Xu, S. Taylor, T. Nittis, S. A. Stewart, J. Leonard and K. L. Wooley, Chem. Commun., 2008, 3579-3581.
22. L. Y. Lin, N. S. Lee, J. Zhu, A. M. Nyström, D. J. Pochan, D. R. B. and K. L. Wooley, J. Control. Release, 2011, In press.
23. Y. Li, K. Hindi, K. M. Watts, J. B. Taylor, K. Zhang, Z. Li, D. A. Hunstad, C. L. Cannon, W. J. Youngs and K. L. Wooley, Chem. Commun., 2010, 46, 121-123.
24. K. Qi, Q. Ma, E. E. Remsen, J. Clark, C. G. and K. L. Wooley, J. Am. Chem. Soc., 2004, 126, 6599-6607.
25. M. J. Joralemon, K. S. Murthy, E. E. Remsen, M. L. Becker and K. L. Wooley, Biomacromolecules, 2004, 5, 903-913.
26. K. Zhang, R. Rossin, A. Hagooly, Z. Chen, M. J. Welch and K. L. Wooley, J. Polym. Sci., Part A: Polym. Chem., 2008, 46, 7578-7583.
27. M. L. Becker, J. Liu and K. L. Wooley, Biomacromolecules, 2005, 6, 220-228.
28. N. S. Lee and K. L. Wooley, Material Matters, 2010, 5, 9-12.
29. G. Sun, N. S. Lee, W. L. Neumann, J. N. Freskos, J. J. Shieh, R. B. Dorshow and K. L. Wooley, Soft Matter, 2009, 5, 3422-3429.
30. D. Benoit, V. Chaplinski, R. Braslau and C. J. Hawker, J. Am. Chem. Soc., 1999, 121, 3904-3920.
31. N. S. Lee, Y. Li, C. M. Ruda and K. L. Wooley, Chem. Commun., 2008, 42, 5339-5341.
32. K. Shirai, A. Yanagisawa, H. Takahashi, K. Fukunishi and M. Matsuoka, Dyes Pigm., 1998, 39, 49-68.
33. G. Sun, H. Cui, L. Y. Lin, N. S. Lee, C. Yang, W. L. Neumann, J. N. Freskos, J. J. Shieh, R. B. Dorshow and K. L. Wooley, unpublished result, 2011.
34. L. E. Euliss, J. A. DuPont, S. Gratton and J. DeSimone, Chemical Society Reviews, 2006, 35, 1095-1104.
35. K. Zhang, H. Fang, Z. Chen, J.-S. A. Taylor and K. L. Wooley, Bioconjugate Chem., 2008, 19, 1880-1887.

### Example 10: Multicompartment Polymer Nanostructures with Ratiometric Dual-Emission pH-Sensitivity

Abstract

Pyrazine-labeled multi-compartment nanostructures are shown to exhibit enhanced pH-responsive blue-shifted fluorescence emission intensities than are their simpler core-shell spherical analogs. An amphiphilic linear triblock terpolymer of ethylene oxide, N-acryloxysuccinimide and styrene, PEO₄₅-b-PNAS₁₀₅-b-PS₄₅, which lacks significant incompatibility for the hydrophobic block segments and undergoes gradual hydrolysis of the NAS units, underwent supramolecular assembly in mixtures of organic solvent and water to afford multicompartment micelles (MCMs) with narrow size distribution. The assembly process was followed over time and found to evolve from individual polymer nanodroplets containing internally-phase segregated domains, of increasing definition, and ultimately to dissociate into discrete micelles. Upon covalent cross-linking of the MCMs with pH-insensitive pyrazine-based diamino cross-linkers, pH-responsive, photonic multicompartment nanostructures (MCNs) were produced. These MCNs exhibited significant enhancement of overall structural stability, in comparison with the MCMs, and internal structural tunability through the cross-linking chemistry. Meanwhile, the complex compartmentalized morphology exerted unique pH-responsive fluorescence dual-emission properties, indicating promise in ratiometric pH-sensing applications.

### Introduction

The development of polymeric nanostructures from block copolymer supramolecular assemblies has gained significant attention [1-11], from which it has been recognized that their chemical composition, size and morphology each require precise tuning. Inspired by the successes from small molecule amphiphiles such as lipids, considerable efforts have been devoted to understand and manipulate the aqueous self-assembly process of amphiphilic block copolymers to obtain nano-scale assemblies with complex morphologies, which has been demonstrated as a promising parameter for addressing their potential biomedical applications [12-15]. For example, non-spherical nanostructures exhibited prolonged blood circulation time [16], more proficient cell targeting [17], and more efficient phagocytosis [18], compared with the corresponding spherical counterparts. Benefiting from the advances of living/controlled polymerization methodologies to afford varied block copolymer structures [19-24], together with extensive investigation of their aqueous assembly [22, 25-34], polymeric nanostructures with diverse morphologies have been established. In addition to conventional morphologies, such as spheres, cylinders and vesicles, bowls [35], discs [36], helices [37], and toroids [38], have been reported. Moreover, Janus [39], multicompartment [40, 41], onion [42], and large compound micelles [43], from higher-order inter- and/or intra-micellar phase segregation, have been created.

Multicompartment micelles (MCMs) represent intra-micellar phase-segregated block copolymer supramolecular assemblies, in which the core domains are heterogeneous and compartmentalized [25, 44]. Utilizing ABC starlike block terpolymers, by Lodge, Hillmyer and co-workers [40], and ABC linear triblock copolymers, by Laschewsky et al. [41] (in both cases, A represents the hydrophilic block segment, B and C represent incompatible hydrophobic block segments), MCMs were realized through the compartmentalization of B and C blocks during the aqueous assembly process. Additional MCMs have been prepared by tuning of polymeric and supramolecular parameters to manipulate the sizes, morphologies [45-53], and internal environments of the compartmentalized cores [54-57], and to generate stimuli-induced responses [58-61]. Meanwhile, the performance of MCMs as delivery vehicles for various cargos has been investigated to address their unique potential for biomedical applications [62].

Whereas a variety of star terpolymers [40, 45-48, 54, 56, 58, 60] and linear block copolymers [41, 50-53, 55, 57, 61, 63] have been explored as precursors to prepare MCMs, the introduction of functionalities into MCMs for facile and practical chemical manipulations [64, 65] remains as a fundamental aspect requiring further investigation [52]. Herein we disclose an approach for the construction of MCMs from aqueous assembly of a linear poly(ethylene oxide)-block-poly(N-acryloxysuccinimide)-block-polystyrene (PEO-b-PNAS-b-PS), 1, amphiphilic ABC triblock terpolymer, to afford nano-scopic assemblies with compartmentalized PS core domains. Borrowing from the terminology that has been developed for multivalent systems, which can be either of homo-multivalency or hetero-multivalency [66], we adopt the term “multicompartment”, for these newly-developed homo-multicompartment materials. The overall process involves an evolution from individual nanodroplets of polymer dispersed in water, to increasingly-defined phase-segregated domains within those nanodroplets, and ultimately to discrete micelles, as the NAS functionalities undergo hydrolysis over time. While still present, the residual NAS functionalities within MCMs can be utilized for covalent incorporation of other molecules to render the MCMs functionalized, through well-established amidation chemistry.

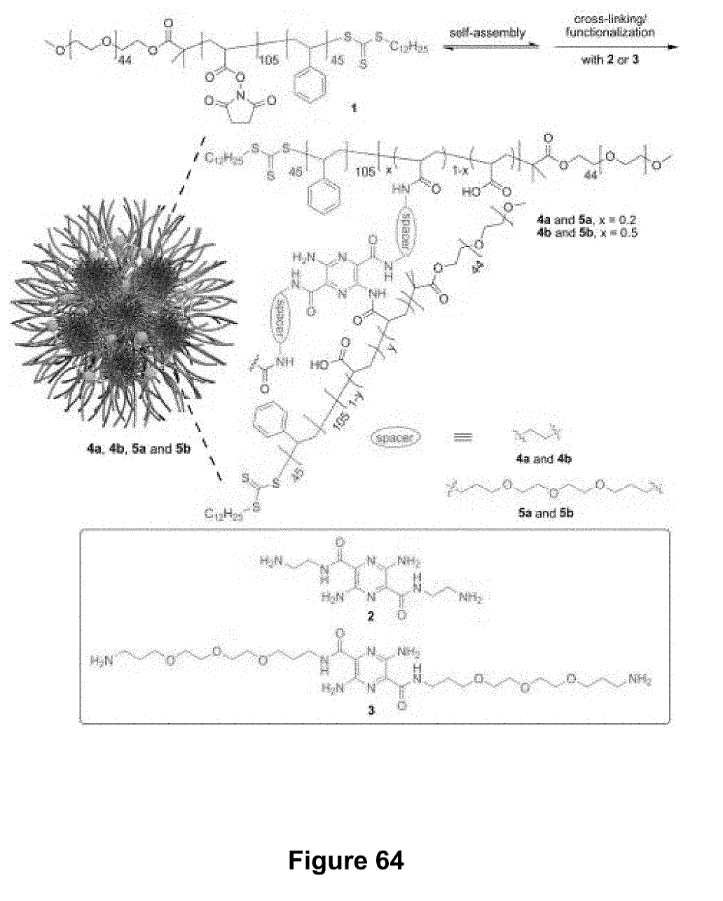

In this Example, photophysically-active pyrazine-based diamino cross-linkers, 2 and 3 of FIG. 64 were used to establish the stabilized photo-active multicompartment nanostructures (MCNs), 4a, 4b, 5a, and 5b, respectively. The cross-linking not only enhances the stability of MCMs to afford MCNs with hydrophilic shells, but also allows for tuning of the MCN internal spacing, through varying the chemical structures and the incorporation stoichiometry of the cross-linkers. These MCNs exhibit unique fluorescence emission characteristics, upon exposure to external environments at different pH values.

Results and Discussion

ABC linear triblock terpolymers have been shown to undergo greater variability in their assembly behaviors, in comparison to diblock copolymers [36-38, 41, 50, 52, 53, 59, 61, 67-70]. Furthermore, orthogonal cross-linking of the reactive groups pre-installed across either the hydrophilic [71, 72] or the hydrophobic [73] block segment have been demonstrated. The particular triblock terpolymer composition and sequence, PEO₄₅-b-PNAS₁₀₅-b-PS₄₅, were selected to provide a hydrophilic PEO end segment for water dispersibility, a central PNAS segment for reactivity, and a terminal hydrophobic PS segment to provide for nucleation of micellar assemblies in water and provide ability to trap initial MCM morphologies kinetically. The activated ester functionalities enable chemical modifications to improve the structural stability by incorporating cross-linkers.

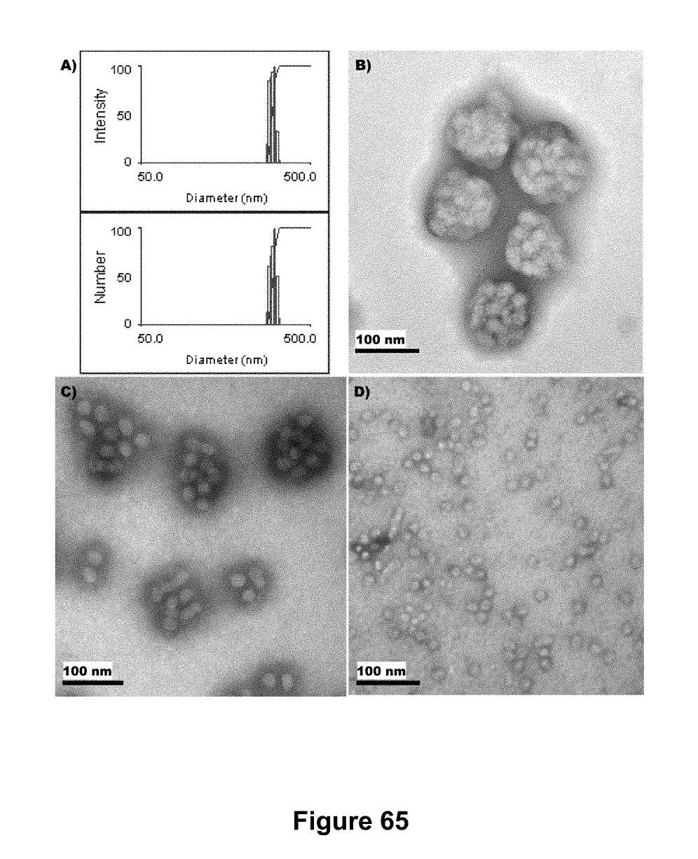

MCMs were assembled from 1 in aqueous solution when the polymers were freshly prepared, by introducing water (a selective solvent for PEO) to solutions of the triblock terpolymer in N,N-dimethylformamide, DMF (a good solvent for all three blocks). The nanoscale MCM assemblies in H₂O/DMF (v:v=1:1) were characterized immediately by dynamic light scattering (DLS) and transmission electron microscopy (TEM). The DLS results confirmed that uniform nanostructures were obtained (PDI<0.1, cumulant analysis) with a hydrodynamic diameter (Dₕ) of 300±20 nm, as shown in FIG. 65, panel A. The internal compartmentalized structure of these assemblies was supported by the TEM image shown in FIG. 65, panel B. Distinct from the previous PEO₄₅-b-PNAS₉₅-b-PS₆₀ triblock terpolymer, which provided discrete spherical micelles after assembly, the relatively longer PNAS and shorter PS block segments in the current terpolymers caused dramatically different assembly behavior. Individual nanodroplets containing internal phase-segregated domains acquired increasing definition until ultimately dissociating into discrete micelles. We attribute the occurrence of compartmentalization to the difference of interfacial tension of hydrophobic PNAS and PS blocks against water, as the immiscibility of the PNAS and PS segments is not as apparent as prior studies involving other block segment pairs, including fluorophilic blocks [40, 41]. Upon inducing the aqueous assembly process, the relatively stronger interfacial tension of PS against water, together with the π-π stacking interactions between aromatic ring moieties, accelerated the formation of dispersed smaller spherical domains in a larger PNAS domain. The overall progress of internal phase segregation was determined by the intrinsic block length ratio between PNAS and PS blocks. A relatively shorter PS block, which can offer stronger tendency to spherical morphology, and a relatively longer PNAS block, which grants sufficient space allowing the reorganization of PS and maintains adequate hydrophobicity during the assembly process (at a rate that is slower than the rate of assembly, the NAS groups undergo hydrolysis to generate hydrophilic acrylic acids, AAs (half-life on the order of a few hours)), facilitate the formation of MCMs.

It was noticed that the integrity of the MCM structures was related to the extent of NAS hydrolysis. With increased amounts of AA residues within MCM shells (after 3 months of storage, >95% of the NAS were hydrolyzed, as confirmed by NMR), enhancement of core domain compartmentalization was observed, as shown in FIG. 65, panel C. Meanwhile, partial dissociation of components within the established MCMs was evidenced by the appearance of smaller aggregates, as shown in FIG. 65, panel C. These phenomena can be attributed to the increased electrostatic repulsions between negatively-charged acrylates. The disassembly of MCMs (without any covalent stabilization) into discrete micellar forms ultimately occurred over long storage times (9 months, as shown in FIG. 65, panel D). The evolution of the entire process of internal compartmentalization and transformation of MCMs to discrete, amphiphilic core-shell micelles is under further investigation.

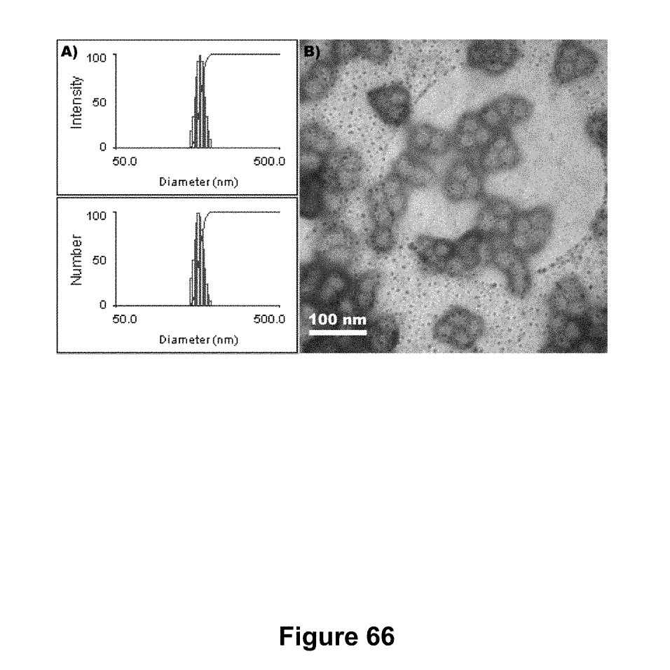

The extent of NAS hydrolysis also affected the self-assembly behavior of the triblock terpolymer precursors. Uniform MCMs with smaller size (Dₕ=160±15 nm, as shown in FIG. 66, panel A) and lower numbers of compartments (as shown in FIG. 66, panel B) were produced through the assembly of PEO₄₅-b-P(NAS₉₅-co-AA₁₀)-b-PS₄₅ precursors, having ca. 10% NAS hydrolysis. These results sustained our hypothesis (vide supra) that the subsistence of charges within MCM shell domains influenced the fate of these supramolecular assemblies, and also provided additional tunability for the construction of diverse MCMs. As a note, the triblock terpolymer precursors became only partially soluble in DMF when greater than 30% of NAS hydrolysis had occurred. Therefore, the self-assembly studies of these polymers were not conducted.

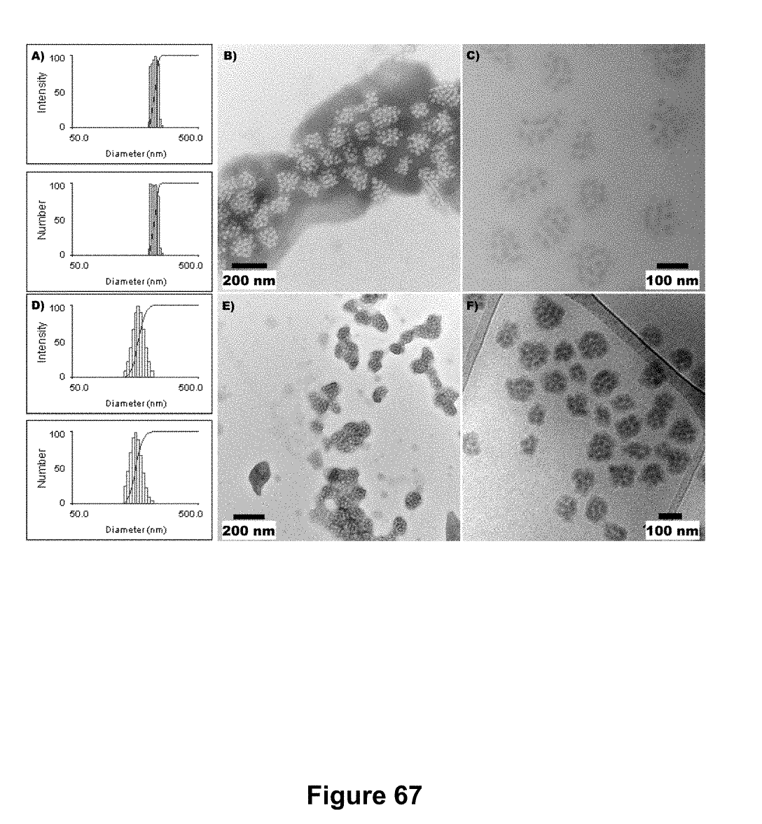

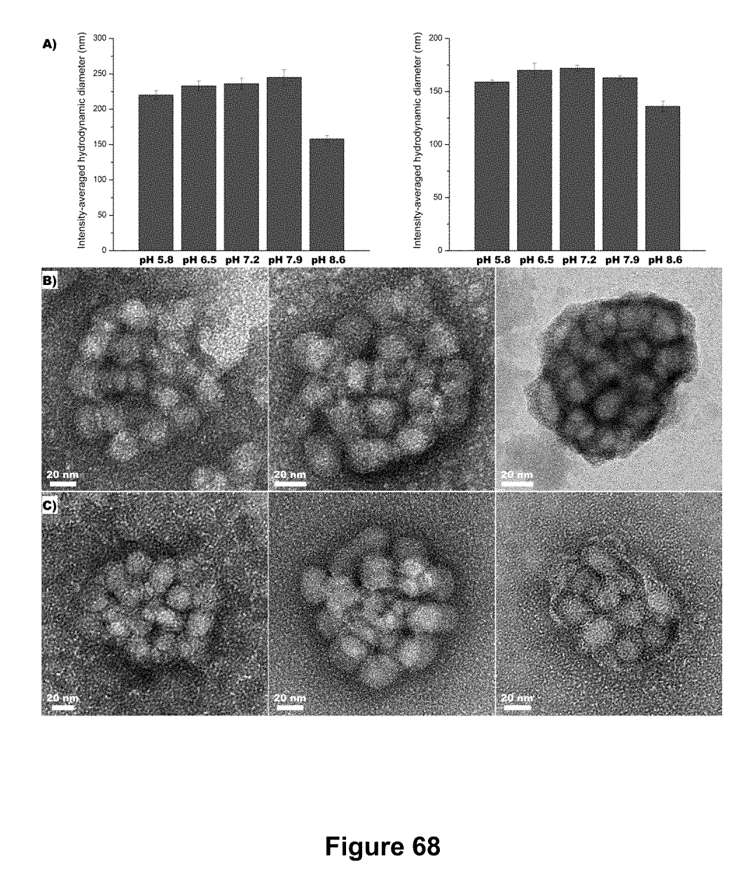

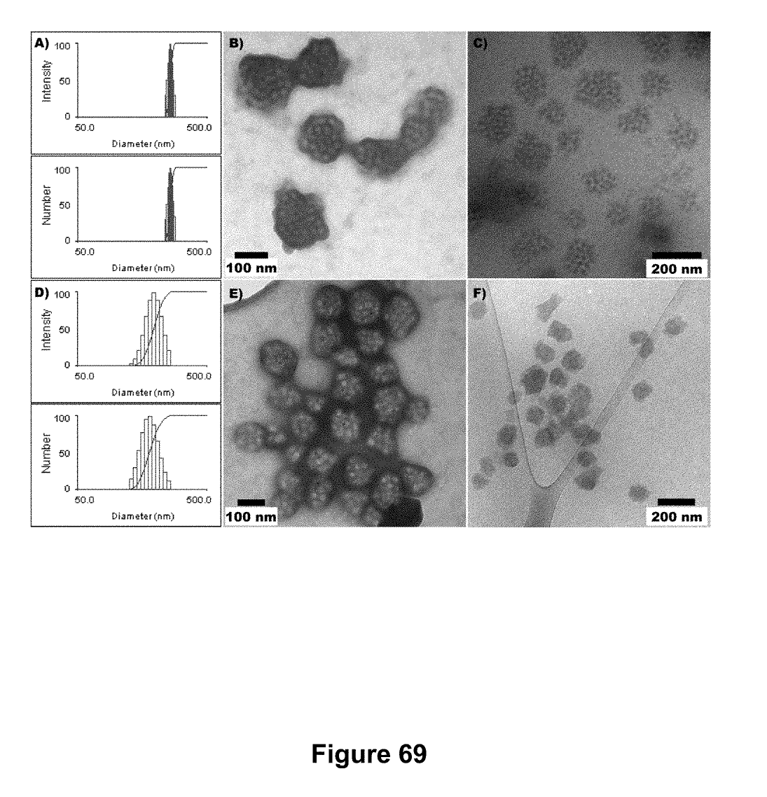

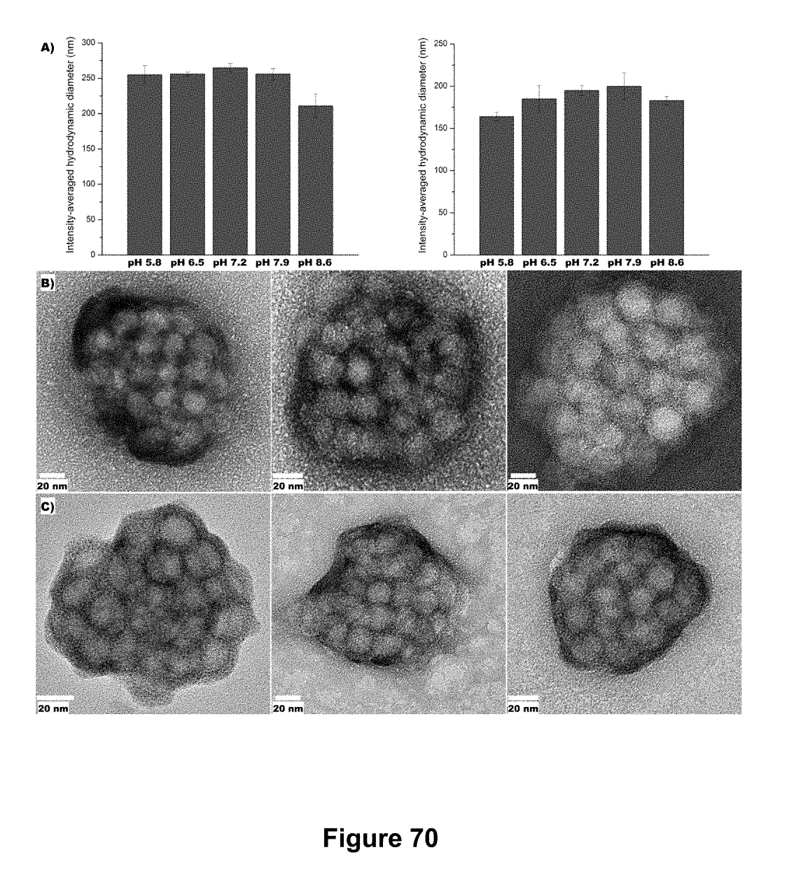

Covalent cross-linking and functionalization of the MCMs were accomplished by a one-step approach, utilizing cross-linkers 2 or 3, designed to also determine the incorporation/cross-linking efficiency [74] and to enable unique pH-driven photo-physical property responses [75]. Compared with the MCM precursors, the hydrodynamic diameters of MCNs with cross-linker 2 decreased, as confirmed by DLS, as shown in FIG. 67, panel A and 67, panel D (also see FIG. 68, panel A). The observed shrinkage effect correlated with the cross-linking extents, i.e., as the extents of pyrazine incorporation increased from 0% to 9% to 17%, the corresponding Dₕ decreased from 300±20 nm to 225±25 nm to 165±30 nm. It also was found that the work-up procedure affected the final size for the MCNs with 9% of cross-linking, as shown in FIG. 68, panel A, left. Although the MCNs retained a similar size of 220 nm over a pH range of 5.8 to 7.9, with further increase of the pH value to 8.6, the hydrodynamic diameter decreased to ˜160 nm. The cause for this reduction in dimension with increase of pH is unknown. The DLS observations were further supported by high-resolution TEM images of the corresponding MCNs, in which the internal PS compartments in MCNs at pH 8.6 showed relatively compacted packing mode, as shown in FIG. 68, panel B. This reduction was tightly associated with the cross-linking extents; at higher degrees of cross-linking, the pH-responsive shrinkage was diminished as shown in FIG. 68, panel A, left vs. right and FIG. 68, panel B vs. FIG. 68, panel C. However, we are unable to determine whether the apparent reduction in size is due to a contraction within established MCNs or due to some degree of dissociation of loosely-cross-linked components within MCNs. These trends were also observed for cross-linker 3, as shown in FIG. 69, panel A and 69, panel D, FIG. 70, panels A-C. Interestingly, the incorporation efficiency of 3 (˜60%) was higher than that of 2 (˜40%) at both examined cross-linking extents, in contrast to constant relative incorporation of each cross-linker within core-shell micelle systems studied previously [74].

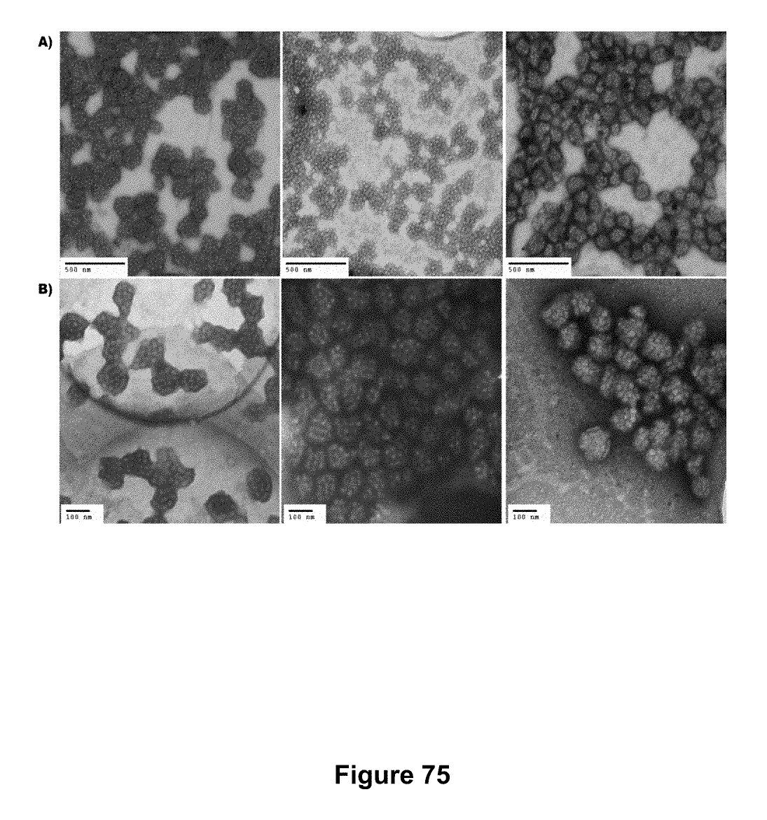

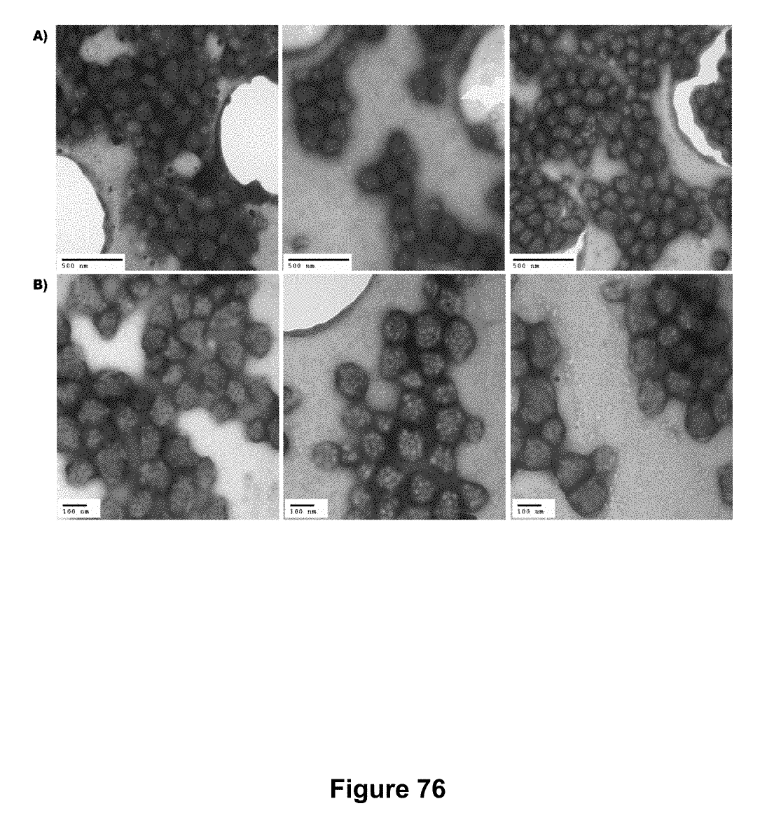

TEM and cryogenic-TEM (cryo-TEM) imaging (middle and right column in FIGS. 67 and 69, respectively) of MCNs gave diameters that were in agreement with the DLS results and provided more structural information (also see FIGS. 68 and 70, 75 and 76 for TEM images at additional pH values). Comparison of MCM and MCN images (FIG. 65 vs. 67-70) demonstrated maintenance of the internal segregated domains and enhanced compartmentalization after cross-linking. However, different packing patterns of the compartments occurred with different cross-linking extents. Noticeably different inter-compartment spacings were detected by cryo-TEM (FIG. 67, panels C and F, and FIG. 69 panels C and F).

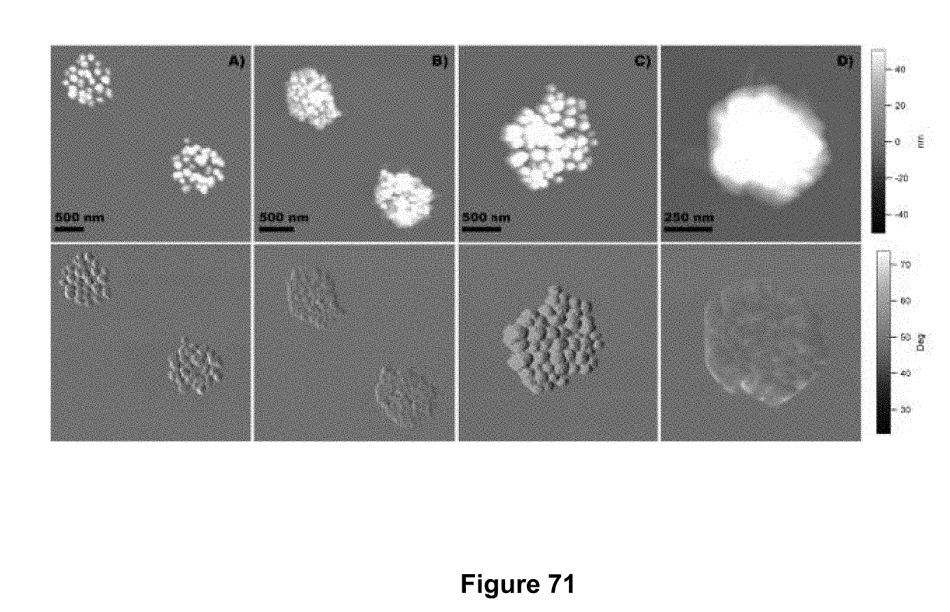

We further characterized the MCNs by atomic force microscopy (AFM). As shown in FIG. 71, MCNs with the highest degree of cross-linking (MCN 5b, maximum ˜30% of cross-linking) displayed the smallest variations between the diameter and height (D/H≈3) after casting onto mica, indicating that 5b had the most discrete and robust structural characteristics. In comparison, the least cross-linked MCN 4a (maximum ˜9% of cross-linking) exhibited a D/H ratio >8. The AFM images of 4a vs. 4b, and 5a vs. 5b (FIG. 71, panel A vs. B and panel C vs. panel D, respectively) also supplied additional verifications for the general trend of MCN internal structures, i.e., decrease of inter-compartment spacings with increase of cross-linking extents.

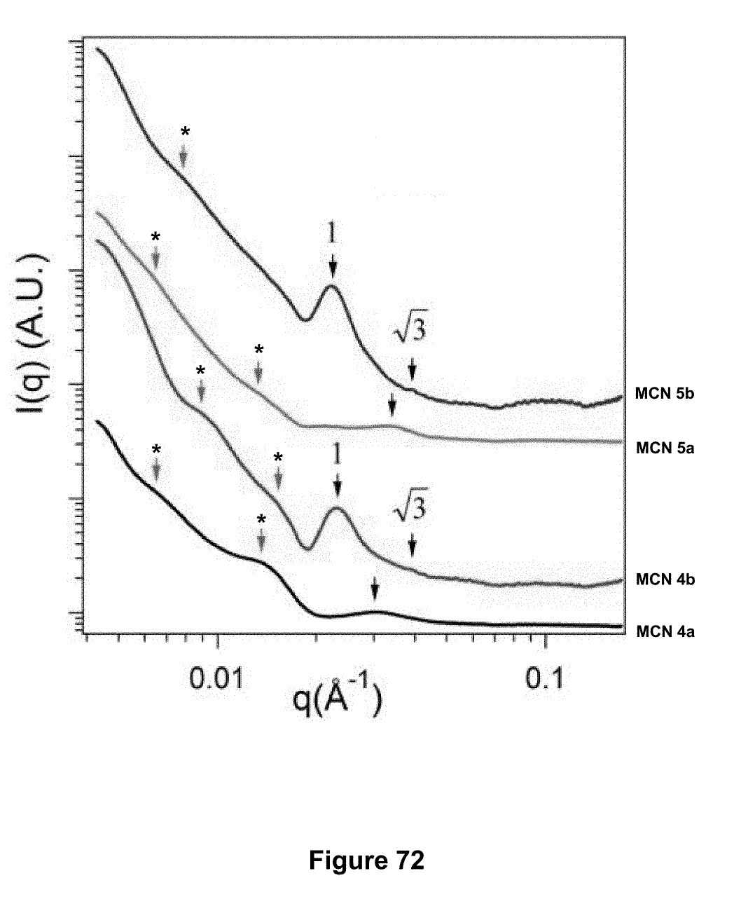

Small-angle X-ray scattering (SAXS) was then used to probe the internal packing orders of these MCNs, as shown in FIG. 72. For both cross-linkers, MCNs 4b and 5b with higher cross-linking extents showed more ordered internal structures than did 4a and 5a, as evidenced by the sharp Bragg peaks (marked with black arrows). The relative positions of the principal Bragg peak (0.024 Å⁻¹ and 0.022 Å⁻¹ for 4b and 5b, respectively) to its higher order reflection indicated hexagonal internal packings [76]. The calculated center-to-center spacing was 30.7 nm for 4b and 33.0 nm for 5b, respectively. The calculation showed that MCNs prepared using 2 had smaller spacing than those prepared using 3, which supported that the internal spacing of MCNs could be tuned by choosing cross-linkers with different chemical structures. For the 20% cross-linked samples (4a and 5a), their SAXS profiles showed broad Bragg peaks, suggesting that these MCNs were less internally ordered, consistent with TEM, cryo-TEM, and AFM images.

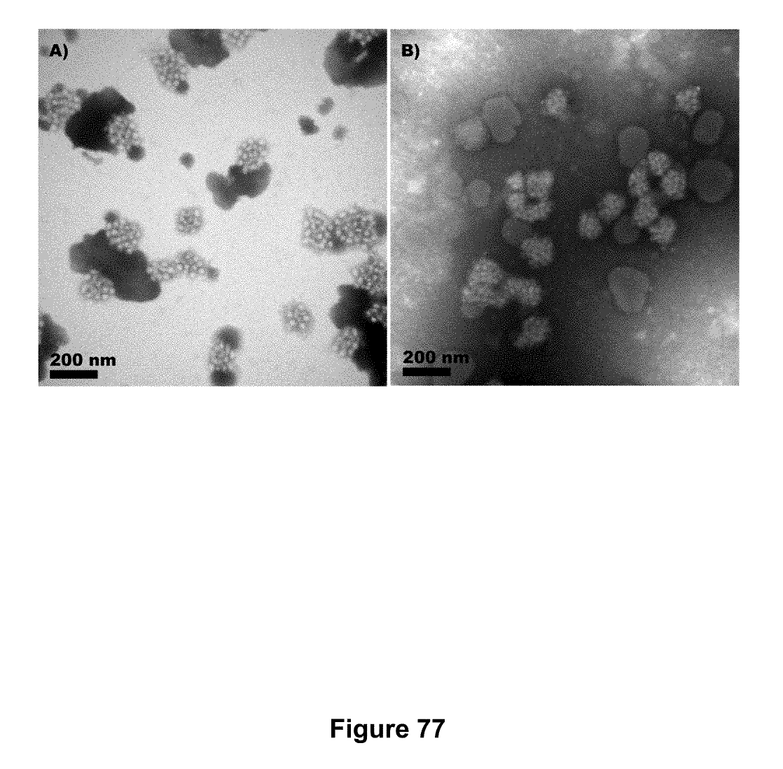

The significant increase of MCN structural stability after cross-linking was verified by comparing morphologies of the pre-established MCMs and 2-cross-linked MCNs (4a and 4b) in mixed organic/aqueous media (DMF/H₂O) over storage times (9 months) at room temperature. While the disassembly of MCMs occurred (vide supra), the MCNs (4a and 4b) did not show appreciable morphology variations (FIG. 77, panels A and B, respectively), even at lower degrees of cross-linking (4a, maximum cross-linking extent less than 10%). The long-term dissociation of MCMs into discrete micelles supports our hypothesis that the overall process involves an evolution from multi-compartment nanostructures, rather than an opposite process of micellar aggregation.

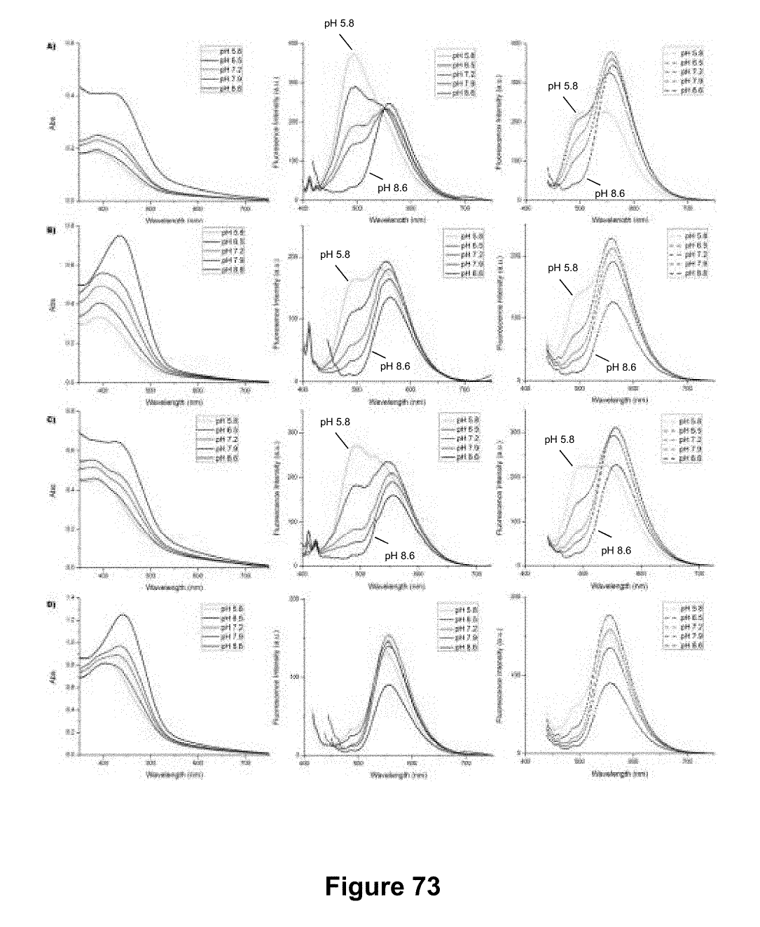

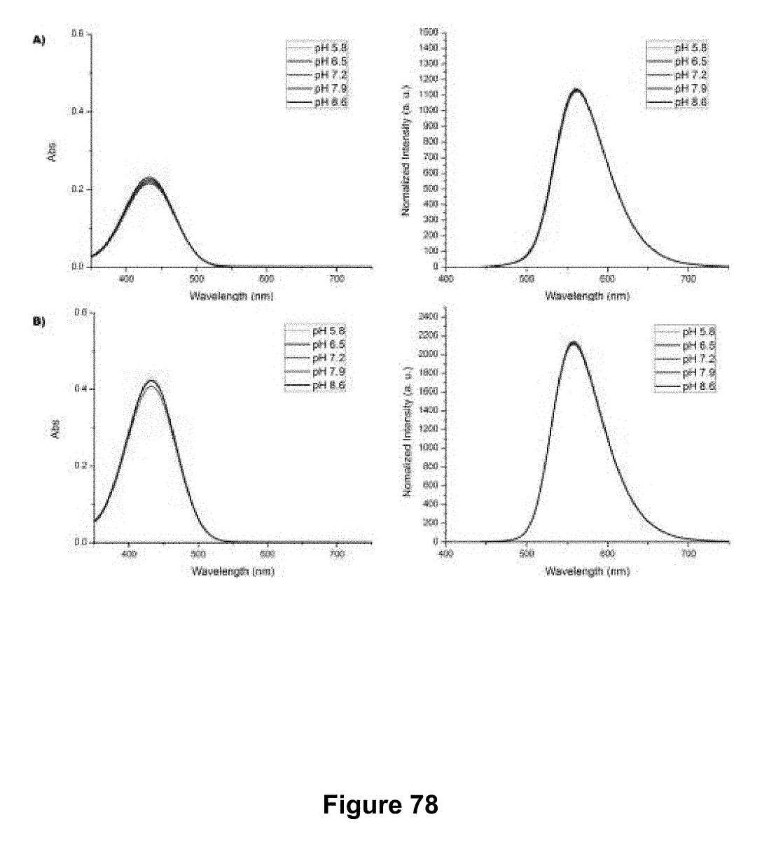

One motivation for these experiments arose from our recently reported fluorophore-shell-cross-linked nanoparticles (SCKs), a pH-driven nano-platform that demonstrated notable enhancement of fluorescent properties within the physiological pH region [75]. Because the MCNs represent sophisticated supramolecular assemblies, it was reasonable to anticipate more complex photo-physical properties of fluorogenic MCNs after covalent installation of the pyrazine chromophores. For 2 and 3 small molecules at the surveyed pH values, no apparent UV-Vis absorbance and fluorescence emission spectra variation was detected, as shown in FIG. 78, which indicated their intrinsic non-pH-responsive properties. As 2 and 3 were incorporated into MCNs through covalent functionalization, the UV-Vis maximum absorbance peaks were blue shifted from 433 nm to ca. 390 nm and 380 nm (4a-b and 5a-b, respectively) at pH 5.8. With an increase of the external pH values, the 433 nm peak began to appear along the UV-Vis profile and, eventually became the equivalent or even dominant absorbance peak, depending upon the incorporation extents, as shown in FIG. 73, panels A-D, left column. More interestingly, the fluorescence emission (excitation at maximum absorbance wavelength, λ_{abs,max}) at corresponding pH values also experienced such a tendency, as shown in FIG. 73, panels A-D, middle column. Upon excitation of 4a in acidic media (pH 5.8 and 6.5, respectively) with λ_{abs,max}, the fluorescence emission peaks were blue shifted to 495 nm as the dominant (pH 5.8) or major (pH 6.5) peak. As the environmental pH values increased to neutral (pH 7.2) and weakly basic (pH 7.9), 4a showed dual-emissions at 495 nm and 555 nm, and the 555 nm emissions became of greater intensity at both pH values. At the highest pH value (pH 8.6) surveyed, 4a only displayed the 555 nm emission. For 5a, a similar evolution of photophysical properties was verified, except that the threshold of fluorescence emission variation began at pH 6.5 and self-quenching of fluorescence emission was boosted. In the case of 4b, apparent fluorescence self-quenching appeared and the 495 nm emission quickly vanished as the external pH values were above neutral conditions, in contrast to 4a. For 5b with the highest incorporation extent of pyrazines, the 555 nm emission always acted as the dominant character across the surveyed pH range.

We also noticed that, for MCNs 4a, 4b, and 5a, the integrations of the fluorescence emission spectra (excitations at the corresponding λ_{abs,max}) decreased with the elevation of pH values, as shown in FIG. 73, panels A-C, middle column, suggesting the existence of two types of fluorogenic species from the covalent installation of pyrazines into the established MCMs. We speculated that these two fluorophores exhibited different photo-physical properties as a function of pH, i.e., one had a higher degree of pH-sensitive fluorescence character, which was responsible for the 495 nm emission; while the other one, that gave the 555 nm emission, had less sensitivity upon pH variations or even was non-pH-sensitive. This hypothesis was supported by the results from studies in which the 433 nm excitations (the original λ_{abs,max} for both 2 and 3) were applied to these fluorogenic MCNs. The reduction of fluorescence emission intensity at 495 nm followed the trend as described above, as shown in FIG. 73, panels A-D, right column, while significant enhancements of the 555 nm emissions (the λ_{em,max} for both 2 and 3) were observed, for 4a and 5a.

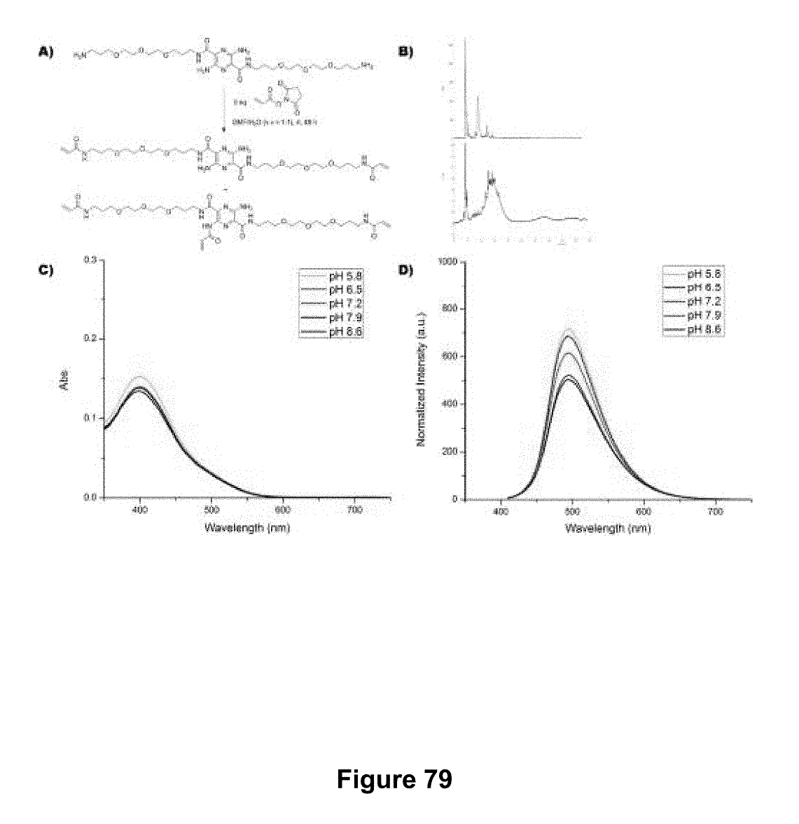

From the chemistry viewpoint, mono-acylation of the pyrazine aromatic amines can introduce asymmetries, which might affect its photo-physical properties. Therefore, we synthesized the tri-acylated derivative of 3, as shown in FIG. 79 and studied its photo-physical properties within the corresponding pH value range. The blue shifts of both the UV-Vis maximum absorbance peak (from 433 nm to 400 nm, FIG. 79, panel C) and fluorescence emission peak (from 560 nm to 495 nm, FIG. 79, panel D) were noticed, which was consistent with an earlier literature report [77]. In addition, pH-responsive fluorescence intensity decreases were observed, in response to the increasing of pH from 5.8 to 8.6. This control experiment demonstrated that the pH-sensitive photo-physical response by MCNs originated from the acylation of pyrazine aromatic amines. However, other factors including photon re-absorption and subsequent photon re-emission, twisted intramolecular charge-transfer [78-80], as well as the ionic strength of the media, could also be factors.

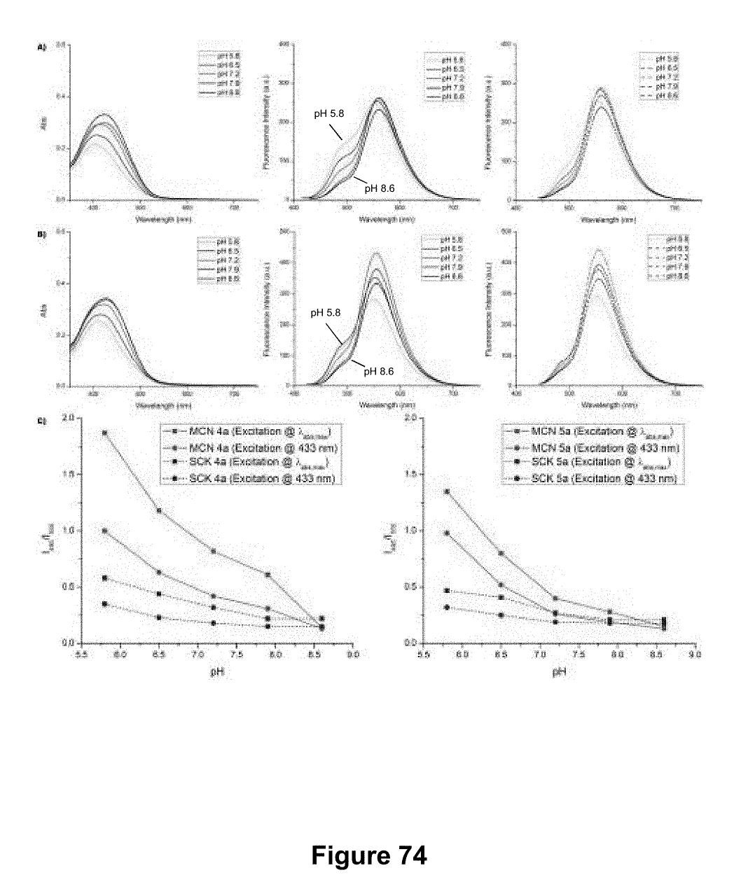

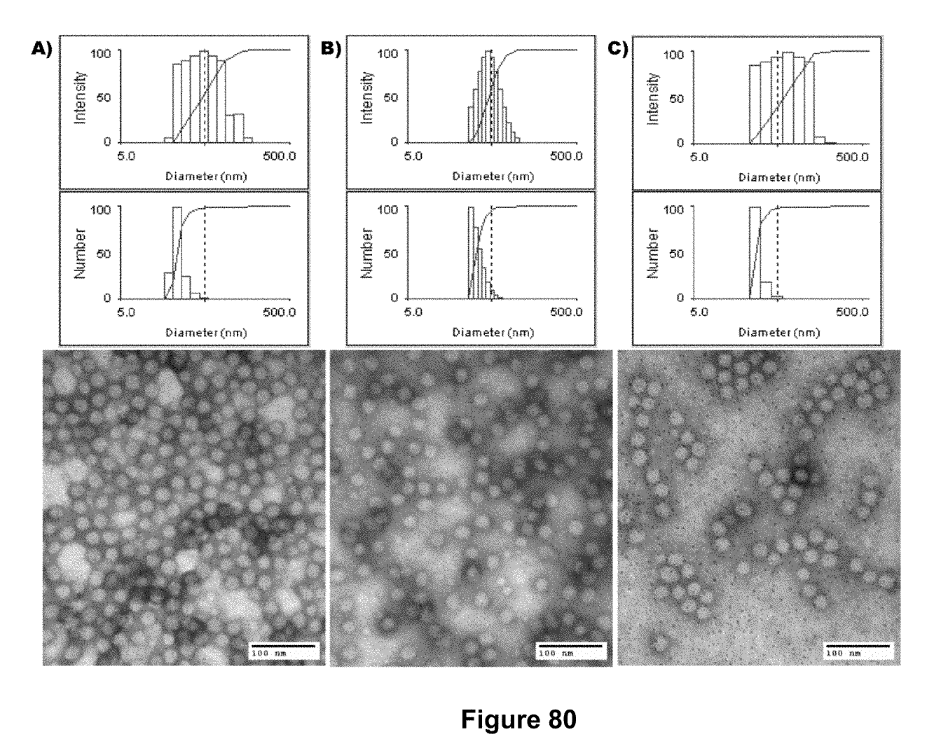

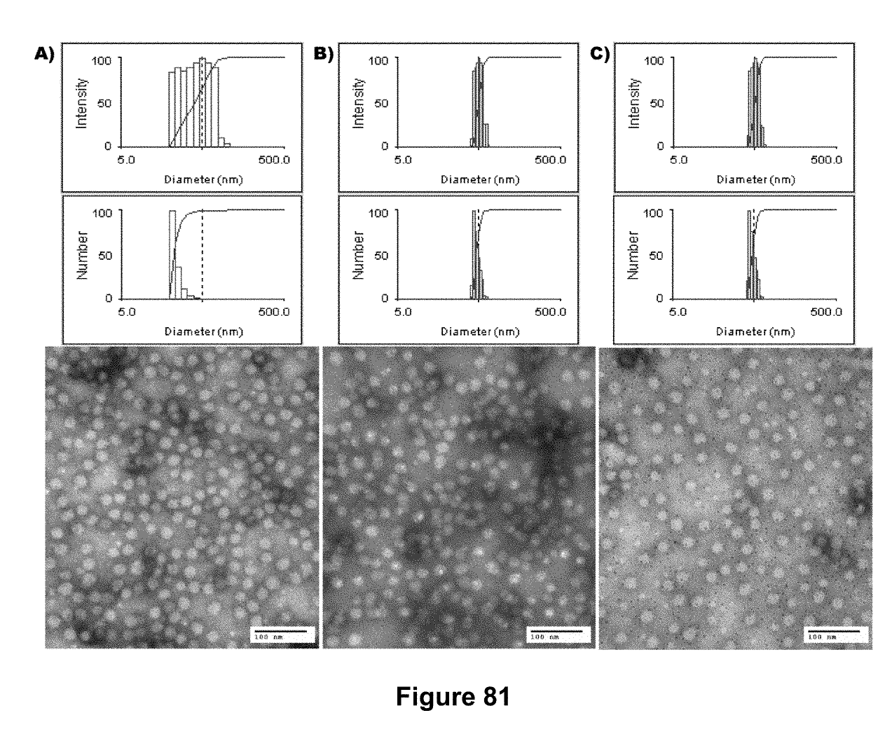

To explore the nanostructure morphological effect on photo-physical properties, we prepared photonic core-shell SCKs (SCK 4a and SCK 5a, at nominal 20% of cross-linking with 2 and 3, control samples for MCN 4a and MCN 5a, respectively) from PEO₄₅-b-PNAS₉₅-b-PS₆₀ triblock terpolymer precursors, by following the established protocol [74]. As shown in FIGS. 80 and 81, these SCK nanoparticles exhibited discrete spherical morphologies and relatively narrow size distributions. Upon exposing these SCKs to photo-physical studies, similar pH-responsive fluorescence emission trends were observed as for the MCNs, i.e., the 495 nm fluorescence emission intensity decreased with the elevated environmental pH values, as shown in FIG. 74. However, the 495 nm emission intensities of SCK 4a and SCK 5a were much less than the corresponding MCN 4a and 5a, especially at acidic conditions, as shown in the profiles in FIG. 74 vs. FIG. 73. In fact, the 555 nm emission always acted as the major fluorescence emission for all SCK samples, which was totally different from the phenomena observed from MCNs, as shown in FIG. 74, panel C.

### Conclusions

In summary, multicompartment nanoasssemblies bearing NHS active ester functionalities have been prepared from linear triblock terpolymer PEO₄₅-b-PNAS₁₀₅-b-PS₄₅ in DMF/H₂O solutions, and transformed into robust, pH-responsive, fluorescent nanostructures. The phase segregation process between the two hydrophobic building blocks was enhanced by the introduction of hydrophilic functionalities across the PNAS domain, upon hydrolysis, which further provided manipulation of the size and number of internal compartments of the assembled MCMs. The active ester functionalities were demonstrated to allow for modifications through facile and practical chemistry, including cross-linking and functionalizing with pyrazine-based cross-linkers to achieve enhanced stability and to enable pH-sensitive photo-physical responses. It is expected that the above unique properties of these MCNs will make them promising materials for fundamental study in biotechnology and other applications.

Experimental Section

Materials

The mono-methoxy terminated mono-hydroxy poly(ethylene glycol) (mPEG2k, MW=2,000 Da, PDI=1.06) was purchased from Intezyne Technologies and was used for the synthesis of macro-CTA without further purification. The PEO-b-PNAS-b-PS triblock copolymer (vide infra) and the cross-linkers 2 and 3 were synthesized according to previous reports [81, 82]. Other chemicals were purchased from Aldrich and Acros were used without further purification unless otherwise noted. Prior to use, N-acryloxysuccinimide (Acros, 99%) was recrystallized from dry ethyl acetate and stored under argon. Styrene (Aldrich, 99%) was distilled over calcium hydride and stored under N₂. The Supor 25 mm 0.1 μm Spectra/Por Membrane tubes (molecular weight cut-off (MWCO) 6-8 kDa), used for dialysis, were purchased from Spectrum Medical Industries Inc. Nanopure water (18 mei cm) was acquired by means of a Milli-Q water filtration system (Millipore Corp.)

Measurements

¹H and ¹³C NMR spectra were recorded on a Varian 600 MHz spectrometer interfaced to a UNIX computer using Mercury software. Chemical shifts are referred to the solvent proton resonance. IR spectra were recorded on an IR Prestige 21 system (Shimadzu Corp.) and analyzed by using the IRsolution software.

The molecular weight distribution was determined by Gel Permeation Chromatography (GPC). The N,N-dimethylformamide (DMF) GPC was conducted on a Waters Chromatography Inc. (Milford, Mass.) system equipped with an isocratic pump model 1515, a differential refractometer model 2414, and a two-column set of Styragel HR 4 and HR 4E 5 μm DMF 7.8×300 mm columns. The system was equilibrated at 70° C. in pre-filtered DMF containing 0.05 M LiBr, which served as polymer solvent and eluent (flow rate set to 1.00 mL/min). Polymer solutions were prepared at a concentration of ca. 3 mg/mL and an injection volume of 200 μL was used. Data collection and analysis was performed with Empower Pro software (Waters Inc.). The system was calibrated with poly(ethylene glycol) standards (Polymer Laboratories) ranging from 615 to 442,800 Da.

Transmission Electron Microscopy (TEM) bright-field imaging was conducted on a Hitachi H-7500 microscope, operating at 80 kV. The TEM imaging at high magnification was carried out on a FEI Tecnai G2 F20 microscope, operating at 200 kV. The samples were prepared as following: 4 μL of the dilute solution (with a polymer concentration of ca. 0.2-0.5 mg/mL) was deposited onto a carbon-coated copper grid, which was pre-treated with absolute ethanol or oxygen plasma to increase the surface hydrophilicity. After 1 min, the excess of the solution was quickly wicked away by a piece of filter paper. The samples were then negatively stained with 4 μL of 1 wt % phosphotungstic acid (PTA) aqueous solution. After 30 seconds, the excess PTA solution was quickly wicked away by a piece of filter paper and the samples were left to dry under room temperature overnight.

Cryogenic Transmission Electron Microscopy (Cryo-TEM) imaging was performed on a JEOL 1230 microscope, operating at 100 kV. A small droplet of the solution (5-10 μL) was placed on a holey carbon film supported on a TEM copper grid within a FEI Vitrobot system. The following procedure for the preparation of a thin film sample to facilitate EM imaging was controlled using instrument software with preset parameters. First of all, the specimen was carefully blotted by approaching two pieces of filter papers from both sides of the TEM copper grid. The blotting parameters (blot times, blot forces, and drain times) were selected to obtain a biconcave, thin water layer, typically less than 200 nm. During the blotting process, the humidity of the operation chamber was maintained above 90%. After blotting and a short waiting time, 1 or 2 second, the sample was plunged into a liquid ethane reservoir cooled by liquid N₂. The vitrified samples were transferred to a Gatan 626 cryo-holder and cryo-transfer stage cooled by N₂. During observation of the vitrified samples, the cryo-holder temperature was maintained below −170° C. to prevent sublimation of vitreous water.

Hydrodynamic diameters (Dₕ) and size distributions for the nanostructures in aqueous solutions were determined by dynamic light scattering (DLS). The DLS instrumentation consisted of a Brookhaven Instruments Limited system, including a model BI-200SM goniometer, a model BI-9000AT digital correlator, a model EMI-9865 photomultiplier, and a model Innova 300 (Coherent Inc., Santa Clara, Calif.) operated at 514.5 nm. Measurements were made at 25±1° C. Scattered light was collected at a fixed angle of 90°. The digital correlator was operated with 522 ratio spaced channels, and initial delay of 5 μs, a final delay of 100 ms, and a duration of 6 minutes. A photomultiplier aperture of 100 μm was used, and the incident laser intensity was adjusted to obtain a photon counting of between 200 and 300 kcps. Only measurements in which the measured and calculated baselines of the intensity autocorrelation function agreed to within 0.1% were used to calculate particle size. The calculations of the particle size distributions and distribution averages were performed with the ISDA software package (Brookhaven Instruments Company), which employed single-exponential fitting, cumulants analysis, and CONTIN particle size distribution analysis routines. All determinations were repeated 5 times.

The Atomic Force Microscopy (AFM) imaging was performed using MFP-3D system (Asylum Research, Santa Barbara, Calif.) in tapping mode using standard silicon tips (AC160TS, 160 μM, spring constant 42 N m 1). Samples were prepared by spin coating (1,500 rpm) onto fresh cleaved mica surface for 1 min and air-dried.

The small-angle X-ray scattering (SAXS) experiments were performed on the Dupont-Northwestern-DOW 5ID-D beamline. The X-ray energy (15 keV) was selected using a double-crystal monochromator. Liquid samples were placed in 2.0 mm quartz capillary tubes and the typical incident X-ray fluxed on the sample was ˜1×10¹² photons/s with a 0.2×0.3 mm² collimator, estimated by a He ion channel. The scattered radiation was detected using a MAR CCD camera and the 1-D scattering profiles were obtained by radial integration of the 2-D patterns, with scattering from the capillaries subtracted as background. Scattering profiles were then plotted on a relative scale as a function of the scattering vector q=(4π/Δ) sin(θ/2), where θ is the scattering angle.

The UV-Vis absorption spectra of MCNs were collected at room temperature using a Varian Cary 100 Bio UV-visible spectrophotometer and plastic cuvettes with 10 mm of light path. For each MCN absorption spectroscopy measurement, the corresponding buffer solution (5 mM with 5 mM of NaCl) outside the dialysis tubing was used as control.

The fluorescence spectra of MCNs were obtained at room temperature using a Varian Cary Eclipse fluorescence spectrophotometer. All fluorescence spectra from MCN solutions were measured at optical densities at the excitation wavelength. If not specially mentioned otherwise, an excitation wavelength of the observed maximum absorption peak was used. Each fluorescence spectrum was normalized with respect to the absorbed light intensity at the excitation wavelength.

Synthesis of PEO₄₅-b-PNAS₁₀₅.

To a 25 mL Schlenk flask equipped with a magnetic stir bar dried with flame under N₂ atmosphere, was added the mPEG2k macro-CTA (0.24 g, 0.10 mmol) and 1,4-dioxane (10 mL). The reaction mixture was stirred 0.5 h at rt to obtain a homogeneous solution. To this solution was added NAS (1.9 g, 11 mmol) and AIBN (0.9 mg, 6 μmol). The reaction flask was sealed and allowed to stir 10 min at rt. The reaction mixture was degassed through several cycles of freeze-pump-thaw. After the last cycle, the reaction mixture was allowed to stir for 10 min at rt before being immersed into a pre-heated oil bath at 60° C. to start the polymerization. After 105 min, the monomer conversion reached ca. 95% by analyzing aliquots collected through ¹H NMR spectroscopy. The polymerization was quenched by cooling the reaction flask with liquid N₂. The polymer was purified by precipitation into 400 mL of cold diethyl ether at 0° C. three times. The precipitants were collected, washed with 100 mL of cold ether, and dried under vacuum overnight to afford the PEO₄₅-b-PNAS₁₀₅ block copolymer precursor as a yellow solid (1.4 g, 68% yield based upon monomer conversion). ¹H NMR (600 MHz, DMSO-d₆, ppm): δ 0.81 (t, J=6 Hz, 3H, dodecyl CH₃), 1.09 (br, 5H, CH₃ and dodecyl CH₂), 1.20 (br, 19H, CH₃ and dodecyl CH₂s), 1.30 (br, 2H, dodecyl CH₂), 1.60 (t, J=6 Hz, 2H, dodecyl CH₂), 2.01 (br, PNAS backbone protons), 2.75 (NAS CH₂CH₂s), 3.09 (br, PNAS backbone protons), 3.20 (s, mPEG terminal OCH₃), 3.47 (m, OCH₂CH₂OO from the PEG backbone), 4.07 (br, 2H from the PEO backbone terminus connected to the ester linkage); 13C NMR (150 MHz, DMSO-d₆, ppm): δ 25.2, 41.2, 69.8, 172.8. Mₙ^{NMR}=24,800 Da, PDI=1.3 (DMF GPC).

Synthesis of PEO₄₅-b-PNAS₁₀₅-b-PS₄₅ (1).

To a 25 mL Schlenk flask equipped with a magnetic stir bar dried with flame under N₂ atmosphere, was added the PEO₄₅-b-PNAS₁₀₅ macro-CTA (1.1 g, 55 μmol), 1,4-dioxane (5.0 mL), and DMF (5.0 mL). The reaction mixture was allowed to stir for 0.5 h at rt to obtain a homogeneous solution. To this solution was added styrene (2.2 g, 21 mmol) and AIBN (0.49 mg, 3.0 μmol). The reaction flask was sealed and allowed to stir for 10 min at rt. The reaction mixture was degassed through several cycles of freeze-pump-thaw. After the last cycle, the reaction mixture was allowed to stir for 10 min at rt before being immersed into a pre-heated oil bath at 58° C. to start the polymerization. After 14.5 h, the monomer conversion reached ca. 13% by analyzing aliquots collected through ¹H NMR spectroscopy. The polymerization was quenched by cooling the reaction flask with liquid N₂. The polymer was purified by precipitation into 500 mL of cold diethyl ether at 0° C. three times. The precipitants were collected and dried under vacuum overnight to afford the block copolymer precursor as a yellow solid (1.0 g, 70% yield based upon monomer conversion). ¹H NMR (600 MHz, CD₂C2, ppm): δ 0.81 (br, dodecyl CH₃), 1.10-2.40 (br, dodecyl Hs, PNAS, and PS backbone protons), 2.75 (NAS CH₂CH₂s), 3.15 (br, PNAS backbone protons), 3.28 (s, mPEG terminal OCH₃), 3.60 (m, OCH₂CH₂O from the PEG backbone), 6.20-7.30 (br, Ar Hs); ¹³C NMR (150 MHz, DMSO-d₆, ppm): δ 25.2, 41.6, 69.8, 125.7, 128.0, 145.2, 172.8. Mₙ^{NMR}=24,800 Da, PDI=1.2 (DMF GPC).

General Procedure for Self-Assembly of 1.

To a solution of PEO₄₅-b-PNAS₁₀₅-b-PS₄₅ block copolymer in DMF (ca. 1.0 mg/mL), was added dropwise an equal volume of nano-pure H₂O within 2 h via a syringe pump at a rate of 15.0 mL/h. The mixture was further allowed to stir for 1 h at rt before used for characterizations and cross-linking/functionalization reactions.

General Procedure for Cross-Linking/Functionalization of PEO₄₅-b-PNAS₁₀₅-b-PS₄₅ Multicompartment Micelles (MCMs).

To a solution of PEO₄₅-b-PNAS₁₀₅-b-PS₄₅ MCMs (30.0 mg of block copolymer precursor, 127 μmol of NAS residues) in 60.0 mL of DMF/H₂O (v:v=1:1) at rt, was added dropwise over 10 min, a solution of cross-linker 2 or 3 (12.7 μmol for nominal 20% of cross-linking and 31.8 μmol for nominal 50% of cross-linking, respectively) in nanopure water. The reaction mixture was allowed to stir for 48 h at rt in the absence of light. The reaction mixture was then divided into five portions (ca. 13 mL each) and transferred into pre-soaked dialysis tubing (MWCO 6,000-8,000 Da) and dialyzed against 5.0 mM buffer solutions (with 5.0 mM NaCl) at pH 5.8, 6.5, 7.2, 7.9, and 8.6, respectively, for 7 days to remove DMF, un-reacted cross-linker, and the small molecule by-products to afford an aqueous solution of cross-linked/functionalized multicompartment nanostructures (MCNs).

Acylation of 3.

To a solution of 3 (25.2 mg, 0.15 mmol) in 4 mL of H₂O at rt, was added dropwise over 5 min, a solution of NAS (127 mg, 0.75 mmol) in 4 mL of DMF. The reaction mixture was allowed to stir for 48 h at rt in the absence of light. The solvent was removed under vacuum. The residues were re-suspending into 5 mL of CH₂Cl₂ and precipitating into 35 mL of dry diethyl ether. The solid product was collected by centrifugation and re-dissolved into 30 mL of nanopure water. The solution was passed through a 5 μm syringe filter to afford an aqueous stock solution of acylated 3. Before photo-physical measurements, the stock solution was diluted (v:v=1:5) with 5.0 mM buffer solution (with 5.0 mM NaCl) at pH 5.8, 6.5 7.2, 7.9, and 8.6, respectively.

Preparation of Photonic SCKs (4a and 5a).

To a solution of PEO₄₅-b-PNAS₉₅-b-PS₆₀ (30.0 mg of block copolymer precursor, 115 μmol of NAS residues) in 30.0 mL of DMF, was added dropwise an equal volume of nano-pure H₂O via a syringe pump at a rate of 15.0 mL/h, and the mixture was further stirred for 1 h at rt. To this micelle solution at rt, was added dropwise over 10 min, a solution of cross-linker 2 or 3 (41.1 mg, 11.6 μmol for 2 and 78.2 mg, 11.6 μmol for 3, respectively) in nanopure water. The reaction mixture was allowed to stir for 48 h at rt in the absence of light. The reaction mixture was then divided into five portions (ca. 13 mL each) and transferred into pre-soaked dialysis tubing (MWCO 6,000-8,000 Da) and dialyzed against 5.0 mM buffer solutions (with 5.0 mM NaCl) at pH 5.8, 6.5, 7.2, 7.9, and 8.6, respectively, for 7 days to remove DMF, un-reacted cross-linker, and the small molecule by-products to afford an aqueous solution of functionalized shell cross-linked (SCK) nanoparticles.

### REFERENCES FOR EXAMPLE 10

(1) Block Copolymers in Nanoscience; Lazzari, M., Liu, G., Lecommandoux, S., Ed.; Wiley-VCH: Weinheim, 2006.
(2) Mecke, A.; Dittrich, C.; Meier, W. Soft Matter 2006, 2, 751-759.
(3) Nishiyama, N.; Kataoka, K. Pharmacol. Ther. 2006, 112, 630-648.
(4) Black, C. T. ACS Nano 2007, 1, 147-150.
(5) Olson, D. A.; Chen, L.; Hillmyer, M. A. Chem. Mater. 2008, 20, 869-890.
(6) Bang, J.; Jeong, U.; Ryu, D. Y.; Russell, T. P.; Hawker, C. J. Adv. Mater. 2009, 21, 4769-4792.
(7) van Dongen, S. F. M.; de Hoog, H.-P. M.; Peters, R. J. R. W.; Nallani, M.; Nolte, R. J. M.; van Hest, J. C. M. Chem. Rev. 2009, 109, 6212-6274.
(8) Segalman, R. A.; McCulloch, B.; Kirmayer, S.; Urban, J. J. Macromolecules 2009, 42, 9205-9216.
(9) Mikhail, A. S.; Allen, C. J. Controlled Release 2009, 138, 214-223.
(10) Kim, H.-C.; Park, S.-M.; Hinsberg, W. D. Chem. Rev. 2010, 110, 146-177.
(11) Tyrrell, Z. L.; Shen, Y.; Radosz, M. Prog. Polym. Sci. 2010, 35, 1128-1143.
(12) Euliss, L. E.; DuPont, J. A.; Gratton, S.; DeSimone, J. Chem. Soc. Rev. 2006, 35, 1095-1104.
(13) Champion, J. A.; Katare, Y. K.; Mitragotri, S. J. Controlled Release 2007, 121, 3-9.
(14) Blanazs, A.; Armes, S. P.; Ryan, A. J. Macromol. Rapid Commun. 2009, 30, 267-277.
(15) Yoo, J. W.; Chambers, E.; Mitragotri, S. Curr. Pharm. Des. 2010, 16, 2298-2307.
(16) Geng, Y.; Dalhaimer, P.; Cai, S.; Tsai, R.; Tewari, M.; Minko, T.; Discher, D. E. Nat. Nanotech. 2007, 2, 249-255.
(17) Zhang, K.; Rossin, R.; Hagooly, A.; Chen, Z. Y.; Welch, M. J.; Wooley, K. L. J. Polym. Sci., Part A: Polym. Chem. 2008, 46, 7578-7583.
(18) Sharma, G.; Valenta, D. T.; Altman, Y.; Harvey, S.; Xie, H.; Mitragotri, S.; Smith, J. W. J. Controlled Release 2010, 147, 408-412.
(19) Bielawski, C. W.; Grubbs, R. H. Prog. Polym. Sci. 2007, 32, 1-29.
(20) Tsarevsky, N. V.; Matyjaszewski, K. Chem. Rev. 2007, 107, 2270-2299.
(21) Sciannamea, V.; Jerome, R.; Detrembleur, C. Chem. Rev. 2008, 108, 1104-1126.
(22) York, A. W.; Kirkland, S. E.; McCormick, C. L. Adv. Drug Delivery Rev. 2008, 60, 1018-1036.
(23) Boyer, C.; Bulmus, V.; Davis, T. P.; Ladmiral, V.; Liu, J.; Perrier, S. Chem. Rev. 2009, 109, 5402-5436.
(24) Kiesewetter, M. K.; Shin, E. J.; Hedrick, J. L.; Waymouth, R. M. Macromolecules 2010, 43, 2093-2107.
(25) Lodge, T. P.; Bang, J.; Li, Z.; Hillmyer, M. A.; Talmon, Y. Faraday Discuss. 2004, 128, 1-12.
(26) Soo, P. L.; Eisenberg, A. J. Polym. Sci., Part B: Polym. Phys. 2004, 42, 923-938.
(27) Harada, A.; Kataoka, K. Prog. Polym. Sci. 2006, 31, 949-982.
(28) Darling, S. B. Prog. Polym. Sci. 2007, 32, 1152-1204.
(29) Ryu, J.-H.; Hong, D.-J.; Lee, M. Chem. Commun. 2008, 1043-1054.
(30) Toomey, R.; Tirrell, M. Annu. Rev. Phys. Chem. 2008, 59, 493-517.
(31) Wurm, F.; Kilbinger, A. F. M. Angew. Chem., Int. Ed. 2009, 48, 8412-8421.
(32) Kale, T. S.; Klaikherd, A.; Popere, B.; Thayumanavan, S. Langmuir 2009, 25, 9660-9670.
(33) Zhou, Y.; Yan, D. Chem. Commun. 2009, 1172-1188.
(34) Hayward, R. C.; Pochan, D. J. Macromolecules 2010, 43, 3577-3584.
(35) Liu, X.; Kim, J.-S.; Wu, J.; Eisenberg, A. Macromolecules 2005, 38, 6749-6751.
(36) Cui, H. G.; Chen, Z. Y.; Zhong, S.; Wooley, K. L.; Pochan, D. J. Science 2007, 317, 647-650.
(37) Dupont, J.; Liu, G. J.; Niihara, K.; Kimoto, R.; Jinnai, H. Angew. Chem., Int. Ed. 2009, 48, 6144-6147.
(38) Chen, Z.; Cui, H.; Hales, K.; Li, Z.; Qi, K.; Pochan, D. J.; Wooley, K. L. J. Am. Chem. Soc. 2005, 127, 8592-8593.
(39) Erhardt, R.; Zhang, M.; Boker, A.; Zettl, H.; Abetz, C.; Frederik, P.; Krausch, G.; Abetz, V.; Müller, A. H. E. J. Am. Chem. Soc. 2003, 125, 3260-3267.
(40) Li, Z.; Kesselman, E.; Talmon, Y.; Hillmyer, M. A.; Lodge, T. P. Science 2004, 306, 98-101
(41) Kubowicz, S.; Baussard, J.-F.; Lutz, J.-F.; Thuenemann, A. F.; von Berlepsch, H.; Laschewsky, A. Angew. Chem., Int. Ed. 2005, 44, 5262-5265.
(42) Talingting, M. R.; Munk, P.; Webber, S. E.; Tuzar, Z. Macromolecules 1999, 32, 1593-1601.
(43) Shen, H.; Zhang, L.; Eisenberg, A. J. Am. Chem. Soc. 1999, 121, 2728-2740.
(44) Lutz, J.-F.; Laschewsky, A. Macromol. Chem. Phys. 2005, 206, 813-817.
(45) Li, Z.; Hillmyer, M. A.; Lodge, T. P. Macromolecules 2006, 39, 765-771.
(46) Li, Z.; Hillmyer, M. A.; Lodge, T. P. Langmuir 2006, 22, 9409-9417.
(47) Mao, J.; Ni, P.; Mai, Y.; Yan, D. Langmuir 2007, 23, 5127-5134.
(48) Stavrouli, N.; Triftaridou, A. I.; Patrickios, C. S.; Tsitsilianis, C. Macromol. Rapid Commun. 2007, 28, 560-566.
(49) Liu, C.; Hillmyer, M. A.; Lodge, T. P. Langmuir 2008, 24, 12001-12009.
(50) Fang, B.; Walther, A.; Wolf, A.; Xu, Y.; Yuan, J.; Müller, A. H. E. Angew. Chem., Int. Ed. 2009, 48, 2877-2880.
(51) Gohy, J.-F.; Ott, C.; Hoeppener, S.; Schubert, U. S. Chem. Commun. 2009, 6038-6040.
(52) Schacher, F.; Walther, A.; Ruppel, M.; Drechsler, M.; Müller, A. H. E. Macromolecules 2009, 42, 3540-3548.
(53) von Berlepsch, H.; Böttcher, C.; Skrabania, K.; Laschewsky, A. Chem. Commun. 2009, 2290-2292.
(54) Saito, N.; Liu, C.; Lodge, T. P.; Hillmyer, M. A. Macromolecules 2008, 41, 8815-8822.
(55) Skrabania, K.; Laschewsky, A.; von Berlepsch, H.; Böttcher, C. Langmuir 2009, 25, 7594-7601.
(56) Walther, A.; Müller, A. H. E. Chem. Commun. 2009, 1127-1129.
(57) Skrabania, K.; von Berlepsch, H.; Böttcher, C.; Laschewsky, A. Macromolecules 2010, 43, 271-281.
(58) Liu, C.; Hillmyer, M. A.; Lodge, T. P. Langmuir 2009, 25, 13718-13725.
(59) Schacher, F.; Walther, A.; Müller, A. H. E. Langmuir 2009, 25, 10962-10969.
(60) Saito, N.; Liu, C.; Lodge, T. P.; Hillmyer, M. A. ACS Nano 2010, 4, 1907-1912.
(61) Walther, A.; Barner-Kowollik, C.; Müller, A. H. E. Langmuir 2010, 26, 12237-12246.
(62) Lodge, T. P.; Rasdal, A.; Li, Z.; Hillmyer, M. A. J. Am. Chem. Soc. 2005, 127, 17608-17609.
(63) Uchman, M.; Stepanek, M.; Prochazka, K.; Mountrichas, G.; Pispas, S.; Voets, I. K.; Walther, A. Macromolecules 2009, 42, 5605-5613.
(64) Hawker, C. J.; Wooley, K. L. Science 2005, 309, 1200-1205.
(65) Iha, R. K.; Wooley, K. L.; Nyström, A. M.; Burke, D. J.; Kade, M. J.; Hawker, C. J. Chem. Rev. 2009, 109, 5620-5686.
(66) Krishnamurthy, V. M.; Estroff, L. A.; Whitesides, G. M. In Fragment-based Approaches in Drug Discovery; Jahnke, W. and Erlanson, D. A., Ed.; Wiley-VCH: Weinheim, 2006, p 11-53.
(67) Zheng, R.; Liu, G. Macromolecules 2007, 40, 5116-5121.
(68) Hales, K.; Chen, Z. Y.; Wooley, K. L.; Pochan, D. J. Nano Lett. 2008, 8, 2023-2026.
(69) Hu, J.; Liu, G.; Nijkang, G. J. Am. Chem. Soc. 2008, 130, 3236-3237.
(70) Sundararaman, A.; Stephan, T.; Grubbs, R. B. J. Am. Chem. Soc. 2008, 130, 12264-12265.
(71) Bütün, V.; Wang, X. S.; de Paz Báñez, M. V.; Robinson, K. L.; Billingham, N. C.; Armes, S. P.; Tuzar, Z. Macromolecules 1999, 33, 1-3.
(72) Ma, Q.; Remsen, E. E.; Clark, C. G.; Kowalewski, T.; Wooley, K. L. Proc. Natl. Acad. Sci. U.S.A. 2002, 99, 5058-5063.
(73) Stewart, S.; Liu, G. Chem. Mater. 1999, 11, 1048-1054.
(74) Sun, G.; Lee, N. S.; Neumann, W. L.; Freskos, J. N.; Shieh, J. J.; Dorshow, R. B.; Wooley, K. L. Soft Matter 2009, 5, 3422-3429.
(75) Lee, N. S.; Sun, G.; Neumann, W. L.; Freskos, J. N.; Shieh, J. J.; Dorshow, R. B.; Wooley, K. L. Adv. Mater. 2009, 21, 1344-1348.
(76) Abbas, S.; Lodge, T. P. Langmuir 2008, 24, 6247-6353.
(77) Shirai, K.; Yanagisawa, A.; Takahashi, H.; Fukunishi, K.; Matsuoka, M. Dyes Pigm. 1998, 39, 49-68.
(78) LaFemina, J. P.; Schenter, G. K. J. Chem. Phys. 1991, 94, 7558-7559.
(79) Gómez, I.; Reguero, M.; Boggio-Pasqua, M.; Robb, M. A. J. Am. Chem. Soc. 2005, 127, 7119-7129.
(80) Cogan, S.; Zilberg, S.; Haas, Y. J. Am. Chem. Soc. 2006, 128, 3335-3345.
(81) Sun, G.; Lee, N. S.; Neumann, W. L.; Freskos, J. N.; Shieh, J. J.; Dorshow, R. B.; Wooley, K. L. Soft Matter 2009, 5, 3422.
(82) Lee, N. S.; Sun, G.; Neumann, W. L.; Freskos, J. N.; Shieh, J. J.; Dorshow, R. B.; Wooley, K. L. Adv. Mater. 2009, 21, 1344.

1-83. (canceled)

84. A method of measuring the pH of a fluid in vivo, the method comprising:
administering to the in vivo fluid an effective amount of an optical agent, the optical agent comprising:
cross-linked block copolymers having a cross-linking density, wherein each of the block copolymers comprises one or more hydrophilic blocks and one or more hydrophobic blocks; and
linking groups covalently cross linking at least a portion of the hydrophilic blocks of the block copolymers, wherein at least a portion of the linking groups comprise one or more pH-insensitive photoactive moieties;
wherein the optical agent forms a supramolecular structure in aqueous solution, the supramolecular structure having one or more interior hydrophobic cores and one or more covalently cross-linked hydrophilic shells, wherein the one or more interior hydrophobic cores comprise the hydrophobic blocks of the block copolymers, and the one or more covalently cross-linked hydrophilic shells comprise the hydrophilic blocks of the block copolymers;
exposing the optical agent to electromagnetic radiation, wherein the optical agent emits fluorescence in response to the exposure to the electromagnetic radiation;
measuring a first fluorescence intensity at a first wavelength from the optical agent exposed to electromagnetic radiation;
measuring a second fluorescence intensity at a second wavelength from the optical agent exposed to electromagnetic radiation, wherein the second wavelength differs from the first wavelength;
calculating a fluorescence intensity ratio of the first fluorescence intensity at the first wavelength to the second fluorescence intensity at the second wavelength; and
comparing the calculated fluorescence intensity ratio to a reference fluorescence intensity ratio to provide the pH of the fluid, wherein the reference fluorescence intensity ratio is generated by measuring fluorescence intensities for one or more reference fluid samples having known pH.

85. The method of claim 84, wherein the optical agent supramolecular structure comprises a nanoparticle or shell cross-linked micelle.

86. The method of claim 84, wherein the first fluorescence intensity and the second fluorescence intensity are measured by measuring a local maximum of the fluorescence intensity.

87. The method of claim 84, wherein the first fluorescence intensity and the second fluorescence intensity are measured by measuring integrated intensities of the fluorescence at a preselected range of wavelengths about the first wavelength and the second wavelength.

88. The method of claim 84, wherein the optical agent is exposed to electromagnetic radiation of a wavelength selected from the range of 350 nanometers to 1300 nanometers.

89. The method of claim 84, further comprising administering the optical agent to a bodily fluid of an animal subject.

90. The method of claim 84, wherein the one or more photoactive moieties comprise a group corresponding to a pyrazine, a thiazole, a phenylxanthene, a phenothiazine, a phenoselenazine, a cyanine, an indocyanine, a squaraine, a dipyrrolo pyrimidone, an anthraquinone, a tetracene, a quinoline, an acridine, an acridone, a phenanthridine, an azo dye, a rhodamine, a phenoxazine, an azulene, an aza-azulene, a triphenyl methane dye, an indole, a benzoindole, an indocarbocyanine, a Nile Red dye, or a benzoindocarbocyanine.

91. The method of claim 84, wherein the one or more photoactive moieties do not comprise a group corresponding to a pyrazine.

92. The method of claim 84, wherein the hydrophilic blocks of the cross-linked block copolymers comprise poly(ethylene oxide) or poly(acrylic acid).

93. The method of claim 84, wherein the hydrophobic blocks of the cross-linked block copolymers comprise polystyrene or poly(p-hydroxystyrene).

94. The method of claim 84, wherein the block copolymers are of formula (FX23): 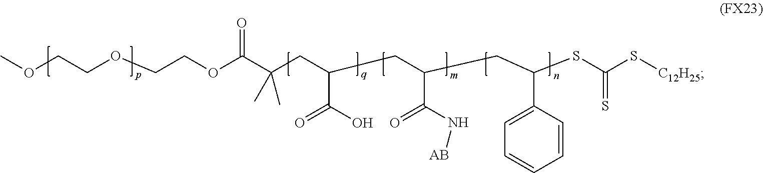
wherein:
each AB is independently LG or Bm;
each LG is the linking group;
each Bm is independently an amino acid, a peptide, a protein, a nucleoside, a nucleotide, an enzyme, a carbohydrate, a glycomimetic, an oligomer, a lipid, a polymer, an antibody, an antibody fragment, a mono- or polysaccharide comprising 1 to 50 carbohydrate units, a glycopeptide, a glycoprotein, a peptidomimetic, a drug, a steroid, a hormone, an aptamer, a receptor, a metal chelating agent, a polynucleotide comprising 2 to 50 nucleic acid units, a peptoid comprising 2 to 50 N-alkylaminoacetyl residues, a glycopeptide comprising 2 to 50 amino acid and carbohydrate units, or a polypeptide comprising 2 to 30 amino acid units;
each m is independently an integer selected from the range of 1 to 500;
each n is independently an integer selected from the range of 1 to 500;
each p is independently an integer selected from the range of 0 to 500; and
each q is independently an integer selected from the range of 0 to 500.

95. The method of claim 84, wherein the linking groups are of formula (FX24) or (FX25): 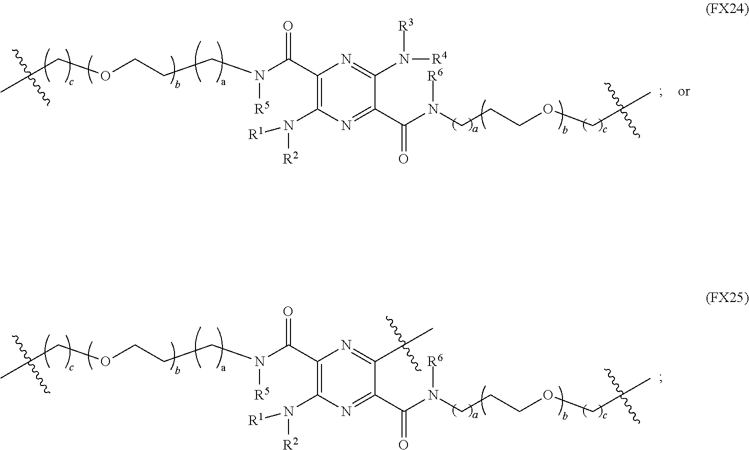
wherein:
each a is independently an integer selected from the range of 0 to 10;
each b is independently an integer selected from the range of 0 to 500;
each c is independently an integer selected from the range of 1 to 10;
each of R¹-R⁴ is independently a hydrogen, C₁-C₂₀ alkyl, C₃-C₂₀ cycloalkyl, C₅-C₂₀ alkylaryl, C₁-C₁₀ polyhydroxyalkyl, C₁-C₁₀ polyalkoxyalkyl, —CH₂(CH₂OCH₂)ₓCH₂OH, —CH₂(CHOH)_{y}R⁶⁰, or —(CH₂CH₂O)_{z}R⁶¹;
each of x, y and z is independently an integer selected from the range of 1 to 100; and
each of R⁵, R⁶, R⁶⁰ and R⁶¹ is independently hydrogen, C₁-C₁₀ alkyl, C₃-C₁₀ cycloalkyl, C₅-C₁₀ heteroaryl or C₅-C₁₀ aryl.

96. The method of claim 84, wherein the linking groups are of formula (FX26) or (FX27):

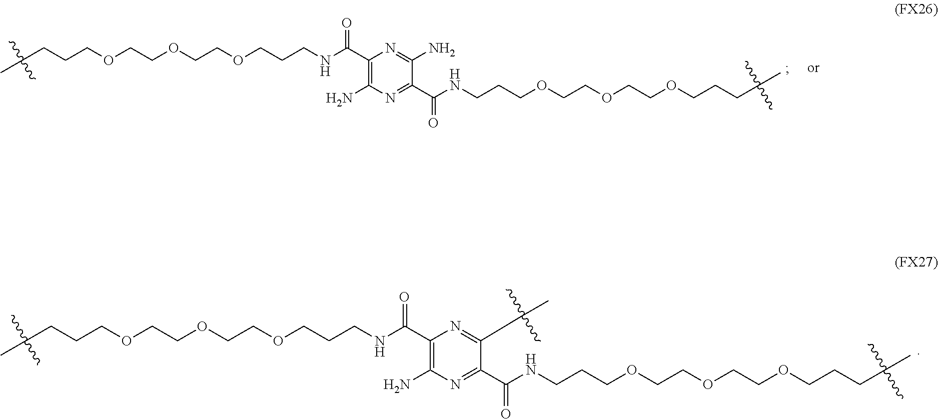 [SMILES: *CCCOCCOCCOCCCNC(=O)c1nc(N)c(C(=O)NCCCOCCOCCOCCC*)nc1N ; *CCCOCCOCCOCCCNC(=O)c1nc(N)c(C(=O)NCCCOCCOCCOCCC*)nc1*]

97. The method of claim 84, wherein the linking groups are of formula (FX28) or (FX29): 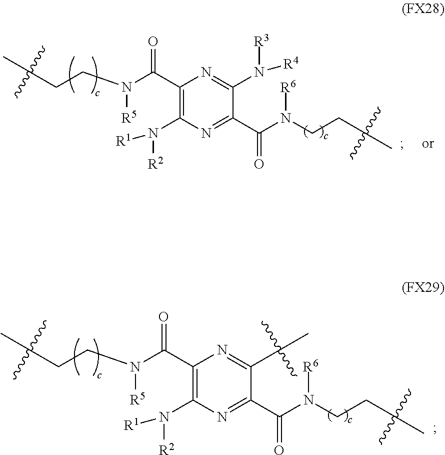
wherein:
each c is independently an integer selected from the range of 1 to 10;
each of R¹-R⁴ is independently a hydrogen, C₁-C₂₀ alkyl, C₃-C₂₀ cycloalkyl, C₅-C₂₀ alkylaryl, C₁-C₁₀ polyhydroxyalkyl, C₁-C₁₀ polyalkoxyalkyl, —CH₂(CH₂OCH₂)ₓCH₂OH, —CH₂(CHOH)_{y}R⁶⁰, or —(CH₂CH₂O)_{z}R⁶¹;
each of x, y and z is independently an integer selected from the range of 1 to 100; and
each of R⁵, R⁶, R⁶⁰ and R⁶¹ is independently hydrogen, C₁-C₁₀ alkyl, C₃-C₁₀ cycloalkyl, C₅-C₁₀ heteroaryl or C₅-C₁₀ aryl.

98. The method of claim 84, wherein the linking groups are of formula (FX30) or (FX31):

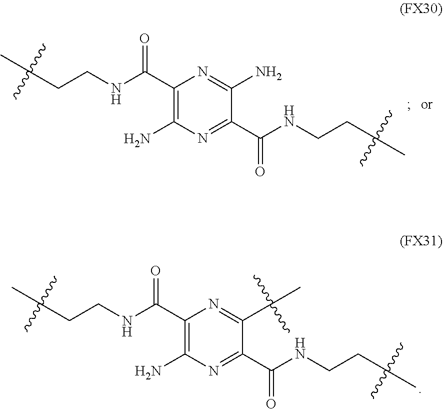 [SMILES: *CCNC(=O)c1nc(N)c(C(=O)NCC*)nc1N ; *CCNC(=O)c1nc(N)c(C(=O)NCC*)nc1*]

99. The method of claim 84, wherein the cross-linking density is less than 20%.

100. The method of claim 84, wherein the cross-linking density is selected from the range of 5% to 10%.

101. A system for measuring the pH of a fluid in vivo, comprising:
(a) an optical agent in liquid communication with a fluid in vivo,
wherein the optical agent comprises:
cross-linked block copolymers, wherein each of the block copolymers comprises one or more hydrophilic blocks and one or more hydrophobic blocks; and
linking groups covalently cross linking at least a portion of the hydrophilic blocks of the block copolymers, wherein at least a portion of the linking groups comprise one or more pH-insensitive photoactive moieties;
wherein the optical agent forms a supramolecular structure in aqueous solution, the supramolecular structure having one or more interior hydrophobic cores and one or more covalently cross-linked hydrophilic shells, wherein the one or more interior hydrophobic cores comprise the hydrophobic blocks of the block copolymers, and the one or more covalently cross-linked hydrophilic shells comprise the hydrophilic blocks of the block copolymers; and
(b) a device for measuring the pH of the fluid,
wherein the device comprises:
an optical source for providing electromagnetic radiation;
an electromagnetic radiation delivery system in optical communication with the optical source for providing at least a portion of the electromagnetic radiation to the optical agent administered to the fluid, thereby exciting fluorescence from the optical agent in the fluid;
an electromagnetic radiation collection system in optical communication with the fluid for collecting at least a portion of the fluorescence from the optical agent and providing at least a portion of the fluorescence to a detector;
a detector for receiving at least a portion of the fluorescence from the electromagnetic radiation collection system;
wherein the detector measures a first fluorescence intensity at a first wavelength and a second fluorescence intensity at a second wavelength;
wherein the second wavelength differs from the first wavelength; and
a processor in optical or electronic communication with the detector;
wherein the processor is programmed to:
calculate a fluorescence intensity ratio of the first fluorescence intensity at the first wavelength to the second fluorescence intensity at the second wavelength; and
compare the calculated fluorescence intensity ratio to a reference fluorescence intensity ratio to provide the pH of the fluid, wherein the reference fluorescence intensity ratio is generated by measuring fluorescence intensities for one or more reference fluid samples having known pH.
